(19) United States
(12) Patent Application Publication
(10) Pub. No.: US 2024/0075084 A1
(43) Pub. Date: Mar. 7, 2024

(54) KLEBSIELLA BACTERIOPHAGE AND USES THEREOF

(71) Applicant: Biomx Ltd., Nes Ziona (IL)

(72) Inventors: Eyal WEINSTOCK, Kfar Shmaryahu (IL); Iddo Nadav WEINER, Tel Aviv (IL); Maya KAHAN-HANUM, Hod Hasharon (IL); Efrat KHABRA, Petach Tikva (IL); Noa BEN-YISHAI, Rehovot (IL); Dana INBAR, Ramat Gan (IL); Rotem EDGAR, Kfar-Saba (IL)

(73) Assignee: Biomx Ltd., Nes Ziona (IL)

(21) Appl. No.: 18/273,295

(22) PCT Filed: Jan. 20, 2022
(86) PCT No.: PCT/IL2022/050090

§ 371 Date: Jul. 20, 2023

Related U.S. Application Data
(60) Provisional application No. 63/139,872, filed on Jan. 21, 2021.
(60) Provisional application No. 63/182,941, filed on May 2, 2021.

(51) Int. Cl. A61K 35/76; A61P 31/04; C12N 7/00

(52) U.S. Cl. CPC A61K 35/76; A61P 31/04; C12N 7/00; C12N 2795/10021; C12N 2795/10032

(57) ABSTRACT

A composition comprising at least two different strains of isolated bacteriophages, each capable of infecting a bacteria of the species Klebsiella pneumoniae, wherein at least one of said at least two different strains of isolated bacteriophages has a genomic nucleic acid sequence at least 90% identical to one of the nucleic acid sequence as set forth in SEQ ID NOs: 1-30. Uses thereof are also disclosed.

### RELATED APPLICATION/S

This application claims the benefit of priority of U.S. Provisional Patent Application No. 63/139,872 filed on 21 Jan. 2021 and 63/182,941 filed on 2 May. 2021, the contents of which are incorporated herein by reference in their entirety.

### SEQUENCE LISTING STATEMENT

The ASCII file, entitled 89723SequenceListing.txt, created on 19 Jan. 2022, comprising 5,563,457 bytes, submitted concurrently with the filing of this application is incorporated herein by reference.

### FIELD AND BACKGROUND OF THE INVENTION

The present invention, in some embodiments thereof, relates to bacteriophage strains capable of infecting bacteria of the genus Klebsiella and more particularly bacteria of the species Klebsiella pneumoniae.

Gut microbiota dysbiosis is associated with the pathogenesis of inflammatory bowel disease (IBD) (Said et al., 2014). Increased stimulation from the gut microbiota can result in inflammation and alter a patient's immune response. In humans, the oral microbiota comprises over 700 species or phylotypes of bacteria (Said et al., 2014). Ingested oral bacteria do not typically colonize the healthy intestine (Chen et al., 2016; Bik et al., 2010; U.S. Provisional Application No. 62/415,759, TH1 Cell Stimulatory Bacteria Colonizing in Human Oral Cavity). However, bacteria of oral origin may be increased in the gut microbiota of patients with IBD (Gevers et al., 2014; Atarashi et al., 2017). Oral microbiota dysbiosis is observed in patients with IBD and could contribute to disease etiology (Said et al., 2014).

IBDs, such as Crohn's disease (CD) and ulcerative colitis (UC), can cause diarrhea, malabsorption of nutrients, malnutrition, anemia, and weight loss. Severe intestinal inflammation can extend beyond the mucosa of the intestines and cause ulceration, bleeding, toxic megacolon, strictures, and fistulas. In addition, chronic inflammation is associated with colon cancer. Extraintestinal complications include, but are not limited to, arthritis, skin rashes, liver disease, and eye disorders such as episcleritis and uveitis.

According to the Crohn's & Colitis Foundation of America, “there is no single ideal therapy” for IBD. Dietary restriction alone has not been shown to ameliorate IBD. For many patients, surgical intervention is necessary. Even with the currently available IBD treatments, many patients experience IBD flares between periods of remission. Thus, there is a significant unmet need for effective, reliable, and long-term treatment and/or prevention of IBD.

Background art includes WO/2019/048930 and de Sousa et al., The ISME Journal (2020) 14:2980-2996.

### SUMMARY OF THE INVENTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

According to an aspect of the present invention there is provided a composition comprising at least two different strains of isolated bacteriophages, each capable of infecting a bacteria of the species Klebsiella pneumoniae, wherein at least one of said at least two different strains of isolated bacteriophages has a genomic nucleic acid sequence at least 90% identical to one of the nucleic acid sequence as set forth in SEQ ID NOs: 1-30 and/or comprises the essential genes as set forth in Example 6.

According to embodiments of the invention, the first of said at least two different strains of isolated bacteriophages has a genomic nucleic acid sequence at least 90% identical to the nucleic acid sequence as set forth in SEQ ID NO: 1 and/or comprises the essential genes of phage KP10-11 as set forth in Example 6, and a second of said at least two different strains of isolated bacteriophages has a genomic nucleic acid sequence at least 90% identical to the nucleic acid sequence as set forth in SEQ ID NO: 2 and/or comprises the essential genes of phage KP10-48 as set forth in Example 6.

According to embodiments of the invention, the composition comprises at least three different strains of isolated bacteriophages, wherein a third of said at least three different strains of isolated bacteriophages has a genomic nucleic acid sequence at least 90% identical to the nucleic acid sequence as set forth in SEQ ID NO: 3 and/or comprises the essential genes of phage 395D2-3-2 as set forth in Example 6.

According to embodiments of the invention, the composition comprises:
(i) a bacteriophage having a genomic nucleic acid sequence at least 90% identical to SEQ ID NO: 1 and/or comprising the essential genes of phage KP10-11 as set forth in Example 6;
(ii) a bacteriophage having a genomic nucleic acid sequence at least 90% identical to SEQ ID NO: 2 and/or comprising the essential genes of phage KP10-48 as set forth in Example 6;
(iii) a bacteriophage having a genomic nucleic acid sequence at least 90% identical to SEQ ID NO: 3 and/or comprising the essential genes of phage 395D2-3-2 as set forth in Example 6;
(iv) a bacteriophage having a genomic nucleic acid sequence at least 90% identical to SEQ ID NO: 4 and/or comprising the essential genes of phage 395D2-3-1 as set forth in Example 6;
(v) a bacteriophage having a genomic nucleic acid sequence at least 90% identical to SEQ ID NO: 22 and/or comprising the essential genes of phage KP10-85 as set forth in Example 6; and
(vi) a bacteriophage having a genomic nucleic acid sequence at least 90% identical to SEQ ID NO: 25 and/or comprising the essential genes of phage KP10-91 as set forth in Example 6.

According to embodiments of the invention, the at least two different strains of isolated bacteriophages in combination target at least 100 different strains of Klebsiella pneumoniae.

According to embodiments of the invention, the at least 100 different strains of Klebsiella pneumonia are targeted by each of said at least two different strains.

According to embodiments of the invention, the composition comprises at least three different strains of isolated bacteriophages, each capable of infecting a bacteria of the species Klebsiella pneumoniae, wherein each of said at least three different strains of isolated bacteriophages (i) has a genomic nucleic acid sequence at least 90% identical to one of the nucleic acid sequence as set forth in SEQ ID NOs: 1-30 and/or (ii) comprises the essential genes of a bacteriophage as set forth in Example 6, wherein said at least three different strains of isolated bacteriophages in combination target at least 200 different strains of Klebsiella pneumoniae.

According to embodiments of the invention, the composition comprises at least three different strains of isolated bacteriophages, each capable of infecting a bacteria of the species Klebsiella pneumoniae, wherein each of said at least three different strains of isolated bacteriophages (i) has a genomic nucleic acid sequence at least 90% identical to one of the nucleic acid sequence as set forth in SEQ ID NOs: 1-30 and/or (ii) comprises the essential genes of a bacteriophage as set forth in Example 6, wherein at least 100 different strains of Klebsiella pneumonia are targeted by each of said at least three different strains.

According to embodiments of the invention, the at least one bacteriophage is genetically modified such that the genome thereof comprises a heterologous sequence.

According to embodiments of the invention, the heterologous sequence encodes a therapeutic agent or a diagnostic agent.

According to embodiments of the invention, the composition comprises no more than 10 different bacteriophage strains.

According to an aspect of the present invention there is provided a recombinant bacteriophage capable of infecting bacteria of the species Klebsiella pneumoniae, wherein said bacteriophage (i) has a genomic nucleic acid sequence at least 90% identical to one of the nucleic acid sequences as set forth in SEQ ID NOs: 1-30 and/or (ii) comprises the essential genes of a bacteriophage as set forth in Example 6, and wherein said bacteriophage is genetically modified such that the genome thereof comprises a heterologous sequence.

According to embodiments of the invention, the heterologous sequence encodes a therapeutic agent or a diagnostic agent.

According to embodiments of the invention, the therapeutic agent comprises an immune modulating agent.

According to an aspect of the present invention there is provided a pharmaceutical composition comprising the recombinant bacteriophage described herein as the active agent, and a pharmaceutical carrier.

According to embodiments of the invention, the pharmaceutical composition is formulated for oral delivery or rectal delivery.

According to embodiments of the invention, the composition is formulated for oral delivery or rectal delivery.

According to an aspect of the present invention there is provided a isolated bacteriophage capable of infecting bacteria of the species Klebsiella pneumoniae, wherein said bacteriophage has a genomic nucleic acid sequence at least 95% identical to one of the nucleic acid sequences as set forth in SEQ ID NOs: 1-30.

According to an aspect of the present invention there is provided a isolated bacteriophage capable of infecting bacteria of the species Klebsiella pneumoniae, wherein said bacteriophage comprises the essential genes of a bacteriophage listed in Table 2, wherein said essential genes are set forth in Example 6.

According to an aspect of the present invention there is provided a method of treating a disease associated with a Klebsiella pneumoniae infection in a subject in need thereof, comprising administering to the subject a therapeutically effective amount of a composition comprising at least one isolated bacteriophage strain capable of infecting bacteria of the species Klebsiella pneumonia causing the infection, wherein said at least one bacteriophage strain (i) has a genomic nucleic acid sequence at least 95% identical to one of the nucleic acid sequences set forth in SEQ ID NOs: 1-30 and/or (ii) comprises the essential genes of a bacteriophage as set forth in Example 6, thereby treating the disease associated with a Klebsiella pneumoniae infection.

According to an aspect of the present invention there is provided a method of treating a disease associated with a Klebsiella pneumoniae infection in a subject in need thereof, comprising administering to the subject a therapeutically effective amount of the composition described herein, thereby treating the disease associated with a Klebsiella pneumoniae infection.

According to embodiments of the invention, the disease is an inflammatory bowel disease (IBD).

According to embodiments of the invention, the inflammatory bowel disease is ulcerative colitis (UC).

According to embodiments of the invention, the inflammatory bowel disease is Crohn's disease.

According to embodiments of the invention, the administering comprises orally administering or rectally administering.

According to embodiments of the invention, the composition comprises no more than 10 different bacteriophage strains.

According to embodiments of the invention, the method further comprises identifying the strain of Klebsiella pneumoniae colonizing the subject prior to the administering.

According to embodiments of the invention, the at least one bacteriophage strain is genetically modified such that the genome thereof comprises a heterologous sequence.

According to embodiments of the invention, the heterologous sequence encodes a therapeutic agent or a diagnostic agent.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIG. 1 is a table summarizing the % sequence homology (based on local BLAST) of the isolated phages.

FIG. 2 is a table summarizing the host range of the phage as profiled according to the capsule type of the infected bacteria.

FIG. 3 is a table summarizing the host range of the phage as profiled according to Multi-locus sequence typing (MLST) of the infected bacteria.

FIG. 4 is a Kaplan Meier survival curve of mice following treatment in K. pneumonia peritonitis infection model. The asterisk (*) indicates significant increase (p<0.05) in the survival rates of the treated animals compared to the vehicle control group at day 7 time point as determined by Fisher's exact test.

FIG. 5 is a box plot summarizing the phage redundancy effect on the time to mutant (TTM) period of the target bacteria. The average TTM is significantly higher between combination populations with different redundancy level.

### DESCRIPTION OF SPECIFIC EMBODIMENTS OF THE INVENTION

The present invention, in some embodiments thereof, relates to bacteriophage strains capable of infecting bacteria of the genus Klebsiella and more particularly bacteria of the species Klebsiella pneumoniae.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have isolated novel bacteriophage strains characterized by having a high specificity to one or more Klebsiella pneumoniae strains. The disclosed bacteriophage are lytic, and as such do not have any capacity to integrate into the DNA of their bacterial host. Such bacteriophages bring about immediate target bacterial eradication through lysis after hijacking the host protein expression machinery to manufacture needed phage protein components.

The present inventors sought to combine particular phage strains and provide them as a cocktail which is capable of lysing a myriad of Klebsiella pneumoniae strains in a single dose. The cocktails can serve as an off-the-shelf therapeutic for the treatment of inflammatory bowel disease (IBD), which is known to be associated with Klebsiella pneumonia infections. Furthermore, it is envisaged that the cocktails will have high therapeutic efficacy for treating IBD at the individual level, since each individual can be infected by a wide range of Klebsiella pneumonia strains.

The combinations disclosed herein are typically synergistic with respect to their inhibitory effect on the target bacteria. This may be quantitated by measuring the time taken to mutation (TTM) i.e. the time taken for a bacteria to mutate and overcome the inhibitory effect of a phage. When two phages X and Y are known to infect a target bacteria strain H, the TTM of each phage separately as well as the TTM of their combination is measured under same growth conditions. The synergistic redundancy effect appears when the TTM of combination [X,Y] is longer than that of both X and Y. Without being bound to theory, the synergy may be derived from different mechanism of infection used by the two phages X and Y.

Thus, according to a first aspect of the present invention, there is provided an isolated bacteriophage capable of infecting bacteria of the species Klebsiella pneumoniae, wherein the bacteriophage has a genomic nucleic acid sequence at least 95% identical to one of the nucleic acid sequences as set forth in SEQ ID NOs: 1-30.

As used herein, the term “bacteriophage” and “phage” are used interchangeably and refer to an isolated virus that is capable of infecting a bacterium. Typically, a phage will be characterized by: 1) the nature of the nucleic acids that make up its genome, e.g., DNA, RNA, single-stranded or double-stranded; 2) the nature of its infectivity, e.g., lytic or temperate; and 3) the particular Klebsiella pneumonia subspecies that it infects (and in certain instances the particular strain of that FN subspecies). This aspect is known as “host range”.

As used herein, the phrases “isolated bacteriophage”, “isolate” or grammatical equivalents refer to a bacteriophage which is removed from its natural environment (e.g. removed from bacteria which it typically infects). In one embodiment, the isolated bacteriophage is removed from cellular material and/or other elements that naturally exist in the source clinical or environmental sample. The term isolated bacteriophages includes such phages isolated from human or animal patients (“clinical isolates” or “clinical variants”) and such phages isolated from the environment (“environmental isolates”).

In one embodiment, the bacteriophages are lytic.

The term “lytic bacteriophage” refers to a bacteriophage that infects a bacterial host and causes that host to lyse without incorporating the phage nucleic acids into the host genome. A lytic bacteriophage is typically not capable of reproducing using the lysogenic cycle.

As used herein, the phrase “phage strain” refers to the deposited or sequenced phage, as described herein.

The bacteriophage have been deposited at the Polish Collection of microorganisms PCM), Institute of Immunology and Experimental Therapy, Polish Academy of Sciences, Ul. Weigla 12, 53-114 Wroclaw, Poland with the deposit numbers provided in Table 2, herein below.

The term “Klebsiella pneumoniae” relates to a species of bacteria of the Klebsiella genus. Klebsiella are gram-negative, nonmotile, and rod-shaped. It will be appreciated that the term “Klebsiella pneumonia” includes bacteria that are currently classified or will be reclassified as Klebsiella pneumoniae bacteria.

Exemplary strains of Klebsiella pneumonia that are infected by the phage strains of the present invention are those that are found in human specimens (e.g. stool specimens, saliva specimens, bronchial specimens, human sewage). Additional Klebsiella pneumonia bacteria that may be infected by the disclosed phages are detailed in WO/2019/048930, the contents of which are incorporated herein by reference.

In some embodiments, the bacteriophages provided herein are capable of lysing deleterious Klebsiella pneumonia bacteria that induce immune and/or inflammatory response(s). In some embodiments, the bacteriophages provided herein are capable of lysing deleterious Klebsiella pneumonia bacteria that disrupt the balance of immune homeostasis in the intestine, e.g., by enhancing the pro-inflammatory activity, ultimately resulting in chronic intestinal inflammation. In some embodiments, the bacteriophages provided herein are capable of lysing deleterious Klebsiella pneumonia bacteria that induce a T helper 1 (TH1) cell response. In some embodiments, the Klebsiella pneumonia bacteria to be lysed by the bacteriophage provided herein are in the lumen. In some embodiments, the Klebsiella pneumonia bacteria to be lysed by the bacteriophages provided herein are associated with the mucosa or epithelium. In some embodiments, the Klebsiella pneumonia bacteria to be lysed by the bacteriophages provided herein are in between the mucosal and submucosal layer in the ileum, e.g., in Crohn's disease.

In a particular embodiment, the phages described herein are capable of infecting at least one, two, three, four, five, six, seven, eight, nine or more Klebsiella pneumonia strains that infect a subject with IBD.

In a particular embodiment, the phages described herein are capable of infecting at least one, two, three, four, five, six, seven, eight, nine or more Klebsiella pneumonia strains present in the gastrointestinal tract of the subject (e.g. stomach, colon, small intestine, and/or mouth).

In a particular embodiment, the phages described herein are capable of infecting at least one, two, three, four, five, six, seven, eight, nine or more Klebsiella pneumonia strains that are causative for IBD.

Klebsiella spp. code for an extracellular capsule that is highly variable within the species. This capsule is a high molecular weight polysaccharide made up of different repeat units of oligosaccharides. Combinations of different oligosaccharides are referred to as serotypes. In K. pneumoniae there are 77 serologically defined serotypes, and numbered from K1 to K77 and more than 130 serotypes were identified computationally. The latter are noted from KL1 to KL130, and are usually referred as capsule locus types (or CLT).

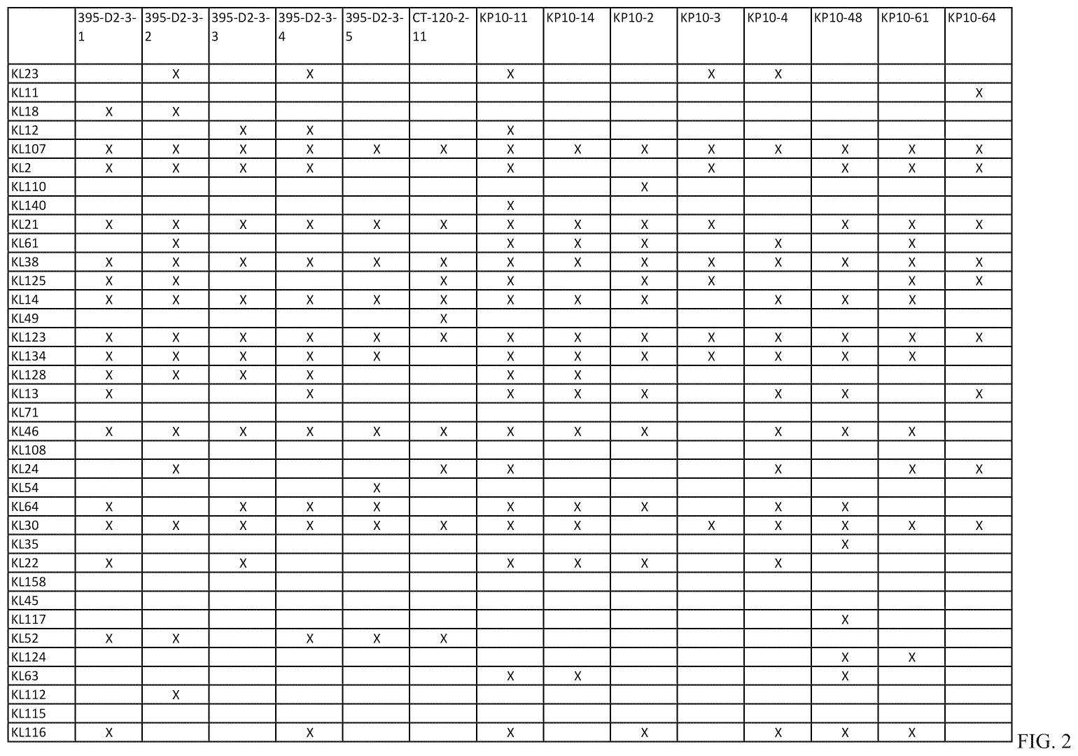

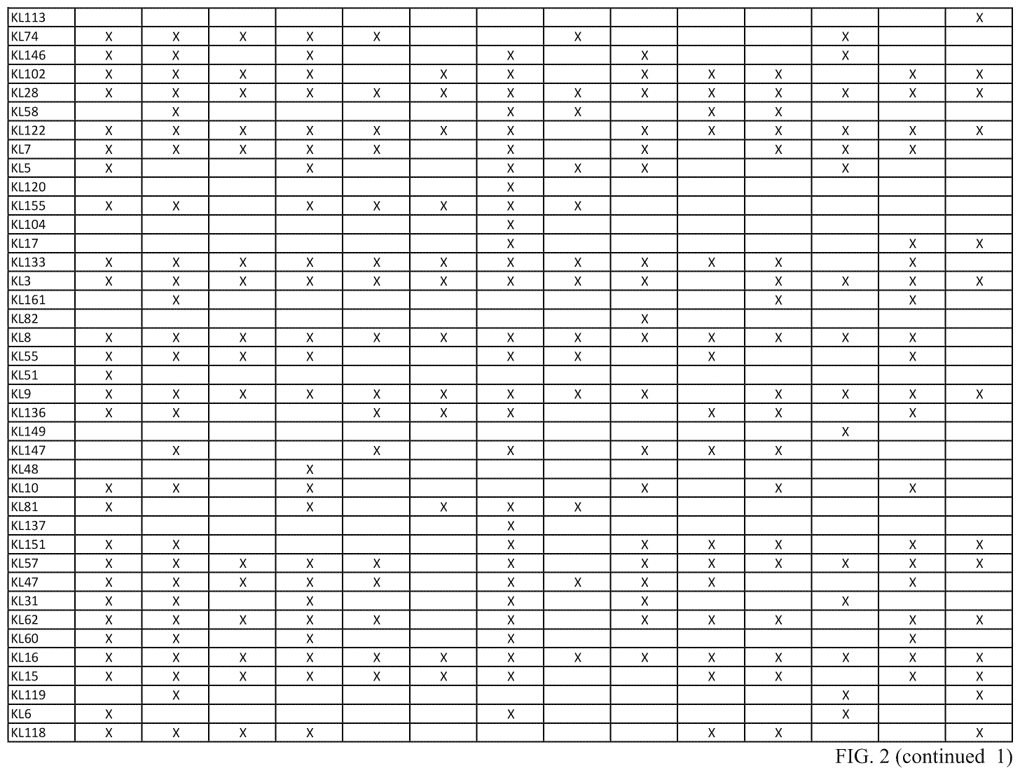

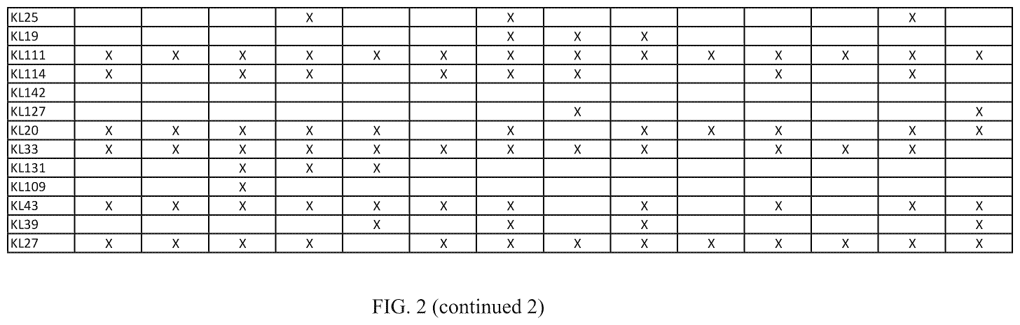

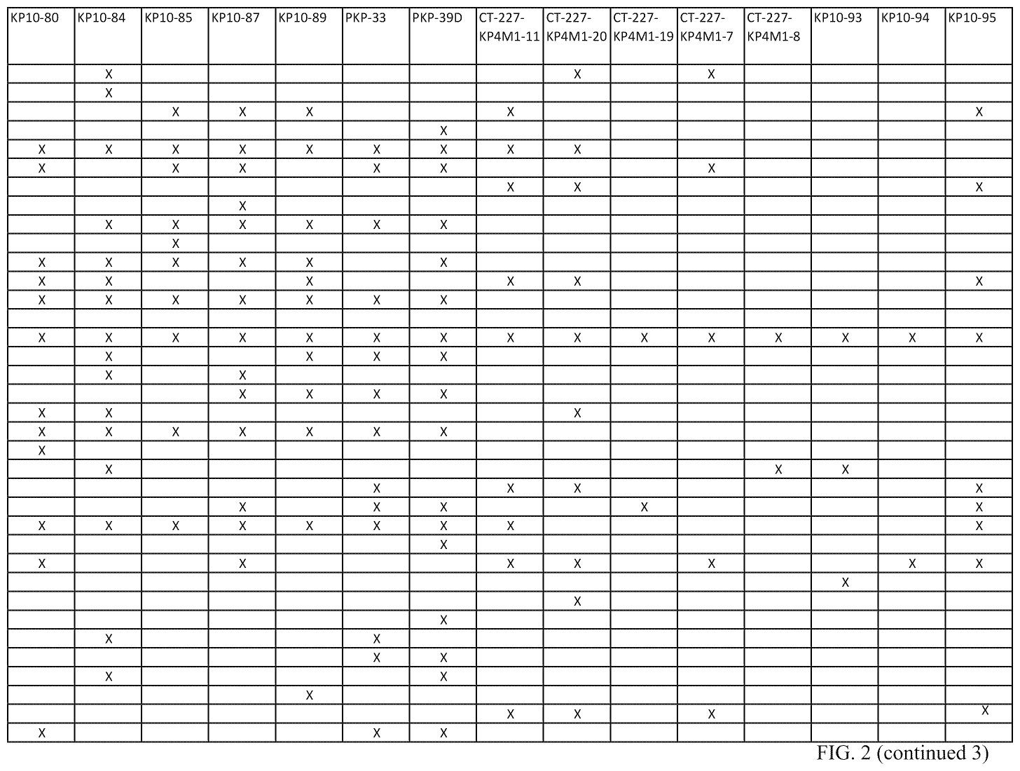

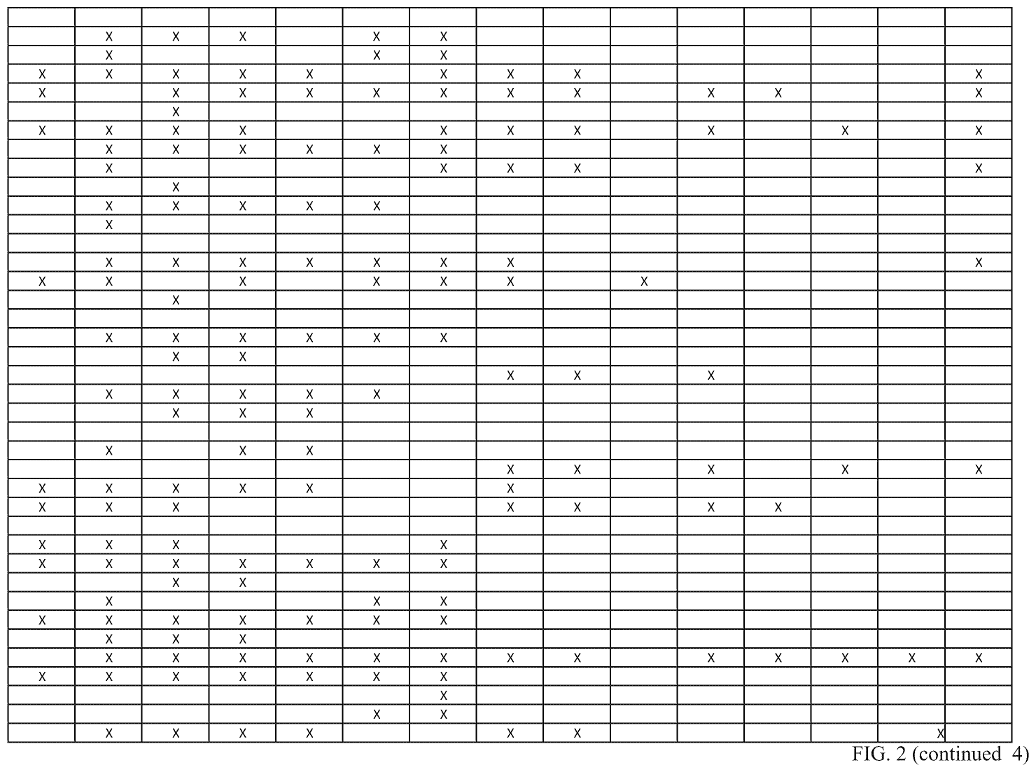

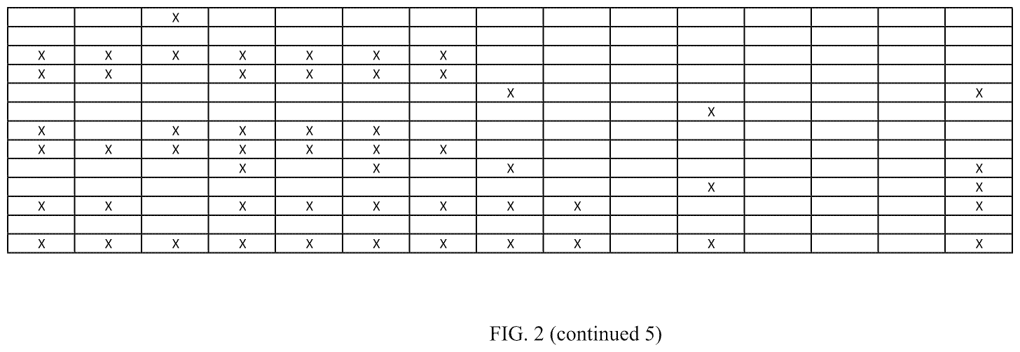

According to a particular embodiment, the phages described herein are capable of infecting Klebsiella pneumonia bacterial strains having a capsule locus type as set forth in FIG. 2.

Also contemplated are progeny of the phages that have a genomic nucleic acid as set forth in SEQ ID NOs: 1-29, wherein the progeny is capable of infecting the same subspecies (or even strain) of Klebsiella pneumoniae as that which the parent bacteriophage having one of the above set forth genomic nucleic acid sequence infects. Such progeny may have genomes having a sequence at least 85% identical, at least 90% identical, at least 91% identical, at least 92% identical, at least 93% identical, at least 94% identical, at least 95% identical, 96% identical, 97% identical 98% identical, or 99% identical to the genome of the parent bacteriophage.

As used herein, the term “or progeny of the bacteriophage” refers to bacteriophages stemming from or derived from the strains identified herein.

Also contemplated are functional homologs of those that have a genomic nucleic acid sequence as set forth in SEQ ID NOs: 1-30, wherein the functionally homologous bacteriophage is capable of infecting essentially the same subspecies (or even strain) of Klebsiella pneumoniae as that which the bacteriophage having one of the above set forth genomic nucleic acid sequence infects.

As used herein “functional homolog” or “functionally homologous” or “variant” or grammatical equivalents as used herein refer to a bacteriophage with a genomic nucleic acid sequence different than that of the sequenced bacteriophage (i.e., at least one mutation) resulting in a bacteriophage that is endowed with substantially the same ensemble of biological activities (+/−10%, 20%, 40%, 50%, 60% when tested under the same conditions) as that of the sequenced bacteriophage and can be classified as infecting essentially the same strain or subspecies of bacteria based on known methods of species/strain classifications.

A bacteriophage “infects” bacteria if it either causes the bacteria to lyse or integrates its nucleic acid sequence into the bacterial genome.

According to a particular embodiment, the bacteriophage disclosed herein lyse their target bacteria.

According to some embodiments, the genomic nucleic acid sequence of the bacteriophages described herein is at least about 85%, at least about 90%, at least about 91%, at least about 92%, at least about 93%, at least about 94%, at least about 95%, at least about 96% least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95% 99.95%, at least about 99.99%, or more identical to the genomic sequence of the genomic sequences as set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

In particular, the bacteriophage has a genomic nucleic acid sequence at least 95% identical (% homologous) to the nucleic acid sequence as set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28,29 or 30.

According to a specific embodiment, the bacteriophage has a genomic nucleic acid sequence at least 95% identical (% homologous) to the full length nucleic acid sequence as set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

According to a specific embodiment, the bacteriophage comprises the essential genes of a bacteriophage selected from the bacteriophages listed in Table 2, wherein the essential genes are genes as set forth for the selected bacteriophage in Example 6.

As used herein, “percent homology”, “percent identity”, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Percent identity can be determined using any homology comparison software, including for example, the BlastN software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

According to an additional or alternative embodiment, a functional homolog is determined as the average nucleotide identity (ANI), which detects the DNA conservation of the core genome (Konstantinidis K and Tiedje J M, 2005, Proc. Natl. Acad. Sci. USA 102: 2567-2592). In some embodiments, the ANI between the functional homolog and the deposited bacteriophage (or that having a genome as set forth in any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 is of at least about 95%, at least about, 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, a functional homolog is determined by the degree of relatedness between the functional homolog and the bacteriophage having a genome as set forth in any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 determined as the Tetranucleotide Signature Frequency Correlation Coefficient, which is based on oligonucleotide frequencies (Bohlin J. et al. 2008, BMC Genomics, 9:104). In some embodiments, the Tetranucleotide Signature Frequency Correlation coefficient between the variant and the bacteriophage having a genome as set forth in any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28 or 29 is of about 0.99, 0.999 or more.

According to an additional or alternative embodiment, the degree of relatedness between the functional homolog and the bacteriophage having a genome as set forth in any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, is determined as the degree of similarity obtained when analyzing the genomes of the parent and of the variant bacteriophage by Pulsed-field gel electrophoresis (PFGE) using one or more restriction endonucleases. The degree of similarity obtained by PFGE can be measured by the Dice similarity coefficient. In some embodiments, the Dice similarity coefficient between the variant and the bacteriophage having a genome as set forth in any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 is of at least about 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, the degree of relatedness between the functional homolog and the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30 is determined by the Pearson correlation coefficient obtained by comparing the genetic profiles of both phages obtained by repetitive extragenic palindromic element-based PCR (REP-PCR) (see e.g. Chou and Wang, Int J Food Microbiol. 2006, 110:135-48). In some embodiments, the Pearson correlation coefficient obtained by comparing the REP-PCR profiles of the variant and the above described (e.g. deposited phage) is of at least about 0.99, at least about 0.999 or more—see for example bmcmicrobioldotbiomedcentraldotcom/articles/10.1186/s12866-020-01770-2.

According to an additional or alternative embodiment, the degree of relatedness between the functional homolog and the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30 is defined by the linkage distance obtained by comparing the genetic profiles of both phages obtained by Multi-locus sequence typing (MLST) (see e.g. Maiden, M. C., 1998, Proc. Natl. Acad. Sci. USA 95:3140-3145). In some embodiments, the linkage distance obtained by MLST of the functional homolog and the phage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30, is of at least about 0.99, at least about 0.999 or more.

According to an additional or alternative embodiment, the functional homolog comprises a functionally conserved gene or a fragment thereof (i.e. an essential gene) e.g., an integrase gene, a polymerase gene, a capsid protein assembly gene, a DNA terminase, a tail fiber gene, or a repressor gene that is at least about 97%, at least about 98%, at least about 99%, at least about 99.1%, at least about 99.5%, at least about 99.6%, at least about 99.7%, at least about 99.8%, at least about 99.9%, or more identical to that of the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

For each of the disclosed bacteriophages, Example 6 provides the gene name of their essential genes.

According to an additional or alternative embodiment, the functional homolog is defined by a comparison of the coding sequence (gene) order.

According to an additional or alternative embodiment, the functional homolog is defined by a comparison of order of non-coding sequences.

According to an additional or alternative embodiment, the functional homolog is defined by a comparison of order of coding and non-coding sequences.

According to some embodiments of the invention, the combined coding region of the functional homolog is such that it maintains the original order of the coding regions as within the genomic sequence of the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30, yet without the non-coding regions.

For example, in case the genomic sequence has the following coding regions, A, B, C, D, E, F, G, each flanked by non-coding sequences (e.g., regulatory elements, and the like), the combined coding region will include a single nucleic acid sequence having the A+B+C+D+E+F+G coding regions combined together while maintaining the original order of their genome, yet without the non-coding sequences.

According to some embodiments of the invention, the combined non-coding region of the functional homolog is such that it maintains the original order of the non-coding regions as within the genomic sequence of the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30, yet without the coding regions as originally present in the original bacteriophage.

According to some embodiments of the invention, the combined non-coding region and coding region (i.e., the genome) of the functional homolog is such that it maintains the original order of the coding and non-coding regions as within the genomic sequence of the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

As used herein “maintains” relate to at least about 90%, 91%, 92%, 93%, 94%, 95%, 96%, 97%, 98%, 99% or 100% of the coding and/or non-coding regions of the functional homolog compared to the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

According to an additional or alternative embodiment, the functional homolog is defined by a comparison of gene content.

According to a specific embodiment, the functional homolog comprises a combined coding region at least about 90%, at least about 91%, at least about 92%, at least about 93%, at least about 94%, at least about 95%, at least about 96%, at least about 97%, at least about 98%, at least about 99%, or more (e.g., 100%) identical to the combined coding region existing in genome of the bacteriophage having a genome as set forth in any one of SEQ ID NO: 11, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

As used herein “combined coding region” refers to a nucleic acid sequence including all of the coding regions of the original bacteriophage yet without the non-coding regions of the original bacteriophage.

In one embodiment, the bacteriophages show up to 85%, 86%, 87%, 88%, 89%, 90%, 91%, 92%, 93%, 94%, 95%, 96%, 97%, 98%, 99% sequence identity with the bacteriophages disclosed herein and share at least one of the following characteristics—similar host range; similar type of infectivity (i.e. lytic or temperate).

In another embodiment, the bacteriophages show up to 85%, 86%, 87%, 88%, 89%, 90%, 91%, 92%, 93%, 94%, 95%, 96%, 97%, 98%, 99% sequence identity with the bacteriophages disclosed herein and share both of the following characteristics—similar host range; similar type of infectivity.

Additional bioinformatics methods that may be used to determine relatedness between two phage genomes include Nucmer and Minimap, both of which are alignment based tools; Win-zip, Jacard distance and MinHash, each of which are information based tools; and Codon usage similarity, pathway similarity and protein motif similarity.

As used herein, “host range” refers to the bacteria that are susceptible to infection by a particular phage. The host range of a phage may include, but is not limited to, a strain, a subspecies, a species, a genus, or multiple genera of bacteria.

Phage isolates may be prepared and phenotyped using methods known in the art, e.g., a plaque assay, liquid media assay, solid media assay. In some embodiments, the solid media assays to quantify and isolate phage are based on plaque assays (S. T. Abedon et al., Methods in Molecular Biology 2009 (Clifton, N.J.), 501, 161-74), ranging from efficiency of plating (EOP) (E. Kutter, Methods in Molecular Biology 2009 (Clifton, N.J.), 501, 141-9) to spot testing (P. Hyman et al., Advances in Applied Microbiology (1st ed., Vol. 70, pp. 217-48) 2010. Elsevier Inc.). In some embodiments, the plate format used for the plaque assay can be modified, e.g., from a petri dish to a 48-well plate.

In some embodiments, a double-layer plaque assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 100 μL of a phage-containing sample (or medium only control) and incubated for 15 minutes. 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 16 hours at 37° C. in anaerobic environment until plaques are identified.

In some embodiments, a modified spot drop assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 30 min at 37 C in anaerobic environment. At this stage, 5 μL of samples containing phage or media only as control may be dropped on the plate, left to absorb, and then may be incubated for 16 hours until plaques are visible for counting.

In some embodiments, a liquid media assay is used to phenotype the bacteriophage. In some embodiments, liquid-based phage infection assays follow the time-course of infection and can provide more than quantitative end-points of infection as compared to the solid-phase plaque assays. In some embodiments, by mixing phage with bacteria in liquid medium, then following the turbidity of the culture over time, one can discern finer differences (e.g., a delay in the time of cell lysis) between how different bacterial strains interact with the phage.

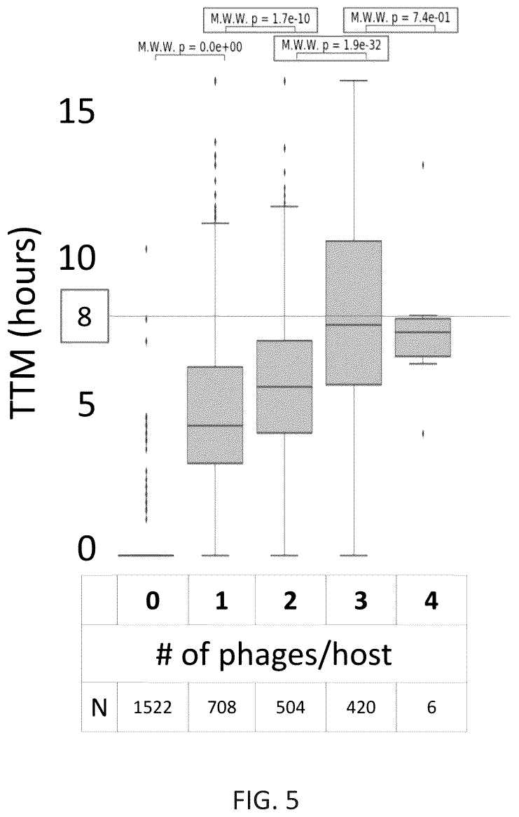

In some embodiments, a liquid-based phage infection assays is used to measure the time duration from the beginning of the experiment, when the bacteria and phages are mixed together until the host bacteria develops resistance to the phages (presumably by mutation). This period is also known as time-to-mutant (TTM). Such TTM assay was used to produce the results presented in FIG. 5.

In some embodiments, the TTM is declared synergistic when the OD reading reaches a predetermined threshold (e.g. 0.1 OD600). Then synergistic redundancy effect is concluded if the TTM of a combination (e.g. X,Y) is for example 50% longer than the longer TTM of the individual member phages (e.g. the TTM of X by itself and TTM of Y by itself).

In one embodiment, the synergistic effect is defined as above 10%, 20%, 30%, 40%, 50%, 60%, 70%, 80% above the longer individual phage member TTM.

In some embodiments, the liquid media assay allows for high-throughput measurements by using 96-well plates and reading optical density in a plate reader.

For example, a bacterial strain may be grown for 16 hours until an OD₆₀₀ of about 1.5-2. This culture may then be diluted using BHIS medium (which may be supplemented with 0.1% g/w L-cysteine) to a starting optical density, typically between 0.03 and 0.05 OD₆₀₀ and kept for grow for 6 additional hours. A volume of 200 μL of culture may then be dispensed into the wells of a Nunclon flat-bottomed 96-well plate. 10 μL of a sample containing phage or 10 μL of medium as control may be added to each well. The wells may be covered with 50 μL of mineral oil to limit evaporation, and a thin sterile optically transparent polyester film may be added to keep the culture sterile and anaerobic. Optical density measurements may be carried out every 15 minutes, e.g., in a Tecan Infinite M200 plate reader connected to a Tecan EV075 robot. Between measurements, the plate may be incubated while shaking at 37° C., e.g., inside the EV075 incubator.

In some embodiments, infectivity is determined by the plaque presence in a solid assay only. In some embodiments, infectivity is determined by a slower bacterial growth (e.g. compared to a control with bacteria only and no phage) in a liquid assay only. In some embodiments, infectivity is determined by both the liquid assay and the solid assay.

The bacteriophages described herein are typically present in a preparation in which their prevalence (i.e., concentration) is enriched over that (exceeds that) found in nature.

The term “preparation” refers to a composition in which the prevalence of bacteriophage is enriched over that found in nature. Since bacteriophages infect bacterial cells, they may be found in specimens or samples which are rich in bacteria—e.g. environmental samples such as sewage, wastewater and biological samples including feces. According to some embodiments of the invention, the preparation comprises less than 50 microbial species, e.g., bacteria and fungi—e.g. less than 40 bacterial species, less than 30 bacterial species, less than 20 bacterial species, less than 10 bacterial species, less than 5 bacterial species, less than 4 bacterial species, less than 3 bacterial species, less than 2 bacterial species or even devoid completely of bacteria.

According to a particular embodiment, the preparation comprises a single strain of bacteriophage (or a functional homolog thereof), no more than two different bacteriophage strains (or functional homologs thereof), no more than three different bacteriophage strains (or functional homologs thereof), no more than four different bacteriophage strains (or functional homologs thereof), no more than five different bacteriophage strains (or functional homologs thereof), no more than six different bacteriophage strains (or functional homologs thereof), no more than seven different bacteriophage strains (or functional homologs thereof), no more than eight different bacteriophage strains (or functional homologs thereof), no more than nine different bacteriophage strains (or functional homologs thereof), or no more than ten different bacteriophage strains (or functional homologs thereof).

In one embodiment, the preparation comprises a plurality of phage strains when at least one of the phage strains is KP10-11 (having the genome sequence at least 90% identical to the sequence as set forth in SEQ ID NO: 1).

In another embodiment, the preparation comprises a plurality of phage strains when at least one of the phage strains is KP10-48 (having the genome sequence at least 90% identical to the sequence as set forth in SEQ ID NO: 2).

In another embodiment, the preparation comprises a plurality of phage strains when at least one of the phage strains is 395-D2-3-2 (having the genome sequence at least 90% identical to the sequence as set forth in SEQ ID NO: 3).

In one embodiment, the preparation comprises at least two different phage strains when at least one of the phage strains is KP10-11 (having the genome sequence at least 90% identical to the sequence as set forth in SEQ ID NO: 1) and the other of the phage strains is KP10-48 (having the genome sequence at least 90% identical to the sequence as set forth in SEQ ID NO: 2).

In one embodiment, the preparation comprises at least three different phage strains when at least one of the phage strains is KP10-11 (having the genome sequence at least 90% identical to the sequence as set forth in SEQ ID NO: 1), the second of the phage strains is KP10-48 (having the genome sequence at least 90% identical to the sequence as set forth in SEQ ID NO: 2) and the third of the phage strains is 395-D2-3-2 (having the genome sequence at least 90% identical to the sequence as set forth in SEQ ID NO: 3).

Exemplary combinations of core phages in a single composition are provided in Table 1 herein below.

Additional contemplated combinations are provided in Example 2, Example 3 and Example 4 herein below.

TABLE 1

| Phage 1 | Phage 2 | Phage 3 |
|---|---|---|
| KP10-11 | KP10-48 | 395-D2-3-2 |
| KP10-11 | KP10-48 | KP10-85 |
| KP10-11 | KP10-48 | 395-D2-3-1 |
| KP10-11 | KP10-85 | 395-D2-3-1 |
| KP10-48 | KP10-85 | 395-D2-3-2 |
| KP10-11 | KP10-85 | KP10-91 |
| KP10-11 | 395-D2-3-1 | KP10-91 |
| KP10-11 | 395-D2-3-2 | KP10-91 |

One exemplary cocktail contemplated by the present inventors is one which comprises the following phages: KP10-11, KP10-48, KP10-85, 395-D2-3-1, 395-D2-3-2 and KP10-91.

Another exemplary cocktail contemplated by the present inventors is one which comprises the following phages: CT-227-KP4M1-20, CT-227-KP4M1-11, KP10-89 and KPL6a-30.

Another exemplary cocktail contemplated by the present inventors is one which comprises the following phages: KP10-11, KP10-85 and 395-D2-3-1 Another exemplary cocktail contemplated by the present inventors is one which comprises the following phages: KP10-11, KP10-85 and 395-D2-3-2.

Another exemplary cocktail contemplated by the present inventors is one which comprises the following phages: KP10-11, 395-D2-3-1 and KP10-91.

In one embodiment, the combination is selected such that more than 20% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 20% of all the strains of Klebsiella pneumonia which infect humans are targeted and lysed.

In another embodiment, the combination is selected such that at least 100, 200, 300, 400, 500, 600, 700, 800, 900 or 1000 different Klebsiella pneumonia strains are targeted.

In one embodiment, the combination is selected such that more than 30% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 30% of all the strains of Klebsiella pneumonia which infect humans are targeted and lysed.

In one embodiment, the combination is selected such that more than 40% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 40% of all the strains of Klebsiella pneumonia which infect humans are targeted and lysed.

In one embodiment, the combination is selected such that more than 45% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 45% of all the strains of Klebsiella pneumonia which infect humans are targeted and lysed.

In one embodiment, the combination is selected such that more than 50% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 50% of all the strains of Klebsiella pneumonia which infect humans are targeted and lysed.

In one embodiment, the combination is selected such that more than 55% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 55% of all the strains of Klebsiella pneumonia which infect humans are targeted and lysed.

In one embodiment, the combination is selected such that more than 60% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 60% of all the strains of Klebsiella pneumonia which infect humans are targeted and lysed.

In one embodiment, the combination is selected such that more than 65% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 65% of all the strains of Klebsiella pneumonia which infect humans are targeted and lysed.

In one embodiment, the combination is selected such that more than 70% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 70% of all the strains of Klebsiella pneumonia which infect humans are targeted and lysed.

In one embodiment, the combination is selected such that more than 75% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 75% of all the strains of Klebsiella pneumonia which infect humans are targeted and lysed.

In one embodiment, the combination is selected such that more than 80% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 80% of all the strains of Klebsiella pneumonia which infect humans are targeted and lysed.

In one embodiment, the combination is selected such that more than 85% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 85% of all the strains of Klebsiella pneumonia which infect humans are targeted and lysed.

In one embodiment, the combination is selected such that more than 90% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted (and lysed). In one embodiment, the combination is selected such that more than 90% of all the strains of Klebsiella pneumonia which infect humans are targeted and lysed.

The combinations described herein can be selected to include phages which have overlapping host coverages. The host coverages can be defined in terms of bacterial strain classification, bacterial capsule type and/or Multi-locus sequence typing (MLST)—see (github(dot)com/katholt/Kleborate).

In one embodiment, the combination is selected such that more than 10% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 10% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that at least 50, 100, 150, 200, 250, 300 specific Klebsiella pneumonia strains are targeted by more than 1 (e.g. 2, 3, 4 or 5) phage strain of the combination.

In another embodiment, the combination is selected such that more than 15% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 15% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 20% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 20% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 25% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 25% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 30% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 30% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 35% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 35% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 40% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 40% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 45% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 45% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 50% of the different strains of bacteria of a mixed population of Klebsiella pneumonia (e.g. comprising more than 100 different Klebsiella pneumonia strains, more than 200 different Klebsiella pneumonia strains and preferably more than 300 different Klebsiella pneumonia strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 50% of all the strains of Klebsiella pneumonia which infect humans are targeted and lysed by more than one phage strain (e.g. at least 2 phage strains of the combination, at least 3 phage strains of the combination, at least 4 phage strains of the combination or at least 5 phage strains of the combination).

It will be appreciated that, throughout the specification, when a phage is specifically named, the present invention also considers those phage that have at least 90% identity to the sequence of their genome, wherein the phage has a similar host range.

According to a specific embodiment, the preparation comprises at least about 10⁶ PFU, 10⁷ PFU, 10⁸ PFU, 10⁹ PFU, or even 10¹⁰ PFU or more of the above described (e.g. deposited) bacteriophages or functional homolog of same or progeny of same.

The bacteriophages described herein may be genetically modified such that their genomes include a heterologous sequence.

In one embodiment, the heterologous sequence serves as a marker signifying whether transformation is successful—e.g. a barcode sequence.

In another embodiment, the heterologous sequence encodes a therapeutic or diagnostic agent (also referred to herein as a payload). The therapeutic or diagnostic agent may be a nucleic acid (e.g. RNA silencing agent), a peptide or a protein. The therapeutic agent is typically selected according to the disease which is to be treated. Thus, for example if the bacteriophage is to be used for treating diseases associated with Klebsiella pneumoniae infection, the therapeutic agent is typically one that is known to be useful for treating that disease.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression. According to an embodiment of the invention, the RNA silencing agent is specific to the target RNA and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

Exemplary RNA silencing agents include but are not limited to siRNA, shRNA, miRNA and guide RNA (gRNA).

The therapeutic agent may be a bacterial protein or peptide (e.g., a small bacterial peptide that could act as a vaccine in the subject treated with the bacteriophage), a therapeutic protein or peptide (e.g., a cytokine, e.g., IL-15), a soluble peptide or protein ligand (e.g., a STING agonist or TRAIL), an antibody or an antibody fragment that recognizes a virulent or disease-causing antigen or is useful in an immunotherapy (e.g., a checkpoint inhibitor), an enzyme that when expressed produces a therapeutic useful product (e.g., a bacterial enzyme or metabolic cassette that produces a therapeutically useful bacterial metabolite or other bacterial antigen; a bacterial enzyme that produces LPS or causes cleavage of LPS from the outer membrane of gram negative bacteria), a shared tumor antigen or an enzyme that when expressed produces a shared tumor antigen, a unique tumor antigen or neoantigen or an enzyme that when expressed produces a unique tumor antigen or neoantigen,

In another embodiment, the therapeutic agent is an agent that is therapeutic in the treatment of inflammatory bowel disease.

According to another embodiment, the therapeutic agent is an immune modulating agent.

Examples of immune modulating agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1.

Examples of diagnostic agents include fluorescent proteins or enzymes producing a colorimetric reaction. Exemplary proteins that generate a detectable signal include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

In another example, the diagnostic agent is a luminescent protein such as products of bacterial luciferase genes, e.g., the luciferase genes encoded by Vibrio harveyi, Vibrio fischeri, and Xenorhabdus luminescens, the firefly luciferase gene FFlux, and the like.

Recombinant methods for inserting heterologous sequences into a phage genome are well-known in the art. The appropriate coding sequence is inserted in one or more of several locations in the phage genome. In one embodiment, the nucleic acid insert that is introduced into the phage genome is approximately no more than 10% of the phage genome length.

The payload coding sequence is inserted either after early, middle or late expressing phage genes and it can be expressed as part of a phage operon, relying on either an existing phage operon, promoter and terminator, or as a distinct operon. In the latter case, a relevant promoter and terminator from the phage is inserted as part of the newly formed operon.

For example, if strong expression of a payload is required, the payload coding sequence is added after the stop codon of the major capsid protein and expressed as part of the major capsid operon. Alternatively, it can be expressed by addition of a major capsid protein promoter and terminator as an individual newly formed operon which can be inserted anywhere in the phage genome that would not damage the functionality of the phage. If low expression of a payload is desired, the payload coding sequence can be added after the terminase gene (or other low expressing gene), which usually has low expression. Moreover, payload levels are tuned by adding a ribosome binding site with a desired strength.

In order to avoid negatively affecting phage infectivity and specificity, the payload coding sequence is typically not inserted inside an existing phage open reading frame. An exception to this is the case when the payload is intended to be expressed as a fusion protein of the phage outer coat. In that latter case of payload display, the payload coding sequence is added in frame to sequence encoding the phage coat protein.

The bacteriophages described herein may be used to treat subjects having diseases associated with Klebsiella pneumoniae infection.

Diseases associated with Klebsiella pneumoniae infection include inflammatory bowel diseases, urinary tract infections, acute liver abscesses, pneumonia, infectious wounds, and dental infections.

Inflammatory bowel diseases (IBDs) are severe gastrointestinal disorders characterized by intestinal inflammation and tissue remodeling, that increase in frequency and may prove disabling for patients. The major forms of IBD, ulcerative colitis (UC) and Crohn's disease are chronic, relapsing conditions that are clinically characterized by abdominal pain, diarrhea, rectal bleeding, and fever

As used herein, the term “subject” includes mammals, preferably human beings at any age which suffer from the pathology. Those in need of treatment may include individuals already having IBD, as well as those at risk of having, or who may ultimately acquire the disease. The need for treatment is assessed, e.g., by the presence of one or more risk factors associated with the development of IBD, the presence or progression of IBD, or likely receptiveness to treatment of a subject having IBD. For example, “treating” IBD may encompass reducing or eliminating associated symptoms, and does not necessarily encompass the elimination of the underlying disease etiology, e.g., a genetic instability locus.

In some embodiments, a subject with IBD is in remission and/or presently asymptomatic, and the bacteriophage described herein may be administered during the remission period to reduce the potential for a flare-up. In some embodiments, the subject in remission and/or presently asymptomatic for IBD is undergoing treatment, e.g., antibiotics and/or steroids, and the bacteriophage described herein may be co-administered with such treatment to reduce the potential for a flare-up.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

The bacteriophage may be used per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the bacteriophage accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient. In one embodiment, the bacteriophage may be administered directly into the tumor of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping, spray drying, coating or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (bacteriophage) effective to prevent, alleviate or ameliorate symptoms of a disorder (e.g., inflammatory bowel disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

In some embodiments, the composition is delivered to a subject in need thereof so as to provide one or more bacteriophage in an amount corresponding to a multiplicity of infection (MOI) of about 1 to about 10. MOT is determined by assessing the approximate bacterial load in the site of infection, or using an estimate for a given type of disease and then providing phage in an amount calculated to give the desired MOI.

In some embodiments, MOI may be selected based on the “multiplicity of 10 rule”, which states that where there are on average in order of 10 phages adsorbed per bacterium, bacterial density reduces significantly (Abedon S T, 2009, Foodborne Pathog Dis 6:807-815; and Kasman L M, et al., 2002, J Virol 76:5557-5564); whereas lower-titer phage administration (e.g., using a MOI lower than 10) is unlikely to be successful (Goode D, et al., 2003, App Environ Microbiol 69:5032-5036; Kumari S, et al., 2010, J Infect Dev Ctries 4:367-377).

In other embodiments, the amount of phage (or combination of phages) is provided so as to reduce the amount of bacteria (e.g. Klebsiella pneumoniae) present in the gastrointestinal tract (e.g. mouth, small intestine, colon, stomach) by at least 10%, 20%, 30%, 40%, 50%, 60%, 70%, 80%, 90% or even 100%.

In certain embodiments, the bacteriophage described herein is administered to ameliorate an IBD in a subject and results in one or more symptoms or physical parameters of the condition or disorder to improve by at least about 10%, 20%, 30%, 40%, 50%, 60%, 70%, 80%, 90%, 95%, or more as compared to levels in an untreated or control subject. In some embodiments, the improvement is measured by comparing the symptom or physical parameter in a subject prior to and following administration of the bacteriophage. In some embodiments, the measurable physical parameter is a reduction in bacterial CFU in the stool.

The measurable physical parameter may be any suitable clinical parameter known in the art, e.g., for Crohn's disease (Sandborn et al., 2013; Best et al., 1976; Warrel—Oxford Textbook of Medicine, 5th edition) or for ulcerative colitis (Tursi et al., 2010; Warrel—Oxford Textbook of Medicine, 5th edition). Crohn's disease parameters include but are not limited to assessment of clinical remission using Crohn's Disease Activity Index (CDAI) (CDAI score of ≤150 points) and CDAI-100 response (≥100-point decrease in the CDAI score from the baseline); inflammation reduction as measured by decrease in serum levels of C-reactive protein (CRP), differential blood count and serum albumin levels; reduced levels of fecal calprotectin and improvement in the appearance of the mucosal layer in terminal ileum and rectum as evaluated by microscopic examination of the biopsies collected during endoscopic procedures (e.g., reduction in neutrophil, macrophage and other immune cell infiltration, granuloma size and number, reduction in the number and size of ulcers, edematous mucosa). In addition the improvement of the disease may be evaluated based on the assessment of the clinical symptoms including diarrhea, abdominal pain, weight loss, fever, vomiting, colic, rectal bleeding, anemia, extraintestinal manifestations (hypersensitivity rash to sulphasalazine, erythema nodosum, pyoderma gangrenosum, oral aphthous ulcers, sore tongue, angular stomatitis, episcleritis or an anterior uveitis, arthritis, acute arthropathy, low back pain, sacroiliitis, ankylosing spondylitis, liver diseases: minor elevations of alkaline phosphatase or transaminases, primary sclerosing cholangitis, chronic liver disease ranging from an autoimmune hepatitis to the classic picture of concentric periductular fibrosis with obliteration of bile ducts, pericarditis with or without an effusion, autoimmune hemolytic anaemia, amyloid, rapidly progressing bronchiectasis).

Ulcerative colitis disease parameters include but not limited to improvement in the ulcerative colitis disease activity index (UCDAI) (UCDAI decrease of 50% or more from the baseline), activity of relapsing UC; remission, considered as UCDAI ≤2; improvement in hemoglobin levels, serum albumin levels, decrease in C-reactive protein levels; improvement in neutrophil leukocytosis (blood differential count); improvement in endoscopic scores (reduction in the edematous mucosa area, reduction in neutrophil, macrophage and other immune cell infiltration, reduction in the number and size of ulcers, edematous mucosa; improvement in the clinical symptoms: rectal bleeding, fever, stool frequency, diarrhea, the passage of mucus, abdominal pain, constipation, anorexia, nausea, weight loss, malaise, lassitude, symptoms of chronic iron deficiency, minor perianal disease, such as a fissure, extraintestinal manifestations described above, and additionally oral candidiasis, finger clubbing.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Compositions described herein may comprise more than one phage strain. In one embodiment, the composition comprises 2 phage strains, 3 phage strains, 4 phage strains, 5 phage strains or more.

In one embodiment, the bacteriophage cocktails comprise a plurality of phages that target a single Klebsiella pneumoniae strain.

In one embodiment, the bacteriophage cocktails comprise a plurality of phages that target more than one Klebsiella pneumonia strains.

Examples of particular combinations of phages are provided herein above.

The pharmaceutical compositions of the present invention also may be combined with one or more non-phage therapeutic and/or prophylactic agents, useful for the treatment and/or prevention of bacterial infections, as described herein and/or known in the art (e.g. one or more traditional antibiotic agents). Other therapeutic and/or prophylactic agents that may be used in combination with the phage(s) or phage product(s) of the invention include, but are not limited to, antibiotic agents, anti-inflammatory agents, antiviral agents, antifungal agents, or local anesthetic agents.

Standard or traditional antibiotic agents that can be administered with the bacteriophages described herein include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef. ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof.

Standard antifungal agents include amphotericin B such as liposomal amphotericin B and non-liposomal amphotericin B.

The present inventors further contemplate administering to the subject a probiotic which comprises “good” bacteria to occupy the niche left by the reduced “negative” bacteria. Such probiotic bacteria may comprise lactobacillus, saccharoinyces boulardii, and/or Bifidobacteriun.

In some embodiments, the bacteriophages and bacteriophage combinations (cocktails) are capable of modulating IBD associated with proton pump inhibitor (PPI) therapy by lysing the one or more Klebsiella bacteria. In some embodiments, the bacteriophage and bacteriophage cocktails are capable of modulating IBD associated with primary sclerosing cholangitis by lysing the one or more Klebsiella bacteria.

The bacteiophages and bacteriophage cocktails of the invention can be used in anti-infective compositions for controlling the growth of bacteria, in particular Klebsiella pneumoniae, in order to prevent or reduce the incidence of nosocomial infections. The anti-infective compositions find use in reducing or inhibiting colonization or growth of bacterial on a surface contacted therewith.

The bacteriophages of the invention may be incorporated into compositions that are formulated for application to biological surfaces, such as the skin and mucus membranes, as well as for application to non-biological surfaces.

Anti-infective formulations for use on biological surfaces include, but are not limited to, gels, creams, ointments, sprays, and the like. In particular embodiments, the anti-infective formulation is used to sterilize a surgical field, or the hands and/or exposed skin of healthcare workers and/or patients.

Anti-infective formulations for use on non-biological surfaces include sprays, solutions, suspensions, wipes impregnated with a solution or suspension and the like. In particular embodiments, the anti-infective formulation is used on solid surfaces in hospitals, nursing homes, ambulances, etc., including, e.g., appliances, countertops, and medical devices, hospital equipment.

In preferred embodiments, the non-biological surface is a surface of a hospital apparatus or piece of hospital equipment. In particularly preferred embodiments, the non-biological surface is a surgical apparatus or piece of surgical equipment.

The present invention also encompasses diagnostic methods for determining the causative agent at the site of the bacterial infection. In certain embodiments, the diagnosis of the causative agent of a bacterial infection is performed by (i) culturing a sample from a patient, e.g., a tumor biopsy, stool sample or other sample appropriate for culturing the bacteria causing the infection; (ii) contacting the culture with one or more bacteriophages of the invention; and (iii) monitoring for evidence of cell growth and/or lysis of the culture. Because the activity of phages tends to be species or strain specific, susceptibility, or lack of susceptibility, to one or more phages of the invention can indicate the species or strain of bacteria causing the infection.

The sample may be a tissue biopsy or swab collected from the patient, or a fluid sample, such as blood, tears, or urine.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Isolating and Characterizing Phage

Materials and Methods

Bacteria Sequencing and Testing:

Each Klebsiella pneumoniae isolate was sequenced using Illumina Nextera sequencing of 150 bp paired-end reads. Adapter removal and quality trimming was done using Trimgalore (www(dot)github(dot)com/FelixKrueger/TrimGalore) and cutadapt (Martin, 2011). Positions with phred scores below 30 were deleted from a read and reads shorter than 55 bp after removing low quality nucleotides were discarded entirely. Assembly was performed using SPAdes (Bankevich et al., 2012). Validation of K. pneumoniae taxonomy was done using Kleborate (www(dot)github(dot)com/katholt/Kleborate) which genotypes assemblies and determines whether they are indeed K. pneumoniae. Results were obtained by comparing the contigs of an assembly to known klebsiella species genomes hosted at: www(dot)bigsdb(dot)pasteur(dot)fr/klebsiella/klebsiella(dot)html. Comparisons were performed using BLAST (Altschul, 1990) and the Klebsiella species was determined from a phylogenetic tree derived from identities between known Klebsiella species, where the distance metric is mash (Ondov et al., 2016).

Phage Amplification and Determination of Phage Titers:

Phage were amplified from liquid broth or from soft agar double agar overlay plaque. Amplification from liquid broth was performed by diluting 50 μL of isolated phage sample into 4 mL log phase host culture at OD=0.5, and incubating at 37° C., overnight. When amplified from a plaque a whole plaque was picked using a 1 uL loop and release the plaque into the culture OD600=0.1−0.2. Tubes were centrifuged, the supernatant was filtered by 0.45 μm filter, and 1 mM of the divalent ions Ca²⁺ and Mg²⁺ were added.

Phage titers were determined by drop plaque assay as follows: host culture was prepared by inoculating 4 mL liquid BHIS with 5-10 colonies of the host and incubating at 37° C., until OD was 1.7 (1.5-2 hrs). 150 μL of host culture were added to 4 mL of molten top agar (BHIS top agar: BHIS media, 0.4% Agarose) with divalent ions Ca²⁺ and Mg²⁺ and dispensed on BHIS agar plats (1.6% Agarose). Plate were left to solidify for 15 min at RT before incubating the plate at 37° C. for 30 minutes. Then dilutions of phage sample were dropped (5 μL). Plates were incubated for overnight before counting plaques (10-50 plaques/drop) and determination of phage titer (number of plaques×200×reciprocal of counted dilution=PFU/mL).

Amplification of phage on soft agar was performed in cases where titers of phage that were produced from liquid BHIS were lower than 1×10⁹ PFU/mL. Log phase host culture at OD=1.7, was diluted to OD=1, and aliquots of 100 μL diluted host culture were added to 100 μL of phage at a titer that was sufficient to result in production of confluent plaques, and incubated at room temperature for 15 minutes. Then, each host-phage mixture was diluted with 3 mL soft molten agar and poured on the top of BHIS bottom agar. Plates were left to harden, and then incubated at 37° C. for 2-3 hrs or until complete lysis. Plates were scraped from the top agar with a spreader and agar was transferred to sterile tubes with BHIS and 10 μL of chloroform. Tubes were left to stand for 30 minutes to enable phage to elute from the soft agar. Tubes were centrifuged, and supernatant was filtered with 0.45 μM syringe filter to eliminate residual agar. Divalent ions Ca²⁺ Mg²⁺ at concentration of 1 mM were added to tubes which were stored at 4° C.

High Throughput Liquid Host Range:

A replicator (VP408) was used to inoculate 200 μL BHIS from glycerol stock plates following incubation overnight (15-16 h) at 37° C., without shaking. The next day, a 2 μL replicator (VP407) was used to dilute the overnight starters 1:100 in a 96-well plate prefilled with 200 μL BHIS+1 mM ions. One plate served as the “no phage control” (NPC) and to the other, 10 μL phage was added, to each well (number of plates as the number of phages tested+1 (NPC)). Plates were incubated in the robot (12plate_OD_Hummus program) at 37° C. with shaking and OD₆₀₀ was measured every 30 min for 4 hours. The ration of OD₆₀₀(NPC)/OD₆₀₀ (treated well), at 4 hours, was calculated. Ratio <1.8 was defined as resistant and ratio >1.8 as sensitive.

High Throughput Solid Host Range:

Inverted Plaque Assay was used as an alternative to the Double Layer Plaque Assay. In this method, phages are incorporated into the top agar and bacteria are dropped on it. Contrary to the double layer plaque assay, no plaque is formed, but rather, sensitive bacteria appeared either as infected lawn (perforated lawn), or single colonies, or no colony at all. The growth of bacteria on the top agar with the tested phage is compared to the growth of the same bacteria on the top agar without phage (No Phage Control (NPC)).

Each phage is diluted to 10⁸ PFU/mL in BHIS, and 10 p L is transferred into 10 mL melted top agar with 1 mM ions (at 56° C.). Following a gentle mix, the tube contents are poured onto a BHIS plate. For NPC plates, only ions are added to the top agar. The plates are left for 20 min to allow the top agar to solidify. A replicator for frozen samples (VP408) is used to inoculate the bacteria into 200 μL BHIS and the plate is incubated overnight (15-16 h) at 37° C., without shaking. The following day, the cultures are diluted 1:10000 in PBS using the 2 uL replicator (VP407) into 200 μL BHIS in 96-well plates with 200 μL of PBS. 2 μL of the diluted bacteria are spotted onto the phage and NPC plate. After drops are fully absorbed on the top agar (a few minutes with cover slightly open), the plates are incubated at 30° C. overnight (15-16 hours). The growth of the bacteria on phage plate is compared to the growth on the NPC and bacteria were designated sensitive (“S”) or resistant (“R”) to the phage.

Results

Sewage (from the Israel National Virology Center and Sourasky Tel Aviv Medical Center) was used to hunt phages using KP isolated from PSC/IBD patients.

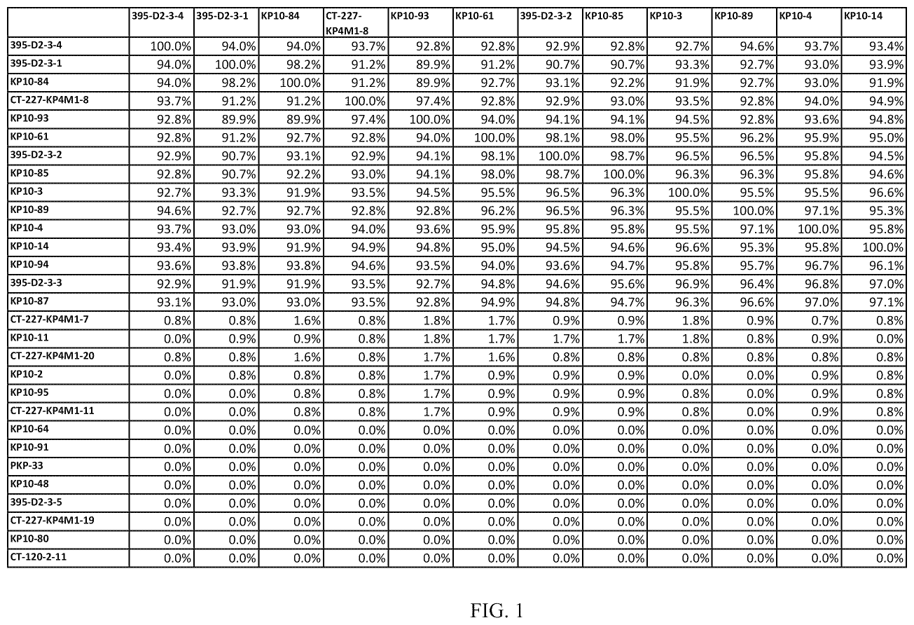

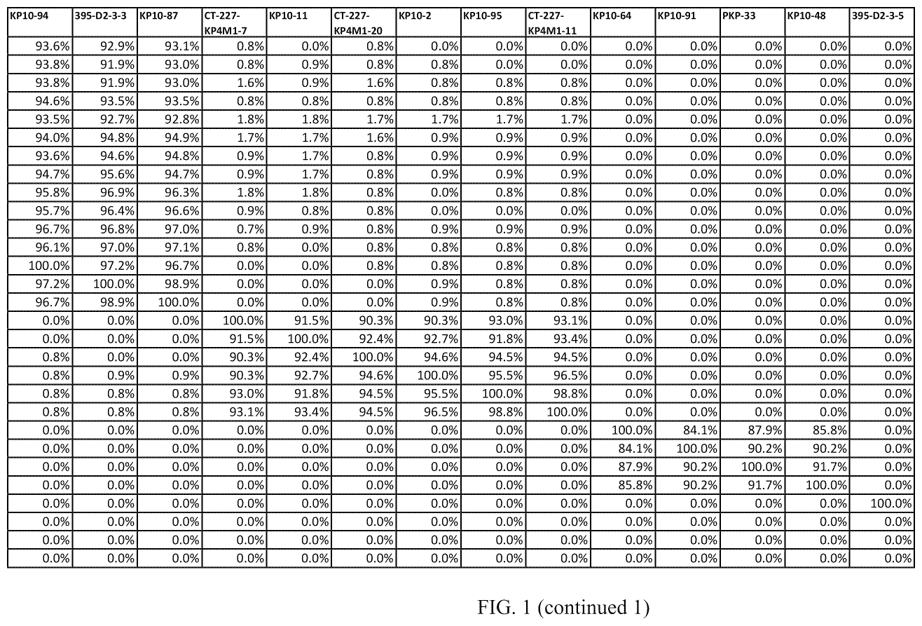

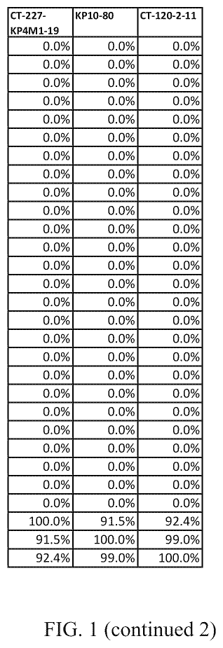

29 phages were isolated, purified, and sequenced. Their taxonomy was deduced from the sequence based on International Committee on Taxonomy of Viruses (ICTV) classification (Table 2). Additionally, the sequence was used to determine the distance (sequence homology) between the phages (FIG. 1).

TABLE 2
Exemplary Isolated Bacteriophage against Klebsiella pneumonia species.

| Single | | | | | | | |
|---|---|---|---|---|---|---|---|
| letter | | | | | SEQ | Deposit | Date of |
| designation | Phage | Family | Subfamily | Genus | ID NO: | number | deposit |
| b | 395-D2-3-2 | Myoviridae | Tevenvirinae | Slopekvirus | 3 | F/00162 | 6 Dec. 2021 |
| a | 395-D2-3-1 | Myoviridae | Tevenvirinae | Slopekvirus | 4 | F/00161 | 6 Dec. 2021 |
| c | 395-D2-3-3 | Myoviridae | Tevenvirinae | Slopekvirus | 5 | | |
| d | 395-D2-3-4 | Myoviridae | Tevenvirinae | Slopekvirus | 6 | F/00177 | 13 Jan. 2022 |
| e | 395-D2-3-5 | Autographiviridae | Studiervirinae | Teetrevirus | 7 | F/00178 | 13 Jan. 2022 |
| f | CT-120-2-11 | Drexlerviridae | | Webervirus | 8 | | |
| g | CT-227-KP4M1-11 | Myoviridae | Tevenvirinae | Jiaodavirus | 9 | F/00163 | 6 Dec. 2021 |
| h | CT-227-KP4M1-19 | Drexlerviridae | | Webervirus | 10 | | |
| i | CT-227-KP4M1-20 | Myoviridae | Tevenvirinae | Jiaodavirus | 11 | F/00164 | 6 Dec. 2021 |
| j | CT-227-KP4M1-7 | Myoviridae | Tevenvirinae | Jiaodavirus | 12 | | |
| k | CT-227-KP4M1-8 | Myoviridae | Tevenvirinae | Slopekvirus | 13 | | |
| l | KP10-11 | Myoviridae | Tevenvirinae | Jiaodavirus | 1 | F/00165 | 6 Dec. 2021 |
| m | KP10-14 | Myoviridae | Tevenvirinae | Slopekvirus | 14 | | |
| n | KP10-2 | Myoviridae | Tevenvirinae | Jiaodavirus | 15 | | |
| o | KP10-3 | Myoviridae | Tevenvirinae | Slopekvirus | 16 | F/00179 | 13 Jan. 2022 |
| p | KP10-4 | Myoviridae | Tevenvirinae | Slopekvirus | 17 | | |
| q | KP10-48 | Demerecviridae | | Sugarlandvirus | 2 | F/00166 | 6 Dec. 2021 |
| r | KP10-61 | Myoviridae | Tevenvirinae | Slopekvirus | 18 | | |
| s | KP10-64 | Demerecviridae | | Sugarlandvirus | 19 | F/00167 | 6 Dec. 2021 |
| t | KP10-80 | Drexlerviridae | | Webervirus | 20 | F/00168 | 6 Dec. 2021 |
| u | KP10-84 | Myoviridae | Tevenvirinae | Slopekvirus | 21 | | |
| v | KP10-85 | Myoviridae | Tevenvirinae | Slopekvirus | 22 | | |
| w | KP10-87 | Myoviridae | Tevenvirinae | Slopekvirus | 23 | F/00180 | 13 Jan. 2022 |
| x | KP10-89 | Myoviridae | Tevenvirinae | Slopekvirus | 24 | | |
| y | KP10-91 | Demerecviridae | | Sugarlandvirus | 25 | F/00169 | 6 Dec. 2021 |
| z | KP10-93 | Myoviridae | Tevenvirinae | Slopekvirus | 26 | | |
| A | KP10-94 | Myoviridae | Tevenvirinae | Slopekvirus | 27 | | |
| B | KP10-95 | Myoviridae | Tevenvirinae | Jiaodavirus | 28 | | |
| C | PKP-33 | Demerecviridae | | Sugarlandvirus | 29 | F/00181 | 13 Jan. 2022 |
| | KPL6a-30 | Ackermannviridae | | | 30 | | |

The % sequence homology (based on local BLAST) of the isolated phages was compared as set forth in FIG. 1.

The host ranges (HR) of the phages was tested. HR analysis of all isolated phage was performed in both liquid and solid. HR was determined based on 352 clinical isolated KP strains from IBD/PSC patients. The 352 clinical strains were selected as being the most representative (in terms of capsule type, genomic sequence and MLST) of thousands of KP bacterial strains that were isolated from IBD and PSC patients. The percent coverage of these 352 isolated KP strains is summarized in Table 3, herein below.

TABLE 3

| Phage | % coverage |
|---|---|
| CT-227-KP4M1-19 | 1% |
| CT-227-KP4M1-8 | 1% |
| KP10-94 | 1% |
| KP10-93 | 3% |
| CT-227-KP4M1-7 | 5% |
| CT-227-KP4M1-20 | 9% |
| KP10-95 | 11% |
| KP10-80 | 12% |
| CT-227-KP4M1-11 | 13% |
| 395-D2-3-5 | 16% |
| CT-120-2-11 | 16% |
| KP10-14 | 16% |
| KP10-3 | 19% |
| KP10-64 | 20% |
| PKP-33 | 20% |
| KP10-89 | 20% |
| KP10-2 | 23% |
| 395-D2-3-3 | 23% |
| KP10-48 | 23% |
| KP10-85 | 25% |
| KP10-4 | 25% |
| KP10-61 | 25% |
| KP10-84 | 26% |
| KP10-87 | 26% |
| 395-D2-3-4 | 27% |
| PKP-39D | 27% |
| 395-D2-3-2 | 34% |
| 395-D2-3-1 | 35% |
| KP10-11 | 37% |

20 cocktail compositions were prepared, each including a combination of 3 members at similar concentrations. The combinations were tested in a liquid assay against 79 representative bacterial strains. The % coverage is presented in Table 4 below.

TABLE 4

| member phage | % coverage in liquid |
|---|---|
| KP10-11 | 59% |
| KP10-48 | |
| 395-D2-3-2 | |
| KP10-11 | 58% |
| KP10-48 | |
| KP10-85 | |
| KP10-11 | 51% |
| KP10-48 | |
| 395-D2-3-1 | |
| KP10-11 | 62% |
| KP10-85 | |
| 395-D2-3-1 | |
| KP10-11 | 59% |
| KP10-85 | |
| 395-D2-3-2 | |
| KP10-11 | 48% |
| 395-D2-3-1 | |
| 395-D2-3-2 | |
| KP10-48 | 51% |
| KP10-85 | |
| 395-D2-3-1 | |
| KP10-48 | 43% |
| KP10-85 | |
| 395-D2-3-2 | |
| KP10-48 | 38% |
| 395-D2-3-1 | |
| 395-D2-3-2 | |
| KP10-85 | 44% |
| 395-D2-3-1 | |
| 395-D2-3-2 | |
| KP10-11 | 46% |
| KP10-48 | |
| KP10-91 | |
| KP10-11 | 59% |
| KP10-85 | |
| KP10-91 | |
| KP10-11 | 54% |
| 395-D2-3-1 | |
| KP10-91 | |
| KP10-11 | 52% |
| 395-D2-3-2 | |
| KP10-91 | |
| KP10-48 | 43% |
| KP10-85 | |
| KP10-91 | |
| KP10-48 | 25% |
| 395-D2-3-1 | |
| KP10-91 | |
| KP10-48 | 43% |
| 395-D2-3-2 | |
| KP10-91 | |
| KP10-85 | 43% |
| 395-D2-3-1 | |
| KP10-91 | |
| KP10-85 | 41% |
| 395-D2-3-2 | |
| KP10-91 | |
| 395-D2-3-1 | 43% |
| 395-D2-3-2 | |
| KP10-91 | |

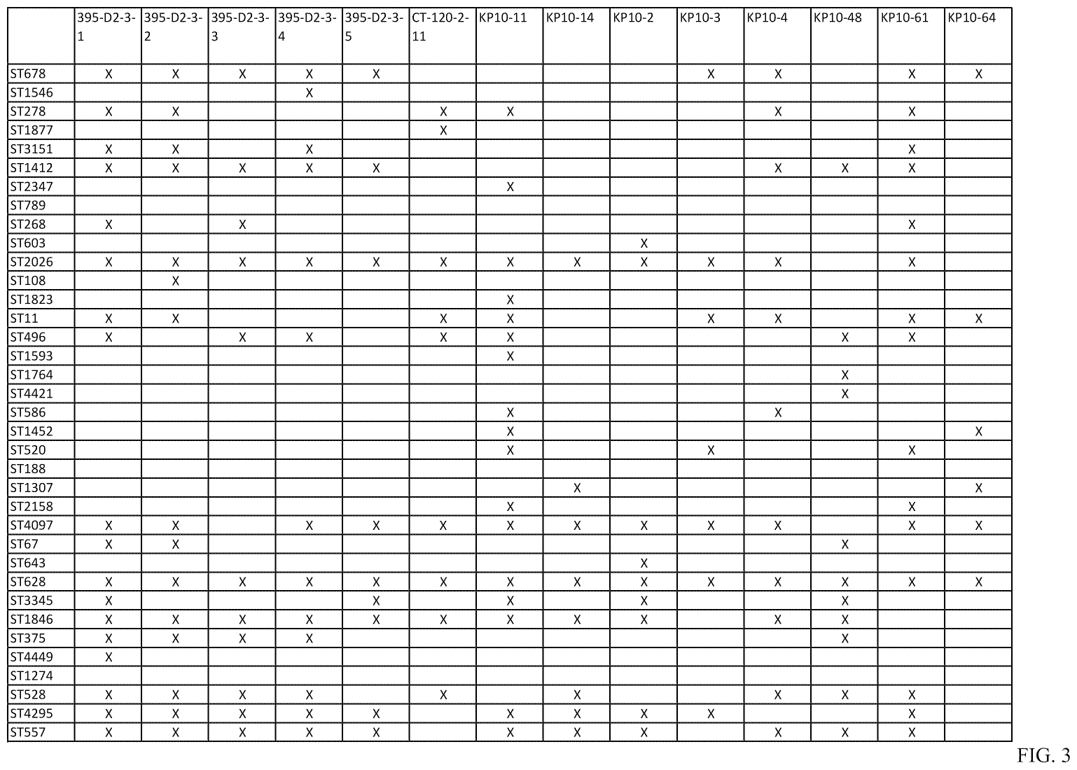

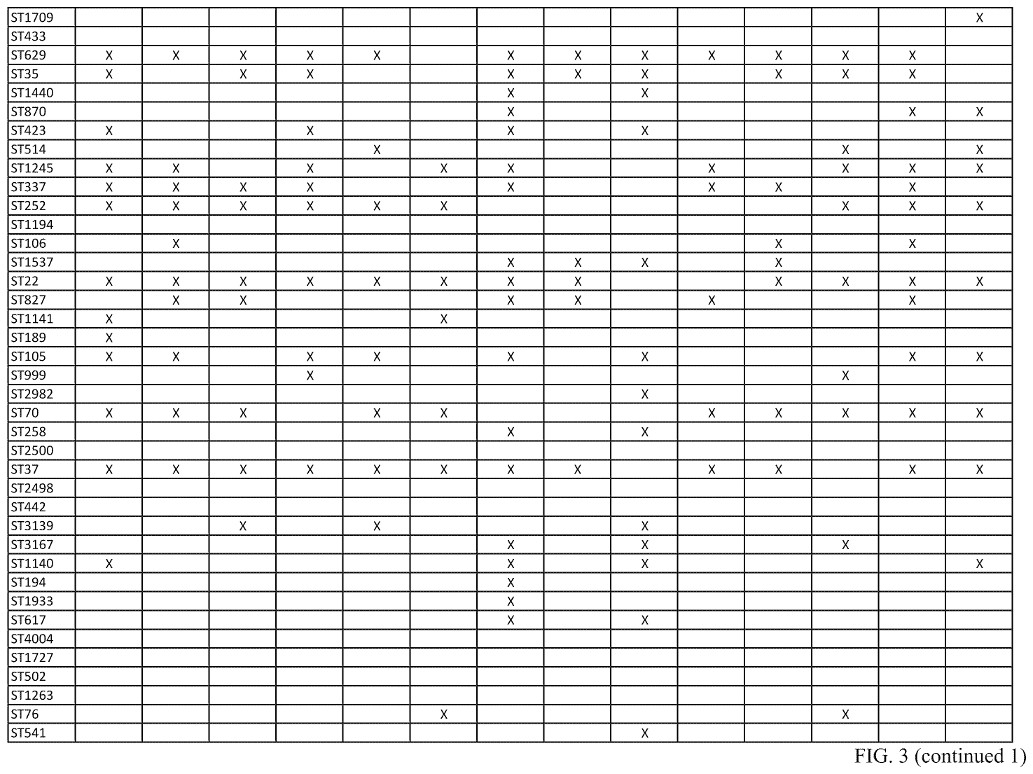

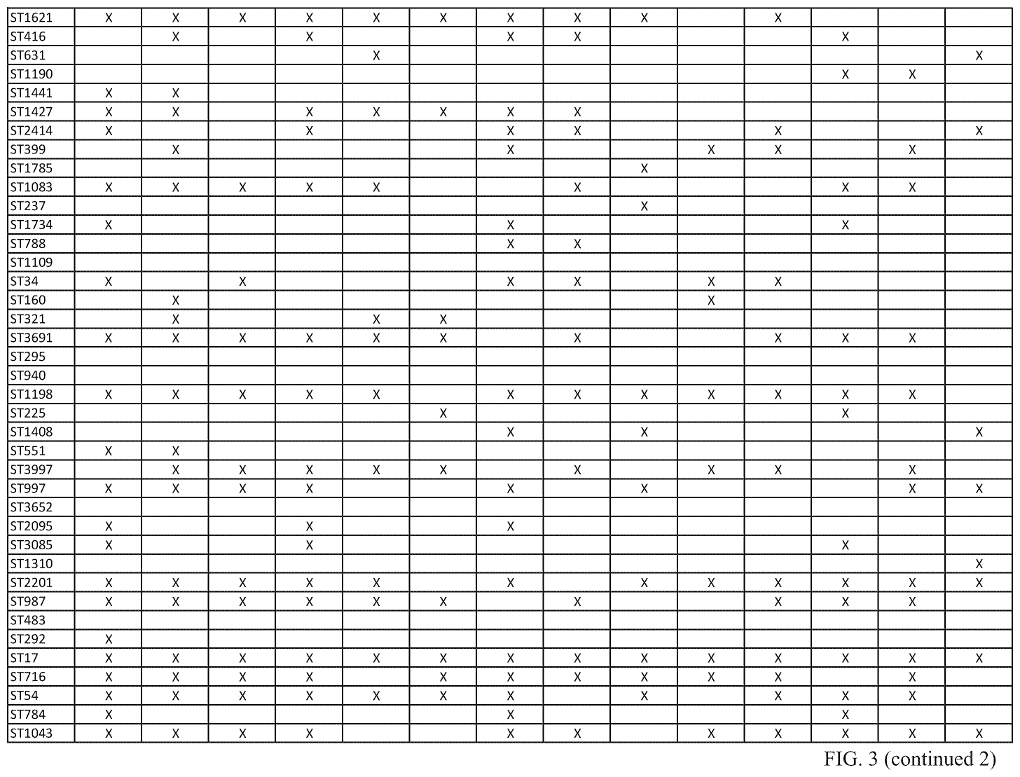

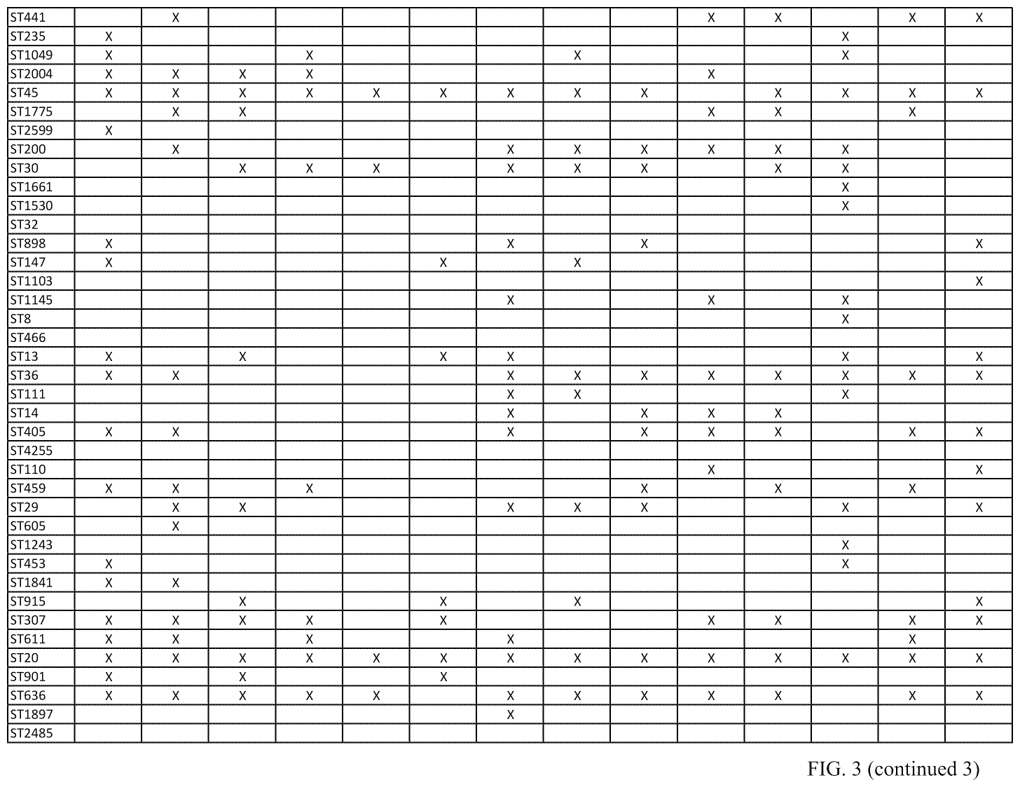

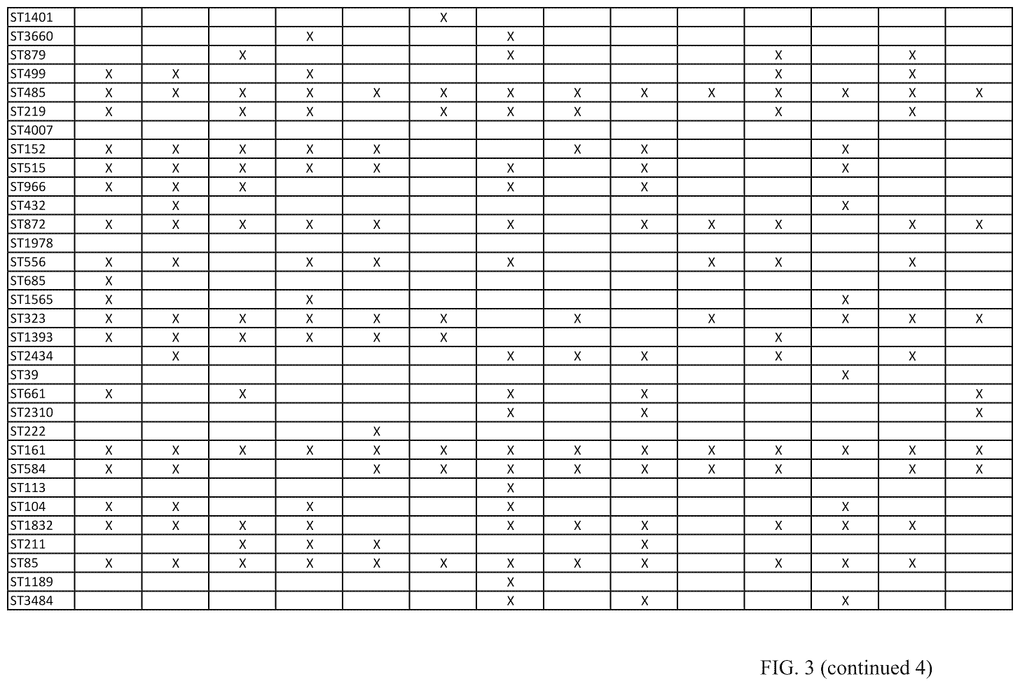

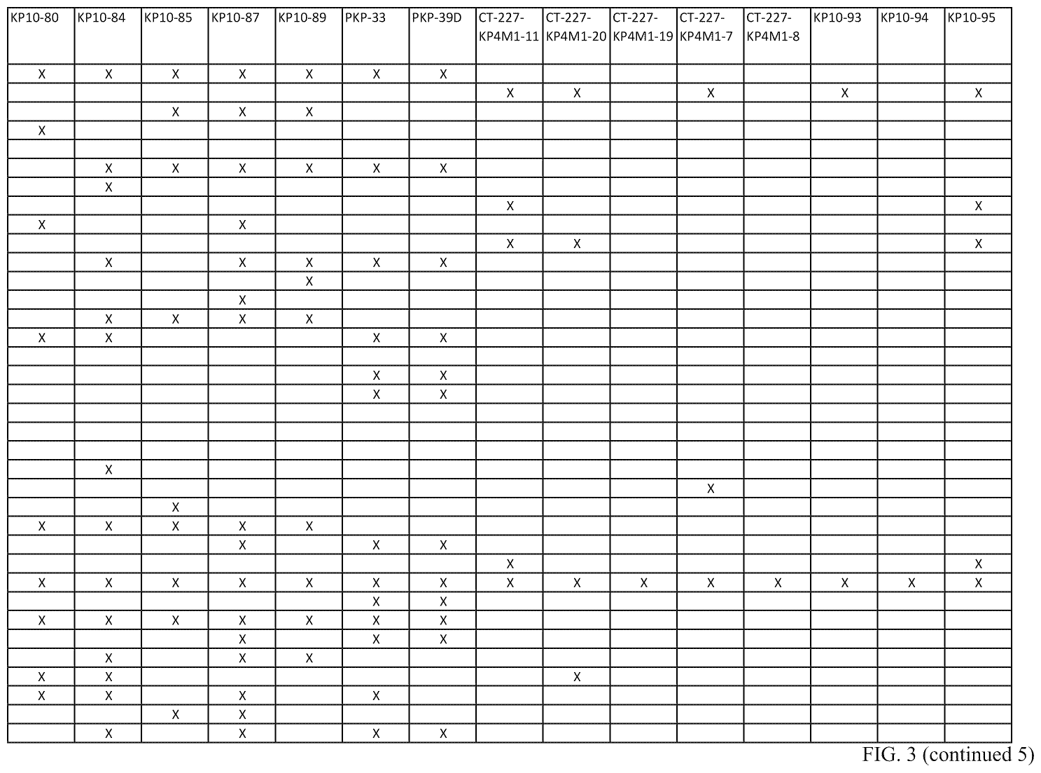

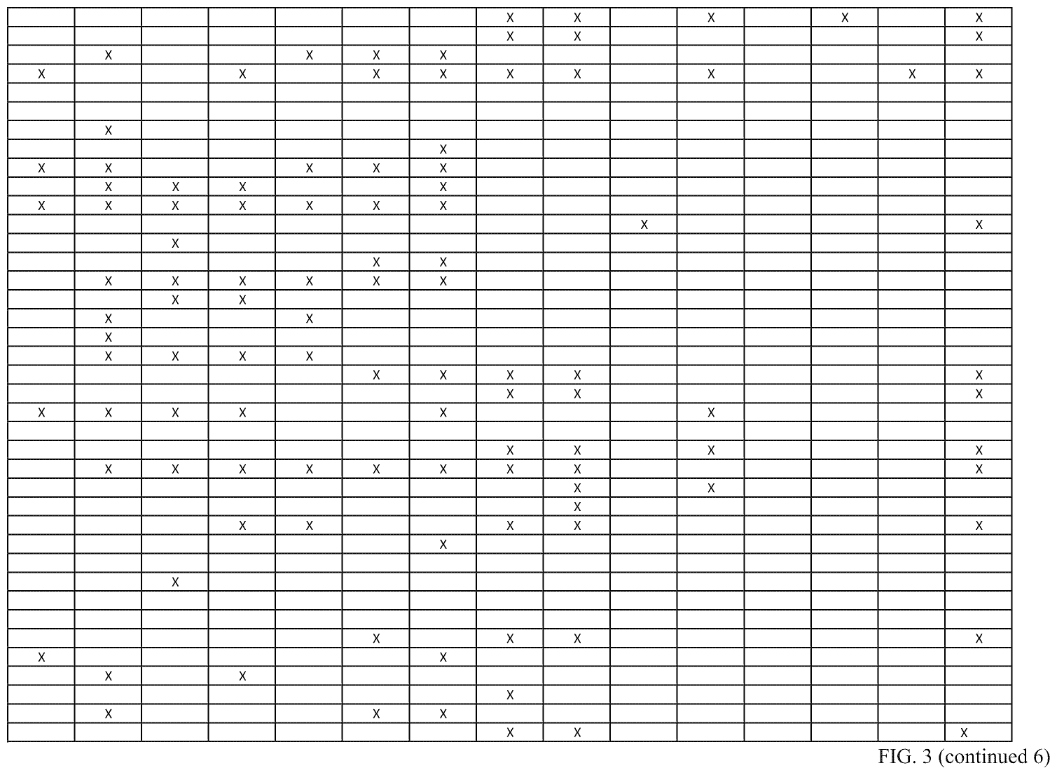

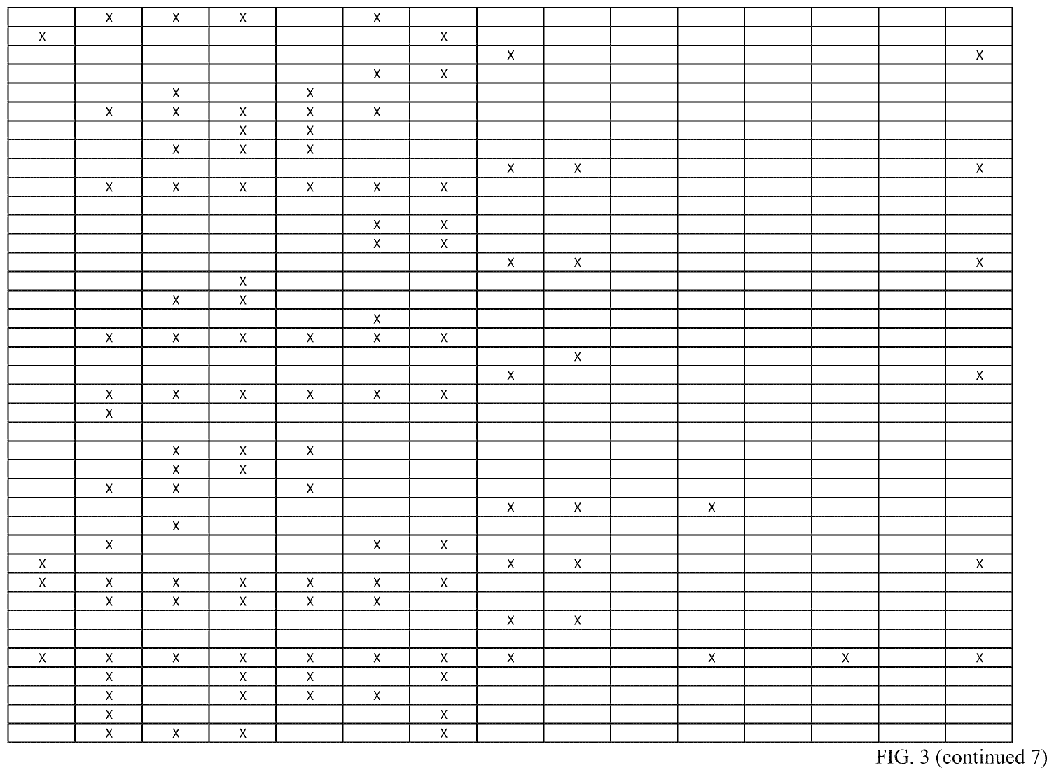

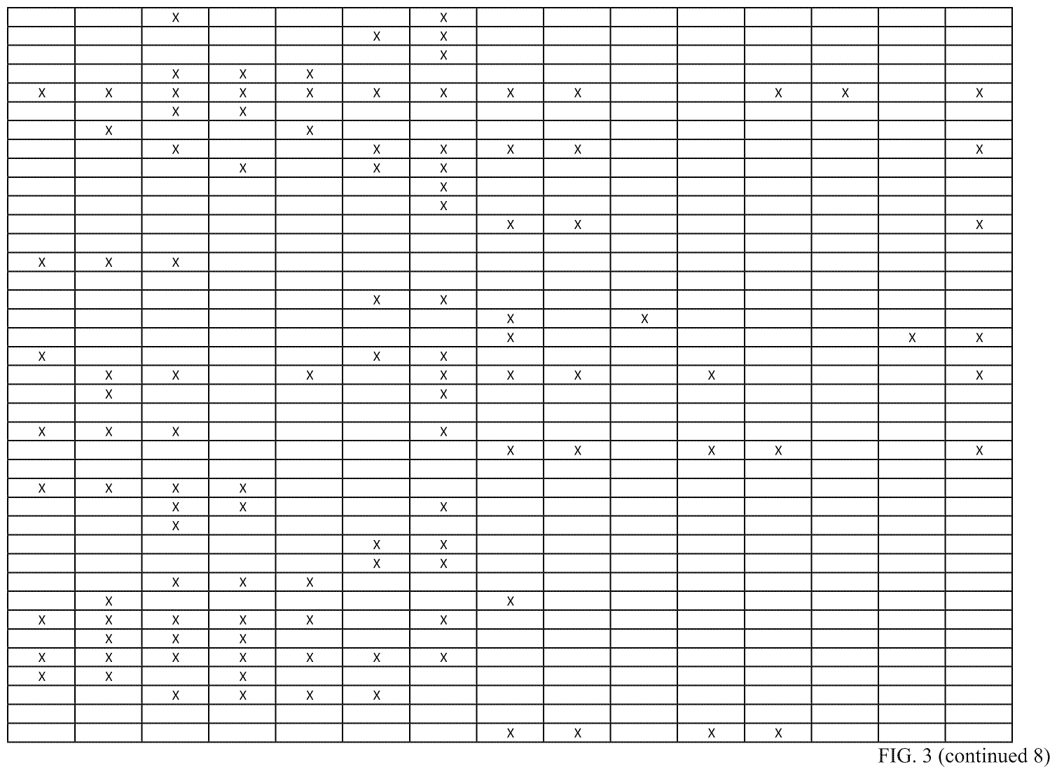

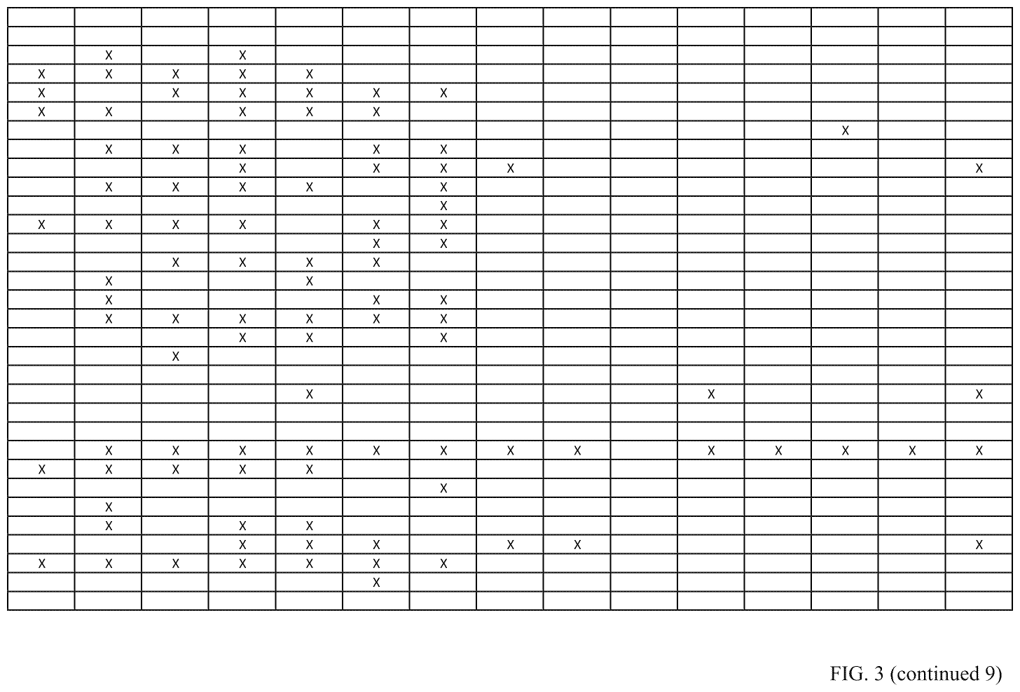

The host range of the phage was also profiled according to the capsule type of the infected bacteria. The results are set forth in FIG. 2. 62 capsule types were found to be infected by the phages, as specified below per each phage. The host range of the phage was also profiled according to the MLST of the infected bacteria. The results are set forth in FIG. 3.

### Example 2

Phage Combinations Selected According to Bacterial Coverage as Defined by Strain

For this example, the particular phages are referred to by the single letter designation in Table 1, herein above.

2 Phage Combinations

2 phage combinations were analyzed in silico for their ability to lyse the 352 different strains of Klebsiella pneumoniae bacteria (as described for Example 1) based on the ability of the single phage to lyse the bacteria as assessed by a liquid media assay.

The combinations that lyse the highest number of bacterial strains are provided herein below (out of 406 possible combinations, the top 200 are provided). This trait is referred to as “at least 1 phage % coverage”. The number following each combination refers to the Percent trait performance—in this case the percent of the 352 strains that are targeted by the phage combination. The combinations are listed in descending performance grade.

Thus, for example in the case of [al;55], which provides the highest percent coverage of all the 2 phage combinations, 395-D2-3-1 and KP10-11 lysed 55% of all the strains of Klebsiella pneumonia analyzed.

[al;55][bl;53][ly;51][lu;50][lq;50][dl;49][lw;49][cl;49][lg;49][lv;49][by;48][lB;48][ay;48][ls;47][bq;47][lC;47][ag;47][lx;46][an;46][lr;46][bg;46][ab;46][li;45][bn;45][aB;45][aq;45][lp;45][lo;45][fl;45][bB;45][as;44][el;44][ap;44][ar;44][ao;43][lt;43][ai;43][wy;43][bC;43][uy;43][lm; 43][ln;43][av;43][vy;43][bi;42][ry;42][bu;42][lj;42][bs;42][qv;42][bd;42][py;41][dy;41][bc;41][n y;41][pq;41][qw;41][xy;41][aw;41][aC;41][bm;40][qr;40][nu;40][ds;40][bw;40][qu;40][dn;40][a d;40][ac;40][nq;40][bp;40][am;40][sy;39][nv;39][aj;39][nw;39][yg;39][bf;39][dv;39][bt;39][af;39][lz;39][qs;39][dq;39][bj;39][cy;39][nr;39][at;39][sw;39][au;39][oq;39][oy;39][br;38][bo;38][ae ;38][uv;38][lh;38][ug;38][qx;38][yB;38][lk;38][ax;38][dr;38][lA;38][pu;38][dp;38][su;38][vC;38][cq;38][dg;38][ru;38][wg;37][uB;37][ou;37][do;37][be;37][sC;37][uw;37][az;37][rg;37][cn;37][bx;37][rC;37][vg;37][pg;37][du;37][dB;37][ns;36][cs;36][rw;36][fy;36][wB;36][cu;36][cv;36][yi ;36][np;36][ah;36][bz;36][dw;36][aA;36][nx;36][uC;36][dx;36][vB;36][ak;36][pB;36][rB;36][ps; 36][qg;35][wC;35][my;35][pC;35][ey;35][nC;35][bh;35][pw;35][no;35][bv;35][pv;35][cr;35][vw ;35][ui;35][di;35][ow;35][oC;35][bA;35][rs;35][bk;35][fn;34][wi;34][Cy;34][qB;34][cp;34][sv;34][cg;34][ri;34][mv;34][ty;34][pi;34][fv;34][mu;33][vi;33][co;33][mr;33][ux;33][pr;33][xC;33][px;33][mq;33][cd;33][df;33][ms;33][rv;33][cx;33][dC;33][eq;32][cC;32][cB;32][sx;32][fq;32].

The percent of host bacterial strains that are infected by two phages of a phage combination are provided. This trait is referred to as “at least 2 phage % coverage”. The phage combinations are ordered in descending performance grade.

Thus, for example, in the case of [al;17], when 395-D2-3-1 and KP10-11 are used in a combination, 17% of the bacterial strains analyzed were targeted by both these phage.

[bv;24][ab;23][au;22][ad;21][aw;20][br;20][bw;20][cw;20][bd;19][bp;19][qy;18][bu;18][ac;18][bx;18][bl;17][ax;17][dw;17][cd;17][rv;17][pr;17][ln;17][al;17][av;17][du;16][pw;16][lp;16][uw;16][vw;16][lr;16][ap;16][ar;16][bc;16][rw;15][bo;15][wx;15][pv;15][cp;14][dC;14][aC;14][vx;14][dl;14][ay;14][dr;14][lw;14][ux;14][dp;14][ru;14][or;14][ly;14][cu;14][op;14][pu;14][by; 13][Cy;13][cr;13][lu;13][qC;13][uv;13][lv;13][rx;13][px;13][dy;12][ov;12][be;12][de;12][dv;12][aq;12][ae;12][cv;12][bs;12][cy;12][np;12][af;12][ew;12][dm;12][dx;12][lo;11][mw;11][fu;11][wC;11][eC;11][an;11][am;11][cl;11][lx;11][ow;11][uC;11][ce;11][cx;11][bn;11][cC;11][dq;11][eu;11][sv;11][bC;11][mp;11][bf;11][py;11][uy;11][lm;11][wy;11][gB;11][lC;11][ao;11][as;11][e p;11][rs;11][lq;11][fw;11][ry;10][df;10][bq;10][cm;10][pC;10][ls;10][fp;10][bm;10][nw;10][cf;10][mu;10][ps;10][qu;10][ox;9][dn;9][cn;9][vy;9][co;9][ex;9][ev;9][os;9][fr;9][cq;9][do;9][nr;9][o u;91[qw;9][nu;9][su;9][er;9][mC;9][fx;9][ny;9][el;9][rC;9][my;9][fl;8][sx;8][at;8][mn;8][mr;8][s y;8][qr;8][sw;8][nv;8][oy;8][gi;8][em;8][en;8][xC;8][tu;8][nC;8][mv;8][ey;8][nx;8][ef;8][pq;7][f C;7][fv;7][fm;7][vC;7][fy;7][no;7][dt;7][tw;7][bt;7][fq;7][pt;7][xy;7][mq;7][ft;7][cs;7][rt;7][iB;7][mx;7][ds;7][eq;7][eo;7][ns;7][qv;6][ty;6][ct;6][mo;6][fs;6][ng;6][fo;6][qx;6][lt;6][ty;6][es;5][st; 5].

3 Phage Combinations

3 phage combinations were analyzed in silico for their ability to lyse the 352 different strains of Klebsiella pneumoniae bacteria based on the ability of the single phage to lyse the bacteria as assessed by a liquid media assay.

The combinations with the highest “at least 1 phage % coverage” are provided herein below (out of 3654 possible combinations, the top third (1224) are provided). The number following each combination refers to the Percent trait performance. The phage combinations are ordered in descending performance grade.

For example, in the case of [alg;66], the combination that provided the highest percent coverage, 395-D2-3-1, KP10-11 and CT-227-KP4M1-11 lysed 66% of all the strains of Klebsiella pneumonia analyzed.

[alg;66][alB;65][blg;65][blB;64][ali;63][lyg;62][bli;62][lug;62][aly;62][lqg;62][alq;62][lyB;61][blq;61][luB;61][bly;61][lqB;61][abl;61][als;61][lvg;61][dlg;60][lwg;60][byg;60][ayg;60][clg;59][alj;59][luy;59][lvB;59][dlB;59][lyi;59][lwB;59][blu;59][alo;59][bqg;59][aln;59][alv;59][blC;59][ayB;59][lui;59][byB;59][lsg;57][lqv;57][lgi;57][lwy;57][lqu;57][alr;57][blj;57][bc;57][ac;57][alC;57][bls;57][clB;57][bln;57][lgs;57][lCg;57][alw;57][bdl;57][lxg;57][abg;57][lvy;57][afl;57][dly;57][bgB;57][lqw;57][adl;56][blw;56][clq;56][cly;56][alt;56][dli;56][agg;56][bfl;56][lrg;56][lsy;56][blt;56][dlq;56][lpg;56][lvi;56][alp;56][lwi;56][lsB;56][dls;56][lxy;56][lsw;56][lqx;56][alu;56][loq;56][bnq;56][flg;56][ael;56][abB;56][clu;56][alz;56][lxB;56][blm;56][lCB;56][cli;56][bny;56][any;56][aqB;56][alm;56][lrB;56][ayi;56][lsu;56][byi;56][lpq;56][alx;56][lry;56][lpB;56][lny;56][flB;56][lgr;56][bel;56][luv;56][loy;56][log;56][luw;56][blo;56][cls;56][alh;56][lpy;56][bqi;55][lyj;55][apg;55][dlv;55][lsC;55][blz;55][dlu;55][arg;55][anq;55][alA;55][blx;55][alk;55][asg;55][luj;55][abn;55][lty;55][aby;55][lnq;55][lsi;55][fly;55][lvC;55][abq;55][ltg;55][loB;55][lou;55][blr;55][elg;55][ely;55][lnu;55][bsy;55][lru;55][luC;55][aoy;55][lCi;55][avg;55][uyg;55][vyg;55][blp;55][clv;55][lgj;55][lgt;55][blh;55][arB;54][abi;54][bCg;54][blk;54][dlx;54][asB;54][lmg;54][lxi;54][blA;54][ang;54][asy;54][aog;54][ary;54][apB;54][lri;54][lwj;54][lwC;54][ap y;54][flq;54][bqs;54][aqi;54][lpi;54][wyg;54][flv;54][dlw;54][lsv;54][dlj;54][elq;54][lvw;54][cl C;54][dln;54][low;54][aoq;54][bng;54][lCy;54][elB;54][blv;54][lmy;54][qvg;54][lpu;54][dlt;54][ryg;54][ans;54][lux;54][apq;54][uyB;54][ags;54][ltB;54][bug;54][dlo;54][lxC;53][aoB;53][cdl; 531[fli;53][clj;53][lnw;53][lvj;53][loi;53][avB;53][ar;53][vyB;53][lrw;53][anr;53][loC;53][clx; 53][dlr;53][buy;53][dfl;53][lnv;53][nuy;53][flw;53][lmB;53][anB;53][flu;53][bnu;53][pyg;53][n qv;53][lsx;53][clt;53][bCB;53][clo;53][bcq;53][bcy;53][avy;53][lqy;53][bwy;53][elu;53][dlC;53][buB;53][dlp;53][wyB;53][lwx;53][clr;53][bsg;53][ltu;53][nvy;53][cln;53][ltw;53][lqC;53][bq w;53][pqg;53][fls;53][lpC;53][qvB;53][aqv;53][bnC;53][cfl;53][bdg;53][bnB;53][lyz;53][ayj;53][ltv;53][byj;53][ryB;53][bqu;53][lmq;53][lmu;53][lrC;53][ari;52][anv;52][luz;52][nry;52][lpw; 52][bpq;52][ano;52][lrs;52][awy;52][api;52][dyg;52][lns;52][asi;52][xyg;52][nwy;52][qrg;52][d ny;52][pyB;52][bdq;52][abs;52][clp;52][bpy;52][bmq;52][aoi;52][els;52][bmg;52][eli;52][elv;52][bdn;52][lyh;52][bsB;52][bdy;52][qwg;52][ltC;52][lms;52][bcg;52][lqz;52][ngr;52][bdB;52][b ns;52][ani;52][flr;52][bpg;52][elw;52][qug;52][bqj;52][aCg;52][lmv;52][bfy;52][lps;52][lvx;52][clw;52][lyA;51][lyk;51][qsw;51][lnC;51][flx;51][lCj;51][flo;51][flC;51][lti;51][acy;51][awg;51][lwz;51][bmy;51][nqu;51][pqB;51][swy;51][dyB;51][amg;51][ltx;51][dsy;51][lov;51][lmw;51][luh;51][abC;51][del;51][anp;51][vyi;51][lnx;51][clm;51][dlm;51][nqw;51][lox;51][avi;51][cel;51][dlz;51][bni;51][lxj;51][bwg;51][bfg;51][luk;51][bty;51][uyi;51][uvy;51][lsj;51][bCi;51][lrx;51][luA;51][nxy;51][boy;51][quB;51][ady;51][adg;51][lgh;51][ouy;51][bry;51][lmi;51][bqt;51][wyi;51][ngs;51][afg;51][qrB;51][bfn;51][aps;51][lrv;51][amy;51][lng;51][xyB;51][clz;51][los;51][lst;51][lnr;51][dnq;51][auy;51][bmB;51][dlh;51][ruy;51][abj;51][bxy;51][axy;51][acg;51][bo q;51][aCB;51][adn;51][bcn;51][syg;51][lpx;51][elo;51][lpv;51][lgk;51][lqA;51][ryi;51][anj;51][bpB;51][puy;51][afy;51][npq;51][lmx;51][noq;51][qwB;51][bsC;51][dqs;51][lwh;51][bfq;51][lv z;51][aey;51][ngx;51][aqj;50][dsg;50][flp;50][dlk;50][qvi;50][lwA;50][bui;50][fln;50][nsy;50][b gr;50][agy;50][aqw;50][amB;50][bcB;50][suy;50][aug;50][dlA;50][elx;50][asC;50][btg;50][afn; 50][anC;50][bqy;50][npy;50][abp;50][lno;50][lgj;50][quv;50][aos;50][elr;50][aty;50][uwy;50][1 wk;50][awB;50][lgi;50][svy;50][clh;50][dvy;50][bfB;50][ars;50][qsg;50][qsv;50][bwB;50][lrj;50][atg;50][aCy;50][adB;50][lpj;50][brg;50][dry;50][acq;50][pyi;50][bqC;50][dqv;50][psy;50][bn t;50][bnw;50][dns;50][bnj;50][noy;50][byz;50][bmn;50][dpy;50][anu;50][afB;50][clA;50][dqg;50][bCy;50][ant;50][ayz;50][dvg;50][uvg;50][bsi;50][anw;50][pqu;50][lgB;50][liB;50][bqx;50][1 gh;50][lvh;50][lrt;50][lnp;50][flj;50][pqs;50][lnB;50][bdi;50][clk;50][lot;50][lmo;49][bsu;49][lv k;49][pqv;49][bey;49][bnr;49][beq;49][qxg;49][lvA;49][dsB;49][amq;49][asw;49][dyi;49][pgi;49][ljB;49][duy;49][cqv;49][lmr;49][auB;49][elt;49][asv;49][nyg;49][nuv;49][lgz;49][oyg;49][ab o;49][adq;49][acn;49][rwy;49][ogg;49][syB;49][lpt;49][elp;49][oqu;49][lmC;49][abc;49][abm;49][aoC;49][acB;49][axg;49][abu;49][lor;49][vwy;49][ads;49][lop;49][arC;49][cvy;49][qsu;49][q rw;49][lgA;49][btB;49][cuy;49][qru;49][pug;49][rsy;49][cyg;49][dgr;49][doy;49][buC;49][dxy; 49][lgk;49][dnv;49][bqz;49][dvB;49][bci;49][dqB;49][elC;49][bog;49][ayh;49][brB;49][dpq;49][pvy;49][abw;49][byh;49][aeq;49][abf;49][owy;49][elj;49][aeg;49][bvy;49][lsz;49][nsu;49][amn ;49][rug;49][ams;49][ari;49][bds;49][efl;49][atB;49][abr;49][fny;49][abt;49][cas;49][asB;49][uv B;49][qvw;49][lCz;49][loj;49][csy;49][oqB;49][aCi;49][cny;49][abd;49][qwi;49][lpr;49][vCg;49][bno;49][bnp;49][bmi;49][eln;49][swg;49][aqx;49][oyB;49][byA;49][lhB;49][lnt;49][byk;49][cnq;49][acs;49][ayk;49][aqC;49][ayA;49][xyi;49][dwy;49][sug;49][nru;49][bpi;49][lsh;48][pwy ;48][lni;48][lmp;48][drg;48][quw;48][apj;48][ngg;48][nyB;48][uxy;48][asu;48][aqu;48][agt;48][apC;48][dpg;48][qui;48][puB;48][qxB;48][avC;48][lkB;48][oqw;48][awi;48][nug;48][lzB;48][d oq;48][asj;48][lxz;48][qrs;48][dnr;48][dnu;48][nsw;48][flt;48][bCj;48][lAB;48][bqh;48][ltj;48][bwi;48][abe;48][bxg;48][boB;48][lsA;48][bcC;48][bcs;48][lsk;48][nvC;48][bqv;48][anz;48][cqr ;48][ruB;48][anx;48][apv;48][wyj;48][agj;48][adi;48][aen;48][pqw;48][adp;48][arj;48][axB;48][ami;48][fyg;48][cry;48][pxy;48][cqg;48][uyj;48][aop;48][beg;48][muy;48][dqx;48][lCh;48][apr; 48][fvy;48][bqk;48][pqx;48][flm;48][agi;48]j[bgA;48][aeB;48][nou;48][cqu;48][suB;48][aor;48][dsi;48][cyB;48][sxy;48][lmt;48][afq;48][afs;48][abz;48][nvg;48][rxy;48][amr;48][aci;48][bfi;48][mqv;48][vCB;47][bcu;47][bnz;47][nuw;47][lCA;47][dpB;47][avj;47][bnx;47][rCg;47][amo;47][bms;47][lCk;47][afi;47][aiB;47][apt;47][ado;47][vyj;47][drB;47][wxy;47][dqu;47][lij;47][ar w;47][lxh;47][pry;47][agB;47][nuB;47][syi;47][ogs;47][afo;47][lrz;47][cxy;47][bwC;47][oug;47][agh;47][swB;47][dng;47][adr;47][aco;47][qsx;47][ast;47][aqz;47][lnj;47][rvy;47][ovy;47][bd u;47][lxA;47][oxy;47][lpz;47][oqv;47][oqi;47][lxk;47][osy;47][agz;47][bdC;47][ajB;47][aru;47][art;47][vxy;47][acp;47][apw;47][ben;47][bgj;47][anh;47][oyi;47][nsC;47][bmC;47][mvy;47][d ps;47][afp;47][dsv;47][dno;47][aov;47][aow;47][aou;47][acr;47][lmn;47][aoj;47][bpC;47][fqv;47][cpq;47][bfC;47][bpu;47][dug;47][nwg;47][bsw;47][nqB;47][uwg;47][cpy;47][dgi;47][ouB;47][aot;47][apu;47][fnq;47][ory;47][rwg;47][dgw;47][bxB;47][cnu;47][bti;47][coq;47][myg;47][m ry;47][buj;47][qsi;47][bvg;47][lrh;47][ank;47][dvi;47][btC;47][swC;47][nsv;47][npu;47][flz;47][aes;47][qrx;47][fyB;47][dnp;47][abh;47][nyi;47][aui;47][dsu;47][liz;47][afr;47][dsw;47][fnv;47][fwy;47][coy;47][bps;47][dog;47][elm;47][sCg;47][bgi;47][pqr;47][bri;47][agk;47][agA;47][q vy;47][bgh;47][amp;47][qrv;47][nrg;47][mgr;47][amv;47][lrk;47][ati;47][atv;47][lih;47][eqv;47][abk;47][abA;47][eny;47][afv;47][auv;47][ery;47][anA;47][bmu;47][qvx;47][bgB;47][boC;47][qvj;47][pvg;47][lrA;47][oqx;47][adv;47][biB;47][dnx;47][boi;47][abx;47][beB;47][bnv;47][aqh; 47][nrC;47][bnh;47][nvB;47][cqx;47][lph;47][dnB;47][cqB;47][lmj;47][nuC;47][duB;47][qwy;46][bnk;46][fry;46][twy;46][mxy;46][bgz;46][uwB;46][mny;46][qux;46][agk;46][rCB;46][brC;46][aqA;46][lpA;46][abv;46][ahB;46][cyi;46][mqs;46][suw;46][suC;46][msy;46][psg;46][uvC;46][ryj;46][dnw;46][uCg;46][lpk;46][bjB;46][tvy;46][nrw;46][bdj;46][euy;46][cvg;46][cug;46][aer ;46][svC;46][nrs;46][vCy;46][asx;46][uvi;46][elz;46][bgA;46][qtv;46][rCy;46][bgk;46][avw;46][evy;46][loz;46][akB;46][doB;46][pCg;46][bst;46][bnA;46][drs;46][csg;46][pui;46][aei;46][dos; 46][bfs;46][dsC;46][buw;46][aep;46][loh;46][pyj;46][suv;46][cns;46][mwy;46][dfy;46][nty;46][fuy;46][acv;46][flh;46][nCy;46][opy;46][eyg;46][nwB;46][fsy;46][liA;46][dyj;46][dwg;46][aeo; 46][qxi;46][azB;46][asz;46][bdp;46][dxg;46][arv;46][qvC;46][dri;46][dpi;46][flA;46][ngi;46][w Cg;46][loA;46][myB;46][cnv;46][psu;46][qwx;46][qwj;46][mqu;46][fpy;46][rui;46][lok;46][csu ;46][flk;46][rwB;46][axi;46][aAB;46][sCB;46][bou;46][tuy;46][nui;46][opq;46][bsj;46][uCy;46][nwC;46][bcj;46][nyj;46][swi;46][dfn;46][vwg;46][bvB;46][lik;46][egr;46][aox;45][aev;45][arz ;45][quy;45][apz;45][bhB;45][pgj;45][rsC;45][sui;45][pvB;45][bxC;45][pwg;45][bdf;45][bdt;45][rsg;45][sCy;45][ash;45][xyj;45][wCy;45][nvw;45][vCi;45][dni;45][fvg;45][ltz;45][epy;45][dsx ;45][dty;45][noC;45][esy;45][nrB;45][foy;45][uCB;45][ogr;45][aCj;45][ewy;45][awj;45][ask;45][bkB;45][nvi;45][qry;45][bcd;45][duv;45][now;45][asA;45][Cyg;45][wyz;45][cdy;45][elh;45][oui;45][fxy;45][bCz;45][ruC;45][bzB;45][nux;45][dxB;45][rty;45][cgi;45][brs;45][ouC;45][bru; 45][bwj;45][psB;45][arx;45][bdr;45][enq;45][cuB;45][qrC;45][dwB;45][dmy;45][crg;45][svg;45][apx;45][npC;45][bcp;45][btu;45][bcm;45][ngt;45][mvg;45][psC;45][aij;45][bdo;45][bdm;45][a ph;45][mnq;45][egs;45][cvB;45][elA;45][elk;45][bdw;45][pqy;45][pCB;45][eyB;45][arh;45][m qx;45][fnr;45][ntv;45][bct;45][quj;45][dnC;45][bei;45][nwi;45][bAB;45][cds;45][dpu;45][dvC;45][doi;45][pCy;45][pty;45][rsu;45][fqs;45][osu;45][csC;45][uyz;45][osC;45][moq;45][tyg;45][m oy;45][ngy;45][cwy;45][awC;45][mpy;45][nuj;45][cnr;45][txy;45][mpq;45][apA;45][vyz;45][ar A;45][mug;45][adj;45][rCi;45][nrv;45][ark;45][epq;45][avz;45][mqw;45][bos;45][lnz;45][bpj;45][ngj;45][acj;45][qtw;45][puC;45][fyi;45][apk;45][aiz;45][aoz;45][bcf;45][aoh;45][qrj;45][beu; 45][beC;45][lth;45][cng;45][qsC;45][fqr;45][bco;45][vwB;45][mrg;45][acC;45][csB;45][bxi;45][wCB;45][oCg;45][fnu;45][lmz;45][oCy;44][fqg;44][dfs;44][nsx;44][amw;44].

The combinations with the highest “at least 2 phage % coverage” are provided herein below. The phage combinations are ordered in descending performance grade.

Thus, for example, in the case of [alg;17], when 395-D2-3-1, KP10-11 and CT-227-KP4M1-11 are used in a combination, 17% of the bacterial strains analyzed were targeted by at least 2 of the 3 phages.

[abl;33][aby;31][abv;31][abd;30][bdv;30][abr;30][bly;30][blp;30][alu;30][alp;30][blv;30][adl;28][alw;28][bdl;28][buv;28][adw;28][abp;28][aly;28][auw;28][abw;28][abc;28][alv;28][abo;28][ad v;28][blw;28][abq;28][adu;28][abu;28][bdw;28][adx;28][lpv;28][blr;28][abs;28][alr;28][ady;28][abm;28][acl;28][bfv;28][auy;28][bru;28][bdp;28][brw;28][bpv;28][bqv;28][aqu;28][brv;28][bdr ;28][bdy;28][blu;28][bvy;28][bcv;27][blq;27][abC;27][bpw;27][bvw;27][acu;27][abx;27][bcl;27][lpr;27][bev;27][buw;27][arv;27][bvx;27][awy;27][dlp;27][acd;27][bvC;27][abn;27][acw;27][a wC;27][adr;27][avy;27][auC;27][lwy;27][bnv;27][dlw;27][lry;26][bdx;26][buy;26][anw;26][aq w;26][apu;26][bmv;26][bcw;26][ads;26][acv;26][bsw;26][bdo;26][aux;26][alq;26][bry;26][bls;26][lvy;26][bqy;26][bov;26][awx;26][lpy;26][lqy;26][dly;26][abf;26][dlv;26][dlr;26][lpu;26][bdu ;26][auv;26][blC;26][alx;26][avw;26][bds;26][alC;26][acx;26][bcr;26][arw;26][bCy;26][abt;26][brx;26][adq;26][bow;26][ado;26][btv;26][bn;26][aln;26][bpr;26][dlu;26][blx;26][aru;26][bou;26][adp;26][bpu;26][bwy;26][brC;26][luv;26][anu;26][ber;25][bwx;25][afu;25][bcd;25][cwx;25][bpy;25][lpw;25][lrv;25][lnr;25][amu;25][alo;25][adC;25][apv;25][cvw;25][pvw;25][ary;25][du w;25][bgr;25][als;25][adn;25][lvw;25][cuw;25][bcp;25][luy;25][apw;25][apr;25][blo;25][luw;25][bnw;25][axy;25][clw;25][bsv;25][axC;25][ruv;25][aew;25][lrw;25][asw;25][pqy;25][agy;25][p rw;25][lps;25][aqv;25][bpx;25][cqy;25][crw;25][bmw;25][abe;25][lqw;25][bor;25][bux;25][aou; 25][adf;25][aeu;24][lnw;24][rvw;24][bfr;24][alm;24][lgr;24][aow;24][dqy;24][brs;24][lnv;24][1 np;24][dvw;24][apy;24][lru;24][acr;24][clp;24][bcu;24][aqx;24][bcx;24][atu;24][avC;24][quy;24][bnp;24][drv;24][asu;24][qry;24][bop;24][cdl;24][adt;24][cpw;24][clv;24][dln;24][cdw;24][bp C;24][bxy;24][apx;24][bwC;24][lxy;24][acC;24][clr;24][prv;24][dpr;24][dlq;24][bqu;24][ade;24][lrC;24][bfu;24][acn;24][bdn;24][bew;24][adm;24][crv;24][lCy;24][uvw;24][dlo;24][bvi;24][by z;24][drw;24][qsy;24][lqv;24][cdu;24][bnr;24][cpv;24][bvk;24][acs;24][bvh;24][bdq;24][bvg;24][ngy;24][anv;24][acy;24][cpr;24][bvA;24][bps;24][lqu;24][bvB;24][arx;24][bvj;24][bfw;24][in u;24][lpq;24][uwx;24][acp;24][dpv;24][qwy;24][cly;23][luC;23][bdC;23][bpq;23][duv;23][aov; 23][bcs;23][opr;23][lpx;23][anp;23][anx;23][bdm;23][bmr;23][bco;23][blm;23][amw;23][brt;23][avx;23][csw;23][box;23][dux;23][qvy;23][dwy;23][dru;23][dls;23][afl;23][afw;23][asy;23][dv y;23][cow;23][rvy;23][bel;23][acq;23][dC;23][lor;23][apC;23][bmp;23][ogy;23][pwy;23][bqw; 23][ruw;23][pru;23][ael;23][lsy;23][lrs;23][dpw;23][puv;23][lmr;23][rwy;23][boC;23][rvx;23][a ui;23][abh;23][arC;23][bsu;23][pwx;23][ars;23][npr;23][amv;23][abz;23][npv;23][aug;23][fqy;23][cdv;23][btw;23][abk;23][aex;23][abA;23][qxy;23][abj;23][lop;23][bnx;23][loy;23][dlx;23][n vw;23][bfl;23][lwC;23][vwx;23][bdf;23][apq;23][abB;23][abi;23][aqr;23][abg;23][qCy;23][lsu; 23][beo;23][cdy;22][dCy;22][lvC;22][auk;22][adg;22][auA;22][lvx;22][ace;22][vwy;22][lny;22][opw;22][dop;22][dpu;22][dnw;22][auB;22][bmu;22][bfp;22][bep;22][cln;22][aCy;22][boq;22][buC;22][lou;22][auj;22][npw;22][auh;22][aop;22][auz;22][cnw;22][puw;22][aor;22][any;22][dw x;22][rvC;22][lpC;22][mqy;22][ovw;22][boy;22][lux;22][duy;22][asv;22][atw;22][rwx;22][lov; 22][cCy;22][bsx;22][bex;22][bqx;22][bcn;22][adB;22][lmp;22][uwy;22][lnq;22][dow;22][lnx;22][frv;22][lrx;22][cwy;22][clu;22][orw;22][cfw;22][mvw;22][lno;22][amx;22][lwx;22][adi;22][cu v;22][prx;22][aco;22][egy;22][prs;22][pvx;22][ouv;22][dgw;22][mpr;22][bmx;22][erv;22][lmn; 22][bxC;22][alt;22][lsv;22][ctw;22][asx;22][uvx;22][aps;22][dou;22][cdr;22][prC;22][dor;22][af v;22][blt;22][nrw;22][cdq;22][vCy;22][bnu;22][cdp;22][adj;22][bde;22][qrv;22][orv;22][lsw;22][mrv;22][nrv;22][cvx;22][anr;22][cpx;22][pux;22][low;22][cwC;22][afy;22][rsw;22][bes;22][ce w;22][oru;22][wCy;22][adz;22][pry;22][bdt;22][lqC;22][lnC;22][btu;22][epr;22][awB;22][lns;22][awg;22][lmv;21][bfx;21][dvx;21][nuw;21][pwC;21][adk;21][dpy;21][adA;21][bcy;21][ruy;21][dry;21][cmw;21][fly;21][dlm;21][bny;21][awi;21][clq;21][uvy;21][afq;21][adh;21][amr;21][bn o;21][vwC;21][acm;21][ouw;21][cop;21][pvy;21][ovx;21][ags;21][acf;21][svw;21][mpv;21][fln; 21][anC;21][cqw;21][brj;21][puy;21][lmy;21][elr;21][bey;21][mpw;21][dsu;21][opv;21][bpt;21][quw;21][del;21][cls;21][dfu;21][amy;21][flp;21][bcf;21][pCy;21][uwC;21][aer;21][amp;21][uC y;21][lmu;21][bwg;21][dov;21][bos;21][bwB;21][qty;21][afx;21][fuw;21][psw;21][aev;21][beu; 21][dsw;21][afr;21][cux;21][cdC;21][npx;21][asC;21][clx;21][evw;21][rCy;21][brB;21][cnp;21][bce;21][euw;21][bwi;21][anq;21][drs;21][los;21][mwx;21][lnB;21][opu;21][pvC;21][dfl;21][at x;21][brg;21][bfo;21][lmw;21][muw;21][wxy;21][dqu;21][brz;21][dwC;21][bcC;21][clo;21][ao x;21][pqw;21][bcm;21][lng;21][cdn;21][elp;21][orx;21][aoy;21][epw;21][rtv;21][rux;20][suw;20][cor;20][awh;20][btx;20][brk;20][cdm;20][lqx;20][dnu;20][bri;20][crx;20][pqr;20][bmy;20][b sC;20][brA;20][awA;20][awz;20][aoC;20][cnv;20][drx;20][cdo;20][brh;20][cru;20][awk;20][bq C;20][dfw;20][fvw;20][awj;20][afC;20][cnr;20][cwB;20][flw;20][dnv;20][flu;20][duC;20][owx; 20][drC;20][cdf;20][dqC;20][rwC;20][amC;20][dew;20][fru;20][bns;20][crs;20][aoq;20][mop;20][elw;20][bmo;20][lpt;20][dmu;20][act;20][eln;20][dtw;20][rsv;20][nvx;20][fpr;20][cdt;20][rsu ;20][npu;20][ely;20][epv;20][cdx;20][uxy;20][bct;20][aep;20][swx;20][bwj;20][wxC;20][bms;20][cwg;20][ano;20][opC;20][bwh;20][erw;20][cpy;20][lmq;20][mrw;20][lgs;20][elv;20][lrt;20][elu;20][prt;20][bwz;20][dtu;20][dmw;20][aey;20][bwk;20][dpq;20][dpC;20][clC;20][dnr;20][bw A;20][cde;20][vxy;20][frw;20][lni;20][bsy;20][deu;20][cpu;20][qvw;20][cov;20][bdg;20][fuv;20][cwj;20][dnp;20][rxy;20][acB;20][cwh;20][fpu;20][dpx;20][nop;20][dqv;20][cps;20][cwA;20][lsx;20][bfy;20][suy;20][afp;20][qwC;20][nwx;20][eux;20][dmr;20][fCy;20][lsC;20][cwk;20][b qs;20][ewx;20][cry;20][nps;20][cds;20][loq;20][qru;20][uxC;20][bdB;20][nwy;20][cwi;20][cwz; 20][psu;20][ans;19][quv;19][ltw;19][lnt;19][eru;19][dvC;19][dps;19][atv;19][fpw;19][flr;19][eC y;19][bfs;19][aty;19][aqC;19][lox;19][sCy;19][mpu;19][dmv;19][opx;19][bdi;19][qrw;19][dxy;19][cuy;19][puC;19][cnu;19][cny;19][fux;19][acg;19][pqu;19][bcq;19][cfu;19][ruC;19][qwx;19][loC;19][nru;19][xCy;19][fwx;19][bpj;19][aes;19][bdz;19][dey;19][bdj;19][dsv;19][quC;19][eov; 19][fop;19][amq;19][lms;19][bpz;19][mvx;19][fpv;19][bot;19][ltu;19][nuv;19][flq;19][bpg;19][f lv;19][cqC;19][dfr;19][bpB;19][lxC;19][doC;19][amn;19][ams;19][tvw;19][bpi;19][ogr;19][afn; 19][pqv;19][lmC;19][cdg;19][cou;19][dmC;19][opq;19][bdk;19][csu;19][bpA;19][bnq;19][bpk; 19][bdA;19][bdh;19][ben;19][cmu;19][owC;19][psv;19][csv;19][bnC;19][cmv;19][bph;19][aen; 19][cep;19][der;19][cvy;19][cmp;19][doy;19][opy;19][oCy;19][dmp;19][owy;19][aci;19][clm;19][cdB;19][dgr;19][evx;19][dep;19][dny;19][mru;19][cpC;19][dfv;19][eop;19][pxy;19][ptw;19][dwg;18][lmo;18][pqC;18][qyk;18][cel;18][nCy;18][dsy;18][swy;18][fvx;18][eqC;18][atC;18][q yh;18][qux;18][cfr;18][ovC;18][uvC;18][qyg;18][orC;18][cfl;18][qyz;18][bui;18][csx;18][qyA;18][qsu;18][nrx;18][ors;18][qyj;18][muv;18][agt;18][rtw;18][ovy;18][nov;18][qyB;18][nux;18][n ow;18][tuw;18][cfv;18][ceu;18][bug;18][ayi;18][cev;18][bxB;18][cvC;18][deq;18][buj;18][buB; 18][psx;18][cuC;18][ltv;18][nwC;18][euv;18][dwi;18][nuy;18][afs;18][for;18][cpq;18][cqu;18][buz;18][acj;18][mnw;18][buh;18][mov;18][eow;18][fuy;18][bxg;18][art;18][dfq;18][cmy;18][a mo;18][cdi;18][elq;18][dfy;18][aeq;18][buA;18][rsx;18][buk;18][dfp;18][bxi;18][dwB;18][dnC; 18][dfC;18][mow;18][dxC;18][pwB;18][prj;18][mwy;18][dug;18][pxC;18][dmy;18][suv;18][rtu ;18][cer;18][nor;18][epu;18][nqw;18][bty;18][aeo;18][fgC;18][tux;18][npy;18][dnx;18][sux;18][ach;18][axi;18][nuC;18][mux;18][rsy;18][aeC;18][cox;18][doq;18][mpx;18][acz;18][oux;18][ax g;18][cfp;18][cdj;18][acA;18][ack;18][pwg;18][lmx;18][axB;18][svx;18][ewy;18][dwj;18][mpy; 18][bxj;18][qvC;18][rvj;18][bxA;18][dwz;18][bxk;18][cdz;18][nvy;18][epx;18][dsC;18][ort;18][els;18][dev;18][mqC;18][fov;18][nqu;18][mCy;18][dox;18][bxh;18][frs;18][oqv;18][nsw;18][a os;18][prB;18][fwy;18][cmr;18][bcB;18][qvx;18][bxz;18][oqw;18][prz;18][uwg;18][prg;18][fry; 18][flC;18][eor;18][apt;18][cfy;18][ory;18][crC;18][cpt;18][aef;18][dno;18][cdA;17][elC;17][bl k;17][dsx;17][dit;17][ptv;17][cdk;17][axk;17][blA;17][pwi;17][cno;17][csy;17][fpy;17][axj;17][cns;17][blh;17][flm;17][blB;17][bli;17][fpx;17][deo;17][axz;17][afo;17][fqu;17][rvB;17][lty;17][blj;17][lnA;17][esw;17][lnj;17][dwk;17][blz;17][blg;17][qrs;17][axh;17][dmn;17][beg;17][bfn; 17][uwB;17][erx;17][frx;17][suC;17][dnq;17][dwh;17][fnp;17][uwi;17][fls;17][vxC;17][esv;17][bfq;17][dui;17][nry;17][rg;17][twx;17][axA;17][ceo;17][beq;17][dwA;17][dex;17][fwC;17][nr C;17][rvz;17][bmn;17][cmx;17][duB;17][vwB;17][cdh;17][vwg;17][dmo;17][mrs;17][den;17][a fm;17][fnw;17][enw;17][enp;17][nqC;17][lnk;17][pri;17][cex;17][prk;17][rvA;17][efw;17][vwi; 17][ctu;17][rvk;17][rvh;17][prA;17][rvi;17][osv;17][fsu;17][cey;17][msw;17][prh;17][lnz;17][in h;17][mps;17][dmx;17][alg;17][cfx;17][ali;17][alj;17][psC;17][elm;17][nrs;17][qrx;17][cnq;17][evy;17][bmC;17][bfC;17][alA;17][mvy;17][alh;17][alk;17][dqs;17][alz;17][rsC;17][cxy;17][sxy ;17][dqx;17][alB;17][mor;17][qrC;17][fqw;17][qsC;17][fnu;17][mrx;17][dpt;17][duz;17][avB;17][duj;17][lrj;17][pwz;17][lpB;17][avg;17][crt;17][avz;17][cms;17][lpj;17][dtv;17][cnx;17][euy; 17][env;17][swC;17][nou;17][ers;17][elx;17][dmq;17][msu;17][avA;17][avj;17][dem;17][ouy;17][avk;17][coC;17][avi;17][cqr;17][lpz;17][cnC;17][dns;17][flo;17][pwj;17][coy;17][lpg;17][av h;17][mry;17][dtC;17][oqu;17][fvy;17][ouC;17][mnp;17][cfC;17][aem;17][fou;17][nsu;17][bci; 17][efu;17][mwC;17][def;17][emw;16][esu;16][duA;16][fow;16][cef;16][pwh;16][fpC;16][rtx;16][lpi;16][duk;16][nsy;16][ptu;16][des;16][pwk;16][opt;16][lrz;16][rwg;16][lpA;16][deC;16][ps y;16][tCy;16][cmn;16][arj;16][lph;16][pwA;16][eou;16][duh;16][ctv;16][eps;16][mnu;16][det;16][lpk;16][lrg;16][lrB;16][dfx;16].

The percent of host bacterial strains that are infected by three phages of a phage combination are provided. This trait is referred to as “at least 3 phage % coverage”. The phage combinations are ordered in descending performance grade.

The phage combinations are ordered in descending performance grade.

Thus, for example, in the case of [abu;18], when 395-D2-3-1, KP10395-D2-3-2 and KP10-84 are used in a combination, 18% of the bacterial strains analyzed were targeted by each of the 3 phage.

[abu;18][abw;17][brv;17][bvw;16][abv;16][abd;16][adu;16][acw;16][bpr;15][abx;15][ad w;15][cdw;15][auw;15][bpv;15][acd;15][abr;14][bcw;14][bvx;14][abp;14][bdw;14][brw;14][abc ;14][bpw;14][apw;14][buw;14][aux;14][bwx;14][avw;14][bdu;14][bcd;13][acu;13][aru;13][awx; 13][cpw;13][blr;13][auv;13][adp;13][buv;13][arw;13][brx;13][apu;13][bdr;13][bov;12][dpw;12][duw;12][prv;12][bpu;12][bux;12][bru;12][avx;12][bor;12][cuw;12][bpx;12][acp;12][bdv;12][ab 1;12][adr;12][blv;12][bcv;12][adC;12][apr;12][prw;12][cdu;12][bcr;12][bdp;12][bcp;12][puw;12][cdp;12][rvw;12][bcu;12][crw;12][adl;12][bop;12][drw;11][vwx;11][arv;11][acr;11][arx;11][al u;11][abe;11][blw;11][lpr;11][cvw;11][blp;11][cdr;11][alw;11][pvw;11][adv;11][adx;11][ruw;11][ade;11][qCy;11][dwC;11][apx;11][bde;11][apv;11][dvw;11][alr;11][pru;11][uwx;11][dew;11][dwx;11][uvw;11][cpu;11][bdx;11][pwx;11][dpu;11][deC;11][duC;11][bsv;11][lpw;11][orv;11][rvx;11][cdC;11][opr;11][cpr;11][lrv;11][auC;11][dpr;11][cde;11][cew;11][rwx;11][bd;11][lnp; 11][aeu;11][dru;11][prx;11][blu;11][afu;10][agy;10][aqC;10][rux;10][cwx;10][bew;10][acy;10][cru;10][adm;10][dmw;10][cwC;10][ady;10][blx;10][acx;10][lrw;10][bdC;10][abC;10][bow;10][acv;10][beu;10][bcx;10][opv;10][cdx;10][abo;10][aew;10][auy;10][pvx;10][bcy;10][clw;10][cw y;10][afw;10][bln;10][deu;10][cmw;10][aln;10][alp;10][amw;10][aeC;10][beC;10][awC;10][adq ;10][acC;10][alx;10][ruv;10][cdy;10][drv;10][abf;10][cdv;10][aCy;10][bep;10][aby;10][ewC;10][uvx;10][acl;9][aep;9][brs;9][ceC;9][box;9][duv;9][ace;9][opw;9][bvy;9][bce;9][cr;9][adf;9][lr u;9][puv;9][lop;9][cdl;9][cdm;9][pux;9][dpx;9][blo;9][dlw;9][dux;9][dqC;9][bev;9][dlr;9][bwy; 9][epw;9][lwx;9][alv;9][lor;9][awy;9][euC;9][dpC;9][drx;9][amu;9][afp;9][lvw;9][luw;9][dlu;9][euw;9][bwC;9][rsv;9][acf;9][acm;9][lrx;9][abn;9][dep;9][dlp;9][bfw;9][cux;9][cfw;9][bry;9][ab s;9][dfw;9][duy;9][clp;9][dqy;9][dpv;9][bpy;9][dmp;9][dmu;9][mpw;9][bc1;9][cpv;9][buC;9][fp w;9][cuC;9][aor;9][asu;9][aow;9][ovw;9][dCy;9][aop;9][bnp;9][uwC;9][orw;9][adn;9][cdf;9][cu y;9][lpx;9][bcC;9][lpu;9][afx;9][lny;9][cuv;9][ceu;9][aou;9][lnr;9][cpy;9][dvx;9][bfp;9][amp;9][fuw;9][abq;9][cep;9][bdy;9][epu;9][bex;9][aev;9][apC;9][clr;9][dwy;9][bnr;9][bco;9][dfu;9][crx ;9][aqu;9][anu;9][bps;9][bdm;9][cow;9][bdf;9][psv;8][pwC;8][abm;8][acq;8][bfu;8][cqw;8][evw ;8][ewx;8][dev;8][cmp;8][bsu;8][bmp;8][cnw;8][lpv;8][opx;8][bmw;8][aov;8][dqu;8][aox;8][pry ;8][muw;8][der;8][bdo;8][ber;8][bou;8][drC;8][cpC;8][apy;8][cpx;8][lqy;8][bdq;8][dln;8][lnw;8][bnu;8][afr;8][bnv;8][cdq;8][lux;8][bsx;8][cop;8][alC;8][asv;8][bfx;8][fpr;8][dfp;8][dlC;8][bfr; 8][arC;8][npw;8][anp;8][aco;8][dmC;8][lvx;8][cor;8][cfp;8][buy;8][dpy;8][lmp;8][aly;8][bcq;8][cvx;8][epC;8][bnw;8][erw;8][ops;8][cry;8][dex;8][aer;8][bqw;8][aex;8][bqy;8][clu;8][npr;8][orx ;8][ado;8][opu;8][dow;8][anw;8][be;8][osv;8][dry;8][cln;81 [aaw;8][bcm;81[dem;81[prs;8][owx; 8][ary;8][cer;8][dnp;8][bmu;8][lnu;8][uwy;8][crC;8][dgw;8][fwx;8][fpu;8][bpC;8][bos;8][dlm;8][bcf;8][bdn;8][bmr;8][cdn;8][cfu;8][lmw;8][svx;8][puC;8][cdo;8][dxC;8][quy;7][bmv;7][oru;7][euv;7][cvy;7][mpu;7][cmu;7][ael;7][cqy;7][anr;7][lpC;7][blm;7][fux;7][lov;7][pwy;7][mwC;7][alm;7][rwC;7][aem;7][mrw;7][cnp;7][suv;7][lpy;7][dmy;7][low;7][epx;7][exC;7][dor;7][emw;7][bvC;7][ovx;7][quC;7][ouw;7][elp;7][epr;7][frw;7][acn;7][ors;7][evC;7][bef;7][dvC;7][dop;7][mpr;7][lmn;7][eru;7][dnu;7][epv;7][muC;7][dgr;7][dlx;7][cCy;7][fpx;7][avC;7][brC;7][cqu;7][a rs;7][dfr;7][bqu;7][dfx;7][ory;7][amr;7][npu;7][afv;7][aef;7][qwy;7][oux;7][dnw;7][asw;7][bem; 7][asx;7][cmC;7][atu;7][dmr;7][eux;7][aps;7][mnp;7][bxy;7][giB;7][afl;7][boy;7][wxC;7][bcn;7][erC;7][cov;7][ruC;7][del;7][dly;7][cev;7][fru;7][cex;7][mpC;7][puy;7][axC;7][flr;7][cqC;7][ae y;7][bly;7][amC;7][cmr;7][bxC;7][uCy;7][elw;7][bfv;7][fmw;7][bsw;7][cly;7][rvy;7][apt;7][lnv; 7][bfl;7][bnx;7][qwC;7][nuw;7][afm;7][bqC;7][dey;7][vwy;7][alq;7][emu;7][def;7][prC;7][emC ;7][quw;7][alo;7][aqr;7][evx;7][pvy;7][lnC;7][cqr;7][lCy;7][atw;7][erx;7][fvw;7][dfC;7][clm;7][lnx;7][dfm;7][fuC;7][bsy;7][luv;7][cxC;7][cfr;7][axy;7][vwC;7][flp;7][npC;7][frx;7][cey;7][dlv; 7][ruy;7][rsx;7][opy;7][cem;7][npy;7][bgr;7][lry;7][wCy;7][lno;7][avy;7][amx;7][svw;7][rs;7][muy;7][epy;7][dmx;7][bls;7][efu;7][cou;7][elC;7][apq;7][dmq;7][uvC;7][fmp;7][cxy;7][mru;7][aeq;7][efp;7][eCy;7][anx;7][rxC;7][mvw;7][sux;7][erv;7][eln;7][afC;7][psw;7][bpq;7][fpv;7][ef w;7][amy;7][emp;7][nop;7][enp;7][uxC;7][dnC;7][cC;7][dov;7][fmu;7][rsu;7][art;7][rwy;7][pC y;7][lwC;7][amv;7][nwx;7][mCy;7][luy;6][mpy;6][clx;6][cfx;6][mwy;6][bmx;6][pxC;6][eqC;6][cmy;6][dnr;6][als;6][ben;6][enw;6][qry;6][dox;6][aen;6][dmv;6][ewy;6][nrv;6][coy;6][mwx;6][a dt;6][cvC;6][ouv;6][dou;6][euy;6][bqv;6][clv;6][psu;6][mux;6][blC;6][qrC;6][suw;6][cnu;6][cpq ;6][den;6][bmC;6][nrw;6][mpx;6][anv;6][qrw;6][tuw;6][bey;6][flw;6][nru;6][ce;6][psx;6][aos;6][lmu;6][cen;6][lmr;6][elu;6][flu;6][lox;6][dpq;6][elr;6][abt;6][lqC;6][mqu;6][flx;6][pqC;6][dfl; 6][cef;6][bcs;6][muv;6][drt;6][bny;6][ptu;6][eop;6][elx;6][bmn;6][fwC;6][cox;6][aoy;6][svy;6][uvy;6][equ;6][amn;6][osw;6][ads;6][wxy;6][dfy;6][pqy;6][bfm;6][aft;6][dmn;6][lsv;6][psy;6][e mv;6][afy;6][lns;6][lmy;6][pqr;6][qru;6][vxC;6][acs;6][uxy;6][bfC;6][mqy;6][bpt;6][enC;6][dny ;6][nor;6][beq;6][fuv;6][dlq;6][nux;6][rsw;6][deq;6][anC;6][npv;6][ptw;6][bfy;6][bno;6][act;6][f vx;6][any;6][pxy;6][ceq;6][dxy;6][mpv;6][pqw;6][amq;6][efC;6][bCy;6][bdt;6][dfv;6][ftu;6][fu y;6][bqx;6][enu;6][dtu;6][beo;6][csv;6][efx;6][mqw;6][cnC;6][luC;6][pvC;6][lrC;6][cmn;6][eor; 6][cfC;6][dvy;6][lps;6][prt;6][brt;6][mnw;6][lwy;6][lmC;6][npx;6][aeo;6][nCy;6][bmy;6][cnr;6][frv;6][csw;6][cfy;6][eqw;6][mrx;6][btw;6][osu;6][swx;6][cnx;6][lnq;6][bmq;6][atv;6][emr;6][c mv;6][clo;6][aqx;6][nrx;6][dpt;6][dtw;6][ovy;6][btu;6][qvw;6][cfm;6][dqx;6][mrv;6][cps;6][nv w;6][cds;6][mqC;6][bds;6][rCy;6][pqu;6][eow;6][cmq;6][cqv;6][bfo;5][vxy;5][rvC;5][bnC;5][cf v;5][fqu;5][mor;5][bfq;5][cqx;5][ouy;5][fwy;5][fpC;5][ery;5][lmx;5][osy;5][elv;5][rtu;5][nuv;5][mpq;5][dnx;5][cmx;5][lou;5][dsw;5][dsu;5][fmx;5]1[btv;5][av;5]1[afo;5][dlo;5][rtw;5][dft;5][ef m;5][fow;5][cfl;5][rxy;5][fpy;5][deo;5][owy;5][efv;5][nwC;5][csu;5][lpq;5][egy;5][bmo;5][blq; 5][cny;5][osx;5][crs;5][ftw;5][dsv;5][nry;5][emx;5][mny;5][qux;5][emy;5][fmC;5][rsy;5][ctw;5][doy;5][qvy;5][dps;5][mrC;5][eny;5][mow;5][lmv;5][fxC;5][cos;5][mnu;5][elm;5][fmv;5][bft;5][egr;5][drs;5][mxC;5][fop;5][fmr;5][lxC;5][mop;5][ctu;5][dfq;5][fqy;5][qwx;5][nuy;5][qxC;5][e fr;5][mgr;5][cdt;5][epq;5][eqx;5][lgr;5][ptv;5][for;5][cft;5][lsw;5][tuv;5][loy;5][dqv;5][mry;5][t vw;5][afs;5][elo;5][afq;5][mnC;5][ceo;5][nvx;5][now;5][dmo;5][eqv;5][enx;5][bfs;5][emn;5][qu v;5][enr;5][dty;5][fpt;5][cmo;5][qrv;5][nqC;5][lvy;5][pqv;5][lmo;5][bns;5][lqu;5][exy;5][mvx;5][nwy;5][eou;5][pqx;5][frC;5][los;5][asy;5][dnv;5][amo;5][dlt;5][flv;5][cfq;5][cpt;5][twx;5][qrx; 5][atx;5][fqw;5][mnr;5][fgC;5][csy;5][nuC;5][crt;5][ely;5][ngy;5][lsx;5][emq;5][npq;5][clq;5][mvC;5][frt;5][fox;5][eox;5][btx;5][dfo;5][cnv;5][ans;5][fou;5][fsw;5][eoC;5][qvC;5][eov;5][efl; 5][dsx;5][xCy;5][flm;5][dos;5][ano;5][bst;5][evy;5][aty;5][lsu;5][doC;5][eps;5][ast;5][fxy;5][fry ;5][dtC;5][alt;5][fps;5][lpt;5][cfo;5][nsv;5][aes;5][bfn;5][bet;5][nrs;5][ptx;5][fnp;5][opt;5][dng;5][lrt;5][npt;5][aoC;5][afn;5][mxy;5][oxy;5][fty;5][rtv;5][fsu;5][nov;5][anq;5][nxy;4][cty;4][cno; 4][moy;4][opC;4][lsy;4][qxy;4][nps;4][blt;4][pty;4][fvC;4][flC;4][dtx;4][owC;4][dsy;4][fqr;4][e mo;4][mqx;4][bty;4][fpq;4][mnv;4][atC;4][lqw;4][flo;4][bnq;4][efy;4][dfn;4][vCy;4][nox;4][lvC ;4][noy;4][dtv;4][qvx;4][ant;4][fmt;4][aot;4][lmq;4][pst;4][mou;4][mvy;4][fsv;4][fCy;4][aet;4][nvy;4][orC;4][coC;4][ngB;4][mnx;4][mqv;4][stu;4][tvx;4][cfs;4][tux;4][esu;4][rtx;4][fmy;4][bes ;4][tuy;4][fln;4][fnu;4][amt;4][nou;4][stw;4][ost;4][boC;4][fqx;4][rty;4][ers;4][ftv;4][nrt;4][dmt; 4][mov;4][ouC;4][fly;4][dfs;4][fov;4][mtu;4][nrC;4][bet;4][nxC;4][lty;4][eno;4][ftx;4][rst;4][etu ;4][suy;4][ctC;4][mtw;4][des;4][dno;4][esv;4][qty;4][bnt;4][etw;4][stv;4][env;4][bot;4][ort;4][fn w;4][fnx;4][frs;4][tuC;4][twy;4][efq;4][swy;4][lnt;4][ept;4][cfn;4][flt;4][esw;4][eoy;4][lxy;4][cs x;4][twC;4][otw;4][clt;4][ltw;4][ogy;4][efn;4][nsu;4][ctx;4][eft;4][qsy;4][oxC;4][mno;4][fnr;4][bqs;4][mnq;4][fst;4][fmo;4][fmn;4][fqt;4][elq;4][otu;4][ctv;4][ltu;4][efs;4][cls;4][fqv;4][fsy;4][f oy;4][ptC;4][tCy;4][dqt;4][otv;4][ces;4][cet;4][mps;4][oCy;4][det;4][asC;4][fsx;4][ftC;4][enq;4][mpt;4][dls;4][eos;4][dnt;4][rtC;4][mox;4][nos;4][cnq;4][fvy;4][ngr;4][fos;4][loC;4][fmq;4][niB ;3][sty;3][efo;3][nvC;3][nqw;3][ntu;3][qtC;3][nsy;3][nsw;3][nsx;3][esC;3][etv;3][agt;3][aoq;3][f lq;3][nqu;3][bmt;3][esx;3][fny;3][ert;3][dsC;3][ltx;3][ams;3][esy;3][coq;3][lqx;3][est;3][fls;3][b qt;3][boq;3][dst;3][ntv;3][ety;3][ngx;3][qsv;3][gjB;3][ntw;3][cst;3][dot;3][otx;3][ngi;3][ogr;3][s vC;3][lqv;3][dms;3][ags;3][csC;3][els;3][msw;3][ltv;3][suC;3][bsC;3][fot;3].

4 Phage Combinations

4 phage combinations were analyzed in silico for their ability to lyse the 352 different strains of Klebsiella pneumoniae bacteria based on the ability of the single phage to lyse the bacteria as assessed by a liquid media assay.

The combinations with the highest “at least 1 phage % coverage” are provided herein below (out of 23,751 possible combinations, the top 3.5% (828) are provided). The phage combinations are ordered in descending performance grade.

For example, in the case of [alyg;74], the 4 phage combination that provided the highest percent coverage, 395-D2-3-1, KP10-11, KP10-91 and CT-227-KP4M1-11 lysed 74% of all the strains of Klebsiella pneumonia analyzed.

[alyg;74][alqg;74][blqg;73][alyB;73][blyg;73][ablg;72][alqB;72][blqB;72][alsg;72][blyB ;72][ablB;71][luyg;71][blug;71][alvg;70][alyi;70][alsB;70][alqi;70][lqvg;70][luyB;70][lqug;70][blqi;70][alrg;70][bluB;70][blCg;70][alog;70][aflg;70][lvyg;70][bflg;69][alvB;69][blyi;69][lquB; 69][alCg;69][blsg;69][lqvB;69][lgsg;69][abli;69][lwyg;69][alrB;69][altg;69][bclg;69][alpg;69][a loB;69][lqwg;68][blCB;68][bdlg;68][alsi;68][bltg;68][aclg;68][dlyg;68][aflB;68][alwg;68][alug; 68][lvyB;68][blwg;68][blmg;68][lqxg;68][lxyg;68][bflB;68][lsyg;68][adlg;68][alCB;68][dlqg;68][lgsB;68][almg;68][clqg;68][lpqg;68][lwyB;68][clyg;68][algj;68][blsB;68][luyi;68][dlyB;68][aluB;68][aelg;68][altB;68][loqg;68][bdlB;68][alpB;68][lryg;68][alxg;68][algi;68][lpyg;67][bclB ;67][aloi;67][blui;67][luvg;67][lqwB;67][adlB;67][algh;67][algB;67][aliB;67][alwB;67][bltB;67][lgrg;67][lsyB;67][flyg;67][aljB;67][lxyB;67][dlqB;67][lsug;67][lqxB;67][aclB;67][blwB;67][a lgz;67][abyg;67][alvi;67][blmB;67][alng;67][alri;67][clug;67][lpqB;67][abqg;67][blxg;67][loqB ;67][lqvi;67][alyj;67][lswg;67][algk;67][blgj;67][almB;67][dlsg;67][alqs;67][algA;67][ablq;66][blCi;66][belg;66][luvB;66][lryB;66][ltyg;66][loyg;66][lqui;66][blrg;66][alxB;66][blgi;66][blog; 66][algj;66][aelB;66][clqB;66][clyB;66][bliB;66][blpg;66][dlvg;66][alny;66][lgrB;66][afli;66][1 uwg;66][lsuB;66][lvCg;66][blgh;66][lpyB;66][blgB;66][alhB;66][bng;66][lvyi;66][blqj;66][lwy i;66][alnq;66][flqg;66][flyB;66][bcli;66][apyg;66][alzB;66][alkB;66][abyB;66][flvg;66][blsi;66][alCi;66][blgz;66][dlsB;66][bljB;66][blnq;66][bfli;66][lgtg;66][alnB;66][lgsi;66][lrug;66][aloq;66][dlug;66][alsy;66][aryg;66][blgk;66][bclq;66][alAB;66][ably;66][bloB;66][acli;66][loyB;66][1 swB;66][lsCg;66][blqs;66][bdli;66][blyj;66][abgB;66][bsyg;66][elyg;66][blvg;66][blgA;66][blx B;66][alpi;66][dlyi;66][cluB;66][ablj;65][ltyB;65][lqwi;65][aoyg;65][dluB;65][alti;65][belB;65][adli;65][loqi;65][blqu;65][apqg;65][asyg;65][blrB;65][blny;65][luCg;65][clsg;65][luwB;65][lou g;65][alwi;65][dlvB;65][lmyg;65][lsyi;65][bngg;65][lqyg;65][blnB;65][clvg;65][clqi;65][alui;65][blhB;65][bqsg;65][clyi;65][blpB;65][avyg;65][blti;65][lvCB;65][lruB;65][lpug;65][alij;65][alo y;65][alqv;65][aqrg;65][blwi;65][lsvg;65][dlqi;65][dlxg;65][lpqi;65][lwCg;65][bcly;65][lgtB;65][blmi;65][agsg;65][blzB;65][alns;65][bluy;65][lqxi;65][blsy;65][lCyg;65][flqB;65][blqt;65][lux g;65][blqw;65][flug;65][aeli;65][elqg;65][lvwg;65][flvB;65][aryB;65][abln;65][buyg;65][louB;65][bnyg;65][blkB;65][algr;65][apyB;65][lxyi;65][lsCB;64][aoyB;64][blAB;64][abls;64][anyg;641[aoag;64][elyB;64][alsj;64][aavg;64][aliz;64][luCB;64][acly;64][blvB;64][almi;64][aclq;64][lr yi;64][alni;64][bsyB;64][dlwg;64][asyB;64][lmqg;64][lgri;64][alvy;64][alxi;64][alpq;64][lgsw;64][lxCg;64][alwy;64][lpyi;64][alry;64][bloi;64][aely;64][lnyg;64][lpuB;64][loyi;64][clCg;64][al ih;64][bflq;64][dlsi;64][lmyB;64][bqug;64][apqB;64][luyj;64][luvi;64][bdlq;64][ltvg;64][bpqg;64][algt;64][dltg;64][luxB;64][bluj;64][lmug;64][alqw;64][aelq;64][blij;64][buyB;64][ltug;64][bn qB;64][fluB;64][clxg;64][lnuy;64][lsui;64][lsvB;64][alsC;64][afly;64][flwg;64][clsB;64][bqsB;64][lrwg;64][agrB;64][lswi;64][lqyB;64][blwy;64][clui;64][alyz;64][avyB;64][dlrg;64][dflg;64][1 ygj;64][dlxB;64][alty;64][cluy;64][aliA;64][dlqs;64][lsxg;64][bcqg;64][agsB;64][ablC;64][aluy; 64][alqz;64][lCyB;64][dlog;64][clvB;64][alqy;64][lvwB;64][alpy;64][blty;64][abyi;64][lqCg;64][lwxg;64][lwCB;64][bwyg;64][bnyB;64][blni;64][bcyg;64][adlq;64][flsg;64][lygi;64][adly;64][flyi;64][angg;64][beli;64][blnu;64][bmqg;64][bfly;64][elqB;64][bpyg;64][bfyg;64][blmq;64][ao qB;64][aflq;63][anyB;63][dlwB;63][blxi;63][lrCg;63][cdlg;63][alsw;63][alnj;63][bqwg;63][lmv g;63][bquB;63][almy;63][lugj;63][bloq;63][bliz;63][alqx;63][lsuy;63][clqu;63][lngg;63][elug;63][lpCg;63][dlpg;63][blqx;63][lswy;63][blqz;63][aqvB;63][lygB;63][abng;63][alvj;63][flrg;63][bl ri;63][dlCg;63][abqi;63][lgsu;63][clqs;63][lyiB;63][alik;63][dlvi;63][alxy;63][lowg;63][belq;63][lygh;63][blCj;63][loCg;63][lgsv;63][blpi;63][abnq;63][lvxg;63][luwy;63][ltuB;63][alqu;63][lqv j;63][lygz;63][acls;63][lrsg;63][afls;63][ltyi;63][bdly;63][alos;63][lyjB;63][cltg;63][alCy;63][clr g;63][dlrB;63][lnqs;63][lqgj;63][lmqB;63][ablt;63][dflB;63][cflg;63][lquv;63][blih;63][dlui;63][bmyg;63][dlsy;63][alyh;63][absg;63][loui;63][lvCi;63][awyg;63][lmuB;63][almq;63][dltB;63][1 xCB;63][lwyj;63][luwi;63][blsu;63][lnyB;63][alqC;63][alno;63][lquj;63][aoyi;63][uvyg;63][abl u;63][dluy;63][lsCi;63][alyA;63][clCB;63][bpqB;63][alsu;63][alyk;63][ltwg;63][clsi;63][bdqg;63][luvy;63][blqC;63][alCj;63][ltvB;63][loqu;63][blnC;63][alqh;63][louy;63][lrui;63][adls;63][bl my;63][dloB;63][alsv;63][apyi;63][alnv;63][luiB;63][lpwg;63][aryi;63][lygA;63][lujB;63][lnqu; 63][elyi;63][lygk;63][blyz;63][lugh;63][blxy;63][lugi;63][lnqv;63][flwB;63][lnug;63][bclj;63][c lqv;63][blsC;63][lugB;63][lqgi;63][bclu;63][elvg;63][abfl;63][lrwB;63][flxg;63][bely;62][blqh;62][bfqg;62][abny;62][alwj;62][bpyB;62][blqy;62][lowB;62][flvi;62][eluB;62][lvyj;62][lmsg;62][ablz;62][bluC;62][algk;62][alst;62][lgsx;62][bliA;62][bfyB;62][blpq;62][lwxB;62][btyg;62][afy g;62][alrj;62][bloy;62][loCB;62][lqgB;62][auyg;62][bryg;62][aclj;62][aloj;62][bmqB;62][amyg; 62][lgti;62][lpsg;62][lqCB;62][abcl;62][lquw;62][flqi;62][bwyB;62][bnqi;62][lsxB;62][clsy;62][dlCB;62][blnj;62][anqB;62][blns;62][dlpB;62][lqgh;62][flsB;62][lqwj;62][flCg;62][aflj;62][lrvg ;62][bsyi;62][bdyg;62][clog;62][lqiB;62][alqA;62][alrs;62][alnr;62][lugz;62][clxB;62][dlqv;62][dlqu;62][bqyg;62][cdlB;62][clwg;62][aels;62][ltCg;62][lqgz;62][dlyj;62][blqk;62][blqA;62][abl o;62][clpg;62][lnsy;62][bflj;62][abel;62][elsg;62][apqi;62][flrB;62][lnqB;62][bcqB;62][lruy;62][asyi;62][agyg;62][lugk;62][bqwB;62][lugA;62][ablw;62][alsz;62][blik;62][abnB;62][blqr;62][it xg;62][batg;62][aloC;62][blwj;62][lpCB;62][blvi;62][lmvB;62][lpvg;62][abCg;62][bnyi;62][bdl j;62][elwg;62][aoqi;62][abdl;62][lnvy;62][lsvy;62][luCi;62][lyhB;62][lrCB;62][flog;62][lgjB;62][dlny;62][clvi;62][bngs;62][lovg;62][bcyB;62][lrxg;62][lnoq;62][lgsj;62][angr;62][bcls;62][lyk B;62][lyzB;62][axyg;62][uvyB;62][bdqB;62][lrsB;62][lmwg;62][quvg;62][puyg;62][elqi;62][lqr u;62][bnqu;62][ablm;62][blsj;62][lqvw;62][acyg;62][lsvi;62][absB;62][lqgA;62][lqgk;62][blyh; 62][bnsy;62][bmyB;62][abpg;62][bfln;62][awyB;62][lmyi;62][flpg;62][luhB;62][afln;62][bclC; 62][loqw;62][dlxi;62][bgrg;62][lnuB;62][bxyg;62][alps;62][clgj;62][alvC;62][adlj;62][ruyg;62][altj;62][bqsi;62][clyj;62][anry;62][lvxB;62][dlqx;62][boyg;62][agri;62][alnC;62][lvgj;62][bdln;62][angs;62][luxy;62][lpui;62][lnwy;62][alms;62][clvy;62][anyi;62][bdls;62][blry;62][apsg;62][a nsy;62][dlnq;62][lqux;62][bltj;62][blCy;62][bdlu;62][lxyj;62][alnt;62][bnuy;62][alsh;62][lqxj;62][ablh;62][lstg;62][lyAB;62][lpuy;62][dlxy;62][dlvy;62][blqv;62][avyi;62][bltC;62][aflo;62][blp y;62][ltwB;62][lnyi;62][lpqu;62][cltB;62][aelj;62][lvgi;62][alsx;62][lCyi;62][aluj;62][dlgj;62][1 gyi;62][lngx;62][elvB;62][bdyB;62][lmxg;62][bflC;62][lnxy;62][cflB;62][blyA;62][clsu;62][lw gj;62][flxB;62][alpj;62][clrB;62][aqsi;62][atyg;62][blnt;62][blyk;62][auyB;62][adyg;62][lwCi;62][flqv;62][lpwB;62][lukB;62][cloq;62][clqx;62][lvwi;62][luzB;62][eluy;62][dlmg;62][dlgj;62][dloi;62][lpxg;62][celg;61][btyB;61][delg;61][alnp;61][ltuy;61][adln;61][qswg;61][lviB;61][lgrw ;61][dlwi;61][anrg;61][lgtv;61][afyB;61][lvgB;61][bfqB;61][luyz;61][bltu;61][clxy;61][lmsB;61][floB;61][dsyg;61][dlng;61][lngw;61][loCi;61][anoq;61][buyi;61][aclo;61][swyg;61][lswC;61][dliB;61][alsk;61][alrC;61][altv;61][alsA;61][bogg;61].

The combinations with the highest “at least 2 phage % coverage” are provided herein below. The phage combinations are ordered in descending performance grade.

Thus, for example, in the case of [ably;41], when 395-D2-3-1, 395-D2-3-2, KP10-11 and KP10-91 are used in a combination, 41% of the bacterial strains analyzed were targeted by at least 2 of the 4 phage.

[ably;41][ablp;40][bqvy;39][ablq;39][bdly;39][alvy;39][abc1;38][ablr;38][abln;38][aluy;38][blqy ;38][alpy;38][blpy;38][bluy;38][blvy;38][blCy;38][ablw;38][alry;38][ablo;38][blry;38][abqy;38][blwy;38][alwy;38][ablv;38][abdl;38][abls;38][ablC;37][ablu;37][adlp;37][adly;37][abwy;37][lq ry;37][abpy;37][lpqy;37][alpv;37][blpq;37][alnu;37][adln;37][abdy;37][abdv;37][bvCy;37][lqvy ;37][ablm;37][blnv;37][blqv;36][bdlq;36][bgry;36][bdls;36][alnw;36][adlv;36][alpu;36][abvy;36][blnw;36][bdlp;36][abfl;36][alqy;36][abmy;36][alpq;36][abry;36][dlpy;36][alpr;36][alsy;36][bl sy;36][abcy;36][adlr;36][abuv;36][bloy;36][acln;36][adlo;36][alqu;36][bpqy;36][bqwy;36][dlvy; 36][lvwy;36][aloy;36][alqv;36][blqr;36][lpvy;36][abny;36][alxy;36][dlry;36][lpwy;36][bcln;36][abCy;36][bdvy;36][aquy;36][aqvy;36][aluw;36][alvw;36][aclv;36][bdlv;36][blpv;36][adls;36][b dln;36][luvy;36][alqw;36][lqwy;36][abqv;36][adlw;36][abru;35][blvC;35][acly;35][blsw;35][ab dw;35][aluv;35][alpw;35][alru;35][blpw;35][biny;35][bday;35][bquy;35][blxy;35][bcly;35][abd p;35][abuy;35][bqxy;35][blqu;35][dlwy;35][alrw;35][aclp;35][brCy;35][abxy;35][adlu;35][abvC ;35][bdlo;35][bdlw;35][abdr;35][bluw;35][bqvC;35][boqy;35][blnr;35][bdvC;35][abcn;35][ablt; 35][abcv;35][lpry;35][bdlr;35][blpC;35][aclr;35][algr;35][blps;35][dlnw;35][bduv;35][bdlC;35][adlq;35][aboy;35][abdn;35][lvCy;35][blqw;35][abdo;35][blrw;35][lrwy;35][ablx;35][alrv;35][ab pq;35][lpqv;35][aqwy;35][qrvy;35][blpu;35][alou;34][abvw;34][alnv;34][alpC;34][blnp;34][alu C;34][alps;34][alsw;34][blqs;34][alnx;34][bdlu;34][bloq;34][abdq;34][lpuy;34][bcdv;34][adqy;34][bclp;34][alpx;34][abqw;34][advy;34][lgsy;34][dlpq;34][aclu;34][lqxy;34][lrCy;34][abqr;34][alCy;34][alov;34][abel;34][blwC;34][bdvw;34][alny;34][luwy;34][abpC;34][alow;34][qvwy;34][bdxy;34][blrC;34][loqy;34][lnuw;34][bdmv;34][alsv;34][adlx;34][buvy;34][abrs;34][blmy;34][bxCy;34][dlpv;34][bluv;34][abds;34][almu;34][bdry;34][blqC;34][blsC;34][abdC;34][alwC;34][bfly;34][alvC;34][abcd;34][abpu;34][cdln;34][abdx;34][bcls;34][blnu;34][bdqv;34][abnw;34][ab fy;34][bdpy;34][abmv;34][aqxy;34][abcp;34][abwC;34][dlsy;34][adxy;34][pqvy;34][blnx;34][lq uy;34][bluC;34][abnp;34][lnwy;34][lruy;34][bcdl;34][alrC;34][dlqy;34][dloy;34][alop;34][abrw; 34][alnr;34][bdrw;34][blsv;34][lnvw;34][lpCy;34][dlps;34][aboq;34][dlpu;34][aduw;34][bloC;34][blvw;34][bdwy;34][algs;34][lwCy;34][abfv;34][arvy;34][blsu;34][afly;34][adr;34][lpqr;34][abpw;34][aquw;34][absy;34][clnw;34][blmv;34][bruy;34][lswy;34][abcr;34][bpCy;34][bvgB;34][avwy;34][aclq;34][alrs;34][adow;34][dluy;34][bwCy;34][alsu;34][blnq;34][abrC;34][abqu;34][dlnr;34][aduy;34][bquv;34][dlnu;34][adsy;34][dlpr;34][lngy;34][acls;34][lpqw;34][acuw;34][ac dl;34][lrvy;34][adlC;33][bclq;33][pqwy;33][clwy;33][blrv;33][lnrv;33][dlov;33][abdu;33][abtv; 33][adwx;33][abmq;33][abpv;33][clpy;33][blty;33][dlpw;33][abvx;33][auwC;33][boCy;33][bfu v;33][dlqv;33][aloq;33][clnv;33][bely;33][alnq;33][abnv;33][lquv;33][abcq;33][blpx;33][bpuy;33][alqx;33][abdm;33][blqx;33][bdpv;33][dlqw;33][aruv;33][almw;33][abuw;33][alor;33][lowy;33][abty;33][bdfv;33][bclv;33][advw;33][almv;33][auwy;33][lnqw;33][buCy;33][abcs;33][aclo;33][blow;33][abmr;33][dlnp;33][agsy;33][dlvw;33][bclC;33][dlqr;33][bcvw;33][absv;33][adlm;33][lnpr;33][blru;33][abco;33][abou;33][acdu;33][lovy;33][lpvw;33][clvy;33][bclr;33][dlsu;33][a dry;33][lwxy;33][dlny;33][blrs;33][apuy;33][bdlx;33][aclw;33][adpr;33][abpr;33][lnvy;33][abop ;33][lqvw;33][abqs;33][blou;33][bdpw;33][anwy;33][abfr;33][absw;33][bflv;33][abrv;33][abmC ;33][alty;33][dqvy;33][apvy;33][arwy;33][dlxy;33][blmp;33][blop;33][cdly;33][lpqu;33][auvy;33][bclu;33][abor;33][lsuy;33][abcx;33][blpr;33][bdCy;33][abnr;33][abnq;33][dlrC;33][ablB;33][lvxy;33][acdx;33][bduy;33][buvC;33][ablg;33][bclw;33][pgry;33][acvy;33][ablz;33][lnrw;33][al xC;33][anuw;33][clnr;33][qvxy;33][cdlp;33][adwy;33][abqx;33][aloC;33][lpuv;33][adov;33][ab gB;33][abcw;33][aclx;33][lnry;33][bdpu;33][alrx;33][buwy;33][bqsy;33][cqwy;33][lnpv;33][ab nC;33][aflv;33][ablA;33][almr;33][abos;33][alnp;33][alux;33][abcC;33][adqu;33][bdoy;33][lsC y;331][dlnv;33][clry;33][aboC;33][aswy;33][dlor;33][dlou;33][anuy;33][ablh;33][bduw;33][luvw ;33][ablj;33][agry;33][bdsw;33][abey;33][dluv;33][dlsv;33][apqy;33][almy;33][augB;33][almp; 33][buvw;33][ablk;33][abcu;33][absu;33][adrw;33][abli;33][bdsy;33][alnC;33][abno;32][agrv;32][lpru;32][louy;32][bdrv;32][aduC;32][qrwy;32][dgwy;32][avCy;32][clpv;32][aflp;32][dlpC;32][adsw;32][lquw;32][lnuv;32][adqv;32][abmp;32][bins;32][alwx;32][lgrv;32][bdnv;32][bdtv;32][adqx;32][abow;32][acuy;32][adpv;32][adnv;32][bflp;32][luxy;32][dCy;32][bcdr;32][auvw;32][clqy;32][abmn;32][aruy;32][bgrC;32][anvw;32][adny;32][bpuw;32][apry;32][acnv;32][opqy;32][bdlm;32][bdvx;32][adpx;32][adoy;32][quvy;32][aowy;32][abfq;32][bdsv;32][abqC;32][abrx;32][bdev;32][bdow;32][belp;32][alsC;32][blov;32][lpsy;32][lprw;32][lpvC;32][dlru;32][advC;32][anvy;32][abdf;32][blms;32][advx;32][aclC;32][acqu;32][bngy;32][aelp;32][blxC;32][almx;32][a wxy;32][bdfl;32][bdnp;32][aflu;32][anxy;32][lnpw;32][dlrs;32][bflw;32][abmu;32][acuC;32][bd ov;32][dlnq;32][blor;32][clpq;32][abov;32][clnp;32][bdny;32][lrsy;32][clnu;32][abps;32][abms; 32][abev;32][lpsv;32][adsv;32][aflw;32][lprs;32][lsvy;32][blnC;32][bcdp;32][bruw;32][apuv;32][lory;32][acdv;32][begy;32][blmr;32][lsuv;32][adrx;32][acdw;32][dlsw;32][abde;32][lpuw;32][loqv;32][absC;32][adnw;32][lxCy;32][buxy;32][bcdx;32][dlvC;32][bcdw;32][apuw;32][bmvy;32][bdrs;32][dlrw;32][bltv;32][cdlr;32][dlow;32][blrx;32][angw;32][bgru;32][arvw;32][bruv;32][btuv;32][abrt;32][abwx;32][dloq;32][dlop;32][belw;32][bcds;32][bclo;32][lpsu;32][lvwC;32][ad qw;32][abmw;32][lrvw;32][brsw;32][dlrv;32][lgru;32][aelw;32][lgrw;32][dlos;32][quwy;32][bc rw;32][abdt;32][lnpy;32][blmw;32][aruw;32][adsx;32][blwx;32][adfl;32][blux;32][adlt;32][bnvy ;32][aduv;32][ador;32][abns;32][bdru;32][abfp;32][lmpv;32][anpu;32][lpsw;32][lnvx;32][lnqr;32][qwxy;32][lnux;32][cdlv;32][apru;32][adpw;32][bdop;32][luCy;32][bcuv;32][bdnw;32][altu;32][luvC;32][bdwx;32][blno;32][rvCy;32][bfqv;32][loCy;32][blmu;32][aprC;32][brwC;32][awCy ;32][bflu;32][adgB;32][clpu;32][acqy;32][apwy;32][cluy;32][lrsu;32][abot;32][dlqs;32][adox;32][lsvw;32][apqv;32][bdel;32][avwC;32][clpr;32][clpw;32][lpqs;32][bdlt;32][adpu;32][arvC;32][clny;32][acxy;32][asuy;32][blmq;32][belv;32][bdpC;32][acrv;32][brwy;32][adpy;32][bpvy;32][dluw;32][abuC;32][lprv;32][aely;32][adru;32][ogry;32][alpt;32][bmuv;32][blvx;32][flgy;32][qu xy;32][abnu;32][lpuC;32][lnru;32][bmvw;32][apqu;32][lnCy;32][oqvy;32][lprC;32][dlqu;32][bp rw;32][cdqy;32][bpqC;32][alvx;32][aber;32][aqwx;32][amvy;32][adrs;32][qsuy;32][aquv;32][bd pr;32][abox;32][aqvw;32][abmo;32][abcm;32][lnuy;32][lgsu;32][alms;32][agrw;32][clqw;32][a opu;32][adxC;32][alqC;32][ogwy;32][bdvg;32][abdg;32][anrw;32][dloC;31][dquy;31][aouy;31][angy;31][npqy;31][buvx;31][bpry;31][lqvC;31][bnpw;31][bpqu;31][aflr;31][lmpy;31][dlwC;31][altw;31][avxy;31][apvC;31][bltw;31][bdux;31][bpuv;31][belr;31][lgsw;31][awxC;31][lruC;31][bdvB;31][bpvC;31][dfly;31][abdB;31][bdpx;31][axCy;31][pquy;31][abfo;31][flvy;31][clps;31][bcdo;31][lpxy;31][bdps;31][aprw;31][aogy;31][acnu;31][aoru;31][bels;31][acry;31][abpt;31][dl px;31][louv;31][aouw;31][apar;31][almo;31][adop;31][acwy;31][lnaC;31][lmry;31][arwC;31][a cds;31][bdpq;31][afln;31][pqxy;31][adno;31][acuv;31][lmvy;31][lmqy;31][blpt;31][lnwx;31][bo uy;31][bcqy;31][lmpr;31][bdos;31][luwC;31][dlno;31][lrvC;31][lnpu;31][auCy;31][aqux;31][ap uC;31][alos;31][qrxy;31][bnuw;31][bcsw;31][aelv;31][abxC;31]

The combinations with the highest “at least 3 phage % coverage” are provided herein below. The phage combinations are ordered in descending performance grade.

Thus, for example, in the case of [abrv:25], when 395-D2-3-1, 395-D2-3-2, KP10-61 and KP10-85 are used in a combination, 25% of the bacterial strains analyzed were targeted by at least 3 of the 4 phage.

[abrv;25][abdv;23][ablv;23][abdu;23][abdl;23][abuw;23][abpv;23][brvw;23][abdw;23][b ruv;23][bpvw;23][abcw;23][abux;22][abdr;22][bdrv;22][abrw;22][brvx;22][bdlv;22][abov;22][b crv;22][abvw;22][blpr;22][bluv;22][ablw;22][abcv;22][abdx;22][adux;22][acvw;22][aduw;22][a bwx;22][bprv;22][abpr;22][blrv;22][blpv;22][ablu;22][acrw;22][adlu;22][adlw;22][blvw;22][abp w;22][abvy;22][abdy;22][abcu;22][abcd;22][auwx;21][bcpv;21][bpvx;21][abds;21][bvwx;21][b puv;21][bovw;21][aduv;21][bdpv;21][bdlw;21][abpx;21][brvy;21][bpru;21][acdw;21][bovx;21][acwx;21][aclw;21][abow;21][abuv;21][abuy;21][buvw;21][berv;21][bcpw;21][ablx;21][bprw;21][absw;21][abdp;21][abcr;21][bouv;21][aluw;21][abfu;21][bvwy;21][bclv;21][bcvx;21][advw;21][blru;21][abnv;21][bnrv;21][bdpr;21][bdlr;21][acpw;21][bclw;21][adwy;21][abvx;21][abdo;21][bnvw;21][abuC;21][blvx;21][abrx;21][bdvw;21][ablr;21][abpu;20][lprv;20][bcuw;20][abqu;20][brvC;20][acdl;20][adru;20][abmw;20][bruw;20][acuw;20][aprv;20][bdru;20][abou;20][bfrv;20][bmrv;20][bdlp;20][abew;20][bcrw;20][ablp;20][bduw;20][abru;20][bcwx;20][abry;20][bdvx;20][auvw;20][bsvw;20][acdy;20][bprx;20][acdv;20][bgrv;20][blpw;20][abqv;20][bcvw;20][pruv;20][buvx;20][bcdw;20][acdu;20][brwx;20][abdf;20][aluv;20][bcpr;20][adwC;20][cdlw;20][bdov; 20][adpu;20][bpwx;20][borv;20][arvw;20][bduv;20][aruw;20][blpu;20][adrw;20][apvw;20][ably ;20][adlv;20][abfw;20][buwx;20][bvxy;20][absv;20][adsu;20][abeu;20][abcx;20][bdpw;20][abor ;20][auvx;20][bdlu;20][bcuv;20][abmu;20][abdq;20][bdrw;20][adqw;20][bpwy;20][adnw;20][ab mv;20][brwy;20][alrv;20][abev;20][apuw;20][abfv;20][adrv;20][blrw;20][cdvw;20][abtu;20][ab vC;20][auwy;20][adwx;20][acuv;20][bnpv;20][abwC;20][alux;20][alpu;20][abnu;20][abc1;20][a bnw;20][adou;20][dluw;20][apux;20][cdrw;20][cpvw;20][acux;20][borw;20][abdm;19][avwx;19][abxC;19][blov;19][adpw;19][alwx;19][adfu;19][bnvx;19][cdpw;19][dprv;19][lruv;19][acnw;19][abrC;19][bclr;19][cpwx;19][bdor;19][apwx;19][bdou;19][abwy;19][acdp;19][bpvy;19][cduw;19][acdC;19][alpw;19][abex;19][prvw;19][bpux;19][bepr;19][bdwx;19][aduy;19][acrv;19][bepv; 19][aprw;19][abqw;19][adlr;19][alvw;19][abps;19][adly;19][abnx;19][aber;19][bopr;19][bdlx;19][bcdu;19][bcdl;19][acsw;19][abcp;19][cprw;19][bcov;19][dlrv;19][adpv;19][arwx;19][borx;19][aclu;19][bopv;19][cuvw;19][bouw;19][cprv;19][bavw;19][bdux;19][abdt;19][bdrx;19][apvx;19 ][adlx;19][afuw;19][acew;19][bcru;19][bcnw;19][abdC;19][bnpr;19][abop;19][blvy;19][bluw;19][bvwC;19][boru;19][acdr;19][bevw;19][bclp;19][abtw;19][ablC;19][bdvy;19][bmpw;19][abqr;19][arvx;19][acow;19][bdwy;19][alvx;19][bcow;19][bdow;19][blwx;19][brtv;19][brsv;19][cvwx; 19][bpsw;19][bopw;19][bpuw;19][aeux;19][cluw;19][ruvw;19][dlrw;19][absx;19][blrx;19][lprw ;19][bmvw;19][clvw;19][crvw;19][adnu;19][adew;19][lpvw;19][absu;19][bprs;19][bcrx;19][bcp x;19][alpv;19][adtu;19][alnp;19][cruw;19][acwC;19][amvw;19][bpvC;18][abrs;18][clwx;18][acr u;18][bdsu;18][acdq;18][aruv;18][aluC;18][ablq;18][bduy;18][advx;18][adfw;18][adqu;18][alpr; 18][auwC;18][dlpr;18][acwy;18][clrv;18][admu;18][anuw;18][adlC;18][acpv;18][bcdv;18][bcsw ;18][abdn;18][pvwx;18][abxy;18][aouw;18][bfvw;18][aquw;18][lpuv;18][abls;18][bfpr;18][advy ;18][bmpv;18][asuw;18][cdwy;18][auxy;18][amuw;18][alru;18][blpx;18][alrw;18][alwy;18][bdo p;18][dpvw;18][adlp;18][afuv;18][acde;18][abcs;18][bpry;18][bmvx;18][dlvw;18][abcf;18][bcn v;18][admw;18][bdnv;18][acmw;18][apwy;18][bnuv;18][abqx;18][lopr;18][adsw;18][buvy;18][beov;18][aovw;18][blor;18][brux;18][cuwx;18][bprC;18][bnpw;18][auxC;18][aewx;18][dprw;18][alwC;18][belv;18][cpuw;18][apuv;18][bevx;18][rvwx;18][dlpv;18][abnr;18][clrw;18][bcew;18][bdsw;18][bcmw;18][afru;18][aeuw;18][abco;18][abox;18][apru;18][abde;18][brsw;18][prvx; 18][bdpu;18][cruv;18][blux;18][anpw;18][bnrw;18][bdew;18][abpy;18][auvy;18][aduC;18][bpq v;18][bdmw;18][cdwx;18][abmx;18][bcdr;18][bopu;18][alqu;18][ablo;18][buwy;18][acfw;18][b dly;18][clpw;18][blop;18][abmr;18][acdn;18][acpr;18][bmpr;18][adlq;18][brxy;18][bsuw;18][al qw;18][clpr;18][lrvx;18][bowx;18][adCy;18][orvx;18][acdm;18][adpy;18][blwy;18][aclv;18][ab mp;18][arux;18][blrs;18][uvwx;18][abnp;18][crwx;18][anwx;18][lpru;18][abfr;18][druv;18][bdn w;18][bfuw;18][bdsv;18][lrvw;18][bcop;18][duvw;18][bwxy;18][aorv;18][acvx;18][bdwC;18][a cdx;18][dpuw;18][afvw;18][bswx;18][duwx;18][bdfw;18][aopw;18][bdpx;18][nprv;18][blry;18][abuB;18][adpr;18][bcux;18][copw;18][bcor;18][prsw;18][avwy;18][bclx;18][dvwx;18][bclu;18][dluv;18][bcdp;18][abcC;18][aowx;18][aepw;18][cdow;18][auCy;18][dlpw;18][afpu;18][bdrs;18][abpC;18][lruw;18][bovC;18][adel;18][puvw;18][abcm;18][abfx;18][awxC;18][aclx;18][abqy; 18][avwC;18][beuw;18][aelu;18][abui;18][bovy;18][adeu;18][clpv;18][nprw;18][aprx;18][aeru; 18][aruC;18][bcdy;18][amwx;18][bpqr;18][afux;18][auvC;18][bcsv;18][cdtw;18][beru;18][bew C;18][blnp;18][bsvx;18][bdfu;18][abuz;18][actw;18][cdnw;18][dlru;18][bewx;18][pruw;18][acq w;18][anux;18][blsv;18][blps;18][blrC;18][bcfw;18][abuj;18][abuA;18][aluy;18][abCy;18][acpu ;18][abuh;18][deuw;18][abce;18][blqv;18][aprs;18][bpsu;18][adtw;18][bqvx;18][bpwC;18][abu g;18][blpy;18][bpsv;18][abuk;18][beuv;17][arwy;17][aouv;17][druw;17][bruy;17][bnov;17][cdf w;17][acfu;17][drvw;17][blvC;17][lprx;17][adrx;17][alsu;17][cnrw;17][bprt;17][adry;17][orvw; 17][cdmw;17][abel;17][blou;17][absy;17][aevw;17][aquv;17][bmov;17][abtx;17][aeuv;17][cdsw :17][abim;17][acip;17][arvy;17][aruy;17][awxy;17][buwC;17][apwC;17][eprv;17][awCy;17][be pw;17][adpq;17][acsu;17][bpqw;17][cepw;17][ampw;17][abeo;17][bdlo;17][belr;17][puwx;17][bwxC;17][bctw;17][bfvx;17][bdgw;17][berw;17][bfpv;17][atuw;17][abtv;17][abn;17][bdnr;17][cduv;17][abep;17][dnuw;17][bowy;17][bmwx;17][lpuw;17][cnuw;17][bcdC;17][bdry;17][bqrw; 17][aoux;17][adow;17][bnwx;17][abwj;17][aopr;17][lqCy;17][anvw;17][abwz;17][aclr;17][bcw y;17][acds;17][lnpv;17][duwy;17][bfov;17][aqvw;17][ouvw;17][bqwx;17][bpsx;17][adfl;17][bc ou;17][puvx;17][aflu;17][blqw;17][acdf;17][bdtu;17][apqw;17][abet;17][oruv;17][blow;17][dpw x;17][amrv;17][boqv;17][bcde;17][bfru;17][cpvx;17][cnvw;17][aorw;17][npvw;17][prwy;17][be ux;17][bsuv;17][acCy;17][amux;17][atux;17][adfr;17][alqy;17][bdps;17][btvw;17][adfv;17][pru x;17][cdpv;17][brsu;17][bdnu;17][ruwx;17][alrx;17][lrvy;17][brwC;17][bdrC;17][aswx;17][abw i;17][abwh;17][cdew;17][acpx;17][bduC;17][cdpr;17][lvwx;17][abwB;17][bfuv;17][mpvw;17][a lpx;17][abpq;17][adpC;17][anuv;17][abwA;17][abwk;17][bflr;17][cdlu;17][bcpu;17][aqux;17][a bwg;17][dopw;17][luvw;17][crvx;17][cdgw;17][mprv;17][adxC;17][alvy;17][bnrx;17][eprw;17][aelw;17][lpvx;17][afrw;17][dlwx;17][acmu;17][bnop;17][duwC;17][bdpy;17][acwg;17][acwB; 17][adop;17][apsw;17][adep;17][celw;17][bruC;17][abrt;17][acou;17][drvx;17][bvxC;17][couw; 17][beop;17][brvj;17][brvi;17][cdlv;17][amrw;17][dlpu;17][pvwy;17][bcsu;17][opru;17][bdeu;17][bevy;17][blmw;17][ampv;17][anpv;17][rvwy;17][adrC;17][depw;17][acdt;17][aboC;17][alou ;17][acrx;17][acdo;17][brvz;17][prsu;17][cduy;17][bfrw;17][bmuv;17][asvw;17][brvk;17][anrv; 17][brvA;17][prvC;17][brvg;17][adqy;17][oprw;17][lnpr;17][alny;17][agru;17][bcdm;17][arsw; 17][bdqu;17][blmv;17][bquw;17][aovx;17][bcps;17][apuy;17][brvB;17][anru;17][bmrw;17][bop x;17][oruw;17][blqy;17][amuv;17][apCy;17][dpwy;17][bnru;17][acnu;17][abfp;17][adqv;17][bd fr;17][brvh;17][bcdf;17][cpwy;17][adrs;17][blqr;17][asuC;17][bcev;16][adov;16][afuy;16][abcy; 16][bdmu;16][cmvw;16][prvy;16][adls;16][afwx;16][blox;16][alxy;16][bcfu;16][bquv;16][bow C;16][blmr;16][bmuw;16][advC;16][beor;16][brsx;16][fprv;16][lpux;16][dvwy;16][lopw;16][bc mp;16][amru;16][alvC;16][bqCy;16][aclC;16]

The percent of host strains that are infected by 4 phages of phage combinations is provided herein below. This trait is referred to herein as “at least 4 phage % coverage”. Thus, for example, in the case of [abvw:14], when 395-D2-3-1, 395-D2-3-2, KP10-85 and KP10-87 are used in a combination, 14% of the bacterial strains tested were targeted by each of the four phage.

[abvw;14][abdu;13][abuw;13][abdw;13][acdw;13][abcw;13][abuv;13][abvx;12][abpw;12][abru;12][abwx;12][bprv;12][abux;12][aduw;12][bcdw;12][abpu;12][abcd;12][brvw;12][abrw;12][acuw;12][acdu;12][adpw;12][abcu;12][bvwx;11][acpw;11][abrv;11][apuw;11][bprw;11][abpr ;11][bcvw;11][abdp;11][bpuw;11][abdr;11][abcp;11][abrx;11][bpvw;11][bduw;11][cdpw;11][ab pv;11][bdrw;11][auvw;11][buvw;11][acdp;11][cduw;11][bdvw;11][bcpw;11][adpu;11][brvx;11][aruw;11][abdv;11][borv;11][bdpw;11][acpu;11][adrw;11][adru;11][bcdr;11[[abpx;11[[aprw;11][aduC;11][bcrw;11][bcuw;11][auwx;11][avwx;11][abde;11][bcdu;11][abdx;11][bcdp;11][bruw; 10][acdr;10][buwx;10][cdwC;10][adwx;10][bopr;10][apru;10][bdpu;10][blrv;10][brwx;10][abcv ;10][aber;10][bprx;10][arux;10][bpwx;10][ablw;10][cdrw;10][cpuw;10][acrw;10][bcpr;10][apv w;10][brux;10][cdew;10][dpuw;10][advw;10][abeu;10][apwx;10][bcpu;10][bdwx;10][bopv;10][bpvx;10][acru;10][abdC;10][ablu;10][abcx;10][adpr;10][bdpr;10][adeu;10][arvw;10][ablr;10][bp ru;10][dewC;10][bdrv;10][cdpu;10][arwx;10][auvx;10][adeC;10][bruv;10][bdru;10][aruv;10][blr w;10][buvx;10][bcdv;10][blpr;10][acvw;10][dprw;9][bdeC;9][bduv;9][bdew;9][cdru;9][acdC;9][cdeC;9][apuv;9][adew;9][cdwx;9][prvw;9][acpr;9][bcdx;9][abew;9][cprw;9][bcrv;9][pruw;9][ac wx;9][bcwx;9][bdeu;9][druw;9][adpx;9][aduv;9][adwC;9][adux;9][bpuv;9][acdx;9][bcde;9][apu x;9][cdvw;9][brsv;9][abep;9][deuC;9][abdl;9][blvw;9][ablp;9][arvx;9][abcy;9][cdpr;9][oprv;9][b pux;9][aeuC;9][bcru;9][bdux;9][cdmw;9][acde;9][cewC;9][prwx;9][blpw;9][admw;9][abeC;9][a prx;9][drvw;9][bopw;9][adrx;9][acuC;9][cduC;9][adlu;9][acwC;9][ablx;9][duwC;9][aprv;9][acw y;9][dpru;9][bduC;9][acew;9][bcwy;9][cruw;9][acmw;9][bcpv;9][aeuw;9][alpw;9][rvwx;9][alrw ;9][bdrx;9][bovw;9][bcew;9][bdpv;9][dpwx;9][bdwC;9][blrx;9][abwC;9][abce;9][acux;9][abuC; 9][cpru;9][alru;9][bdpx;9][ablv;9][abop;9][bdep;9][borw;9][aqCy;9][bdvx;9][aceu;9][aduy;9][du wx;9][afpw;9][duvw;9][acdm;9][lprw;9][blwx;9][ruwx;9][alpr;9][abev;9][abfw;9][abwy;9][acuy ;9][prvx;9][acfw;9][auwC;9][adep;9][blor;9][adpC;9][admu;9][crvw;9][aewC;9][abor;9][apvx;9][beuC;9][puvw;9][adlw;9][beuw;9][bcuv;9][acdv;9][puwx;9][bluw;9][bepw;9][aluw;9][drwx;9][acuv;9][bcux;9][bcdC;9][cdeu;8][dpvw;8][abc1;8][blpv;8][bdlr;8][aepu;8][blru;8][acdl;8][cdwy; 8][cpvw;8][adrv;8][cuwC;8][adlp;8][cdep;8][adfw;8][pvwx;8][acdy;8][adlr;8][cdpC;8][aceC;8][bcwC;8][abox;8][abcC;8][abow;8][adfu;8][bpsv;8][acdf;8][abov;8][aclw;8][advx;8][bevw;8][de pw;8][cdrx;8][abfu;8][dpwC;8][abfp;8][afuw;8][deuw;8][bcrx;8][absu;8][adqC;8][aepw;8][ruvw ;8][uvwx;8][cdux;8][acrx;8][clpw;8][bopx;8][bepu;8][dmpw;8][adpv;8][acpx;8][bdev;8][bewC; 8][acpv;8][adqu;8][dlpw;8][blop;8][bceu;8][cuwx;8][alvw;8][abdm;8][prux;8][acep;8][adrC;8][oprw;8][cdrv;8][bder;8][bcep;8][bfpw;8][bdlw;8][abou;8][amuw;8][ruvx;8][cuvw;8][blpx;8][dm uw;8][alrx;8][alwx;8][absv;8][ceuw;8][abdf;8][ceuC;8][depC;8][blvx;8][dpux;8][bcpx;8][drux;8][blnr;8][bdlu;8][bclw;8][cmpw;8][cepw;8][ampw;8][cdlw;8][cdfw;8][bcvx;8][cdpx;8][euwC;8][dvwx;8][abln;8][cdmp;8][bcdy;8][abex;8][lrvw;8][abfx;8][abuy;8][cpwC;8][abnu;8][admp;8][b ceC;8][dprx;8][bcqw;8][buwC;8][aber;8][berw;8][bpry;8][crux;8][aclr;8][abpC;8][cdrC;8][acfu; 8][adCy;8][blnp;8][acpy;8][acfp;8][aclu;8][bdex;8][abdq;8][abdn;8][abdy;8][ader;8][orvw;8][de vw;8][bosv;8][epuw;8][acrv;8][opvw;8][lpwx;8][cder;8][borx;8][cfpw;8][alux;8][bcdl;8][adln;8][apwC;8][adev;8][abmw;8][aepC;8][apuC;8][bewx;8][cpwx;8][bdmw;8][acpC;8][abpy;8][alpu;8][blux;8][bcor;8][aopr;8][bcpy;8][lpuw;8][bowx;8][bcuy;8][aclp;8][abmu;8][crwx;8][bsvx;8][ad fp;8][aevw;8][derw;8][bcow;8][bcuC;8][blpu;8][acvx;8][afwx;81 [depu;8][lrwx;8][aopw;8][bclr; 8][dpuC;8][bdpC;8][cdlp;8][dfpw;7][acow;7][adow;7][aopu;7][dpuv;7][bcry;7][dfuw;7][bdfw;7][dewx;7][acry;7][acmp;7][cduy;7][bprs;7][bsuv;7][cprx;7][adex;7][bcfw;7][cpwy;7][dexC;7][lo pr;7][cdlr;7][bovx;7][cdmu;7][acdq;7][abcq;7][cdpy;7][bcmw;7][cdpv;7][blov;7][dprv;7][cprv;7][abcf;7][bcdm;7][adwy;7][cpuC;7][drwC;7][aorw;7][cvwx;7][lprx;7][bdmu;7][alnu;7][bevC;7][afux;7][blnw;7][bfwx;7][abnw;7][bopu;7][acmu;7][bclp;7][devC;7][afpu;7][dqCy;7][aerw;7][clr w;7][cduv;7][bepx;7][abco;7][lpru;7][auwy;7][acqw;7][cpux;7][cdgw;7][bdvC;7][epwC;7][bevy ;7][alrv;7][pruv;7][abfr;7][cepu;7][lprv;7][abnp;7][cdfp;7][asuv;7][bcer;7][aopv;7][beuv;7][lruw ;7][abqw;7][dmwC;7][aeuv;7][aoru;7][bcop;7][cuwy;7][alpx;7][bepC;7][acrC;7][bdlp;7][cepC;7][adqy;7][ampu;7][boux;7][apuy;7][adpy;7][bdxC;7][copw;7][opwx;7][aorv;7][bcfp;7][bcev;7][bops;7][dwxC;7][aruC;7][adlC;7][abdo;7][cruC;7][afpx;7][corw;7][cmuw;7][bcpC;7][abnr;7][p uvx;7][adnu;7][afpr;7][cpuv;7][bdmp;7][beux;7][bmuw;7][bpuC;7][bcdq;7][adfx;7][bdwy;7][pu wC;7][aeru;7][abrs;7][acer;7][bdow;7][lorv;7][admC;7][dquC;7][oprx;7][bpwC;7][adem;7][abc n;7][bepr;7][advC;7][bnpu;7][blnu;7][lnpr;7][dlpr;7][opsv;7][bfuw;7][abel;7][aewx;7][fprw;7][a ovw;7][acdo;7][cdmC;7][dmpu;7][bdrC;7][aouw;7][cdry;7][cdvx;7][boru;7][abvC;7][afrw;7][bl ow;7][cerw;7][bnpr;7][duvx;7][aevC;7][demw;7][adxC;7][clpr;7][dmuC;7][bfrw;7][bexC;7][drv x;7][aopx;7][copr;7][epuC;7][bcdf;7][arwC;7][cdow;7][abcm;7][cdfu;7][bcdo;7][lnpw;7][cdlu;7][abmp;7][bdor;7][bclu;7][cdln;7][bdfp;7][acqu;7][bdfu;7][bcov;7][druC;7][bfpr;7][adnp;7][der C;7][druv;7][prsv;7][bepv;7][crwC;7][clpu;7][abqu;7][cevw;7][cfuw;7][dlrw;7][aepx;7][adlx;7][ewxC;7][dprC;7][bdry;7][cdnp;7][bcln;7][abem;7][acor;7][mpuw;7][acop;7][bmpw;7][bsvw;7][amuC;7][aexC;7][afrx;7][bdop;7][cdex;7][cewx;7][abef;7][fpwx;7][bpuy;7][dfwx;7][aruy;7][bv wy;7][depr;7][lrvx;7][brvy;7][anpu;7][apry;7][aquC;7][beru;7][apwy;7][cluw;7][aepv;7][bvwC; 7][bdqu;7][bluv;7][berC;7][deru;7][aeux;7][aprC;7][aorx;7][bdem;7][clru;7][alnw;7][acln;7][cde m;7][aepr;7][clnp;7][abvy;7][lvwx;7][duCy;7][cvwy;7][adry;7][bevx;7][evwC;7][dlpu;7][cdqu;7][dlnp;7][bcrC;7][amrw;7][acqC;7][bmpu;7][abrC;7][bpvy;7][cpry;7][cdnw;7][aemw;7][bors;7][dvwC;7][adqw;7][aemu;7][opvx;7][dmrw;7][belw;7][berx;7][bfpx;7][dlmw;7][ablo;7][bdnp;7][a owx;7][bdpy;7][bduy;7][clnw;7][brsx;7][alnr;7][dpvx;7][cerC;7][lrux;7][dlru;7][alvx;7][buwy;7][bfvw;7][belp;7][acdn;7][adlm;7][bdel;7][bdfx;7][abxC;7][cmrw;7][bdgw;7][abxy;7][almw;7][dluw;7][bmpr;7][fpuw;7][bfux;7][aerC;7][bouw;7][abry;7][cdmr;7][rsvx;7][aluv;7][orsv;7][deuv ;7][opuw;7][luwx;7][bmrw;7][euvw;7][bcdn;7][erwC;7][lpvw;7][admr;7][bdln;7][bnuw;7][afru; 7][cdev;7][abqC;7][bnpw;7][cdCy;7][cpuy;7][oprs;7][lopw;7][lorw;7][bfpu;7][abfv;7][cdxC;7][brwC;7][duwy;7][demC;7][arsv;7][cruy;7][absx;7][aoux;7][cdqC;7][cmwC;7][blnv;7][cnpw;7][r uwC;7][euwx;7][asvx;7][fuwx;7][dquw;7][bpwy;7][dfpu;7][delw;7][afvw;7][dlpC;7][alpv;7][ep wx;7][alnp;7][dopw;7][admx;7][cmpu;7][lnpy;7][bemw;7][adel;7][ador;7][acvy;7][aevx;7][buy C;7][ceru;7][cdlC;7][adqr;7][adfC;7][berv;7][bdov;7][acnw;7][opry;7][acqr;7][bdef;7][dnpw;7][bcmp;7][bdfr;7][erwx;7][acou;7][abps;7][alor;7][aemC;7][aerx;7][epvw;7]

5 Phage Combinations

5 phage combinations were analyzed in silico for their ability to lyse the 352 different strains of Klebsiella pneumoniae bacteria based on the ability of the single phage to lyse the bacteria as assessed by a liquid media assay.

The combinations with the highest “at least 1 phage % coverage” are provided herein below (out of 118,755 possible combinations, the top 0.4% (476) are provided). The number following each combination refers to the Percent trait performance—in this case the percent of the 352 strains that are targeted by the phage combination. The phage combinations are ordered in descending performance grade.

For example, in the case of [ablqg;78], the 5 phage combination that provided the highest percent coverage, 395-D2-3-1, 395-D2-3-2, KP10-11, KP10-48 and CT-227-KP4M1-11 lysed 78% of all the strains of Klebsiella pneumonia analyzed.

[ablqg;78][algsg;78][ablyg;78][ablqB;77][alqvg;77][alsyg;77][blqug;77][alogg;77][blqsg ;77][algsB;77][ablyB;76][bluyg;76][alqrg;76][bflqg;76][blqtg;76][alvyg;76][bclqg;76][blquB;76][aflyg;76][alryg;76][alsyB;76][aloqB;76][alpqg;76][alqvB;76][aloyg;76][blsyg;76][alqyg;76][a blsg;76][bluyB;76][blqsB;76][blqwg;76][bflyg;76][bclyg;75][blmqg;75][aluyg;75][aflgg;75][aly gj;75][algtg;75][alpyg;75][altyg;75][algrB;75][alygi;75][bflqB;75][alqgj;75][bltyg;75][aclyg;75][alvyB;75][blqtB;75][aclqg;75][alwyg;75][aelyg;75][ablqi;75][almyg;75][aloyB;75][alqwg;75][blwyg;75][alqgi;75][ablCg;75][blqgj;75][alyiB;75][alqxg;75][alpqB;75][blsyB;75][alqug;75][al ygB;75][alygh;75][alqsi;75][abflg;75][alxyg;75][alryB;75][aflyB;75][lquvg;75][aelqg;75][almqg ;75][bclqB;75][blqxg;75][bdlqg;75][aluyB;74][alqgB;74][alqyB;74][blqgi;74][blmyg;74][adlqg; 74][blqyg;74][alqgh;74][blqwB;74][luvyg;74][ablug;74][bflyB;74][alqiB;74][alnyg;74][alygz;74][abltg;74][adlyg;74][ablsB;74][alyjB;74][alogi;74][aflsg;74][lsuyg;74][alngg;74][lgsvg;74][ab lyi;74][belqg;74][alpyB;74][algjB;74][alCyg;74][alsCg;74][lgsug;74][aflqB;74][alqgz;74][altyB ;74][blogg;74][blygj;74][blqiB;74][blqgB;74][blxyg;74][blmqB;74][alygk;74][alqCg;74][lgswg; 74][alygA;74][blpqg;74][blngg;74][algtB;74][blqgh;74][blqCg;74][alsyi;74][alqgA;74][bclyB;74][alquB;74][blqjB;74][bltyB;74][blygi;74][lquvB;74][bdlyg;74][aelyB;74][blsug;74][alwyB;74][almyB;74][alqgk;74][cluyg;74][alsvg;74][blqrg;74][bdlqB;74][blqgz;74][ablgj;74][alqvi;74][bl nyg;74][aloyi;74][ablmg;74][luwyg;74][alqwB;74][alyhB;74][luvyB;74][adlyB;74][blyiB;74][al qxB;74][lruyg;74][blqgA;74][aclqB;74][abluB;74][alxyB;74][blqsi;74][ablgi;74][aelqB;74][alm qB;74][blygB;74][alsug;74][blqxB;74][blqgk;74][blqui;74][blwyB;74][aclyB;74][flqvg;74][blqv g;74][ablCB;74][clqug;74][dlasg;74][adlaB;74][abflB;74][blygh;74][alyzB;73][lsuyB;73][alnyB ;73][alrsg;73][alykB;73][belyg;73][bloyg;73][louyg;73][blqyB;73][blmyB;73][bloqB;73][blyjB; 73][algri;73][lswyg;73][blygz;73][blpyg;73][alqhB;73][alswg;73][aflvg;73][dluyg;73][clqvg;73][loqug;73][abliB;73][bclqi;73][ablgB;73][ablgh;73][lgsuB;73][lgrug;73][abltB;73][bluCg;73][al stg;73][dlqvg;73][lsvyg;73][blryg;73][blqhB;73][abljB;73][luxyg;73][flvyg;73][alyAB;73][abdl g;73][fluyg;73][alnqB;73][ablrg;73][ablgz;73][clqsg;73][aflsB;73][bluyi;73][belqB;73][alqzB;73][lpuyg;73][ablwg;73][lgsvB;73][dlsyg;73][algkB;73][bclug;73][blygA;73][lpqug;73][blsuB;73][alCyB;73][ablog;73][lgsxg;73][ablng;73][lquwg;73][abclg;73][lgswB;73][blpqB;73][blygk;73][alosg;73][blvyg;73][blnqB;73][bdlyB;73][alsCB;73][alvCg;73][alqCB;73][blsCg;73][abelg;73][blxyB;73][blqkB;73][bflqi;73][ablxg;73][aclsg;73][blqrB;73][blqti;73][blqzB;73][bflCg;73][lqv wg;73][blsyi;73][altvg;73][blqCB;73][alpsg;73][blCyg;73][cluyB;73][louyB;73][ablgA;73][alm sg;73][lruyB;73][ablgk;73][aflrg;73][alsgi;73][dlqsB;73][alsuB;73][adlsg;73][dlqug;73][loquB;73][alsvB;73][luwyB;73][alryi;73][dluyB;73][lgtvg;73][lmuyg;73][lquxg;73][alpqi;73][alvyi;73][alqAB;73][alsgh;72][dlvyg;72][ablmB;72][ablvg;72][dlqvB;72][ablpg;72][alyij;72][ablsi;72][ltu yg;72][bltug;72][alsgB;72][bflug;72][aflyi;72][blyhB;72][flqvB;72][blmug;72][bloyB;72][dlqxg ;72][binyB;72][blqAB;72][lqvxg;72][alsiB;72][lgruB;72][lquyg;72][blqvB;72][blqwi;72][clvyg; 72][aflog;72][bclyi;72][alsgj;72][luygj;72][clquB;72][aluvg;72][bluCB;72][aelsg;72][alsxg;72][blpyB;72][aclqi;72][luxyB;72][aclyi;72][blykB;72][alswB;72][clsyg;72][blryB;72][ablhB;72][al rsB;72][bclCg;72][flqsg;72][bltCg;72][bflyi;72][elqvg;72][lpquB;72][fluyB;72][abdlB;72][lsvy B;72][eluyg;72][alrCg;72][lswyB;72][belyB;72][aelyi;72][abloB;72][flqug;72][lpuyB;72][alnsg; 72][lpqsg;72][alpyi;72][blugj;72][alsgz;72][blyzB;72][dlquB;72][alqij;72][alosB;72][clqxg;72][dlsyB;72][aflvB;72][aloCg;72][lquwB;72][bflsg;72][dlxyg;72][alstB;72][alqyi;72][bdlug;72][clx yg;72][flsyg;72][ablzB;72][lnuyg;72][lgrsg;72][ablkB;72][alsgk;72][ltvyg;72][ablwB;72][bloqi; 72][lsxyg;72][ablnB;72][lvwyg;72][alvgj;72][adlsB;72][altyi;72][aflqi;72][blsCB;72][blvyB;72][aelgi;72][fivyB;72][ablrB;72][blqij;72][alrtg;72][luyiB;72][luyjB;72][bcluB;72][alwyi;72][lmq vg;72][blyAB;72][aluyi;72][alyiz;72][lmuyB;72][alqti;72][absyg;72][bdlqi;72][blmqi;72][lgstg; 72][alvCB;72][alrug;72][luygB;72][luygi;72][lquxB;72][alsgA;72][luygh;72][lgsxB;72][abelB;72][lgrwg;72][clqvB;72][alnyi;72][lgtvB;72][alnqi;72][lpsyg;72][loqwg;72][dlvyB;72][lgtxg;72][alotg;72][almvg;72][lwxyg;72][alsjB;72][lrsyg;72][bltuB;72][bdlsg;72][lqvgj;72][blnqi;72][alms B;72][flxyg;72][blugh;72][blstg;72][aflpg;72][dlqxB;72][blugi;72][loqxg;72][bluiB;72][blnug;72][blugB;72][aflrB;72][lpqxg;72][afloB;72][lgrxg;72][abqsg;72][bluwg;72][alqiz;72][lqvwB;72][lqwxg;72][lqvyg;72][bclsg;72][clqsB;72][blmCg;72][abclB;72][adlyi;72][lvxyg;72][ablCi;72][bdluB;72][ablxB;72][adlqi;72][alpsB;72][bltyi;72][blwCg;72].

The combinations with the highest “at least 2 phage % coverage” are provided herein below. The phage combinations are ordered in descending performance grade.

Thus, for example, in the case of [ablqy;47], when 395-D2-3-1, 395-D2-3-2, KP10-11, KP1048 and KP10-91 are used in a combination, 47% of the bacterial strains tested were targeted by at least 2 of the 5 phage.

[ablqy;47][ablpy;46][ablpq;46][ablry;46][blpqy;45][lpqvy;45][ablwy;45][blqvy;45][ablu y;45][abfly;45][ablCy;45][ablny;45][abloy;45][alqvy;45][abcly;45][ablvy;45][alpqy;45][ablqr;44][ablsy;44][blqry;44][bdlqy;44][aluvy;44][blvCy;44][lpqry;44][blqwy;44][blquy;44][alquy;44][alpvy;44][abdly;44][alruy;44][ablpu;44][blpCy;44][lqvwy;44][blqsy;44][abcln;44][bdlpy;43][ab lnr;43][bdlsy;43][lpqwy;43][ablmy;43][adlpy;43][alpuy;43][abloq;43][algry;43][ablqw;43][blrC y;43][blwCy;43][ablqu;43][ablty;43][alpry;43][blogy;43][ablqv;43][ablqs;43][lquvy;43][bdqvy; 43][abdlp;43][abclq;43][bdlny;43][ablxy;43][alpqv;43][ablnq;43][alrwy;43][abclp;43][alqwy;43][abflq;43][bdlpq;43][ablnw;43][bquvy;43][abqwy;43][abpqy;43][ablqC;43][ablrC;43][bdlvy;43][blngy;43][bdlCy;43][abqvy;43][blnwy;43][ablpv;43][ablps;43][bdlry;43][adlvy;43][alrvy;43][dlpqy;43][alvwy;43][ablnv;43][adloy;43][blsCy;43][alouy;43][ablpC;43][bluvy;43][alogy;43][bl qxy;43][ablnp;43][ablnu;43][blnCy;43][alsuy;43][abdln;43][ablgB;43][adlry;43][lpqsy;43][aflvy ;43][alpwy;43][bdlqs;43][abdlq;43][ablpw;43][alpsy;43][lpquy;43][blqvC;42][bloCy;42][dlqvy; 42][abqry;42][blpqC;42][alowy;42][aluwy;42][loqvy;42][adlqy;42][abdqy;42][alory;42][algsy;42][alvCy;42][ablpr;42][adlsy;42][alnvy;42][lgrwy;42][adlpv;42][alnuy;42][bdloy;42][bdlwy;42][lgrvy;42][ablop;42][alsvy;42][bluwy;42][lngry;42][alswy;42][abely;42][logry;42][dlqry;42][ab cls;42][ablwC;42][blpvy;42][lnpqy;42][blxCy;42][ablmq;42][alovy;42][alpCy;42][adlnw;42][bl puy;42][alpuv;42][adlwy;42][aclvy;42][bqvCy;42][alqxy;42][ablno;42][lnqvy;42][blnvy;42][bln ry;42][bdlqv;42][abcqy;42][ablru;42][alrsy;42][abclC;42][blnuy;42][alpqr;42][alnwy;42][ablvC; 42][adlny;42][abdlr;42][abflp;42][abclr;42][alnuw;42][adlpq;42][adlnu;42][blsvy;42][lngwy;42][alnry;42][lpvCy;42][blnqv;42][ablng;42][ablns;42][abclv;42][abmqy;42][lgsvy;42][ablrw;42][a dluy;42][blrsy;42][alpxy;42][bclqy;42][blruy;42][blsuy;42][blpsy;42][ablnB;42][blrwy;42][bluC y;42][beqvy;42][ablmp;42][blnuw;42][alpru;42][loqwy;42][bgruy;42][bnqvy;42][adlnv;42][bdq xy;42][ablou;42][blswy;42][bcdly;42][abluv;42][blnrw;42][bpqvy;42][bdlqr;42][ablmr;42][abcl o;42][ablsC;42][bmqvy;42][aclpy;42][aboqy;41][clpqy;41][aclnu;41][bdluy;41][ablpt;41][bqsvy ;41][bdpqy;41][abdlo;41][abfln;41][ablyh;41][ablms;41][ablsu;41][adlps;41][ablyj;41][bluxy;41][ablor;41][abloC;41][ablyB;41][ablyz;41][lqvxy;41][blpqu;41][lgrsy;41][ablyi;41][ablsw;41][dl psy;41][bfqvy;41][lnvwy;41][blnpy;41][aflpy;41][lvwCy;41][abclu;41][abngy;41][aclry;41][bdl nq;41][lgswy;41][bdlxy;41][bdlnv;41][adlxy;41][acluy;41][blouy;41][abdlv;41][ablyk;41][ablyg ;41][adlpr;41][alnqu;41][alpqu;41][bdloq;41][lpqxy;41][abdlC;41][ablyA;41][alquv;41][alrxy;41][ablnC;41][ablpx;41][bdlnw;41][alrCy;41][bdgry;41][blpwy;41][boqvy;41][abqxy;41][alwCy;41][ablvw;41][ablos;41][bpquy;41][abnwy;41][blpnv;41][bdawy;41][dloy;41][aflry;41][alaru;41 ][abdls;41][dlpvy;41][blory;41][blrvy;41][dlqwy;41][blmvy;41][adlov;41][adlqv;41][blqsC;41][abdlw;41][aclnv;41][bquwy;41][lgruy;41][abluw;41][lquwy;41][alngw;41][adlpw;41][blqrC;41][alngy;41][abluC;41][bflvy;41][ablow;41][abquy;41][alpqs;41][algsv;41][bdlsC;41][abpuy;41][b lnsw;41][clqvy;41][ablrs;41][adlnr;41][blquv;41][bclny;41][almvy;41][adlos;41][algrw;41][blpr y;41][adlpx;41][dlpCy;41][blnqw;41][adlqr;41][bdfly;41][adlnx;41][lnrwy;41][acdln;41][bdvCy ;41][aluCy;41][blvwy;41][ablmv;41][lopqy;41][alqrv;41][abygB;41][bdlqC;41][bqrvy;41][lgsuy ;41][ablqx;41][abdlu;41][abdpy;41][dlpry;41][luvwy;41][bclnv;41][aqrvy;41][adlnq;41][loquy;41][abcny;41][adlpu;41][abdvy;41][aclny;41][bgrwy;41][alsCy;41][lqwxy;41][bdlvC;41][abflr;41][aclqy;41][blnqr;41][bclCy;41][alnxy;41][blqCy;41][bfluy;41][abclx;41][dlory;41][aqvwy;41][altvy;41][bloqC;41][clqwy;41][alnug;41][alquw;41][ablni;41][ablmo;41][alopy;41][adlng;41][bl nvw;41][abvgB;41][ablsv;41][adlnB;41][bpqwy;41][blpqw;41][dlqsy;41][alpqw;41][bdqsy;41][alqvw;41][aclnr;41][abflo;41][aloCy;41][ablqt;41][bclns;41][bcqvy;41][bquxy;41][clqry;41][bp gry;41][lprCy;41][bdlnp;41][abvCy;41][bdlps;41][alnru;41][bdvgB;41][loqsy;41][blqsw;41][abr uy;41][alnuv;41][abelp;41][adlsv;41][bcluy;41][ablnx;41][bqvwy;41][aclsy;41][alnuB;41][dlnry ;41][bqvxy;41][aluxy;41][dlpwy;41][abcfl;41][dlvCy;41][blpqs;41][abqsy;41][alvxy;41][blquw; 41][bdogy;41][blnvC;41][dlovy;41][bqtvy;41][alnrw;41][blmqy;41][acloy;41][blopy;41][blnxy; 41][afloy;41][blqwC;41][abflC;41][adlnp;41][aloqv;41][lquxy;41][alnvw;41][blpxy;41][blnuv;41][lpwCy;41][adlvw;41][bcdln;41][blnpq;41][adlns;41][abdny;41][adloq;41][bdlpC;41][adqxy;41][bdlsw;41][bdlns;41][abdgB;41][abwCy;41][dlnvy;41][abdry;41][ablmn;41][adlow;41][bdlqw ;41][adqvy;41][almry;41][ablpz;40][cdlry;40][bdlnu;40][adlsw;40][alpsv;40][dlvwy;40][alnuC;40][dlrvy;40][blqrw;40][dlpqv;40][belqy;40][alwxy;40][dlowy;40][alrwC;40][blrxy;40][alrty;40][dlrsy;40].

The combinations with the highest “at least 3 phage % coverage” are provided herein below. The phage combinations are ordered in descending performance grade.

Thus, for example, in the case of [abdly;30], when 395-D2-3-1, 395-D2-3-2, 395-D2-3-4, KP10-11 and KP10-91 are used in a combination, 30% of the bacterial strains tested were targeted by at least 3 of the 5 phage.

[abdly;30][abdlv;30][ablvy;28][abdrv;28][abdlw;28][abdls;28][abdvy;28][abdlp;28][abdl o;28][abcdl;28][abruv;28][abprw;28][ablwy;28][abdpv;28][abrvy;28][ablpy;28][ablrv;28][abqvy ;28][abdov;28][abduw;28][abcdw;28][ablvw;28][abdpr;28][abrvw;28][abluw;28][abdlr;28][abqr v;28][ablrw;27][bdlpv;27][ablqv;27][bdlvw;27][ablpv;27][bdlvy;27][abluy;27][abdsy;27][abdv w;27][abcrv;27][abdln;27][abdrw;27][abrvC;27][abluv;27][abduv;27][abdlu;27][abcuw;27][abd ow;27][abdlx;27][abdmv;27][abdlq;27][ablry;27][abclv;27][ablsv;27][abvCy;27][blpvw;27][abp vw;27][abdwy;27][ablpw;27][abinw;27][abdux;27][abdsv;27][bdlpr;27][abquy;27]1[bdlpu;27][ab dlm;27][adlvy;27][abclp;27][abuvw;27][bdlrv;27][abcdv;27][ablsw;27][abclw;27][abdxy;27][ab cwx;27][abfrv;27][bdpru;27][ablpr;27][abuvy;27][abdwx;26][bdrvw;26][ablrs;26][bdlpy;26][ab drx;26][abdry;26][abdpw;26][acduw;26][bdlov;26][bdlvx;26][ablvC;26][abdqv;26][bdpvw;26][a bouv;26][abpru;26][bcpvw;26][bcdlv;26][abcdu;26][blrvw;26][ablru;26][ablov;26][blpwy;26][b dlwy;26][abcpr;26][bdluv;26][blqvy;26][adluw;26][abduy;26][ablux;26][abdor;26][bdluy;26][bl prv;26][blruv;26][abruw;26][abrtv;26][abcpw;26][abdlC;26][blvwy;26][blprw;26][abdpx;26][ab ovw;26][abopv;26][bdruv;26][aberv;26][blpvy;26][ablmv;26][brvwy;26][absvw;26][abcdx;26][a cdlu;26][ablxy;26][bruvw;26][abcly;26][abprv;26][abdrs;26][blqry;26][ablqy;26][abmuw;26][bl puy;26][abpuv;26][aduwx;26][alvwy;26][aluwy;26][abdru;26][abclr;26][acdlv;26][bprvw;26][ac dlr;26][abnrv;26][adlvw;26][blnpr;26][ablvx;26][bdrvy;26][aborw;26][bdprw;26][bcdlw;26][blu vy;26][bcrvw;26][abdfu;26][blprs;26][blpru;26][abdqu;26][abrsw;26][abpvy;26][abdnw;26][abc nw;26][adluy;26][bdlpw;26][blpry;26][abuwx;26][bdlru;26][ablow;26][abrvx;26][ablpC;26][adl ux;26][adruw;26][ablqw;26][bclpv;26][abcvw;26][abloy;26][abdvx;26][adlwy;26][abcov;26][ab dnv;26][abdgw;26][acdvw;26][aprvw;26][bcdlp;26][abcdr;26][bpvwy;26][abdoy;26][acdlw;26][abcpv;26][abpuw;26][aluwC;26][abdwC;26][ablsy;26][aborv;26][bdlry;26][abdpy;26][abdvC;26][abrCy;26][ablCy;26][aluwx;26][bclvw;26][abflv;26][aduvw;26][abrsv;26][abdpu;26][bdluw;26][bpuvw;26][acpvw;26][abcow;26][abcwC;26][abdox;26][abcsw;26][bclpr;26][adlwx;26][aluv w;26][ablnp;26][bopvw;26][abuwy;26][ablpu;26][abosw;26][aclwx;26][abcrw;26][adlry;26][abc uv;26][blpvC;26][aduvx;26][abquw;26][bervw;26][aclvw;26][abmvw;26][adlrv;26][abmrv;26][bcdrv;26][abrwy;26][ablmw;26][abcln;26][abdmu;26][abdfv;26][ablwC;26][bcprw;26][bdrvx;26][abuxy;26][abpsw;26][bluvw;26][abcew;26][blprC;26][bdlop;26][ablqu;26][bprwy;26][abpry;26][abdmw;26][abdqy;26][bdlor;26][brvwx;26][adlpr;26][adlpu;26][abclu;26][abrux;26][bruvx;26][ablnv;26][abouw;25][abeov;25][brvwC;25][abdmr;25][bcrvx;25][bduvy;25][bdlou;25][blsvw ;25][bdouv;25][acdwy;25][abcsv;25][blovw;25][abdsx;25][adrvw;25][bclrw;25][abdtw;25][adv wy;25][abcdy;25][ablmu;25][blrwy;25][bluwy;25][bcdlr;25][abcls;25][alnvw;25][abrwx;25][blp vx;25][bcluv;25][ablmy;25][alrvw;25][bdvwy;25][aboru;25][abdel;25][bclrv;25][blpqv;25][abdq x;25][bdlsv;25][blruy;25][alqwy;25][bdlrw;25][abltv;25][aclnw;25][bcruv;25][acuwx;25][adlqw ;25][abdou;25][abruy;25][alprw;25][alnpu;25][abqwy;25][bdovw;25][bduvw;25][abmpv;25][blr su;25][abluC;25][bdlps;25][ablop;25][abprx;25][blpuv;25][abpvC;25][acdru;25][bmpvw;25][bdr uw;25][bpsvw;25][acvwx;25][aboqv;25][alpvw;25][buvwy;25][abpqv;25][ablrx;25][abvwy;25][abovC;25][acrwx;25][acdux;25][bdlow;25][bnpvw;25][abdtv;25][abdsw;25][abopw;25][lprvw;25][bprwx;25][aduwy;25][alprv;25][blvCy;25][abnrw;25][bpvwC;25][abqxy;25][ablqx;25][ablnu ;25][ablou;25][bfrvw;25][abdCy;25][bprvx;25][alpuw;25][blouv;25][acuvw;25][abopr;25][abps v;25][alpwy;25][abqux;25][blprx;25][abelv;25][acnvw;25][bdlay;25][bdpvx;25][bdlsu;25][abdp s;25][bcduw;25][abcux;25][bcdlu;25][bclpw;25][adpuw;25][alquw;25][aclpv;25][bdqrv;25][blm pr;25][abuCy;25][ablny;25][abcdp;25][blopv;25][aluvx;25][abovy;25][abuxC;25][bpvwx;25][bo prw;25][acpwx;25][adluv;25][ablnx;25][abvwx;25][blvwx;25][adlrw;25][bcnvw;25][abpvx;25][acprv;25][abmrw;25][abuwC;25][bcpwx;25][bouvw;25][abqry;25][abfpv;25][ablsu;25][abuvx;25][brvxy;25][bdopr;25][blpqr;25][alpvy;25][abdop;25][bdoru;25][abfuw;25][abnvw;25][aclwy;25][bruvy;25][bdpvy;25][abdtu;25][abprC;25][abcvy;25][ablor;25][adlwC;25][ablps;25][acduv;25][blnpv;25][bdpuv;25][bdsvw;25][bgrvw;25][adprv;25][borvw;25][adlpv;25][bcorw;25][dlprv; 25][bdprv;25][bdlsw;25][abcrx;25][abcru;25][acvwC;25][bcrvy;25][abrvh;25][aclpr;25][bdorv;25][bclpy;25][abdnp;25][blpqy;25][alwxy;25][abnuw;25][bgrvy;25][abcdn;25][abqrw;25][abdos; 25][abxCy;25][abdny;25][bervx;25][abloC;25][abpux;25][abnpv;25][ablmp;25][bdlwC;25][bcov w;25][blmvw;25][bluCy;25][adelw;25][absuv;25][abrvk;25][lpruv;25][adlpy;25][abduC;25][acr vw;25][abrvg;25][bluvx;25][boruv;25][bdlrs;25][acdwx;25][blrvy;25][bepru;25][abcds;25][abrv z;25][blrvC;25][abnov;25][alpuC;25]

The combinations with the highest “at least 4 phage % coverage” are provided herein below. The phage combinations are ordered in descending performance grade.

Thus, for example, in the case of [abprv;20], when 395-D2-3-1, 395-D2-3-2, KP10-4, KP10-61, and KP10-85 are used in a combination, 20% of the bacterial strains analyzed were targeted by at least 4 of the 5 phage.

[abprv;20][bpruv;19][abcrw;19][abrvw;19][abuwx;19][abcvw;19][bdprv;19][bprvw;19][abduw;19][abdvw;18][abrvx;18][abpvw;18][blprv;18][abdlu;18][bcprv;18][abvwx;18][abdru;18][abpvx;18][abdux;18][ablrv;18][abcrv;18][abduv;18][abpwx;18][ablwx;18][abdrv;18][abcuw;18][abuvw;18][abuvx;18][bpvwx;18][brvwx;18][bruvw;18][borvx;18][abdlw;18][abpux;18][abru w;18][abrwx;18][abluv;18][bcvwx;18][abcdw;18][blruv;18][bprvx;18][abdwx;18][bcrvw;18][ab dlv;18][abdsu;18][abcwx;18][ablpv;18][abcuv;18][abdou;18][abcvx;18][abdrw;18][abclw;18][a bcpw;18][abovw;18][acdlw;18][ablvw;18][abdvx;18][aborv;18][adluw;18][bdlrv;18][blpuv;18][abcdu;18][ablvx;18][bcruw;17][bcpvw;17][abdpv;17][acdvw;17][acruw;17][abcpv;17][acdrw;17][bclrv;17][bnprv;17][abruv;17][bcuvw;17][blrvx;17][abdpw;17][abouw;17][abluw;17][acrvw;17][buvwx;17][abpuv;17][abpuw;17][abcdv;17][ablpu;17][abduy;17][acuvw;17][abdpu;17][abeu w;17][acdpw;17][abfuw;17][abcux;17][bcruv;17][abprx;17][beprv;17][abdlr;17][absuw;17][abv wy;17][abcru;17][blpvw;17][borvw;17][acluw;17][bdruv;17][blrvw;17][abdsw;17][abovx;17][a uvwx;17][abdlx;17][bcprw;17][abeux;17][ablpr;17][bdvwx;17][abdfw;17][abprw;17][acduw;17][abcdr;17][ablux;17][aberu;17][bpuvx;17][bpruw;17][abevw;17][acuwx;17][aduwx;17][abfuv;17][bcdvw;17][adluv;17][abnvw;17][bcpvx;17][blruw;17][abuwy;17][bclvw;17][bcdpw;17][blpru :17][bdrvw;17][bdpvw;17][abvwC;17][aduvw;17][boruv;17][bcrvx;17][bpuvw;17][abavw;17][a bdwy;16][bluvw;16][bdrvx;16][bcpwx;16][abouv;16][blprw;16][ablpw;16][abuwC;16][bdlrw;16][ablpx;16][bcrwx;16][abnrv;16][bovwx;16][acpvw;16][bprux;16][aruvw;16][ablru;16][abclv;16][bdlpv;16][apuwx;16][bmprv;16][apvwx;16][abdew;16][bcdlw;16][bduvw;16][abcmw;16][adru v;16][abdnu;16][bcduw;16][blvwx;16][bouvw;16][brvwy;16][bdlvw;16][abeuv;16][acprw;16][a brvy;16][bcdrw;16][abdfu;16][abruC;16][bnrvw;16][abpru;16][acpuw;16][abewx;16][aduwy;16][bruvx;16][acvwx;16][bprvC;16][bdor;16][abfru;16][abvxy;16][bdlpr;16][arvwx;16][ablrw;16][abmvw;16][abowx;16][abfvw;16][bdluv;16][adpuw;16][abnuv;16][abopw;16][bruwx;16][abnv x;16][acdwx;16][bdluw;16][abdrx;16][bclpw;16][abevx;16][blpvx;16][acduv;16][abnpv;16][abc lx;16][abcsu;16][abclu;16][advwx;16][abrux;16][cruvw;16][abmrv;16][bpuwx;16][abdpr;16][ab cow;16][abprs;16][bclpv;16][acdwy;16][aborw;16][abwxC;16][alvwx;16][adpvw;16][abduC;16][abcfu;16][abcfw;16][boprv;16][abcsw;16][abcdp;16][aberv;16][acruv;16][acdew;16][alpuv;16][aclvw;16][boruw;16][bdprw;16][abwxy;16][abcrx;16][bvwxy;16][apruw;16][abdtu;16][abpwy;16][bprwx;16][apruv;16][absvw;16][abswx;16][bclwx;16][bdlpw;16][adlru;16][bpvwy;16][adfuw ;16][apuvw;16][abcpx;16][bprvy;16][adnuw;16][bclrw;16][abepu;16][blrvy;16][bcpuw;16][abeo u;16][brsvw;16][bduwx;16][aclpw;16][abepw;16][bdeuw;16][aclrw;16][bprsw;16][abdmw;16][a cpwx;16][abdwC;16][bprsv;16][adlrw;16][bnpvw;16][bdlru;16][abcd;16][bcluw;16][alprv;16][c dprw;16][bcdwy;16][ablrx;16][bcuwx;16][adlvw;16][abnuw;16][alruw;16][abrwy;16][abmpw;16][bcduv;16][abnwx;16][bdruw;16][bluvx;16][adruw;16][bpgrv;16][lpruv;16][abopr;16][abdow; 16][adprw;16][abcdy;16][acdwC;16][abcpu;16][abcou;16][bnruv;16][blorv;16][abdfr;16][abmuv ;16][abdqw;16][abdov;16][adpuv;16][abdnw;16][abcew;16][blpuw;16][bcopw;16][cprvw;16][bc nrv;16][bclpr;16][bdnrv;16][bdpvx;16][bluwx;16][aclwx;16][bfprv;16][alruv;16][adrvw;16][bdp uv;16][abnpw;16][brvxy;16][bopvw;16][aberw;16][acdmw;16][abquv;16][abuvy;16][acdnw;16][pruvw;16][alpuw;16][bdpwx;16][blprx;16][abmwx;16][abcde;16][abcpr;16][cdluw;16][adeuw; 16][bcdpv;16][abdrC;16][abuxC;16][abopv;16][bdpuw;16][acdpv;16][abdeu;16][cdpvw;16][blp ux;16][abpsx;16][aprvx;16][abdvy;16][adpwy;16][abctw;l6][abdlp;l6][abdqu;16][bdlwx;l6][ad lux;16][aruwx;16][bnrvx;16][abcdf;16][adlpw;16][abquw;16][abfpu;16][aprvw;16][abuvC;16][b mrvw;16][bcdrv;15][bcprx;15][blrwx;15][abpwC;15][bevwx;15][bclvx;15][abnpr;15][blopr;15][bdovw;15][bduvx;15][abpvy;15][borwx;15][abruy;15][bcdew;15][bnpvx;15][adprv;15][apuvx;15][acdrv;15][bepvw;15][abdop;15][abpsw;15][acdpr;15][aduvx;15][absvx;15][abfux;15][abdor;15][adepw;15][admpw;15][abdmu;15][abmvx;15][abcdC;15][cduvw;15][abrwC;15][acdfw;15][a bepv;15][abpsu;15][acopw;15][bcouw;15][abtuw;15][acrwx;15][aluvw;15][abvxC;15][blrux;15][bdlux;15][cdruw;15][abdpx;15][abfvx;15][abqru;15][acdlu;15][bprsu;15][adsuw;15][bcdmw;15][alrvx;15][belrv;15][bcorv;15][abnux;15][afuvw;15][abqwx;15][adlrv;15][abmuw;15][abdry;15][abrvC;15][alpru;15][dlprv;15][abmru;15][abelu;15][aduwC;15][abclr;15][blovx;15][bcovw;15][bdrwx;15][aluwx;15][abder;15][bcpuv;15][bnprw;15][acdtw;15][bnvwx;15][acouw;15][bdpwy; 15][lprvw;15][bnpuv;15][bruvy;15][bmpvw;15][bdlvx;15][acmpw;15][bdfuw;15][acdpy;15][adf vw;15][boprx;15][abctu;15][acpuv;15][bopru;15][bcluv;15][bcdru;15][abcwC;15][abrsw;15][ab oru;15][acdpu;15][acuvx;15][pruvx;15][abrsv;15][adluC;15][bdvwy;15][abmpv;15][bprsx;15][a dfpw;15].

### Example 3

### Phage Combinations Selected According to Host Coverage as Classified by Bacterial Capsule Type

For this example, the particular phages are referred to by the single letter designation in Table 1, herein above.

2 Phage Combinations

2 phage combinations were analyzed in silico for their ability to lyse the 352 different strains of Klebsiella pneumoniae bacteria as profiled by their capsule type, based on the ability of the single phage to lyse a bacteria of a particular capsule type as assessed by a liquid media assay.

The combinations that lyse the highest number of bacterial strains (as profiled by their capsule locus type) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[lg;78][lB;77][li;76][lu;74][lq;74][dl;74][lw;73][lv;73][ls;73][lC;73][ly;73][lj;73][cl;72][lx;72][el;72][ln;70][lp;70][lr;70][lt;70][lz;69][fl;69][lm;69][lo;68][lh;67][lA;67][lk;67][dn;65][ds; 65][ru;64][uv;64][uw;64][vy;64][dp;64][di;64][du;64][dg;64][dB;64][nu;64][dr;64][dv;62][uy;62][dy;62][nv;62][uB;62][wy;62][qu;62][dq;62][mu;61][dm;61][qw;61][ug;61][qv;61][pu;61][cu;61][nw;61][uC;61][ri;60][dt;60][ry;60][mr;60][vC;60][rg;60][qr;60][dj;60][wi;60][su;60][do;60][d w;60][rB;60][yg;60][dx;60][df;59][yi;59][eu;59][de;59][py;59][ui;59][pq;59][rw;59][nr;59][ny;59][uj;59][vi;57][vg;57][rC;57][wg;57][cn;57][vB;57][sw;57][yB;57][nC;57][my;57][dC;57][ou;57][mp;56][nq;56][mw;56][mv;56][ms;56][cd;56][tu;56][mn;56][wB;56][ey;56][dz;56][ng;56][ns; 56][pv;56][sv;56][wC;56][pC;56][ux;56][pw;56][xy;56][sy;56][Cg;56][Ci;56][qg;56][pg;56][wj; 56][sC;56][cq;56][dk;56][dA;56][rj;56][er;56][qx;56][pB;56][cy;56][np;56][nB;56][fn;56][ni;56][cr;56][pi;56][cv;56][oy;56][vw;56][en;55][mC;55][tw;55][no;55][nx;55][yj;55][dh;55][qB;55][n j;55][rs;55][cp;55][qi;55][oq;55][fy;55][CB;55][oC;55][ps;55][pr;55][rv;55][cg;55][ci;55][qs;55][ev;55][rx;53][ei;53][fu;53][cs;53][ty;53][mx;53][ow;53][eq;53][fw;53][qt;53][tv;53][mt;53][eg; 53][ep;53][mg;53][nt;53][em;52][cC;52][fv;52][vj;52][mi;52][fq;52][vx;52][uz;52][mB;52][cm; 52][Cy;52][tC;52][mo;52][ew;52][es;52][mq;52][pj;52][xg;51][xi;51][uA;51][Cj;51][cB;51][cw; 51]

The percent of host bacterial strains (as profiled by their capsule locus type) that are infected by two phages of a phage combination are provided. This trait is referred to as “at least 2 phage % coverage”. The phage combinations are ordered in descending performance grade.

[dl;48][lu;43][lr;43][dw;42][ln;42][lw;42][du;41][lp;41][cd;40][ly;40][cw;39][lv;39][cl;38][dC;38][dr;38][dy;38][rv;36][vw;36][dv;36][qy;36][pr;36][lm;35][pw;35][dn;35][lC;35][dp;35][rw;35][wx;34][np;34][uw;34][Cy;34][ru;33][de;33][nr;33][uy;33][ew;33][pu;33][cr;33][el;33][lx;33][nw;32][ny;32][cy;32][ry;32][lq;32][dx;32][nu;32][wC;32][px;32][pv;32][cp;32][py;31][ce; 31][cv;31][ux;31][eC;31][dm;31][lo;31][rx;31][wy;31][cu;31][dq;31][uv;31][cC;31][fl;30][rC;30][qC;30][uC;30][vx;30][cn;30][lt;28][fu;28][pC;28][er;28][nC;28][mw;28][cx;28][eu;28][s;28][en;28][nx;28][ep;28][df;27][xC;27][ev;27][nq;27][fr;27][cm;27][ex;27][or;27][ov;27][nv;27][rt;27][do;26][pt;26][ey;26][ow;26][op;26][vy;26][qu;26][mn;26][mu;26][rs;26][fw;26][dt;26][xy;26][tw;25][qr;25][fp;25][my;25][fx;25][mv;25][mp;25][tu;25][co;25][qw;25][cq;25][vC;25][ps;24][ou;24][fv;24][ds;24][ct;24][cf;24][ty;24][sw;24][pq;24][mr;24][mC;24][su;24][ox;24][mq;24][nt; 24][ef;24][cs;23][em;23][tv;23][fy;23][no;23][eq;23][sy;23][gB;23][ns;23][fC;23][tx;22][os;22][qv;22][fm;22][eo;22][sv;22][mx;22][sx;22][oy;22][fn;22][tC;20][gi;20][ft;20][qx;20][st;20][dg;19][es;19][fs;19][sC;19][oC;18][ot;18][qs;18][fo;18][fq;18][wg;18][et;18][mo;17][iB;17][lg;17][n g;17][ug;17][qt;17][pg;16][dB;16][mt;16][cB;16][oq;16][wB;16][cg;16][rg;15][tg;15][fg;15][xg; 15][di;15][ms;15][nB;15][li;15][ui;15][vg;15][lB;15][ni;14][yg;14][sg;14][pB;14][xB;12][yB;12]

3 Phage Combinations

3 phage combinations were analyzed in silico for their ability to lyse the 352 different strains of Klebsiella pneumoniae bacteria as profiled by their capsule type, based on the ability of the single phage to lyse a bacteria of a particular capsule type as assessed by a liquid media assay.

The combinations that lyse the highest number of bacterial strains (as profiled by their capsule locus type) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[lqg;85][lyg;84][lqB;84][lsg;83][luB;83][lqi;83][lug;83][lyB;83][lsB;82][lCg;82][lyi;82][lui;81][dlg;81][dlB;81][liB;81][lCi;81][lgj;81][dli;81][elg;81][lCB;81][ltB;81][lsi;81][lmg;81][lg i;81][lvi;81][lrB;81][lvg;81][lvB;81][lrg;81][ltg;81][flg;81][lwi;81][clg;81][lwB;80][lxB;80][lwg ;80][lxi;80][lpB;80][lng;80][lnB;80][lpg;80][eli;80][elB;80][flB;80][luj;80][lmB;80]lri;80][lgj;80][lxg:80][liB:80[[dla:80][cli:80[[lgB:80][lgz:80][lau:80[[mi:781 [1sC:78[[lti:78[[lvi:78][lni:78[[f li;78][luy;78][lgh;78][lni;78][lgk;78][lzB;78][lgA;78][lqx;78][lwj;78][dls;78][lCj;78][lvj;78][dly ;78][log;78][dlj;78][lqv;78][lqw;78][lij;78][lgs;78][c1B;78][clq;77][lhB;77][luC;77][elq;77][liz;77][lsv;77][lpq;77][loB;77][lsw;77][lkB;77][lsu;77][lAB;77][lsy;77][els;77][lgt;77][lnq;77][lxj;77][lxC;77][lqC;77][elj;77][lvy;77][lvC;77][lsj;77][lwy;77][lxy;77][ltj;76][loi;76][ely;76][liA;76][dlu;76][lCy;76][dln;76][elx;76][lns;76][luv;76][luw;76][cly;76][clu;76][lux;76][luz;76][lsx;76][1 st;76][clj;76][lrj;76][cls;76][lik;76][lgr;76][lpy;76][dlx;76][lnC;76][dlv;76][elu;76][dlt;76][dlC;76][lpj;76][lny;76][lwC;76][elv;76][lpC;76][ltC;76][lrs;76][lru;76][lqz;76][lty;76][dlr;76][flq;76][lih;76][lnj;76][lnu;76][lrC;75][lvz;75][cdl;75][elt;75][dlw;75][lyz;75][cln;75][flw;75][dfl;75][lsz ;75][lnw;75][lnv;75][flj;75][elp;75][elr;75][lry;75][eln;75][fly;75][lrw;75][lmq;75][de;75][ltv;75][dlz;75][ruB;75][vyi;75][fls;75][lpu;75][dlm;75][lwz;75][flu;75][clr;75][lps;75][loq;75][flv;75][ltu;75][clv;75][elw;75][clC;75][clx;75][dlp;75][ltw;75][lCz;75][lmu;75][clt;75][elz;74][lrx;74][1 uk;74][lgk;74][clz;74][lqA;74][lpw;74][lmw;74][lmv;74][lmx;74][clp;74][lvw;74][lgh;74][lmC; 74][cel;74][lmy;74][quv;74][lqy;74][luh;74][lmj;74][flx;74][wyi;74][uvy;74][cfl;74][loj;74][ljz;74][vyB;74][qvi;74][lwx;74][dlo;74][uvB;74][clw;74][vyg;74][lxz;74][ltx;74][lnx;74][lou;74][flC ;74][loy;74][rug;74][lvx;74][luA;74][dlh;74][lrv;74][lms;74][loC;74][dlk;74][dlA;74][lrz;73][nu v;73][dnq;73][los;73][ryg;73][uyB;73][flp;73][lvk;73][uyg;73][flt;73][lwh;73][flr;73][lnr;73][lmt ;73][lnt;73][low;73][uvg;73][quB;73][lnp;73][lov;73][dsB;73][qwi;73][lrt;73][dsi;73][qvg;73][is h;73][lnz;73][dsg;73][lvh;73][lwk;73][lmr;73][lwA;73][lpt;73][ltz;73][efl;73][elm;73][lyh;73][lp v;73][lpz;73][lCA;73][lCk;73][elC;73][lpx;73][lmn;73][elo;73][clm;73][ljA;73][lhj;73][ljk;73][fl n;73][ryi;73][lsA;73][qvB;73][lsk;73][lmp;73][lvA;73][qug;73][qri;73][lyA;73][qrg;73][lCh;73][lyk;73][clh;72][lmz;72][vyj;72][lpr;72][clA;72][clk;72][nuB;72][clo;72][qrB;72][nqu;72][qru;72][flz;72][lno;72][eiA;72][uvi;72][lox;72][uvC;72][flm;72][lor;72][lot;72][uwy;72][wyg;72][vCi; 72][svy;72][nuy;72][suB;72][nvy;72][nug;72][elh;72][nru;72][dgi;72][ixA;72][dqg;72][ryB;72][dri;72][quw;72][drg;72][dyg;72][dsv;72][drB;72][dyi;72][dqB;72][dug;72][ruy;72][rui;72][dyB; 72][dns;72][dps;72][dqs;72][dny;72][dpB;72][dpi;72][elk;72][dpg;72][dpq;72][duB;72][dvi;72][1 xh;72][lxk;72][lrh;70][dvg;70][myg;70][dui;70][lrk;70][uvj;70][lrA;70][lmo;70][mru;70][pqg;70][mrg;70][nwi;70][mri;70][ruj;70][duv;70][muy;70][loz;70][mug;70][qsw;70][qui;70][swy;70][it h;70][pyg;70][ipk;70][dms;70][sug;70][mqu;70][suv;70][pA;70][ph;70][dnr;70][qsv;70][dnu;70][rCi;70][dvB;70][mrB;70][uwB;70][pug;70][dsw;70][dpy;70][inA;70][rwi;70][qwg;70][cqu;70][lop;70][uyi;70][dqv;70][dqu;70][dgr;70][ink;70][ruw;70][itk;70][nuj;70][flo;70][muB;70][lnh;70][wyB;70][puB;70][nqv;70][tA;70][wyj;70][dsy;70][nuw;70][dng;70][dni;70][dnB;70][nvi;70][dnv;70][uwg;70][uwi;70][cru;69][muv;69][dnj;69][nvj;69][dmB;69][suw;69][dmg;69][nwy;69][cuy;69][dpu;69][pqu;69][eyi;69][puy;69][nui;69][muC;69][ouB;69][nsv;69][uCB;69][syg;69][qv j;69][dmi;69][uCg;69][uwj;69][eyg;69][svC;69][nwj;69][rCg;69][cug;69][dmu;69][nvB;69][nyg; 69][qvw;69][nyi;69][lmA;69][ryj;69][nvC;69][dnp;69][dmr;69][nvg;69][rwy;69][nuC;69][equ;69][myB;69][cuv;69][rsu;69][nsu;69][drs;69][uyj;69][dru;69][nri;69][flk;69][nrg;69][dry;69][flA; 69][qwB;69][uvw;69][ruC;69][flh;69][pgi;69][drj;69][qwj;69][pqB;69][dsx;69][euy;69][dsu;69][cqv;69][pyi;69][dsj;69][cnu;69][lmk;69][lmh;69][myi;69][quj;69][swi;69][duy;69][qrj;69][duj;69][pyB;69][vCg;69][vCB;69][lkz;69][lzA;69][lhz;69][nqw;69][dvy;69][evy;69][qrw;69][vwy;69][csu;68][pvy;68][pwy;68][eqv;68][dos;68][doq;68][uwC;68][dnC;68][dnx;68][uCj;68][cpu;68][eru;68][puv;68][qtv;68][drw;68][cuB;68][dmn;68][dgj;68][dvj;68][doi;68][dog;68][lok;68][doB; 68][dmy;68][dqx;68][cnv;68][oyg;68][dst;68][loA;68][duw;68][nou;68][dsC;68][vCj;68][mgr;68][loh;68][pqv;68][pqw;68][cvy;68][tvy;68][pwi;68][pru;68][pui;68][puj;68][uCi;68][dnw;68][dq w;68][nyB;68][xyi;68][svB;68][rCB;68][ouy;68][nrB;68][nsw;68][svi;68][oqu;68][dxy;68][dwi; 68][dyj;68][fwy;68][mvi;68][mry;68][sui;68][fvy;68][qsg;68][dwy;68][dmq;68][eri;68][dfn;68][syi;68][mwy;68][mui;68][dqt;68][cui;68][ogg;68][psu;68][puw;68][oug;68][des;68][mvy;68][su y;68][dpj;68][dxi;68][fqv;68][qsu;68][msu;68][euv;68][muw;68][rwg;68][wCi;68][nwg;68][dnt; 67][mpB;67][dnz;67][mqs;67][deB;67][suC;67][mnr;67][vwi;67][mqv;67][syB;67][mCi;67][dty; 67][tuv;67][dxB;67][npu;67][cyg;67][dno;67][cqg;67][dpr;67][dfi;67][dpt;67][svg;67][mCg;67][mpg;67][dfg;67][cqr;67][dfB;67][dfq;67][dfs;67][dmp;67][cgi;67][dmv;67][mpy;67][dmx;67][m wi;67][dwg;67][cuC;67][sCi;67][msy;67][dxg;67][dtB;67][dwB;67][msg;67][dux;67][msC;67][r vy;67][duC;67][mxy;67][sCg;67][swg;67][swC;67][vyz;67][cyi;67][mvg;67][xyg;67][mnv;67][1 hk;67][lhA;67][diB;67][tuB;67][rCj;67][cvi;67][twy;67][mnu;67][muj;67][mvB;67][lkA;67][cds; 67][rwB;67][rwj;67][mqw;67][dfy;67][cmu;67][ruv;67][pwg;67][puC;67][rsi;67][nyj;67][nrw;67][qux;67][nrv;67][nrj;67][ery;67][eug;67][emu;67][pCg;67][ngi;67][pCi;67][ngg;67][nvw;67][ov y;67][qvC;67][rux;67][dtg;67][qrv;67][pvg;67][nux;67][qvx;67][eyB;67][euB;67][egi;67][erg;67][egr;67][enu;67][pvi;67][qtw;67][egg;67][rsg;67][pvB;67][fqw;67][vxy;67][drv;67][drx;67][mq g;67][cri;67][vCy;67][deu;67][uxB;67][der;67][deq;67][den;67][uxy;67][dtv;67][mrC;67][nCi;67][mpq;67][drt;67][dei;67][euj;67][rsB;67][fyg;67][dey;67][nwB;67][fnv;67][nry;67][ngr;67][qxi; 67][mvC;67][doy;67][cni;67][deg;67][dpx;67][dpC;67][dij;66][epu;66][diz;66][epy;66][dev;66][qtu;66][djB;66][qsB;66][dmt;66][dnk;66][dgi;66][doj;66][drC;66][esu;66][dvC;66][dmw;66][dp v;66][dpw;66][owy;66][dgz;66][mrs;66][dmo;66][oCg;66][cng;66][cnr;66][cnq;66][mCB;66][m qB;66][oui;66][emy;66][pyj;66][dvw;66][dCi;66][quC;66][dgj;66][dfp;66][dfm;66][env;66][rsy; 66][dor;66][dtu;66][rxy;66][dti;66][qrx;66][dsA;66][ewy;66][rvB;66][rvi;66][riB;66][tuy;66][sw B;66][dvx;66][suj;66][oyi;66][epq;66][qrs;66][euw;66][tug;66][evi;66][dfv;66][qsi;66][rtu;66][d ou;66][dxj;66][erB;66][dfu;66][dCg;66][dCB;66][emr;66][tuw;66][dep;66][eui;66][dzB;66][dem ;66][rxi;66][qxg;66][dsz;66][mqi;66][mrw;66][fny;66][cpi;66][crg;66][psy;66][uCy;66][cuw;66][cux;66][uwz;66][ruz;66][cpg;66][mny;66][oaB;66][msw;66][oai;66][naB;66][oaw;66][wyz;66][nrC;66][cuj;66][cry;66][msB;66][pCB;66][ouC;66][uxg;66][eyj;66][psg;66][npy;66][mni;66][cs v;66][npq;66][pwB;66]i[mns;66][uvz;66][mnB;66][mnw;66][mng;66][npv;66][npw;66][msi;66][uvx;66][mtu;66][mpi;66][mrj;66][fnw;66][prg;66][pry;66][mpu;66][mpC;66][pgj;66][ujB;66][m sv;66][pqs;66][pqr;66][fnq;66][oyB;66][fnu;66][fyB;66][mqx;66][wCg;66][cpq;66][nuz;66][ntv; 66][nsC;66][nsy;66][nCj;66][nCg;66][fyi;66][nsi;66][xyj;66][ntu;66][nsg;66][mux;66][nwC;66][nvz;66][cdu;66][xyB;66][cdn;66][cdr;66][nCB;65][enr;65][dwj;65][opu;65][dyz;65][frB;65][dtw ;65][mwB;65][ogr;65][cfn;65][fuv;65][rty;65][fry;65][dvz;65][ruh;65][nwz;65][ntw;65][nvx;65][rvg;65][dgB;65][dgk;65][pri;65][ceu;65][dik;65][cdq;65][moy;65][esy;65][mou;65][qtB;65][prB ;65][dej;65][psv;65][psw;65][psC;65][qtg;65][psi;65][psB;65][det;65][fpq;65][qwz;65][dtx;65][n sB;65][nxy;65][cdp;65][oqv;65][rwC;65][rsw;65][nqx;65][oru;65][dtj;65][fqr;65][rsC;65][fqu;65][ory;65][vyA;65][fqg;65][nrs;65][wCy;65][wxy;65][ngs;65][eny;65][vyk;65][duA;65][osu;65][wCB;65][ngj;65][wCj;65][fpy;65][rxg;65][duz;65][cdv;65][cdy;65][rxB;65][wyk;65][fuB;65][cd i;65][mwC;65][dkB;65][rCy;65][Cyi;65][fuy;65][mwg;65][Cyg;65][fuw;65][cdg;65][fru;65][mn q;65][ouw;65][cmr;65][dfr;65]

The percent of host bacterial strains (as profiled by their capsule locus type) that are infected by two phages of a phage combination are provided below. This trait is referred to as “at least 2 phage % coverage”. The phage combinations are ordered in descending performance grade.

[dlu;59][luv;57][luw;57][dlp;57][dlv;56][dln;56][dlw;56][dlr;56][lru;56][lnu;56][lnv;56][1 pu;56][lmu;55][dlg;55][clu;55][lnr;55][dlm;55][del;55][lvy;55][lpv;55][lnw;55][dls;55][dlo;55][1 mr;55][lry;53][dlB;53][luC;53][dlx;53][lwy;53][lvw;53][lou;52][lgr;52][dlt;52][dfl;52][lrw;52][d ly;52][lpr;52][lmy;52][elu;52][luy;52][dlC;52][lpw;52][dru;52][lsu;52][dli;52][cdl;52][lmv;51][1 mw;51][lvC;51][lqw;51][lqv;51][lmp;51][lrC;51][lmn;51][lqu;51][ltu;51][lnp;51][dnu;51][lms;51][duw;51][dpv;51][dlq;51][lux;50][lrv;50][dpu;50][duy;50][dnw;50][dvy;50][dlj;50][clv;50][fln ;50][cdu;50][cln;50][dlz;50][lwC;50][lny;50][lwg;50][dou;50][lno;50][lpy;50][dsu;50][ely;49][ls w;49][clp;49][dwy;49][lnt;49][lnC;49][lvx;49][lpq;49][dvC;49][low;49][duv;49][dtu;49][dnv;49][lmo;49][lnx;49][dlA;49][dlk;49][duC;49][dpw;49][lmC;49][loy;49][clw;48][mnu;48][dpr;48][d wg;48][elr;48][ltw;48][lns;48][lps;48][dmp;48][dgw;48][dmu;48][clr;48][lmt;48][loC;48][dwi;48][dlh;48][cly;48][lwx;48][lrx;48][lri;48][lwi;48][dry;48][nuw;48][deu;48][drC;48][lui;48][lwB;48][lvg;48][clm;48][lrg;48][dqu;48][lop;48][lug;48][luB;48][lsy;48][mpu;48][cdn;48][lsv;48][elm ;48][lpC;48][ruv;48][lng;47][lpx;47][clC;47][dmn;47][dwB;47][ltv;47][lor;47][lrB;47][drw;47][1 uj;47][drv;47][eln;47][fly;47][lvB;47][dwj;47][npu;47][elC;47][dmv;47][dux;47][lmx;47][lpg;47][flu;47][elv;47][dmr;47][clq;47][duB;47][lnq;47][lxy;47][dvw;47][dCi;47][elw;47][lrj;47][dmw ;47][lrt;47][lqy;47][dCg;47][dnr;47][loq;47][clo;45][lmq;45][muw;45][lrs;45][uwC;45][dmy;45][dno;45][cdq;45][dui;45][cdp;45][lpi;45][puC;45][lCy;45][dtw;45][dug;45][lnj;45][dav;45][nrw; 45][lvi;45][cdv;45][cls;45][nvw;45][puv;45][flw;45][ruw;45][ouw;45][elp;45][cdg;45][puw;45][cdi;45][lpt;45][lni;45][uwy;45][cdy;45][flr;45][flp;45][dgr;45][dnp;45][nry;44][drg;44][dnC;44][lwk;44][dpy;44][dng;44][lnk;44][mru;44][lrA;44][lrk;44][lrh;44][ruC;44][uvw;44][drs;44][cnu;44][duj;44][pqu;44][puy;44][nwy;44][nuC;44][drB;44][quw;44][dri;44][lov;44][nuv;44][doy;44][dfr;44][lgs;44][flC;44][mrw;44][rwC;44][lnB;44][lpB;44][lCi;44][rvy;44][dCB;44][clx;44][elq;44][flv;44][dpC;44][cdr;44][dep;44][lty;44][lqx;44][nwC;44][uCy;44][dnt;44][lCg;44][cuw;44][d ev;44][lwj;44][der;44][den;44][lsC;44][luh;44][lyi;44][dey;44][luk;44][luA;44][dfu;44][cel;44][c 1B;43][ltC;43][flq;43][tuw;43][dor;43][dyg;43][qru;43][dvx;43][nru;43][dmt;43][flm;43][vwy;43][lgt;43][rwy;43][dns;43][lyj;43][dny;43][nrC;43][suw;43][muv;43][ruy;43][lpj;43][dxy;43][dnx; 43][drx;43][lyg;43][cfl;43][dnB;43][dni;43][enu;43][rvg;43][pru;43][pvy;43][dsy;43][suC;43][df n;43][clg;43][lCB;43][lnz;43][duz;43][mnw;43][luz;43][euw;43][suy;43][cli;43][dvg;43][npv;43][lwz;43][lrz;43][oru;43][dvB;43][elt;43][lnh;42][dms;42][rsw;42][dfv;42][mou;42][lpk;42][uwg ;42][wCy;42][deg;42][dmo;42][nsu;42][eru;42][uwB;42][rwB;42][mnv;42][dty;42][mnr;42][pwy ;42][dsC;42][dtv;42][qrv;42][uxy;42][nrv;42][dei;42][vCy;42][ngr;42][dfy;42][dqx;42][npy;42][pqr;42][qrw;42][epu;42][dfp;42][quC;42][ouC;42][cru;42][pry;42][mvy;42][nvC;42][ntu;42][mr y;42][msu;42][mny;42][npw;42][uwi;42][cuv;42][nuy;42][lyB;42][elo;42][nvy;42][lxg;42][clt;42][uvy;42][uvC;42][lox;42][lsx;42][elg;42][ltx;42][lwA;42][els;42][lqC;42][lwh;42][dyi;42][lyk; 42][flo;42][dpq;42][lnA;42][dsw;42][eli;42][fuw;41][dnj;41][cuy;41][drj;41][pqw;41][qtu;41][cu C;41][lmi;41][cds;41][efl;41][lyA;41][lyz;41][dvi;41][uCg;41][rvB;41][rvi;41][eny;41][clj;41][t uy;41][dem;41][dyB;41][lvj;41][dpg;41][duA;41][fuy;41][dsv;41][pqv;41][doq;41][nqw;41][wx y;41][nrs;41][nux;41][dxg;41][qux;41][elB;41][uvx;41][fnu;41][prB;41][lpz;41][det;41][cmu;41][pri;41][cpu;41][nxy;41][qsu;41][rwg;41][uCi;41][mwy;41][dyj;41][deq;41][nsy;41][dmx;41][lot ;41][lpA;41][lxC;41][rCy;41][lvh;41][cnr;41][muy;41][drt;41][lph;41][euv;41][wCg;40][mux;40][dqt;40][des;40][dmg;40][rtu;40][pui;40][dmq;40][lvA;40][uwj;40][esu;40][cry;40][nCg;40][mr s;40][owy;40][lvk;40][mtu;40][nsw;40][ouy;40][msw;40][los;40][qvw;40][env;40][rui;40][lCj;40][nwg;40][prg;40][lmB;40][psu;40][dps;40][dfq;40][euy;40][clk;40][vwi;40][lxi;40][nvx;40][fu v;40][ntw;40][suv;40][eug;40][deB;40][opu;40][enr;40][tuv;40][mpy;40][dpt;40][nwB;40][ceu;40][psw;40][nqv;40][uyi;40][dpx;40][lyh;40][quv;40][lmj;40][Cyg;40][lmg;40][dxB;40][npq;40][dpj;40][dpi;40][rsy;40][dpB;40][rsu;40][eui;40][lxB;40][uCB;40][ewy;40][dqg;40][nou;40][cnv; 40][rux;40][nui;40][lvz;40][nqu;40][vxy;39][mrC;39][cui;39][dnq;39][clz;39][fls;39][cvy;39][dv j;39][crg;39][psy;39][csu;39][mns;39][nsC;39][emu;39][cuB;39][elx;39][ery;39][uyg;39][euB;39][lqg;39][lxj;39][nuB;39][dtx;39][nCB;39][cug;39][dyz;39][lgi;39][cmr;39][fqu;39][nvg;39][wC B;39][dqs;39][dmi;39][mwC;39][dmB;39][Cyi;39][muC;39][mwg;39][rxy;39][elj;39][rsC;39][de j;39][puB;39][msv;39][dxi;39][fnw;39][pug;39][rwi;39][cqu;39][nas;39][nug;39][nrB;39][psv;39 ][oqu;39][cnq;39][fru;39][nrg;38][qvC;38][uyj;38][ngx;38][msy;38][pwg;38][uvg;38][clh;38][cl A;38][dvz;38][lmz;38][rug;38][wyg;38][cqr;38][ruj;38][lCk;38][psC;38][mvC;38][uvi;38][ory;38][flx;38][nsv;38][ogw;38][nyg;38][cux;38][flt;38][dqB;38][cng;38][cuj;38][log;38][lqB;38][dxj ;38][pwB;38][uyB;38][epy;38][fny;38][nvB;38][uxg;38][cpq;38][pvg;38][qrs;38][lCz;38][dgi;38][wCi;38][qrx;38][ntv;38][mqw;36][pqs;36][dfm;36][moy;36][loB;36][esy;36][mpC;36][cri;36][dtB;36][mwB;36][cyg;36][swC;36][rwj;36][uvB;36][ruB;36][twy;36][mnq;36][lst;36][dfs;36][m qv;36][emr;36][fry;36][ogr;36][loi;36][dnk;36][dnz;36][nuj;36][rCB;36][swy;36][pCg;36][equ;36][evy;36][ngg;36][wyB;36][rCg;36][vCg;36][puj;36][nCi;36][pvB;36][pwi;36][pvi;36][mgr;36][mui;36][fnv;36][nwi;36][nri;36][lCA;36][lmk;36][nyB;36][dsx;36][qtw;35][lxz;35][qug;35][oq v;35][egr;35][nyi;35][lCh;35][ryg;35][mqu;35][qui;35][mwi;35][ryB;35][pCB;35][erg;35][cyi;35][mng;35][dog;35][lxk;35][nrj;35][fpy;35][lmh;35][nvi;35][uxB;35][dti;35][uCj;35][qwg;35][py g;35][dtg;35][pyi;35][nqB;35][lgj;35][rty;35][lmA;35][muB;35][rxg;35][uwz;35][elk;35][erB;35][lxh;35][pCi;35][mug;35][qvx;35][emy;35][wyi;34][dst;34][dfB;34][csv;34][cfn;34][tvy;34][rx B;34][mxy;34][lsi;34][ryi;34][cpg;34][pyB;34][quB;34][lsB;34][uvj;34][dqj;34][fwy;34][rCi;34][cni;34][elz;34][mpq;34][lsg;34][evi;34][dfg;34][epq;34][lgk;34][lxA;34][vCB;34][nyj;34][vyg; 34][qwB;34][xyg;34][elA;34][wCj;34][svy;34][cqv;34][pyj;34][fqr;34][cvi;34][fvy;34][svC;34][mvg;34][ovy;34][ltB;33][lsj;33][xyB;33][ltg;33][flB;33][lqz;33][rxi;33][eri;33][mpi;33][osu;33][nuz;33][fnq;33][lti;33][nwj;33][elh;33][fli;33][eqv;33][qsw;33][ruz;33][mni;33][fqw;33][ngi;33][mnB;33][loh;33][lok;33][cqg;33][dfi;33][doB;33][mpg;33][msC;33][euj;33][loj;33][cpi;33][fpq; 33][mvB;33][lqA;33][mrg;33][dos;33][ruh;33][flj;33][tuB;32][qrB;32][loA;32][eyB;32][vCi;32][dsg;32][eyg;32][qtv;32][mri;32][ryj;32][pqg;32][mrB;32][vyB;32][qrg;32][uvz;32][qsv;32][wyj ;32][nCj;32][nwz;32][loz;32][lgh;32][flg;32][tug;32][doi;32][muj;32][qxg;32][fuB;32][mCg;31][nvj;31][cgi;31][ltz;31][dtj;31][swB;31][rCj;31][myi;31][nsB;31][mpB;31][eyi;31][ltk;31][vyi;31][quj;31][flA;31][myg;31][pqB;31][pgi;31][flh;31][wyk;31][swg;31][mvi;31][xyi;31][qvg;31][q wi;31][ltj;31][dsB;31][wyz;31][oug;31][nsg;31][mrj;30][fqv;30][ltA;30][lsA;30][sug;30][suj;30][myB;30][dsi;30][egg;30][ngj;30][lth;30][flk;30][flz;30][sui;30][qri;30][svg;30][lsk;30][oui;30][mCi;30][mCB;30][mqs;30][lsh;30][rsg;30][mqx;30][psB;28][dsj;28][dgB;28][mqg;28][syB;28][r sB;28][eyj;28][doj;28][psg;28][lsz;28][xyj;28][qvB;28][oCg;27][vCj;27][lgB;27][vyj;27][frB;27][nsi;27][nvz;27][pgj;27][oyB;27][mqB;27][mqi;27][sCg;27][dgi;27][oyi;27][suB;27][egi;27][vyk ;27][psi;27][qxi;27][oyg;27][rsi;27][ouB;27][syg;27][svB;26][vyz;26][fyg;26][qwj;26][fyi;26][q vi;26][fyB;26][qtg;26][swi;26][vyA;26][syi;26][dsz;26][msB;25][qtB;25][qrj;25][qwz;25][diB;25][msg;25][sCi;25][fqg;25][qsB;25][svi;25][dgj;25][lgi;25][liB;24][qsg;24][dsA;24][qsi;24][msi; 24][oqB;23][lgj;23][qvj;23][djB;23][ogg;23][ljB;22][oqi;22][ujB;20][riB;20][dgk;19][lgz;19][dg z;19][dij;19][lij;19][lgk;18][lgh;18][lgA;17][1 kB;17][lzB;17][lih;17][liz;17][dkB;17][lhB;16][dz B;16][lik;16][liA;15][lAB;15][diz;15][dik;15][ljz;12][lhj;12][ljk;11][ljA;10][lhk;8][lkz;7][lkA;7][lhz;7][lhA;6][lzA;6]

The percent of host bacterial strains (as profiled by their capsule locus type) that are infected by three phages of a phage combination are provided below. This trait is referred to as “at least 3 phage % coverage”. The phage combinations are ordered in descending performance grade.

[dlw;38][dly;36][cdl;36][dlu;36][dlr;36][clw;35][dC;34][drw;34][lrv;34][lrw;34][lpr;34][dvw;34][dln;34][duw;33][dlp;33][dlv;33][clr;33][lnp;33][cdr;33][lpw;33][lvw;32][lru;32][lCy;32][luy;32][dpw;32][lnr;32][drv;32][cdy;32][lny;32][duy;31][cdu;31][lnu;31][lpu;31][lwx;31][luw; 31][dny;31][lpy;31][lry;31][dpr;31][cdv;31][lqy;31][dwy;31][clp;31][cly;31][lnw;31][elw;31][cd p;31][dpy;30][dpx;30][dpu;30][cln;30][dlm;30][lwC;30][lrx;30][lwy;30][dnw;30][dnp;30][pru;30][duv;30][dry;30][duC;30][dru;30][cuw;30][dlx;30][dlq;30][del;30][dnr;30][npw;30][lpx;30][lp v;28][cel;28][ruw;28][clv;28][uvw;28][pry;28][dnC;28][clC;28][cdn;28][drC;28][puw;28][clu;28][elp;28][dpC;28][lux;28][cry;28][lpC;28][lnC;28][lrC;28][dux;28][drx;28][elr;28][dnu;28][eln;28][rwy;27][rCy;27][flu;27][cru;27][cuy;27][wCy;27][nuy;27][ruy;27][elx;27][npy;27][dvx;27][d er;27][deu;27][flr;27][cnr;27][lor;27][nrv;27][nrw;27][rux;27][lnx;27][lmw;27][luv;27][pwy;27][lxC;27][lqC;27][dmw;27][clx;27][lnq;27][cpu;27][nru;27][luC;27][dnq;27][lvx;26][elC;26][lmn; 26][uwy;26][clm;26][lnv;26][dfl;26][lrt;26][flw;26][rwC;26][lov;26][dep;26][elu;26][elv;26][dp v;26][cux;26][ruv;26][dnx;26][lxy;26][dev;26][nry;26][dey;26][dxy;26][den;26][npu;26][euw;26][lrs;26][dfu;26][uCy;26][puy;26][flx;25][lpt;25][nux;25][lmp;25][nvw;25][dlo;25][flp;25][low;25][cuC;25][uvx;25][npv;25][enr;25][drt;25][rvy;25][rxy;25][cdq;25][ewy;25][cuv;25][lqu;25][cv y;25][dlt;25][ely;25][vwy;25][dvy;25][dvC;25][fru;25][nuw;25][dnv;25][lop;25][dqu;25][nwy;25][dmu;25][dgw;25][wxy;25][lmu;25][puv;25][lvy;25][uwC;25][ceu;25][epu;24][lmy;24][lmv;24][cfl;24][nwC;24][nrC;24][dor;24][epy;24][ruC;24][dtw;24][lmr;24][dpt;24][uxy;24][rty;24][eu y;24][cnv;24][dmy;24][dpq;24][clo;24][eru;24][dgr;24][dsw;24][cnu;24][fuw;24][ery;24][dfr;24][lty;24][lvC;24][ltw;24][uvy;24][lqw;24][lpq;24][flv;24][clt;24][lgr;24][clq;24][fwy;23][dfp;23][cmr;23][fly;23][lsw;23][dty;23][nuv;23][pvy;23][lou;23][mwC;23][efl;23][lnt;23][dem;23][dls;23][dfv;23][euv;23][nqu;23][rtu;23][lno;23][lox;23][ltu;23][dmq;23][deq;23][lmq;23][dmv;23][lp s;23][eny;23][dmn;23][nuC;23][dmr;23][cqr;23][lns;23][fry;23][dfy;23][npq;23][puC;23][nxy;23][nvx;23][enu;23][cds;23][lmC;23][lsy;22][dmp;22][elm;22][ltv;22][rsu;22][dqv;22][dtv;22][elq; 22][cqu;22][nvy;22][loy;22][env;22][uvC;22][nqw;22][evy;22][cqv;22][drs;22][lmx;22][qrw;22][flm;22][dmx;22][dtu;22][psw;22][mnw;22][cmu;22][mwy;22][dps;22][lsu;22][cnq;22][flC;22][pqr;22][fuy;22][qvw;22][twy;22][muw;22][elo;22][fuv;22][mrw;22][vxy;22][muy;22][vCy;22][c pq;22][opu;22][quC;22][fpy;22][rsw;22][pqw;22][ngr;22][dfm;22][fln;22][cls;22][ory;22][dou;2 0][lst;20][mpC;20][mqu;20][swy;20][fnu;20][lsx;20][dfn;20][equ;20][lsv;20][dsy;20][csv;20][m nr;20][muC;20][mpy;20][dnt;20][dsx;20][fnw;20][qrv;20][flt;20][dqx;20][quw;20][nrs;20][oru;20][mnq;20][los;20][ltC;20][lqv;20][lqx;20][doy;20][ntw;20][ltx;20][cfn;20][egr;20][dtx;20][ovy; 20][dsv;20][mny;20][tuy;20][epq;20][mqw;20][psu;20][rsy;20][tuw;20][qru;20][nou;19][qrx;19][dsC;19][dns;19][tvy;19][suw;19][eqv;19][fny;19][nvC;19][owy;19][mru;19][mux;19][muv;19][t uv;19][ntu;19][dsu;19][pqu;19][mxy;19][swC;19][emu;19][quv;19][psv;19][lsC;19][psy;19][qvC ;19][dos;19][emr;19][mry;19][mrC;19][emy;19][fvy;19][ngx;19][fls;19][els;19][nsw;19][csu;19][dno;19][ouy;19][ouw;19][mpq;19][nsu;18][qvx;18][ntv;18][fqw;18][rsC;18][osu;18][flo;18][ns y;18][flq;18][det;18][fnv;18][qux;18][svy;18][loC;18][dfq;18][nqv;18][suv;18][mvC;18][des;18][mqx;18][lot;18][elt;18][mnv;18][mgr;18][mpu;18][pqv;18][suy;18][fqr;18][dst;18][mnu;18][fqu ;17][fqv;17][lgt;17][lmo;17][mqv;17][fnq;17][psC;17][dqs;17][dfs;17][lgs;17][qsw;17][nsv;17][f pq;17][mvy;17][dqt;17][qtw;16][svC;16][doq;16][dwg;16][pwg;16][nsC;16][esu;16][dug;16][qrs ;16][ogr;16][esy;16][lmt;16][dmo;16][loq;16][qsv;15][pqs;15][ouC;15][dlg;15][suC;15][lug;15][dmt;15][dgB;15][lng;15][flg;15][dpg;15][qtv;15][oqv;15][cdg;15][ngs;15][cpg;15][ogw;15][oqu; 14][dng;14][qsu;14][lgi;14][msw;14][clg;14][nwg;14][rwg;14][lms;14][lyg;14][dgi;14][dfg;14][uwg;14][mqs;14][mtu;14][nug;14][qtu;14][pug;14][nrg;14][lpg;14][lwg;14][lnB;14][moy;14][dr g;14][dyg;14][lgB;14][lrg;14][ltg;14][pwB;14][dms;14][rug;14][prg;14][dwB;14][dvg;14][psg;12][uxg;12][crg;12][dtg;12][dyB;12][uvg;12][uyg;12][tug;12][wyg;12][dpB;12][mns;12][ryg;12][mou;12][lui;12][dsg;12][lwB;12][sug;12][lpB;12][nyg;12][fyg;12][msy;12][msC;12][dui;12][lni; 12][rsg;12][lmg;12][cng;12][lsg;12][cyg;12][dxg;12][pvg;12][lyB;12][mrs;12][cug;12][rxg;12][d 1B;12][swg;12][dli;12][clB;12][pyg;12][lti;11][lpi;11][dog;11][nwB;11][frB;11][luB;11][rvg;11][log;11][deg;11][pwi;11][fli;11][flB;11][cpi;11][liB;11][lxg;11][syg;11][nyB;11][diB;11][dnB;11][pyB;11][dmg;11][duB;11][dCg;11][msv;11][msu;11][wyB;11][lrB;11][oug;11][dpi;11][wCg; 11][lvg;11][nsg;11][cli;10][rwB;10][nsi;10][wCB;10][fyB;10][pCg;10][psB;10][psi;10][prB;10][Iri;10][mng;10][lxB;10][lmB;10][rsi;10][rsB;10][cni;10][xyg;10][ltB;10][swB;10][uxB;10][mpg; 10][dCB;10][dfB;10][lsi;10][lwi;10][Cyg;10][mwg;10][cdi;10][svg;10][fuB;10][rxB;10][elg;10][deB;10][lCg;10][lsB;10][oui;10][lyi;10][dxB;10][dfi;10][lqg;10][myg;10][ryB;10][doB;10][doi;10][ouB;10][uyB;10][suB;10][ruB;10][dsB;10][cuB;10][dsi;10][nuB;10][nvg;10][vyg;10][nyi;10][drB;10][dyi;10][eyg;10][loB;10][rCg;10][dqg;10][nrB;10][dwi;10][sui;10][pui;10][nui;10][dvi;10][mug;10][dni;10][nwi;10][erg;10][dvB;10][puB;10][loi;10][swi;10][nri;10][oyg;10][uwB;10][p vB;10][xyB;9][nCg;9][vwi;9][pqg;9][fyi;9][riB;9][rvB;9][mrg;9][oyB;9][qsg;9][dti;9][lvi;9][uCg ;9][pyi;9][cvi;9][uyi;9][sCg;9][myB;9][lCB;9][qwg;9][lxi;9][pCB;9][mpB;9][dij;9][rwi;9][mnB; 9][uvi;9][uwi;9][cyi;9][dmB;9][mvg;9][rxi;9][lqB;9][syB;9][syi;9][lmi;9][cri;9][mqg;9][uvB;9][r yi;9][lvB;9][qrg;9][mwB;9][nsB;9][cui;9][eyB;9][wyi;9][ngg;9][pvi;9][eug;9][vyB;9][pri;9][dlj; 9][qug;9][dri;9][dxi;9][fag;9][mCg;9][rui;9][elB;9][svi;9][cag;9][svB;9][lij;9][daB;9][maB;8][cl j;8][tuB;8][dgj;8][nvi;8][rvi;8][nCB;8][nvB;8][lgi;8][vCg;8][oCg;8][mCB;8][mni;8][oyi;8][nqB; 8][qwB;8][qtg;8][ngi;8][rCB;8][egg;8][loj;8][pqB;8][lgj;8][xyi;8][lwj;8][dgi;8][qvg;8][vyi;8][dm i;8][erB;8][lpj;8][dvj;8][lvj;8][dtB;8][msg;8][ltj;8][doj;8][oqg;8][Cyi;7][qsB;7][dwj;7][dtj;7][qrB ;7][qxg;7][ljB;7][qsi;7][luj;7][mpi;7][vyj;7][lCi;7][djB;7][muB;7][qri;7][dCi;7][qwi;7][rCi;7][qu B;7][lmj;7][mqi;7][qui;7][mirB;7][qvB;7][qvi;7][wyj;7][lrj;7][flj;7][lyj;7][dsj;7][dyj;7][lgj;7][mv B;7][dei;7][dqj;7][dpj;7][vCB;7][mui;7][lnj;7][duj;7][wCi;7][qrj;7][cgi;7][oqB;7][eli;7][myi;7][mvi;7][eri;7][nwj;7][mwi;7][ryj;7][uCB;7][sCi;7][eyi;7][rwj;7][lsj;7][qvj;7][pgi;7][qwj;7][drj;7][nCi;7][oqi;7][euB;7][pCi;7][nyj;6][puj;6][ngj;6][qtB;6][lCj;6][qxi;6][nrj;6][rCj;6][dnj;6][vCi;6][nvj;6][eui;6][egi;6][evi;6][mCi;6][uvj;6][msi;6][pgj;6][mri;6][flk;6][vCj;6][uCi;6][msB;6][pyj;6][wCj;6][lvk;5][lxj;5][dzB;5][elj;5][muj;5][lrz;5][uwj;5][lsk;5][lgk;5][flz;5][lsz;5][lpz;5][dej;5][dl k;5][suj;5][dik;5][ljk;5][dgk;5][xyj;5][luk;5][ruj;5][quj;5][nuj;5][uyj;5][ujB;5][eyj;5][dgz;5][irk; 5][lmk;5][mrj;5][luz;5][diz;5][lik;5][nCj;5][ruz;5][cuj;5][dxj;5][lpk;5][dkB;3][uwz;3][lyk;3][lCh ;3][ltk;3][lok;3][lwA;3][liA;3][lwh;3][wyz;3][lwk;3][nwz;3][lnz;3][wyk;3][ljA;3][dvz;3][euj;3][clz;3][nvz;3][lgA;3][lpA;3][elz;3][lgh;3][lph;3][dlh;3][lgz;3][nuz;3][qwz;3][lzB;3][lyh;3][lgk;3][dyz;3][lCk;3][ljz;3][lAB;3][clk;3][duz;3][elh;3][lxk;3][dlz;3][elk;3][lmh;3][lnA;3][loz;3][lmA;3][lkB;3][uvz;3][lwz;3][dnk;3][dsz;3][lnk;3][lqz;3][clA;3][clh;3][vyk;3][dnz;3][lvz;3][lkz;3][lnh;3][vyz;3][liz;3][uCj;3][lyz;3][vyA;2][flA;2][ltz;2][dsA;2][elA;2][luh;2][lzA;2][luA;2][lyA;2][lkA; 2][lhB;2][lCz;2][lmz;2][lqA;2][lsh;2][lth;2][lgh;2][lxA;2][lsA;2][loA;2][dlA;2][flh;2][duA;2][lr A;2][lCA;2][lvA;2][lrh;2][ltA;2][ruh;2][lxz;2][lih;1][lhj;1][lxh;1][lhz;1][lhA;1][lhk;1][lvh;1][loh ;1]

4 Phage Combinations

4 phage combinations were analyzed in silico for their ability to lyse the 352 different strains of Klebsiella pneumoniae bacteria as profiled by their capsule type, based on the ability of the single phage to lyse a bacteria of a particular capsule type as assessed by a liquid media assay.

The combinations that lyse the highest number of bacterial strains (as profiled by their capsule locus type) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[lquB;89][lqug;89][lgsg;89][luyg;88][lsyg;88][lqiB;88][lgtB;88][flgg;88][lgtg;88][lqgj;88][lqgi;88][lgsB;88][luyB;88][lqxg;86][lyiB;86][dlqg;86][lsug;86][lqvB;86][lqvi;86][lqxB;86][lq Cg;86][lqvg;86][lqwi;86][lqgz;86][lqui;86][lqxi;86][lgjB;86][lygi;86][lqgB;86][lygj;86][lgsi;86][flqB;86][dlqi;86][flyg;86][lpqg;86][dlqB;86][lgrg;86][elqg;86][lsuB;86][lpqB;86][clqg;86][ltyg; 86][lmag;86][lsCg;86][lsyB;86][larB;86][ltyB;86][lnaB;86][lng;86][lqgh;85][lswi;85][lsvB;85][lsvi;85][lsvg;85][loqg;85][lqgk;85][lryB;85][lstg;85][clyg;85][lpyg;85][lruB;85][lryg;85][lpyB; 85][clsg;85][lqyg;85][lpqi;85][lnyB;85][lsCi;85][dlyB;85][clqi;85][dlsB;85][dlsg;85][dlsi;85][dl yi;85][dlyg;85][luyi;85][lsCB;85][elqi;85][lsiB;85][lsgi;85][lqCi;85][lsyi;85][lqCB;85][lrug;85][lrsg;85][lqgA;85][lqwg;85][lquj;85][lvyB;85][lyjB;85][lvyg;85][lmqB;85][lujB;85][luiB;85][lgti ;85][lqwB;85][lmyg;85][elsg;85][elqB;85][lvyi;85][lygz;85][lrsB;85][flyB;85][lugj;85][flsg;85][elyg;85][lqij;85][lwyi;85][lstB;85][lqzB;85][lgri;85][lCyg;85][lnyg;85][lxyB;85][lxyi;85][lxyg;85][lygB;85][flqi;85][lnqi;85][ltug;84][lqwj;84][ltuB;84][lnyi;84][lpug;84][lpuB;84][lsui;84][clyi; 84][lqvj;84][lqAB;84][lqxj;84][lqyB;84][lqiz;84][lghB;84][lgkB;84][lCiB;84][lrsi;84][clqB;84][1 sgB;84][loqB;84][lygh;84][lsgz;84][lsgj;84][clug;84][lygA;84][elsB;84][lygk;84][lyij;84][elsi;84][lyzB;84][lnsg;84][lCgj;84][lswg;84][lpsB;84][lpsg;84][lswB;84][lsxg;84][lsxi;84][loyg;84][lsx B;84][clsi;84][lwyB;84][lryi;84][luzB;84][lmsg;84][lmug;84][lnug;84][lnsB;84][luvg;84][luvB;84][luxg;84][lmyB;84][luxB;84][flyi;84][luCB;84][luyj;84][lugi;84][lugz;84][lugB;84][lmuB;84][elyB;84][lpyi;84][lxCi;84][luCg;84][dlgj;84][lCyi;84][lwyg;84][ltCB;84][ltCg;84][lnuB;84][lmq i;84][ltyi;84][lCyB;84][lriB;84][dliB;84][lCgi;84][elyi;84][lviB;84][fluB;84][flug;84][lvCi;84][fl sB;84][lxiB;83][eliB;83][lmiB;83][lniB;83][quvB;83][ltjB;83][lxyj;83][lngj;83][ltCi;83][elgi;83][lxCB;83][lqyi;83][lnCg;83][lCij;83][lnCi;83][lCgB;83][uvyB;83][lCgz;83][cCg;83][lnsi;83][lqi A;83][lgik;83][lCjB;83][luCi;83][lmCg;83][lvCj;83][luCj;83][lsti;83][lvCg;83][eltB;83][lnCB;83][clsB;83][elgj;83][ltiB;83][ltvg;83][lqih;83][dlqs;83][ltgj;83][lwij;83][lgtj;83][lszB;83][clri;83][clrg;83][lrui;83][dljB;83][elrB;83][lsjB;83][dlxi;83][elri;83][elvi;83][flmg;83][lxCg;83][flrg;83][lsgA;83][lwyj;83][lxCj;83][loyB;83][loqi;83][losg;83][eltg;83][dlrB;83][elxi;83][uvyg;83][ltvB; 83][lqCj;83][lgsj;83][dlij;83][flsi;83][quvg;83][dlri;83][luvi;83][lpiB;83][lrtg;83][lwiB;83][lrtB; 83][dlrg;83][lvCB;83][flvg;83][dlyj;83][lrwi;83][flwi;83][flvB;83][flgi;83][elrg;83][frB;83][luw g;83][lugA;83][luwi;83][lyiz;83][lwCi;83][lsgk;83][fltg;83][fltB;83][clui;83][lsgh;83][dlvi;83][is Cj;83][dlgj;83][dltB;83][lrgi;83][lrjB;83][lugh;83][clvi;83][lrCi;83][lrCB;83][cluB;83][louB;83][lvij;83][lvgi;83][lrgj;83][luij;83][lrCg;83][flCg;83][lvgj;83][luxi;83][dluB;83][luwB;83][lmgi;83][dlug;83][lukB;83][lpsi;83][lmtg;83][cflg;83][lykB;83][luhB;83][clyB;83][lyAB;83][dlgi;83][ip Cg;83][flgj;83][cltg;83][clxi;83][lpCi;83][lvyj;83][fliB;83][lmsB;83][lmtB;83][lugk;83][clgi;83][flvi;83][lvjB;83][lmyi;83][dltg;83][elug;83][luAB;83][elqj;83][lpCB;83][lgij;83][lmrg;83][lmrB; 83][lpqj;83][loug;83][eluB;83][lyhB;83][lwxi;82][lmvg;82][lmui;82][lmpB;82][dlCB;82][lmsi;82][elui;82][eluj;82][lmri;82][lvwi;82][lvxg;82][lvxi;82][lmvi;82][elvB;82][lyih;82][lwCg;82][elx B;82][elgB;82][lxij;82][elgz;82][elyj;82][lxjB;82][elvg;82][elCg;82][lCyj;82][elxj;82][lxgj;82][d lCg;82][lCzB;82][dlCi;82][lvgB;82][lmvB;82][flxB;82][lCgk;82][lgjz;82][lmxi;82][lmnB;82][lm ng;82][fljB;82][flgB;82][celg;82][flgz;82][flCB;82][flCi;82][lvgz;82][flxi;82][lmwi;82][flxg;82][lviz;82][flwB;82][lyik;82][flwg;82][flui;82][dlzB;82][lmpg;82][lvxB;82][luiz;82][dlxB;82][lqv C;82][lCgh;82][lnqu;82][lCgA;82][ltgz;82][lmgB;82][elti;82][lnqs;82][luvj;82][dlxg;82][lmxB;82][lnrg;82][lnrB;82][lntg;82][lgiB;82][lntB;82][ltgB;82][elsj;82][lnui;82][elxg;82][lnuj;82][ltgi;82][lnvg;82][lnvi;82][ltyj;82][lnvB;82][lmgj;82][eflg;82][lmCB;82][lmCi;82][lgiz;82][luxj;82][lri j;82][luwj;82][lmgz;82][lnsC;82][ltzB;82][lCiz;82][elvj;82][elwi;82][elij;82][eljB;82][lwCj;82][f lpB;82][lwCB;82][dlgz;82][flri;82][flgj;82][lwgi;82][dlgB;82][lwgj;82][lwiz;82][flpg;82][lxgi;82][flnB;82][lwjB;82][flng;82][flmB;82][qrug;82][qruB;82][quvi;82][dliz;82][lmxg;82][lyiA;82][ruyg;82][lvzB;82][dlsy;82][elng;82][ltwg;82][ltwi;82][lngj;82][lpCj;82][lijB;82][dlpB;82][dlqu;82][lgsC;82][lgsx;82][lgsw;82][lgsv;82][dlqv;82][lgsu;82][clng;82][lquz;82][clni;82][lgst;82][dlq x;82][lgrj;82][elni;82][lpjB;82][clpg;82][clpi;82][clqu;82][clgj;82][dlgt;82][dlpi;82][lptg;82][uvy i;82][cdli;82][lsxC;82][svyi;82][lrgB;82][dlvg;82][lnCj;82][elqx;82][lnwi;82][losB;82][ltxB;82][ltCj;82][cfli;82][ltxg;82][lsij;82][ltwB;82][lswj;82][lnyj;82][lsiz;82][lskB;82][dlvB;82][lsAB;82][lsyj;82][lnjB;82][loCg;82][dlui;82][ltui;82][lngi;82][loyi;82][lshB;82][ltvi;82][dlpg;82][cdlg;82][dflg;82][lsuj;82][lpwi;82][lsvC;82][dlti;82][lsvj;82][elqu;82][lpui;82][elqv;82][lptB;82][clxg;82][dlwi;82][clwi;82][lrxg;82][lrvi;82][lrvB;82][clti;82][lpgj;82][lrwB;82][lpgi;82][cltB;82][lrxi;82][elpi;82][lrxB;82][lpyj;82][clvg;82][lrgz;82][lquC;82][elpg;82][cliB;82][lrwg;82][elqs;82][dlni; 82][lqux;82][lquw;82][clgz;82][dfli;82][dflB;82][lquv;82][ruyB;82][dlmg;82][dlmi;82][dlmB;82][dlnq;82][elmg;82][dlng;82][lqxC;82][clmg;82][lrzB;82][clgj;82][lizB;82][elpB;82][dlsj;82][cl Ci;82][lstC;82][lrvg;82][clrB;82][lruj;82][elnB;82][dlnB;82][cdlB;81][lwxg;81][lvik;81][lihB;81][qsvi;81][loqi;81][lnqv;81][dlsv;81][flvj;81][loCi;81][flwj;81][swyi;81][dlsx;81][cluj;81][lorg;81][lvkB;81][lvAB;81][lrsj;81][lrhB;81][ltxi;81][cdlq;81][dlsC;81][lvgh;81][lrAB;81][lvgk;81][lr kB;81][lvih;81][lCAB;81][lChB;81][lCiA;81][lviA;81][lCik;81][lCih;81][lvhB;81][lxzB;81][lor B;81][dlgk;81][dlvj;81][dlst;81][dlgA;81][elog;81][lpgB;81][dlxy;81][lpgz;81][lnzB;81][lpsC;81][lmwg;81][dlkB;81][loqj;81][flti;81][lnqC;81][ngx;81][fluj;81][ltgh;81][elrs;81][elst;81][losi;81][lnri;81][lnpB;81][lnpg;81][dlxj;81][lotg;81][flyj;81][dlgh;81][clvB;81][flgh;81][elpq;81][clwg ;81][cluy;81][lnqw;81][dlih;81][lnij;81][lpij;81][lrsC;81][ltAB;81][celi;81][cflB;81][luwy;81][lik B;81][lqwC;81][luyz;81][lsvy;81][luih;81][ltkB;81][luik;81][lqwz;81][luiA;81][lswC;81][lswy;81][lujz;81][lgjB;81][luvy;81][lqvz;81][lgzB;81][lvwg;81][luCy;81][lvCy;81][lgjz;81][lgih;81][ls uy;81][lthB;81][lgik;81][ltij;81][lgiA;81][lstj;81][lghj;81][ljzB;81][lsuC;81][lqxz;81][elrj;81][lux y;81][lgjA;81][lgjk;81][ltgk;81][lwgz;81][lwih;81][lwik;81][wiA;81][lsCz;81][lwzB;81][lxCy;81][lsiA;81][lsik;81][lsih;81][lrwj;81][lrti;81][lxiz;81][lsxy;81][ltwj;81][ltgA;81][lwgB;81][lrCj;81][lvwB;81][lryj;81][liAB;81][lsty;81][lvgA;81][lCkB;81][ltvj;81][lsxj;81][lwxB;81][lriz;81][lx gB;81][lrgA;81][lrgk;81][ltuj;81][lrgh;81][lxgz;81][lsCy;81][flgk;81][cljB;81][lmzB;81][lnxB;81][dlwB;81][lnwB;81][flqv;81][dlqr;81][elgA;81][elnj;81][dlqz;81][clog;81][lgru;81][lgrs;81][lp zB;81][drs;81][lprg;81][flni;81][lprB;81][svyB;81][lnxi;81][flog;811 [eluy;81][eflB;81][deli;81][delB;81][dflq;81][elwj;81][lnwj;81][elwB;81][logj;81][elwg;81][elxy;81][lnvj;81][rsuB;81][elsy; 81][lnxg;81][dlmq;81][lnsy;81][clqs;81][delq;81][dlwg;81][uvyj;81][loCB;81][efli;81][elzB;81][dlAB;81][quvj;81][clnq;81][elns;81][elsx;81][dlqw;81][dlik;81][qswi;81][lmwB;81][lnsj;81][flm i;81][elnq;81][svyg;81][lngz;81][eliz;81][clqt;81][flpi;81][elsv;81][clqv;81][clqx;81][lngB;81][fl qs;81][elqt;81][clpB;81][lpqC;81][dlqC;81][clnB;81][dluy;81][elmB;81][lnuy;81][delg;81]

The percent of host bacterial strains (as profiled by their capsule locus type) that are infected by two phages of a phage combination are provided below. This trait is referred to as “at least 2 phage % coverage”. The phage combinations are ordered in descending performance grade.

[luvy;64][dlvg;62][dlvB;62][dlui;62][lquv;62][dluB;62][dlug;62][dluj;61][luwy;61][dlnB; 61][lsvy;61][delg;61][dlpg;61][dlsv;61][dlpq;61][lgru;61][dlng;61][dlpB;61][lnvy;61][luvB;61][dluy;60][deli;60][dlni;60][dlny;60][luvg;60][delB;60][lswy;60][dlns;60][lnuy;60][dlvi;60][lwyg; 60][lvCg;60][lvyg;60][dlwg;60][lnwg;60][dlgB;60][luwg;60][dlnq;60][luwB;60][dlqr;60][luvi;60][lnsu;60][lnuC;60][dlmg;60][lpuy;60][dlpi;60][dlqu;60][dlwB;60][dlrB;60][dlrg;60][lquw;60][lnsv;59][lgsw;59][dlsg;59][luCg;59][dlCg;59][lruB;59][dlgi;59][dlsy;59][dlyg;59][dlqw;59][lnqu ;59][lsuy;59][lnui;59][lpqu;59][lnvC;59][luvj;59][dlqv;59][luwi;59][dlog;59][dlvy;59][lgsv;59][d lvj;59][dlsC;59][lnwB;59][dlmB;59][lnwi;59][lrui;59][lvyi;59][lpqw;59][dlqs;59][lnvB;59][dlwi; 59][lnvi;59][dlri;59][lqvx;59][lvyB;59][lnqv;59][lvCB;59][lnvg;59][dlyB;57][lrCg;57][lpvg;57][lnug;57][dlnj;57][luwj;57][lmqu;57][luCB;57][dlsB;57][dlxg;57][lwyB;57][dlrj;57][lsuC;57][lpv B;57][dlxB;57][dlmi;57][dlqg;57][lqvg;57][dlsj;57][lnuB;57][dflg;57][lrwg;57][luCi;57][clqu;57][lqwg;57][elsu;57][lwyi;57][dlsx;57][lrgB;57][dlCi;57][lpwg;57][eluy;57][cluy;57][lnqw;57][ln ri;57][lvCi;57][lrug;57][dlCB;57][elug;57][dlrs;57][lwCg;57][dlxy;57][lnsw;57][lgsu;57][clqv;57][dloB;57][lmvg;56][lrCi;56][cluB;56][dlqx;56][dflB;56][lruj;56][lsvC;56][elqu;56][lpui;56][lr wB;56][lvyj;56][clug;56][eluB;56][lmui;56][elui;56][lvwi;56][lwCi;56][lrwi;56][lwgi;56][dlgj;56][lnrg;56][lnuj;56][lnvj;56][lmuj;56][lryi;56][lvwB;56][dloi;56][lvwg;56][lryg;56][lswC;56][dl qB;56][lmwg;56][lmug;56][elvy;56][pwB;56][lgtv;56][lpvi;56][lpuj;56][luxy;56][lqvi;56][lqvB; 56][lugB;56][dlsi;56][luxC;56][dlyi;56][dliB;56][dlst;56][lryj;56][lnwj;56][delq;56][dlti;56][dlty ;56][lrvB;56][lgri;56][lvgB;56][lpCg;56][dlxj;56][lmvB;56][cdlB;56][lvCy;56][elwy;56][dfli;56][lwgB;56][dlgz;56][lqwi;56][lqwB;56][lwCB;56][clvB;56][luCy;56][clui;56][elvg;56][dlgk;56][1 mvi;56][dltB;56][dltg;56][clvg;56][lpwi;56][lmrg;56][lmri;56][cluj;56][luyi;56][clng;56][lvyz;56][lmqv;56][lqux;56][luyg;56][cdlg;56][lprg;56][lpug;56][lpuB;56][lwyj;56][dljB;56][lgtw;56][flq v;56][dflq;56][dlmq;56][dlxi;56][lgrg;56][lmqw;56][lrgi;56][lpsy;56][lpri;56][dlqi;56][dlgt;56][1 mwB;56][muB;56][nrB;56][lgrj;56][lrCB;56][lgtu;56][lsug;55][loCg;55][luCj;55][dlij;55][lrwj; 55][lquC;55][lmCg;55][lnCg;55][cdlq;55][lqug;55][dlyj;55][luyB;55][lrCj;55][lrvi;55][lsuB;55][elav;55][lnpq;55][lnog;55][cdli;55][clpg;55][lmax;55][lsuj;55][lwiB;55][lrvg;55][lqui;55][clni;5 5][lowg;55][lmrB;55][lsui;55][dlkB;55][lmyi;55][lryB;55][louB;55][loug;55][lswg;55][lpyg;55][clqs;55][elrg;55][lpgB;55][lwgj;55][lngB;55][lpqs;55][loui;55][dCj;55][dlqC;55][lvgi;55][eluj;55][lprB;55][lmpg;55][dlgA;55][lnpg;55][lmCi;55][lriB;55][elyg;55][elvB;55][lmCB;55][dlzB;55][clnB;55][lqvC;55][lmwi;55][dlgh;55][flqw;55][elwg;55][lgrs;55][elyi;55][lugi;55][elCi;53][dlh B;53][lnsy;53][clpB;53][flqu;53][luCz;53][dlAB;53][lngi;53][dlqz;53][lnxi;53][lnxg;53][elsy;53][loCB;53][elpg;53][clnq;53][clCi;53][lrxB;53][lrxi;53][loCi;53][lmng;53][lrxg;53][elpq;53][clw g;53][lmpB;53][lmsi;53][lqwC;53][dlik;53][elwi;53][lwjB;53][dliz;53][elqw;53][lnpB;53][dltj;53][lmpi;53][lpqx;53][louj;53][lmni;53][lquB;53][lnCi;53][ltuB;53][lnyi;53][clrg;53][lqvj;53][lqw j;53][clvi;53][lpyi;53][dlgj;53][elvi;53][lmyB;53][lmyg;53][luxB;53][luyj;53][elyB;53][luiB;53][lpCB;53][lvCj;53][lpCi;53][lgrB;53][lnyg;53][lpqg;53][elri;53][ltug;52][lwgz;52][lvxg;52][lvxi; 52][lsxy;52][lsvB;52][lujB;52][lswi;52][elyj;52][lpqi;52][elCg;52][lugj;52][luxg;52][lsyi;52][clr B;52][clCB;52][lwCj;52][lsyj;52][elmg;52][elmB;52][loyg;52][clqx;52][lnsi;52][lrsC;52][clwi;5 2][lnCB;52][lpqC;52][dlih;52][clyB;52][lmnB;52][lpyB;52][lxyg;52][lygB;52][lvxB;52][clyg;52][lorg;52][lnoB;52][lpsC;52][lquj;52][lrjB;52][lmsB;52][lnqC;52][lngx;52][lpgi;52][clCg;52][lsv g;52][celq;52][elsv;52][lswB;52][lrgj;52][ltwB;52][luxi;52][lvgj;52][elty;52][luij;52][lowB;52][1 twg;52][elng;52][lnti;52][lqwz;52][lnsg;52][ltwi;52][lngj;52][elwB;52][flni;52][lnxB;52][cflq;52][lpqt;52][luxj;52][elsw;52][ltui;52][elrB;52][flng;52][clpi;52][elmi;52][lswj;52][clyi;52][loyi;52][lvCz;52][clri;52][lmsg;52][lviB;52][luyz;52][lnyj;52][lqvz;52][dliA;52][elrj;51][clqi;51][flwg; 51][logg;51][lnsj;51][lrsi;51][flnB;51][elqt;51][[nsC;51][lrsg;51][elCB;51][lmxg;51][lowi;51][lm og;51][lnyB;51][clmi;51][elnq;51][elqi;51]lsCi;51][lpsg;51][Cgi;51][lngs;51][Cyi;51][lovg;51][lsvi;51][lwxi;51][clgB;51][lvjB;51][clqB;51][clyj;51][lxCg;51][lwij;51][elpi;51][lwxg;51][lpgj; 51][lnjB;51][logi;51][loyj;51][ltvi;51][celi;51][clmg;51][lsvj;51][ltuj;51][lgsx;51][lniB;51][elqs; 51][elni;51][ltvB;51][lygi;51][elpB;51][loyB;51][lsCy;51][lquz;51][lnCj;51][elqg;51][lsyg;51][lq yi;51][lmoB;51][lpqB;51][lCgB;51][clqg;51][lxyi;51][lCyg;51][lrij;51][lorB;51][lsCg;51][lpyj;51][lvxj;51][lpjB;50][lpsj;50][flvB;50][lnsB;50][lyiB;50][fluB;50][clgj;50][lqxg;50][lmxB;50][flu g;50][lrsB;50][lCyB;50][loCj;50][flgs;50][celB;50][clqt;50][loqB;50][lsyB;50][elns;50][elnj;50][lCiB;50][lntg;50][lvgz;50][lgsC;50][elxy;50][lngg;50][lpiB;50][lmqi;50][lntB;50][elqB;50][lwiz; 50][celg;50][elnB;50][ltvg;50][flvg;50][lrti;50][cliB;50][lovB;50][lrsj;50][clgi;50][lwzB;50][lpqj ;50][lqyg;50][lsCB;50][lmti;50][lpsi;50][lnqi;50][clxg;50][flui;50][lsty;50][lxgB;50][lxyj;50][lw xB;50][lpxg;50][lmxi;50][flwB;50][lxyB;50][lpij;50][lnij;50][lngz;49][lvgh;49][lpqz;49][lpti;49][flyj;49][clog;49][lvgk;49][elog;49][elrs;49][fluj;49][clsg;49][lqyB;49][loqj;49][elsg;49][lmqB;49][lrgh;49][luih;49][lugz;49][lqxi;49][lriz;49][lxCi;49][flyi;49][lgsi;49][lpxi;49][lqCg;49][lpxB;49][clxB;49][ltwj;49][lqCi;49][loxg;49][lwik;49][lgtx;49][flrg;49][lxCy;49][lswz;49][elwj;49][luz B;49][clsi;49][lmag;49][lvzB;49][lCjB;49][flCi;49][lxCB;49][lasg;49][lnij;49][flwi;49][lsCj;49][luiz;49][lugh;49][lmgB;49][lmtg;49][flCB;49][lvij;49][lxgi;49][flpg;49][lgsj;49][elsi;49][flrB;49][flri;49][lpsB;49][clsB;49][flCg;49][lCyj;49][lCgj;49][clgj;49][lpCj;49][elgB;49][lrgz;49][flvi; 49][lrtB;49][elvj;49][elgi;49][clxi;48][flyB;48][lsxg;48][lviz;48][luhB;48][lmtB;48][lpgz;48][lvk B;48][lukB;48][lugk;48][elqj;48][luAB;48][clij;48][cljB;48][lovi;48][lxgj;48][lqCB;48][lyjB;48][lwih;48][lvih;48][eltg;48][lygj;48][lgst;48][flyg;48][lxyz;48][lrtg;48][lsvz;48][lnqB;48][lptg;48][lsxC;48][lsyz;48][nvyi;48][qrui;48][elqx;48][lgsB;48][lqxB;48][cflB;48][lrkB;48][lrgA;48][lptB ;48][elgj;48][lwiA;48][eliB;48][lrzB;48][lrgk;48][ltvj;48][lCij;48][lvgA;48][lujz;48][lqgB;48][lu iA;48][ltCi;48][luik;48][elxg;48][lvhB;48][lugA;48][lqxj;48][elti;48][lmgi;48][elpj;48][eljB;48][lqtC;48][flpi;48][flpB;48][ltCg;48][lyij;48][elsB;48][eltB;47][lrAB;47][clti;47][lsxi;47][elij;47][1 txi;47][lrhB;47][lvAB;47][cltB;47][lvik;47][flvj;47][flwj;47][lyiz;47][cltg;47][elsj;47][lmiB;47][ltCB;47][qrwi;47][lqgi;47][lmij;47][flCj;47][lsxB;47][ltxB;47][cfli;47][nuyg;47][logB;47][lstC;47][loxB;47][lCgz;47][lqxC;47][lsxj;47][lxiB;47][lmgj;47][lnzB;47][elst;47][ltyi;47][lmjB;47][flq x;47][cflg;47][lCiz;47][lxjB;47][ltyj;45][uvyi;45][ruyB;45][lCkB;45][eflg;45][cliz;45][ruyg;45][flqi;45][lgtg;45][lCzB;45][lCgk;45][lxCj;45][lgti;45][uvyB;45][lqiB;45][clgz;45][lygz;45][ltxg;45][nuyB;45][lCik;45][lpzB;45][qruB;45][loxi;45][elxB;45][lqCj;45][lyik;45][lviA;45][qrug;45][f lqj;45][flog;45][efli;45][elxi;45][lqxz;45][eflB;45][ltyg;45][lCgA;44][lxij;44][lyih;44][ltCj;44][1 otB;44][lsCz;44][elgz;44][lxgz;44][quvB;44][ltyB;44][flmi;44][lgtj;44][lyzB;44][qvwi;44][uvyg; 44][lygk;44][lCgh;44][elsx;44][eltj;44][clgk;44][flqB;44][lgsz;44][flqg;44][lgtB;44][eliz;44][quv i;44][lyiA;44]

The percent of host bacterial strains (as profiled by their capsule locus type) that are infected by three phages of a phage combination are provided below. This trait is referred to as “at least 3 phage % coverage”. The phage combinations are ordered in descending performance grade.

[dlvy;48][dluy;47][dlwg;44][dlqw;44][dlqu;44][dlwi;43][dlqv;43][luwy;43][cdlq;43][lqu w;42][dlwB;42][dlqr;42][luCy;42][cdli;42][dlrs;42][cdlg;42][dlrg;41][dlny;41][dluB;41][dlns;41][cdlB;41][lpqu;41][dlug;41][dlri;41][lnuC;41][lpuy;41][lvCy;41][lrvg;40][dlxy;40][dlty;40][clu y;40][dlng;40][dlui;40][luvy;40][lnuy;40][dlCi;40][dlCg;40][lquC;40][dlrB;40][lnvy;40][lnvC;40][dlqC;40][dlsy;40][lnsy;40][lvwg;40][dlmq;40][lgru;40][dlsv;39][lnqw;39][lwxg;39][dlpq;39][dlyi;39][dluj;39][dlCB;39][dlrj;39][lpug;39][dlnj;39][delq;39][dlvg;39][luwg;39][luwi;39][dlyj;39][lnsC;39][dlni;39][eluy;39][suC;39][sCy;39][elwy;39][dlpg;39][dlqx;39][lsuy;39][lnsu;39][lu xy;39][lnpq;39][lqwC;39][lpuB;38][lpqw;38][dlyg;38][luwB;38][dlvi;38][elrs;38][lprB;38][lxCy ;38][lprg;38][clwg;38][dlpi;38][dlgt;38][clqu;38][lquv;38][dflq;38][lpui;38][dlCj;38][dlvB;38][lr wB;38][lrwg;38][lrvi;38][lpri;38][clnq;38][lnsw;38][lgtu;38][lwxB;38][lnqs;38][dlxg;38][dlnB;38][lqux;38][dlsC;38][lnqu;38][lvwB;38][lnqv;38][lrsC;38][lnpg;38][lqvC;36][dlnq;36][lgsu;36][clui;36][lnug;36][dlxi;36][clwi;36][luwj;36][lmav;36][lpsy;36][lrvB;36][lrwi;36][dlpB;36][lvwi; 36][lpxg;36][elqw;36][elsw;36][luxg;36][lrug;36][flqu;36][lngx;36][lnpB;36][lruj;36][elnq;36][e lns;36][luyi;36][dlyB;36][lqxC;36][lnrB;36][lpsC;36][luyj;36][lnrg;36][lrui;36][lpqC;36][lmqw; 36][elvy;36][clrB;36][delg;35][lgtC;35][lrwj;35][lnvg;35][dlqs;35][lwxi;35][luyB;35][lnuj;35][d eli;35][elsx;35][luyg;35][dlxB;35][elwB;35][elwg;35][elxy;35][cluB;35][clri;35][luCi;35][elsu;3 5][lnCB;35][luxi;35][dlmi;35][lgrs;35][clrg;35][luxC;35][lnsv;35][lnui;35][lnCg;35][lnuB;35][el ng;35][lpwi;35][elqu;35][lpwg;35][luvg;35][lpvg;35][lpxB;35][lCyg;35][clug;35][lpxi;35][dlsx;35][lmqu;35][lnyg;35][lnwg;35][lnqC;35][lpqx;35][lnyB;35][lCyB;35][elnB;35][lCyi;35][lpvi;34][lruB;34][lpuj;34][cluj;34][lpqt;34][lryB;34][luvi;34][lnqB;34][dlqi;34][elsv;34][dlvj;34][lpwB;34][lnyi;34][dlxj;34][luCB;34][luCg;34][dlmg;34][lqyg;34][lnri;34][clvg;34][lpyj;34][lnwB;34][el wj;34][elui;34][lnCi;34][lpqs;34][dlqg;34][dlmB;34][lqyB;34][lnyj;34][lvxg;34][lsxy;34][delB;34][lpyi;34][lwCg;34][lngg;34][elni;34][lwCB;34][elwi;34][lgsC;34][lnxg;34][elsy;34][elxB;33][clvB;33][lnvB;33][clyB;33][lqyi;33][celg;33][lswy;33][lswC;33][elpq;33][elxg;33][lpyB;33][lm ui;33][lpCi;33][clng;33][lmwi;33][clyg;33][lmng;33][lpyg;33][luCj;33][lCyj;33][lryg;33][clCg;33][luxj;33][elty;33][dlqB;33][lnwi;33][luyz;33][elpi;33][clxB;33][lrCB;33][elqx;33][clvi;33][clC B;33][lryi;33][lwCi;33][clpi;33][lgtx;33][lgtw;33][elqv;33][clxg;33][lrxg;33][clyi;33][lpvB;33][1 nxB;33][lmwg;33][luxB;33][lmuB;33][clnB;33][clqx;33][clqv;33][elrB;33][eluB;33][elug;33][lu vB;33][clqt;33][celB;33][lsxC;33][lpCj;33][lmnB;32][clpg;32][clmg;32][elpB;32][lrtg;32][elxi;32][uvyg;32][clCi;32][lnCj;32][lsvC;32][lvxB;32][clni;32][elri;32][elpg;32][ruyg;32][lquB;32][fl qx;32][lmug;32][lmrB;32][lmrg;32][clpB;32][lnqi;32][dltB;32][elrg;32][celi;32][lqCB;32][dlst;32][lrCg;32][lrCi;32][dltg;32][lnwj;32][lwyB;32][lwyg;32][dlog;32][clmi;32][lmni;32][dlgj;32][fl qw;32][elpj;32][lmpi;32][lwyi;32][lpCB;32][lmwB;32][lqCg;32][lqug;32][celq;32][lmri;32][lvxi ;32][nuyB;32][elvg;32][nuyg;32][lwCj;32][lnvi;32][clyj;32][lpCg;32][clxi;32][lqui;32][lowg;31][lxyg;31][dlti;31][lowi;31][lryj;31][lsvy;31][lovi;31][elqs;31][ltyg;31][lxyB;31][lptg;31][lstC;31][lgrB;31][lgsx;31][lowB;31][elrj;31][lqvx;31][lqCi;31][ltyB;31][lxCB;31][ruyB;31][lnxi;31][luvj ;31][elvB;31][lmpg;31][clqs;31][lxCg;31][elnj;31][lwyj;31][elCg;31][lrxB;31][elvi;31][dloB;31][eluj;31][elCB;31][dloi;31][flui;31][lrxi;31][elxj;30][lmvB;30][elvj;30][lmvg;30][lntg;30][lpti;30][luCz;30][cflq;30][lmxg;30][lvxj;30][flmB;30][lptB;30][flwg;30][lorg;30][lgtv;30][elCi;30][lnvj ;30][lovg;30][elqt;30][fluj;30][flwj;30][lsty;30][dlqz;30][clog;30][lrti;30][lmpB;30][lrCj;30][flw B;30][flpi;30][lmuj;30][dflB;30][lquj;30][flwi;30][ltvg;30][flug;30][lgrg;30][ltui;30][lpqB;30][lx yi;30][lqwg;30][lvyg;30][lpqg;30][lrtB;30][ltyi;30][lqwB;30][ltug;30][lmyg;30][lvCg;30][clqB;30][dfli;30][clqg;30][fluB;30][lngj;30][lpqi;30][loug;30][lxCi;30][uvyi;30][lmyi;30][ltwg;30][lqs w;30][lgst;30][ltCg;28][lvCi;28][flxg;28][flri;28][ltCB;28][elyB;28][dlsB;28][dltj;28][dlsg;28][d lsi;28][flpg;28][uvyB;28][lxyj;28][lovB;28][ltuj;28][lvyB;28][loui;28][quvg;28][flpB;28][lorB;28][elyg;28][laCj;28][elyi;28][lari;28][arug;28][lmvi;28][lmyB;28][lnsB;28][clai;28][lvyi;28][lq wi;28][lmxi;28][lswB;28][dflg;28][lnoB;28][lnog;28][lmqx;28][elmg;28][lgsv;28][flmi;28][flqv; 28][cltg;28][ltwB;28][lpsB;28][ltxg;28][lntB;28][elst;28][lmxB;28][lxCj;28][ltuB;28][lqxg;28][fl mg;28][elmi;28][flrg;28][lqxB;28][lvCB;28][cltB;28][clsB;27][flqs;27][qruB;27][elog;27][elmB; 27][lsui;27][elyj;27][lmCg;27][lpqj;27][lpsi;27][lswg;27][cflg;27][flvg;27][flrB;27][lpsg;27][lm Ci;27][eflg;27][ltyj;27][louB;27][eflB;27][lnsg;27][ltCi;27][lxyz;27][ltvB;27][ltwi;27][flxj;27][1 mqg;27][cfli;27][clti;27][lsug;27][lxCz;27][loxi;27][cflB;27][flxi;27][efli;27][lsvg;27][lrsg;27][lr sB;27][uvyj;27][lsuB;26][lswi;26][loyg;26][clsg;26][lvCj;26][louj;26][loyB;26][lvyj;26][flyg;26][lsyB;26][elqg;26][flCg;26][lnti;26][qrui;26][mCB;26][lsuj;26][loyi;26][flvB;26][ltvi;26][flvi;26][lquz;26][loxB;26][flyi;26][loxg;26][ltwj;26][lmqi;26][flxB;26][flCi;26][flCB;26][eltg;26][fin B;26][flyB;26][elqB;26][lmqB;26][flng;26][eltB;26][lrsi;26][lpsj;26][lqxi;26][lqvg;26][lrsj;26][1 svB;25][lvyz;25][flni;25][lsyg;25][flqg;25][flCj;25][lgtg;25][lgtB;25][flvj;25][elqi;25][rsuB;25][lqti;25][lqwj;25][lmog;25][elsB;25][lvCz;25][flyj;25][flqB;25][lotg;25][lpqz;25][lsvi;25][lsxg;25][ltxi;25][lqvB;25][lsCB;25][clsi;25][lmtB;25][lnsi;25][ltCj;25][quvB;25][clgj;25][elti;25][lnsj;25][ltxB;25][quvi;25][dlsj;25][losg;25][elsg;24][lsCg;24][lqwz;24][lmoB;24][lstj;24][qrwi;24][cq vi;24][nvyi;24][loCB;24][ltvj;24][eltj;24][lswz;24][flqi;24][lsti;24][loCg;24][loCi;24][lstg;24][lo si;24][lsyi;24][lsxB;24][lgrj;24][lmtg;24][lswj;24][lqvi;24][elqj;24][fltg;24][fltB;24][losB;24][qv wi;24][lstB;23][lsxi;23][lotB;23][lqxj;23][lsxj;23][quvj;23][lsyz;23][elsi;23][suvB;23][loyj;23][1 msB;23][lgsB;23][svyg;23][lmsg;23][flti;23][lsCi;23][lmti;23][lgsg;22][loqB;22][lgsi;22][lqvj;22][flsg;22][lsCz;22][svyB;22][flog;22][lmsi;22][lgiB;22][swyi;22][elsj;22][lqxz;22][lsvj;22][lsyj; 22][dlgB;22][logg;22][lsvz;22][dliB;20][svyi;20][flsB;20][lgtj;20][flgj;20][lsCj;20][loCj;20][flsi; 20][lwgB;20][dlgi;20][elsz;20][logi;20][qswi;20][lqvz;20][clgB;19][dlgj;19][lugi;19][elgB;19][1 pgi;19][lCgB;19][lgsz;18][lpgB;18][lCgi;18][qsvg;18][lvgB;18][dljB;18][lugB;18][lgij;18][lugj; 18][logi;18][lngB;18][luiB;18][elgi;18][lmgB;18][clgi;18][lwgi;18][lpiB;18][eliB;17][lgsj;17][in gi;17][cliB;17][loqj;17][qsvi;17][lygB;17][lsgi;17][lwiB;17][qsvB;17][lqgB;17][lygj;17][lmiB;17][lgjB;17][flgB;17][lxgB;17][lygi;17][lsgB;17][lxgi;17][lrgi;17][lviB;17][lmgi;17][ltgB;17][loi B;17][lrgj;17][lCiB;17][lvgi;17][flgi;17][lugk;17][lwgj;16][lpgj;16][lyiB;16][dlgz;16][lniB;16][d lgh;16][dlij;16][flgA;16][lrgB;16][clgj;16][lsgj;16][fliB;16][lugA;16][ltgj;16][lxiB;16][flgz;16][1 rgk;16][lngj;16][flgj;16][dlgA;16][lghB;16][lijB;16][logB;16][logj;16][lugz;16][lqgi;16][flgk;16][ltgi;16][lujB;16][lygh;15][lrgA;15][lygA;15][lgiz;15][luik;15][lugh;15][lukB;15][lrij;15][ltgA;15][lygk;15][ltgk;15][lyij;15][luij;15][ltgz;15][lgih;15][lgzB;15][lCgj;15][lyjB;15][ltij;15][lqiB;15][flgh;15][lrgz;15][lsgk;15][lsij;15][cljB;15][lxgj;15][ltiB;15][dlgk;15][lpgz;15][lsiB;15][lnij;15][dlih;15][clgk;15][lngz;15][lriB;15][clgh;15][lgkB;15][flhB;14][lnjB;14][lrgh;14][clgz;14][lmgh; 14]

The percent of host bacterial strains (as profiled by their capsule locus type) that are infected by four phages of a phage combination are provided below. This trait is referred to as “at least 4 phage % coverage”. The phage combinations are ordered in descending performance grade.

[dlny;31][dluy;30][dlnq;27][lnuy;27][dlqC;26][cluy;26][dlxy;26][lpuy;26][luwy;25][luCy ;25][luxy;24][luxC;24][dlqw;24][dlpq;24][dlqr;24][dlqu;24][dlvy;24][lnqC;24][lxCy;24][elwy;24][cdlq;24][dlty;23][lnuC;23][eluy;23][lnpq;23][lnqu;23][luvy;23][lnvy;22][lpqC;22][lnqw;22][c elq;22][delq;22][dlrs;22][lpqw;22][lqwC;22][dlmq;22][clnq;22][elxy;22][dlqv;20][dlqx;20][clqu; 20][lvCy;20][clqv;20][dlsy;20][lquC;20][lswy;20][lgru;20][elvy;20][elpq;20][elnq;20][elqw;20][lpsy;19][dlsC;19][lpqx;19][lpqu;19][dlns;19][lmqu;19][lmqw;19][lswC;19][dlsv;19][dlsx;19][lnq x;19][clqx;19][lnvC;19][lqxC;19][lquw;19][lnsw;19][elqu;19][lrsC;18][lnsu;18][lmqx;18][elrs;18][dlst;18][flqw;18][elqv;18][lsvy;18][lqvC;18][cflq;18][dflq;18][elqx;18][lsty;18][lqux;18][lquv ;18][elsw;18][lsuy;18][lnsy;18][lnqv;18][lqvx;18][flqv;17][dlqs;17][lsxy;17][lnsv;17][elns;17][fl qu;17][elty;17][lpsC;17][dlgt;17][lgsw;17][lsCy;17][lsxC;17][elsx;16][clqs;16][lsvC;16][lgrs;16][lnsC;16][elsu;16][elsv;16][clqt;16][flqx;16][elsy;16][lpqt;16][lmqv;16][lgtw;16][lstC;15][lngs;15][lpqs;15][lgtC;15][lgsv;15][lgsC;15][elst;15][lsuC;15][lgtv;15][lgsu;14][flrg;14][flqs;14][clwg; 14][dflg;14][lpwg;14][lgsx;14][dlug;14][lgtu;14][elqt;14][flug;14][dlyg;14][elqs;14][clpg;14][lqt x;12][lngB;12][flpg;12][dlpg;12][lpwB;12][cflg;12][lnwg;12][flyg;12][lprg;12][fltg;12][lrwg;12][luyg;12][flwg;12][dlng;12][clpB;12][fing;12][lnyg;12][dlyB;12][dlrg;12][clrg;12][lngi;12][lnrg; 12][lgst;12][cdlg;12][lryg;12][lnpg;12][lrsg;12][lpyg;12][lrug;12][lnug;12][clng;12][clyg;12][fis g;12][lwyg;12][dlwg;12][dlwB;11][lorg;11][flgB;11][lwgB;11][dlgB;11][luwg;11][lrgB;11][ruy g;11][flrB;11][flgi;11][lnwB;11][nuyg;11][dlui;11][lrxg;11][dlvg;11][flvg;11][lrtg;11][clnB;11][cltg;11][dlgi;11][clyB;11][dlpB;11][lptg;11][ltwg;11][dlmg;11][lniB;11][cdlB;11][ltgi;11][dlnB; 11][dltg;11][lgiB;11][flog;11][lntg;11][flxg;11][lpsg;11][lnsg;11][lpug;11][dlsg;11][lugB;11][lsy g;11][lsug;11][lnyB;11][lugi;11][lnpB;11][clug;11][lygB;11][lswg;11][lpgB;11][ltug;11][lstg;11][clsg;11][lpyB;11][clgB;11][lwyB;11][lygi;10][lnsi;10][flsB;10][dfli;10][dflB;10][ltgB;10][lnui; 10][lovg;10][lwCg;10][dlCg;10][eflg;10][luvg;10][dlqg;10][lrsB;10][lwxg;10][dlni;10][lrvg;10][clmg;10][lpvg;10][luxg;10][lmug;10][clrB;10][clgi;10][dlpi;10][lnrB;10][lowg;10][lruB;10][flpB ;10][loxg;10][lryB;10][firi;10][lwgi;10][lCyg;10][elyg;10][lnwi;10][lpxg;10][lxyg;10][flimg;10][f InB;10][lqyg;10][dlxg;10][flyB;10][clni;10][lsgi;10][lvwg;10][losg;10][lvyg;10][dlrB;10][dlyi;10][clpi;10][lnog;10][clCg;10][lmyg;10][flsi;10][fluB;10][dlog;10][flni;10][clog;10][lnyi;10][lrxB ;10][lprB;10][lmpg;10][clvg;10][elpg;10][loug;10][lsgB;10][elwg;10][flti;10][flui;10][loyg;10][1 sxg;10][lmng;10][lrwB;10][lmwg;10][elrg;10][celg;10][lnti;10][cflB;10][luyB;10][dluB;10][flw B;10][lnxg;10][flCg;10][lrCg;10][flxB;10][lrgi;10][lpgi;10][lxgB;10][lnri;10][lpCg;10][lnuB;10][clwi;10][logB;10][clxg;10][lorB;10][lrsi;10][delg;10][lpwi;10][ltyg;10][lvxg;9][clCB;9][lpxB;9][lwyi;9][elpB;9][lsCg;9][clvB;9][lqwg;9][lovB;9][lsyB;9][lxyB;9][elCg;9][ltwi;9][dlCB;9][lmpB ;9][dlti;9][lmvg;9][lwxB;9][cdli;9][lqug;9][lvgB;9][flxi;9][cliB;9][lstB;9][elng;9][lptB;9][lpui;9][lmnB;9][dlqB;9][clti;9][lgrg;9][lpvB;9][cltB;9][lrxi;9][dlmB;9][lrvB;9][lnoB;9][dlxB;9][loui;9][lsuB;9][elsg;9][lmqg;9][clqg;9][luiB;9][lyiB;9][lpqg;9][flqg;9][dlwi;9][lvwB;9][clxB;9][lvyB;9][lqsg;9][ruyB;9][lngg;9][cluB;9][losi;9][flpi;9][delB;9][lpti;9][lpsi;9][lmtg;9][lqyB;9][clsi;9][lryi; 9][lpyi;9][lriB;9][fliB;9][lCyB;9][dliB;9][dloB;9][loyB;9][dloi;9][mCg;9][lsti;9][svyg;9][flyi;9][clsB;9][ltiB;9][ltvg;9][lpri;9][clyi;9][lsui;9][logi;9][lmrg;9][lnxB;9][lmgi;9][lnxi;9][nuyB;9][lvgi ;9][rsuB;9][flwi;9][flvB;9][lpsB;9][lswB;9][lrwi;9][lsxi;9][fltB;9][lsxB;9][dlvi;9][luwB;9][celB; 9][flvi;9][elwB;9][lpCB;9][elug;9][swyi;9][lpuB;9][lnsB;9][lxCg;9][lrtB;9][uvyg;9][elyB;9][dls B;9][dlri;9][lmgB;9][lpiB;9][dlsi;9][losB;9][lwiB;9][lsvg;9][luyi;9][lsiB;9][clri;9][lrui;9][lowB;9][lxgi;9][lsyi;9][lotg;9][lswi;9][lrti;9][lnCg;9][lnvg;9][lmyB;9][luxB;9][lmwB;9][ltui;9][dlvB;9][loxB;9][louB;9][lwCB;9][ltwB;9][ltxg;9][lntB;9][cfli;9][luCg;9][elog;8][lmog;8][svyB;8][lotB;8][lpxi;8][elvg;8][lpij;8][qrug;8][lmni;8][lowi;8][lvwi;8][lmCB;8][lnvB;8][lnvi;8][eCB;8][ltxi;8][dlvj;8][suvB;8][lpvi;8][lovi;8][loiB;8][elgB;8][eflB;8][lwxi;8][lmxg;8][lvxB;8][elxg;8][svyi;8][lt xB;8][luvB;8][eltg;8][clqB;8][ltCg;8][ltyi;8][elrB;8][dlxi;8][lmsg;8][lvCg;8][lnqi;8][loxi;8][lsvB ;8][loCg;8][lsvi;8][logg;8][loyi;8][ltvi;8][luvi;8][dlij;8][ltuB;8][nvyi;8][uvyB;8][clxi;8][flCB;8][qsvg;8][lgij;8][dltB;8][clui;8][lxiB;8][lxCB;8][luwi;8][lqyi;8][luxi;8][lCgB;8][lrCB;8][clvi;8][lx yi;8][lnCB;8][elmg;8][lqgB;8][lrvi;8][lpqB;8][elqg;8][elnB;8][ltyB;8][dlmi;8][lnqB;8][lgtg;8][dl qi;8][lqvg;8][lqgi;8][lvyi;8][lqwB;8][lqCg;8][lmqB;8][luij;7][lvij;7][flqB;7][lvgj;7][dlgj;7][lmoB ;7][clij;7][lrCi;7][lnij;7][elsB;7][lgrB;7][flqi;7][lwCi;7][qrwi;7][lqvB;7][lmyi;7][loyj;7][eluB;7][1 qiB;7][lmrB;7][loqj;7][lpCi;7][lgsB;7][lvyj;7][dlyj;7][lvCB;7][elmB;7][elsi;7][lqvj;7][loqB;7][in wj;7][clmi;7][luCB;7][lmuB;7][qCB;7][sCB;7][dlrj;7][sCi;7][lpqi;7][lmpi;7][elqB;7][clyj;7][fl ij;7][clqi;7][lgri;7][lmij;7][lxCi;7][lmti;7][lviB;7][lqwi;7][lqvi;7][dltj;7][lqwj;7][deli;7][lqxg;7][c ljB;7][dlgj;7][elyi;7][dluj;7][lmqi;7][lgsi;7][lqui;7][lCyi;7][lCgi;7][flCi;7][flgj;7][lquB;7][elxB;7][lnCi;7][ltgj;7][uvyi;7][lrsj;7][lwgj;7][qvwi;7][lgjB;7][ltvj;7][clCi;7][cqvi;7][ltvB;7][lvxi;7][lwy j;7][lnmxB;7][lngj;7][lgsj;7][clgj;7][quvg;7][lpjB;7][elni;7][elri;7][lwij;7][logi;7][lgrj;7][celi;7][it wj;7][lwjB;7][flmB;7][ltij;7][lijB;7][dlsj;7][lmui;7][lsyj;7][flvj;7][lrwj;7][loCB;7][efli;7][lnjB;7][elvB;7][qsvi;7][elwi;7][lmiB;7][lswj;7][lryj;7][elpi;7][lpgj;7][lsvj;7][qswi;7][qsvB;7][lmwi;7][1 mvB;7][dlCi;7][flmi;7][lmvi;7][elgi;7][lmgj;7][clgj;7][lrCj;6][lCyj;6][lpsj;6][flyj;6][lqij;6][lqxi;6][lyjB;6][lygj;6][fljB;6][lmxi;6][lqgj;6][lrij;6][lgtB;6][lpyj;6][fluj;6][lstj;6][lqxB;6][lugj;6][lmsi; 6][elui;6][lmri;6][lwCj;6][lgjB;6][lpuj;6][lmjB;6][lgti;6][dlCj;6][eltB;6][lqCj;6][ltjB;6][lsgj;6][in vj;6][elxi;6][lnyj;6][lyij;6][elvi;6][lgtj;6][lmtB;6][lrgj;6][louj;6][ltCi;6][lsCj;6][ltyj;6][quvB;6][1 mCi;6][lngj;6][lvjB;6][lnsj;6][lojB;6][lpgj;6][flwj;6][logj;6][lmsB;6][luvj;6][loCi;6][flgj;6][lqCi; 6][elai;6][isij;6][eCi;6][lvCj;6][luCi;6][irjB;6][loCj;6][dinj;6][elti;6][lsjB;6][aruB;6][lvCi;6][qu vi;6][dljB;6][ltCB;6][lCiB;6][qrui;6][eliB;6][dlxj;5][lgik;5][lvik;5][cluj;5][lygk;5][lgjk;5][lsxj;5][flxj;5][ltuj;5][lvxj;5][flCj;5][lmuj;5][elyj;5][eljB;5][elwj;5][elnj;5][lnCj;5][elij;5][uvyj;5][elqj;5][quvj;5][dlik;5][lsuj;5][lnuj;5][elmi;5][elgj;5][lruj;5][lpCj;5][lxyj;5][lCij;5][lCjB;5][elsj;5][ltCj;5][luwj;5][lCgj;5][elvj;5][lxgj;5][luyj;5][dlgk;5][lxij;5][lxjB;5][flgk;5][elrj;5][lujB;5][lquj;5][lqxj; 5][lxCj;5][elpj;5][lmgk;5][luik;3][lgjz;3][lviz;3][lygz;3][lsiz;3][lskB;3][lqvz;3][luyz;3][lgjA;3][1 quz;3][lgjz;3][eluj;3][flgz;3][lrzB;3][lizB;3][lujz;3][lgiA;3][clgz;3][lyik;3][ljzB;3][luiz;3][lgzB;3][lrgz;3][dlzB;3][lqwz;3][lnzB;3]

5 Phage Combinations

5 phage combinations were analyzed in silico for their ability to lyse the 352 different strains of Klebsiella pneumoniae bacteria as profiled by their capsule type, based on the ability of the single phage to lyse a bacteria of a particular capsule type as assessed by a liquid media assay.

The combinations that lyse the highest number of bacterial strains (as profiled by their capsule locus type) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[lgsgi;91][lgstg;91][lgsuB;91][lquiB;91][lqujB;91][lqugj;91][lgsug;91][flqsg;91][lgsiB;91][lgstB;91][lpquB;90][lstyg;90][lgsvi;90][lgsgj;90][lpqsB;90][lpqug;90][lgsgz;90][fiqiB;90][lmq ug;90][lgsgB;90][lmquB;90][lgruB;90][lgriB;90][lquzB;90][lgtuB;90][dlqsB;90][lgtjB;90][lsuyg ;90][lgtgj;90][lgsvg;90][clqsg;90][lnqiB;90][lnqsg;90][dlqiB;90][lquvB;90][lnqug;90][lqxiB;90][lnqsB;90][lquvg;90][lnquB;90][lgtug;90][lsuyB;90][clqug;90][flqgj;90][lgtiB;90][flqgi;90][luyjB ;90][lquxB;90][lgsxB;90][elqsg;90][lqugz;90][lgrsB;90][dlqsg;90][lsygi;90][lgsxg;90][lqugi;90][luygj;90][luyiB;90][lsyiB;90][lqgij;90][flqsB;90][lgsxi;90][lgrug;90][lgswi;90][flsyg;90][flqtg;90][flqtB;90][dlqsi;90][flqug;90][flquB;90][lqviB;90][lpqiB;90][lqugB;90][lgsCg;90][lgsvB;90][1 styB;90][lquxg;90][lgrsg;90][lpqsg;90][lgtgB;89][luvyg;89][lrsiB;89][ltyjB;89][elqgj;89][lnggj;89][lnggi;89][elqxi;89][ltyiB;89][elqgi;89][elqiB;89][lgtzB;89][luvyB;89][elqvi;89][lnqjB;89][lty gj;89][clsyg;89][elquB;89][lgtgi;89][lgtgz;89][lngtB;89][elqug;89][lqvjB;89][flnqB;89][lgtCg;89][flngg;89][lngtg;89][elqsi;89][loqug;89][lgtCB;89][lquwB;89][lquxi;89][luxyg;89][lquCB;89][1 qtxB;89][logsg;89][luxyB;89][flqvi;89][lruyg;89][flmqg;89][lquCg;89][lgrsi;89][flpqg;89][lruyB ;89][lsuiB;89][lquvi;89][elqtB;89][lrsyB;89][flpqB;89][lquyB;89][lquyg;89][lqugA;89][lqxgj;89][fluyg;89][luygB;89][lqugh;89][lgtvB;89][lqugk;89][dlsiB;89][luygz;89][fluyB;89][luygi;89][luy zB;89][lquhB;89][lquwi;89][lgrtB;89][lmqsg;89][lpqtB;89][lnyiB;89][lpqtg;89][lpqsi;89][lgrtg;89][lgtxg;89][dlgjB;89][lmuyB;89][lrsuB;89][flqvg;89][lpgjB;89][lmqtB;89][lquij;89][lrsug;89][d lsyB;89][latvg;89][lnsyg;89][dlsyi;89][dlsyg;89][lnsyB;89][cluyg;89][ltuyB;89][flarg;89][lmatg; 89][ltuyg;89][flqrB;89][lpqgi;89][lpqgj;89][flqsi;89][lpuyB;89][lstCg;89][lmuyg;89][elqtg;89][1 mqgi;89][loquB;89][lgsjB;89][lgrgj;89][clqxi;89][flqxi;89][flqxB;89][lqxgi;89][lswyi;89][lqukB; 89][dlqtg;89][lqvij;89][lqijB;89][lqxij;89][lnuyg;89][lygij;89][clqvi;89][lqxjB;89][dlqgi;89][fityg ;89][lxyiB;89][lpsyB;89][lsCiB;89][lgszB;89][lnuyB;89][flqxg;89][flqwi;89][lqvCi;89][flgjB;89][lquwg;89][elsyg;89][lqwij;89][clqui;89][dlqtB;89][lqxCi;89][lgsgA;89][lquAB;89][dlqug;89][is vyg;89][lstCB;89][clqgi;89][flqgB;89][lvyiB;89][lsvyi;89][clquB;89][flqgz;89][lsvyB;89][lmqiB ;89][lgrgi;89][lsvCi;89][lgizB;89][fltyB;89][lpuyg;89][dlqxi;89][clqsi;89][lgsgh;89][lqvgj;89][lp syg;89][lqCgi;89][lqgiB;89][lgsyg;89][lgrjB;89][lsygB;89][lqgiz;89][lqCgj;89][lgsti;89][lqvgi;89][lsygz;89][lqwiB;89][flyiB;89][cflqg;89][flqvB;89][elqsB;89][lpyiB;89][lnqsi;89][lgsCi;89][lrsy g;89][dlyiB;89][dlqvi;89][lsviB;89][flygi;89][lsCyg;89][lsxyg;89][lsCgi;89][dlqij;89][lqgjz;89][1 sxyi;89][lsxyB;89][lgsui;89][fisyB;89][lgswg;89][dlqgj;89][lgswB;89][lqCiB;89][clqtg;89][flygj; 89][lsxCi;89][lgsCB;89][lsygj;89][dlquB;89][lryiB;89][lgsgk;89][flsCg;88][elsvi;88][flswi;88][1 psiB;88][lsCyi;88][flsvi;88][flsvB;88][flvyg;88][flsvg;88][lsxiB;88][lngxB;88][flsyi;88][clqrg;88][lptyg;88][qsuvB;88][flsgi;88][lptyB;88][lsxCB;88][qsuvg;88][lwyij;88][lsxCg;88][lwyiB;88][fl siB;88][lrswi;88][clqri;88][lqygi;88][lqgih;88][dlqwi;88][lrujB;88][lqCgB;88][flsuB;88][lnpqg;88][eluyB;88][lqvxB;88][dlqvB;88][lmgrB;88][dlqgB;88][dlstg;88][dlqgz;88][lmgrg;88][lqwgi;88][lqvCg;88][lruiB;88][lqwgj;88][lqCjB;88][dlvyi;88][lqvCB;88][lqygj;88][dlqxB;88][dlrsg;88][1 qvCj;88][dlqxg;88][lrugj;88][elsxi;88][cflsg;88][clqsB;88][lsCyB;88][dlngg;88][lsuzB;88][clstg; 88][clsug;88][lsujB;88][lCygi;88][lCygj;88][clsvi;88][lCyiB;88][lsugB;88][lsugz;88][clsxi;88][is ugj;88][clsCg;88][lsugi;88][clsyi;88][clsgi;88][lsuyi;88][lsuCB;88][dltyB;88][lsuCg;88][lsuxB;88][cflqi;88][dlnqi;88][lswiB;88][clqtB;88][clqti;88][elpqi;88][lpsCB;88][clpqi;88][elpqg;88][flqz B;88][lvyjB;88][flrsg;88][flrsB;88][lsyzB;88][elstB;88][lsyjB;88][lpsCg;88][psCi;88][lpsyi;88][flsug;88][lsygA;88][lsygk;88][lsygh;88][flryg;88][firyB;88][lsCgB;88][lsCgz;88][flstg;88][flstB; 88][lsCgj;88][clpqg;88][lqCij;88][lqvgz;88][eflgg;88][lmqvB;88][lnqvB;88][lstuB;88][lnquj;88][lnqui;88][dlqri;88][lnqrg;88][lqxgz;88][dluyg;88][lqxzB;88][lgtkB;88][lngwi;88][dluyB;88][lnqr B;88][lquCi;88][elxyi;88][lquwj;88][lmngg;88][elvyi;88][lqwjB;88][dlsxi;88][lrsCB;88][lmqwi; 88][dflqi;88][dflgg;88][lmygi;88][lquxj;88][lqxiz;88][lqxgB;88][lmtyB;88][lsvCg;88][lrtyg;88][1 quvj;88][lqxCg;88][lrsyi;88][lrsgi;88][elygj;88][dlqrB;88][lmnqB;88][lstug;88][lqxCB;88][lqCg z;88][lgthB;88][lqxCj;88][lquiz;88][lmtyg;88][lgtAB;88][eluyg;88][elygi;88][lrtyB;88][dlqrg;88][lquCj;88][lnqvi;88][lnqvg;88][lqwxi;88][lqgjk;88][dlqti;88][lrstB;88][lmqCg;88][dlsvi;88][lqvz B;88][lmqxB;88][lqgjB;88][lmqxi;88][lmqxg;88][lqgzB;88][lnpqB;88][lqgik;88][lqyiB;88][lqwi z;88][dlqvg;88][lqvxi;88][lqvxg;88][lmpqB;88][dlqui;88][lrsCg;88][lmpqg;88][lqvwi;88][elyiB; 88][lrsCi;88][dltyg;88][lnaxg;88]

The percent of host bacterial strains (as profiled by their capsule locus type) that are infected by two phages of a phage combination are provided below. This trait is referred to as “at least 2 phage % coverage”. The phage combinations are ordered in descending performance grade.

[lvygB;67][lwygi;67][lrugB;67][lvygi;67][lsuvB;66][dlsug;66][luvyg;66][luvyB;66][dlsu B;66][luvyi;66][dlsvB;66][dlsvg;66][lruyi;66][lwygj;65][lquvi;65][dlqvB;65][dlryB;65][lswyg;65][lrwyi;65][dlnqB;65][lvygj;65][lnvyB;65][elvyg;65][lruyB;65][lruyg;65][lquvg;65][lnvyi;65][1 nvyg;65][dlvyB;65][lquvB;65][lqvgi;65][lrugi;65][dlqvg;65][dlvyg;65][lvwyi;65][dlngg;65][lsvy g;65][dlryg;65][lvyiB;65][lqvgB;65][lgrui;65][dlqrB;65][lgrgB;65][lruiB;65][dlpqB;65][lqwgi;65][luvyj;65][dlnyg;65][dlqrg;65][dlpqg;65][luvjB;65][lwyiB;65][lrygi;65][lsuvg;65][lquvj;64][ls vyB;64][eluyg;64][lgrgi;64][elvyB;64][dlqri;64][dlqui;64][lmsug;64][lqvwi;64][dluyB;64][dlpqi; 64][dlquB;64][dlygi;64][lsugB;64][lgswg;64][dlpsg;64][lmruB;64][lnwyi;64][dlriB;64][lmrug;64][luwyg;64][luygB;64][lsvyi;64][lnuyi;64][lgrwi;64][dlsyg;64][dlswi;64][dlnyi;64][dlnqi;64][dls vi;64][lvCyg;64][lwyjB;64][dlsgi;64][lvCiB;64][dlryi;64][luwyB;64][lsvCg;64][dlnsB;64][dluyg ;64][dlqgB;64][lvyjB;64][lgrug;64][lgruB;64][dlqsg;64][dlqug;64][lvyij;62][lswyB;62][elsug;62][dlqsB;62][lryiB;62][eluyB;62][lqugB;62][lqviB;62][lmryg;62][elqug;62][elvyi;62][lgsvg;62][dl qvi;62][elqvg;62][clqvB;62][lqvgj;62][clqvg;62][lmsuB;62][dlpsi;62][dlxyB;62][dlsxg;62][lqwi B;62][lsuCg;62][dlvyi;62][lvCyB;62][lrujB;62][lquwg;62][lqwgj;62][lnugj;62][lquwB;62][luwyi ;62][dlqgi;62][lnujB;62][lsvCB;62][dlsyB;62][lswyi;62][lnqvg;62][lqvCg;62][lvygz;62][lnqvB;62][luygi;62][elqrg;62][lnqvi;62][luCiB;62][lnqui;62][luCgj;62][lrsuB;62][lngwi;62][lnuyB;62][ln uyg;62][lnuiB;62][louyg;61][lmsyB;61][lwCyi;61][lsuyg;61][lmryB;61][lmriB;61][dlsiB;61][lvC ij;61][lrygj;61][lwyij;61][lgswi;61][lrsug;61][lgriB;61][lvyiz;61][lswCi;61][clqrg;61][dlqsi;61][d lsyi;61][flvyg;61][lpuyi;61][lmsCg;61][lmgrg;61][dlmqg;61][lgsvi;61][lgsvB;61][lmqvg;61][dlrs B;61][lnsuB;61][lnsug;61][lqvjB;61][elquB;61][lmuyg;61][dlyiB;61][dlqxB;61][lqugi;61][dlygj; 61][luCyg;61][luwyj;61][lqvxi;61][elgri;61][clsug;61][lmsyg;61][lrugj;61][lnquB;61][lgswB;61][elqvB;61][dlrsg;61][elygi;61][lruxB;61][elygB;61][lqwjB;61][lpqwi;61][dlqxg;61][lsuyi;61][lsu gi;61][lsuCB;61][lmygi;61][lquwi;61][lvCyi;61][lsvCi;61][elqui;61][dlqwi;61][lpqgB;61][lnqug; 61][elsuB;61][lqvCB;61][lvyzB;61][dflqg;61][lrxyg;61][lrxyi;61][dlyjB;60][elgrB;60][clqri;60][1 rtuB;60][lnqrg;60][lpqui;60][lmqsg;60][luCyB;60][lgrCg;60][ltvyB;60][lpsug;60][dlrsi;60][lgsug ;60][lquwj;60][lmqvi;60][fivyB;60][lnygi;60][lryjB;60][lpsuB;60][louyB;60][lrsgB;60][lmqvB;60][clvyi;60][lqvgz;60][firuB;60][lmyiB;60][dlxyi;60][dlmqB;60][lgtvi;60][lmqsB;60][lmqui;60][lrsyi;60][luyiB;60][dlsxi;60][flvyi;60][ltvyg;60][dflqB;60][cluyB;60][lpygi;60][lgrxg;60][lgrxi;60][clquB;60][elpqg;60][lqvxB;60][clqrB;60][clpyg;60][lsuiB;60][lquCg;60][clqug;60][lsuyB;60][logrg;60][loqug;60][dlqiB;60][lwxyi;60][lqvxg;60][dlqgj;60][lruxg;60][lgsui;60][loqui;60][lqvC i;60][loavg;60][ltwyB;60][clavi;60][lrsCg;60][luCyi;60][lmpag;60][lavij;60][clpag;60][elyiB;60 ][lgrgj;60][lpuyg;60][ltwyg;60][ltwyi;60][elqvi;60][lrsyg;60][lnuyj;60][lmqug;60][elqwg;60][lqu yg;60][lpuyB;60][lmgrB;60][flqvg;60][cluyg;60][lwyiz;60][lmuyB;60][lrsCi;59][luygj;59][lmpq B;59][dltyi;59][lmpqi;59][dlstg;59][dlgjB;59][dlqtB;59][cluyi;59][elygj;59][clqui;59][elqwi;59][dlqxi;59][lsugj;59][lqwij;59][lqygB;59][elsyg;59][clquj;59][lmqwi;59][dlqti;59][dflqi;59][lpqug; 59][lgrjB;59][losug;59][lsvgi;59][dlyij;59][dltyg;59][dlyzB;59][lngrB;59][lnquj;59][lrugz;59][lp qCg;59][dlqtg;59][lrtug;59][loggB;59][lquyB;59][elsvg;59][flqwg;59][clqwg;59][lpqgi;59][lsujB ;59][luyij;59][lquiB;59][loqvB;59][lsygB;59][lquCi;59][lrswg;59][lpquB;59][clpqB;59][flrug;59][lmrsg;59][flqvi;59][lxygi;59][lgrsg;59][lgrsi;59][dlsti;59][elsyi;59][dlmqi;59][elqyg;59][clngg;59][lgtwB;59][lgtwi;59][lgtwg;59][lgsuB;59][dlstB;59][lqvzB;59][lgrCi;59][lmsgi;59][losuB;59][1 qviz;59][dlqgz;59][clnyg;59][flwyi;59][lmnyg;59][luxyi;59][elvyj;59][luxyB;59][luxyg;59][lqw Ci;59][lgtvB;59][flqvB;59][loquB;59][lgrxB;59][lgrij;59][luxiB;59][lswiB;59][lquCB;59][lgtvg; 59][lmquB;59][dltyB;59][clygi;59][lqwxi;57][flryg;57][lrswi;57][clpqi;57][losyg;57][clqiB;57][1 suxB;57][lmqxg;57][lrsCB;57][lpsCg;57][elpqi;57][elqgB;57][clqwi;57][lmqgB;57][dlqzB;57][e lquj;57][lqwiz;57][clsvi;57][lCygi;57][lnpqg;57][lqygi;57][clnqi;57][lnsyg;57][lpsyg;57][lsviB;57][lpyiB;57][lquxi;57][lrsyB;57][flryi;57][clpyi;57][lquij;57][ltuyB;57][cdlqg;57][ltuyg;57][fluy g;57][lmsiB;57][elsvB;57][lnyiB;57][lpygj;57][lgrCB;57][lnsCg;57][dlqij;57][clnsg;57][loqvi;57][loqwi;57][lqugj;57][lmnyB;57][lsygi;57][luyjB;57][elxyg;57][lquxB;57][losCg;57][lquxg;57][e lqwB;57][logrB;57][lrswB;57][luxjB;57][clqgi;57][luxyj;57][elyij;57][loqCg;57][dlyiz;57][lvCyj ;57][elqgi;57][lmrsB;57][flqwB;57][lpswi;57][lnsyi;57][lrsgi;57][elpqB;57][elsyB;57][lnygj;57][lstuB;57][elmqg;57][lnqCg;57][clnyi;57][lnggB;57][lnyjB;56][lsyjB;56][lrtyB;56]

The percent of host bacterial strains (as profiled by their capsule locus type) that are infected by three phages of a phage combination are provided below. This trait is referred to as “at least 3 phage % coverage”. The phage combinations are ordered in descending performance grade.

[dluyg;50][dlvyi;50][dlvyg;50][dlvyB;49][dlqwi;49][luwyg;49][luwyi;49][dluyB;49][dlry g;48][lquwg;48][lquwi;48][dlryi;48][cdlqg;48][cdlqi;48][dlqug;48][dlsuB;47][dlsug;47][luwyj;47][lpuyg;47][dlrsi;47][dlqvg;47][lpqug;47][dlrsg;47][dlqui;47][dlquB;47][luwyB;47][dlqvi;47][1 quwj;45][dlqvB;45][dlnyi;45][dlriB;45][dlqri;45][dlnyg;45][dlryB;45][dlqrg;45][dlrsB;45][luCyi ;45][lpqui;45][cluyi;45][lquwB;45][lpuyi;45][lpuyB;44][lpquB;44][cluyg;44][luCyj;44][dlsyi;44][lnvyg;44][dlswi;44][lnuyj;44][luCyg;44][dlmqi;44][dlpsg;44][lvwyi;44][lquCi;44][lmrug;44][1 mruB;44][dlxyi;44][luvyB;43][dlqxi;43][lnqvg;43][lrswB;43][clnyi;43][dltyg;43][lmryg;43][clqu i;43][dlyiB;43][lnvyi;43][dlqrB;43][lquCg;43][lnuyg;43][lquyg;43][lnwyi;43][dlpsi;43][luvyg;43][luvyi;43][lvCyi;43][lvCyg;43][dlpqg;43][luCyB;43][dlmqg;43][lnsug;43][dlnsB;43][dlmqB;43][lrswg;43][clnyg;43][lnuyi;43][cluyB;43][lrwyi;43][dltyi;43][dlygi;43][dlqxg;43][lmuyB;43][lm nyg;43][lmuyg;43][dlsyg;43][lruxg;42][claCg;42][loawi;42][lawCi;42][dlpai;42][dltyB;42][ltCy g;42][lnvyB;42][lruyi;42][lquCB;42][lpsug;42][lnqrg;42][luxyi;42][luxyg;42][lsuCB;42][ltCyi;42][dlsvi;42][lrswi;42][lquyB;42][dlxyB;42][lgrwi;42][lgrui;42][lpsyi;42][lnsuB;42][lmryB;42][lq tyg;42][lgtCg;42][lquvi;42][luxyj;42][clvyi;42][lsuyi;42][dlsyB;42][lntyg;42][lsuCg;42][lruyg;42][lnquj;42][lwCyi;42][lqvwi;42][lquvB;42][lmnyB;42][dlqtg;42][lvCyj;42][dlsvB;42][lvCyB;42][dlsvg;42][lmrsg;42][lquCj;42][lquvg;42][clqug;42][lnqvi;42][clqwg;42][lgrug;42][lmrsB;42][cl gyg;42][dlqtB;41][lmqui;41][lpsCi;41][lmsuB;41][lwxyi;41][luvyj;41][clqCi;41][lsCyB;41][lnqv B;41][lgrCg;41][lsCyi;41][lnsyg;41][dlqti;41][lsuyg;41][lqvCg;41][lgrCB;41][dlqxB;41][lsuyB;41][lnpqg;41][lrsug;41][lnsyB;41][lnqug;41][lgrCi;41][lgtuB;41][lnsCg;41][lxCyg;41][dlygj;41][d lyjB;41][lqCyg;41][lpqCi;41][lpqxi;41][ltCyB;41][lruxB;41][dlpqB;41][dlyij;41][lsuxg;41][lpsu B;41][lpqwi;41][lpqxg;41][lxCyi;41][clqwi;41][lgruB;41][lgsyB;41][lgtui;41][dlnqi;41][lquxg;41][lnsyi;41][lnsCB;41][lnsCi;41][dlngg;41][lxCyB;41][clquj;41][lgtyB;41][lgtCi;41][lgtug;41][el qwB;41][lqxCi;41][lquxj;41][lquxi;41][lgtCB;41][lruyB;41][clpyi;41][lpswi;41][lqxCg;41][lrtuB ;41][lnqui;41][luxyB;41][lpsyg;41][lqvCi;41][lnuyB;41][lpsyB;41][lnqrB;41][elqwg;41][lsCyg;41][eluyg;41][lqxCB;41][lqwxi;41][clquB;41][lngtg;41][lrtug;41][lgsyg;41][lCygi;40][clsug;40][1 rsxg;40][loquB;40][flpyg;40][loqug;40][dlqsi;40][clrsg;40][dflqi;40][clrsi;40][elngg;40][lntyB;40][dlqsg;40][lpqsi;40][clnsi;40][lpqtg;40][lmsug;40][lpqsB;40][dlqgi;40][lmqug;40][clpsi;40][cl nsg;40][lnpqB;40][lquxB;40][lpsCB;40][lmquB;40][dlyiz;40][flqCi;40][elnqB;40][lngwi;40][lpq sg;40][elnqi;40][lpsCg;40][lmngg;40][clsyi;40][lgsuB;40][luCiB;40][lmqwi;40][fluyB;40][elqui; 40][flqCg;40][ltuyB;40][lgsug;40][lnqsB;40][luyjB;40][eluyB;40][dlqiB;40][lrugi;40][lnqsg;40][elswi;40][loqui;40][ltuyg;40][clsyg;40][lpqCB;40][irsyB;40][flnyi;40][clpyg;40][lmqCg;40][lqv xg;40][lmngi;40][elqwi;40][flnyg;40][dlqgB;40][louyB;40][flgyg;40][luyiB;40][lmgrg;40][lqvC B;40][lpqCg;40][lpqxB;40][lnuiB;40][louyg;40][lrugj;40][lgsui;40][lgsCB;39][elquj;39][lrsCB;39][dlnqB;39][elvyg;39][lngxj;39][luygj;39][lrsxi;39][finyB;39][iCyiB;39][lxCyj;39][lgsCg;39][1 oqvg;39][luyij;39][clxyi;39][dlsxi;39][clmqg;39][loqvi;39][lgrsB;39][lrsxB;39][ltxyg;39][clnqi;39][clngg;39][elvyi;39][luxgj;39][lsuxB;39][flpyB;39][clqvg;39][elrsB;39][loqCg;39][clqvi;39][fl pqi;39][logrB;39][lrxyg;39][lmqvB;39][lnqCg;39][clpqi;39][clqri;39][clqrg;39][elsuB;39][lptyg; 39][lgtxg;39][lgrtg;39][figyB;39][lnqCi;39][dlqsB;39][luygi;39][lmnqB;39][lrsyi;39][luygB;39][fluyg;39][irsuB;39][dflqB;39][elqyg;39][lnquB;39][ruiB;39][lrujB;39][lmgrB;39][lquvj;39][clps g;39][elqug;39][lngtB;39][ingxg;39][flrug;39][lmqvg;39][ltwyg;39][lrugB;39][lmqvi;39][lgtwg;39][flqCB;39][elsug;39][dlsxg;39][clqrB;39][lrsyg;39][inqsi;39][cltyg;39][dflqg;39][lsuvg;39][lnq CB;39][dlstg;38][elsxB;38][clpqg;38][lrtyg;38][nuvyg;38][lqvxB;38][flryB;38][elpqi;38][cloqg; 38][lqCgB;38][clsCg;38][lmsCg;38][lrxyi;38][lmsyg;38][lCygj;38][lrsCi;38][elsCg;38][elqvg;38][lrsCg;38][clqxB;38][nuvyB;38][flruB;38][flswg;38][flryg;38][elrsi;38][elvyj;38][lswyB;38][is wyg;38][iaxCj;38][elvyB;38][lmtyB;38][inaxi;38][itxyi;38][impai;38][distB;38][elsvi;38][flswi; 38][lngxB;38][elrsg;38][lqygi;38][lmtyg;38][elsvg;38][lnugj;38][clpqB;38][lnujB;38][lqvCj;38][clqvB;38][flswB;38][flryi;38][ltxyB;38][elxyi;38][clqxg;38][lgrtB;38][lpqtB;38][loqvB;38][flpq g;38][elqvi;38][elquB;38][ltwyi;38][elxyg;38][luykB;38][lnygj;38][lnygi;38][dlgjB;38][lgtxB;38][luCgj;38][lgrxB;38][lsuvB;38][lgrxg;38][lpygi;38][flquB;38][flwyi;38][eltyg;38][lgrsg;38][elsv B;38][dlyzB;38][dlqgj;38][lqCgi;38][elqwj;38][clqtg;38][lswyi;38][lgsCi;38][logrg;38][flqui;38][lsxyB;38][clqxi;38][lptyB;36][lsxCB;36][flpsg;36][lsxyi;36][elsxi;36][lnyiB;36][clqsB;36]

The percent of host bacterial strains (as profiled by their capsule locus type) that are infected by four phages of a phage combination are provided below. This trait is referred to as “at least 4 phage % coverage”. The phage combinations are ordered in descending performance grade.

[dlngg;32][dlnqB;32][dluyB;32][dluyg;32][lnuyB;32][dlryB;32][dlnyg;32][lnuyg;31][dlr yi;31][dlryg;31][lruyi;31][lqCyg;31][lnquB;31][dlnyi;31][lruxg;31][lruyB;31][luCyB;31][lruyg;31][dlquB;30][dlnqi;30][luCyi;30][lnqug;30][lnqCg;30][dlqug;30][luxyg;30][luCyg;30][lnuyi;30][lpuyi;30][cluyB;30][lnqCB;30][dlqrB;30][dlqui;30][lnpqB;30][lnpqg;30][lnqrB;30][lquyB;30][lp uyB;30][dlxyB;30][lruxB;28][lpuyg;28][luvyg;28][dlvyg;28][cluyi;28][dlqrg;28][dlpqB;28][dlpq g;28][lnqrg;28][clngg;28][lwCyi;28][luxyB;28][dlpqi;28][luCyj;28][clnyg;28][lquyg;28][cluyg;28][lnuyj;28][dlxyi;28][luwyB;28][luxyi;28][clpyi;28][lrxyg;28][lngxB;28][clpyg;28][lngxg;28][e luyB;28][cdlqg;28][clqwg;27][clquB;27][luvyi;27][lrwyi;27][nuvyg;27][lntyB;27][lnqCi;27][luw yg;27][clqrB;27][luwyj;27][cdlqi;27][dlvyB;27][ltuyg;27][luwyi;27][lquCB;27][dlqxg;27][lrtyB; 27][lgruB;27][eluyg;27][clqyg;27][lgrug;27][lquxg;27][dltyg;27][flrug;27][lptyB;27][lrxyi;27][ln wyi;27][dlqwi;27][lmrug;27][dlqxB;27][dlvyi;27][lrtyg;27][clnyi;27][dltyB;27][lnqui;27][dlqri;27][lrtug;27][clqwi;26][lmuyg;26][clqug;26][lptyg;26][lquCg;26][lmnyg;26][lngwi;26][lmruB;26][lnvyg;26][lgrCB;26][clpqB;26][lnvyB;26][lgrui;26][lmuyB;26][fluyg;26][elnqB;26][lxCyg;26][elngg;26][lgrxg;26][lquxB;26][lquwB;26][firuB;26][luxyj;26][clqrg;26][lxCyB;26][luvyB;26][el quB;26][lpquB;26][lwxyi;26][clpqg;26][elxyg;26][lmnyB;26][ltwyB;26][dlqvg;26][clnqi;26][luv yj;26][ltuyB;26][clxyi;26][lntyg;26][dltyi;26][lvwyi;25][elqug;25][flnqB;25][elvyB;25][lmryB;25][lmngg;25][lmnqB;25][fluyB;25][ltCyB;25][lgrCg;25][clqCg;25][dlnsB;25][lvCyg;25][dlsxg;25][lngxi;25][lpqxB;25][lpqCg;25][lpqCB;25][dlqvB;25][elgrB;25][lxCyi;25][elvyg;25][lxCyj;25][dlsvg;25][louyg;25][lvCyB;25][lpqui;25][lpqwi;25][lpqxg;25][ltvyg;25][dlsug;25][clqxB;25][cl pqi;25][lquCi;25][clvyi;25][clqri;25][nuvyB;25][dlmqg;25][celqg;25][cltyg;25][lsuvg;25][lgrxB; 25][clqxg;25][elxyi;25][lquxi;25][flqug;25][lquwg;25][elqyg;25][lpqug;25][lrtuB;25][ltwyg;25][clqui;25][lquvg;25][dlqxi;25][elqui;24][elqxg;24][lrswg;24][dlqvi;24][lmryg;24][lnqvB;24][elqx B;24][lnvyi;24][lrsuB;24][louyB;24][dflqg;24][ltxyg;24][ltCyg;24][lpsuB;24][lnqvg;24][elqwg;24][lnsyB;24][flmyg;24][lvCyi;24][finyB;24][flqui;24][dlmqB;24][lgrCi;24][flxyg;24][lgrwi;24][1 awCi;24][elnai;24][larxi;24][clmag;24][ltwyi;24][lnquj;24][elarg;24][clavB;24][flryi;24][lrswB; 24][lpqCi;24][lpsug;24][elpqB;24][dflqB;24][dlswi;24][clrsg;24][dlmqi;24][flquB;24][lngtB;24][elxyj;24][dlsuB;24][flngg;24][lquCj;24][lquwi;24][dlrsB;24][elpqg;24][clqCi;24][elvyi;24][elqw B;24][lmquB;24][flryg;24][lmqug;24][lmpqg;24][firyB;24][dlrsg;24][dlsyB;24][lmpqB;24][lrsug ;24][clqvg;24][dlsvi;23][lgtyB;23][clqxi;23][ltvyB;23][lsuxg;23][dlqtg;23][flpyB;23][ltCyi;23][1 pswi;23][lmtyB;23][fixyB;23][lmgrB;23][lrsCg;23][elmqg;23][lqxCg;23][lnsCB;23][lqxCB;23][elsxg;23][fltyg;23][dlpsg;23][eltyB;23][dlqti;23][flxyi;23][lstvg;23][lswyB;23][elgri;23][elpqi;23][dlsvB;23][lsuyB;23][lquvB;23][elqwj;23][lsuyg;23][dlqtB;23][cflyg;23][lnsug;23][elrsg;23][dlr si;23][clsug;23][flqyB;23][elrsB;23][flqxg;23][losug;23][losyg;23][elqwi;23][flnyg;23][lmngi;23][lmtyg;23][flqwB;23][dlpsi;23][clrsi;23][flvyg;23][lrswi;23][lpqxi;23][ltxyB;23][lrsxg;23][lnsu B;23][dlsyg;23][lrsCB;23][clquj;23][clpsg;23][lrsyg;23][flqrg;23][lmqwi;23][clnsg;23][ingtg;23][lrsyB;23][dflqi;23][flqrB;23][lquvi;23][lmpqi;23][lsuvB;23][lnsyg;23][lqwxi;23][elvyj;23][flwy i;23][dlsxi;23][lmqui;23][dlsyi;22][lswyg;22][lswCi;22][lngsB;22][lotyg;22][elsuB;22][dlqsB;22][flmqB;22][elpgj;22][elqvg;22][lpqtB;22][flqwg;22][lgrtB;22][flqri;22][elquj;22][suvyg;22][elq vB;22][ipstB;22][lpstg;22][flngi;22][lsvyg;22][flqvg;22][elrsi;22][suxB;22][lsvCg;22][flpqB;22][lsuyi;22][clsCg;22][lrsxB;22][lqvCg;22][iqvCB;22][lmgrg;22][dlstg;22][lmqCg;22][qvxB;22][1 quvj;22][lrsyi;22][lrsCi;22][lqvwi;22][flmqg;22][losCg;22][lqvxg;22][lmqxg;22][elqxi;22][losyB ;22][lquwj;22][clqtB;22][clsxi;22][lqrtg;22][clsvi;22][ivCyj;22][clstg;22][ltxyi;22][cflqg;22][lsx Cg;22][flsug;22][flnyi;22][ipsCB;22][losuB;22][isCyB;22][lgtug;22][flgyg;22][lmqxB;22][flqxB ;22][lrstg;22][elsvg;22][flpyg;22][clpsi;22][lsxyg;22][flvyi;22][lgtyg;22][ipsyB;22][lrstB;22][ins Cg;22][elsxB;22][elsug;22][lmqvg;22][igtwB;22][eltyg;22][fltyB;22][lgtui;22][clqvi;22][dlsti;22][isuCB;20][igswB;20][suvyB;20][flsuB;20][lqxCi;20][dlqsi;20][lnsyi;20][lswyi;20][lquxj;20][is xCB;20][elsxi;20][isvCB;20][igtuB;20][clnsi;20][isuCg;20][cflqi;20][lstyg;20][gsyB;20][sCyg; 20][lstyi;20][clqti;20][clsyg;20][flrsg;20][dlstB;20][elsyB;20][psCg;20][lmsug;20][flqxi;20][lnq sg;20][flrsB;20][lpsyg;20][flqCB;20][logrg;20][flpqg;20][clqtg;20][lrsxi;20][eflqg;20][lgtwi;20][lsvyB;20][flqwi;20][elsvB;20][istvB;20][loqug;20][isxyB;20][lqvxi;20][dlqsg;20][lstug;20][lpqtg ;20][iqxCj;20][istyB;20][inqvi;20][elswi;20][elqxj;20][flmqi;20][lgtwg;20].

### Example 4

### Phage Combinations Selected According to Host Coverage as Classified by Bacterial MLST

For this example, the particular phages are referred to by the single letter designation in Table 1, herein above.

2 Phage Combinations

2 phage combinations were analyzed in silico for their ability to lyse the 352 different strains of Klebsiella pneumoniae bacteria as classified by MLST, based on the ability of the single phage to lyse a bacteria of a particular MLST as assessed by a liquid media assay.

The combinations that lyse the highest number of bacterial strains (as classified by MLST) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[ly;63][lq;63][lu;62][lw;62][lg;62][lC;61][dl;59][lv;59][li;59][B;59][lx;57][c1;57][wy;56][lr;56][fl;56][el;56][qw;56][lt;55][vy;55][lp;55][qv;55][uy;55][ls;55][ny;54][ry;54][wg;53][nu;53][xy;53][lm;53][ug;52][qu;52][lo;52][ln;52][qr;52][nw;52][nq;52][dy;52][lj;52][yg;52][wC;51][q x;51][vC;51][uC;51][uB;51][wB;51][wi;51][pq;51][py;51][uw;51][ui;50][yB;50][dn;50][nC;50][dq;50][sw;50][nv;50][nr;50][yi;50][sy;49][dg;49][uv;49][cy;49][rC;49][rg;49][lz;49][qs;49][vg;49][cq;48][qg;48][lk;48][fy;48][lh;48][dv;48][oq;48][su;48][du;48][ds;48][lA;48][my;48][sC;48][pu;47][cu;47][rB;47][dB;47][di;47][ru;47][oy;46][vB;46][qi;46][xC;46][nx;46][qB;46][pC;46][e y;46][ri;46][dw;46][cn;46][rw;46][Cg;46][vi;46][ou;46][mu;46][dC;45][cv;45][cg;45][vw;45][pg ;45][fn;45][fq;45][mw;45][eu;45][dr;45][mq;45][np;45][ux;45][Cy;44][eq;44][fw;44][dp;44][ty; 44][cC;44][xg;44][oC;44][tw;44][wj;44][ow;44][wx;44][Ci;44][pw;44][ci;44][do;44][CB;44][dx ;44][ns;43][cs;43][cB;43][pB;43][pi;43][df;43][mv;43][yj;43][mr;43][cr;43][mC;43][sv;43][rx;43][rs;43][qy;43][qC;43][qt;43][ng;42][ps;42][pv;42][vx;42][fv;42][no;42][dm;42][rv;42][cx;42][mn;42][cd;42][uj;42][cw;42][ew;42][xB;42][xi;42][mx;41][fr;41][px;41][fC;41][er;41][cp;41][tu ;41][fu;41][dt;41][nt;41][mg;41][ni;41][dj;41][sx;41][en;41][ev;41][tC;41][wz;40][ms;40][tv;40][co;39][wk;39][wh;39][yz;39][de;39][nB;39][cm;39][eg;39][wA;39][yk;39][yh;39][pr;39][mB;39][qj;39]

The percent of host bacterial strains (as classified by MLST) that are infected by two phages of a phage combination are provided. This trait is referred to as “at least 2 phage % coverage”.

The phage combinations are ordered in descending performance grade. [qy;28][cw;28][dw;27][vw;26][rw;26][Cy;26][ln;25][pr;25][pw;25][du;24][cd;24][rv;24][uw;24][qC;24][wx;24][dr;24][ru;23][lr;23][lw;23][dl;23][lp;23][cr;22][dC;22][ly;22][lu;22][ux;21][pv;21][cu;21][dy;21][dp;21][ew;21][lv;21][dv;21][uv;21][dx;21][cp;21][de;20][lm;20][uy;20][pu;20][cy;20][cl;20][vx;20][rx;20][wC;19][cC;19][dq;19][mw;19][wy;19][lC;19][cv;19][ce;19][dm;19][px;18][lq;18][cm;18][eC;18][mp;18][qu;18][cx;18][lx;18][or;18][fu;18][uC;18][ry;18][p y;17][cq;17][er;17][mu;17][rC;17][ex;17][nw;17][op;17][ov;17][mr;17][ev;17][aw;16][lo;16][o w;16][pC;16][cn;16][gB;16][vy;16][dn;16][fw;16][np;16][mv;16][my;16][eu;16][ep;16][ls;16][mC;16][ey;16][qr;15][el;15][xC;15][mq;15][cf;15][df;15][rs;15][mn;15][en;15][ny;15][nr;15][fx; 15][nu;15][em;15][fr;15][eq;14][vC;14][fp;14][nv;14][sv;14][xy;14][mx;14][gi;14][nC;14][co;14][fC;14][nx;14][pq;14][fm;13][ef;13][oy;13][nq;13][fv;13][su;13][tu;13][do;13][rt;12][ox;12][iB; 12][sx;12][qv;12][fl;12][sy;12][ou;12][sw;12][fy;12][ps;12][tw;12][qx;12][os;12][cs;12][ns;11][dt;11][ds;11][mo;11][fq;11][es;11][ct;11][eo;11][pt;11][ft;11][ty;11][tv;10][no;10][oC;10][lt;10][ms;10][fs;10][tC;9][qs;9][st;9][sC;9][tx;9][fo;9][nB;9][qt;9][ng;8][oq;8][mt;8][nt;8][et;8][fn;8][o t;7][ni;6][Cg;6][ij;6][gj;6][CB;6][cB;5][yB;5][sg;5][yg;5][lB;5][UB;5][dg;5][sB;5][cg;5][qg;5][d B;5][qB;5][eB;5][Ci;5][xB;5][lg;5][wg;5][eg;5][wB;5]

3 Phage Combinations

3 phage combinations were analyzed in silico for their ability to lyse the 352 different strains of Klebsiella pneumoniae bacteria as classified by MLST, based on the ability of the single phage to lyse a bacteria of a particular capsule type as assessed by a liquid media assay.

The combinations that lyse the highest number of bacterial strains (as classified by MLST) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[lyg;77][lug;76][lqg;76][lwg;76][lyi;75][lgi;75][lyB;75][lvg;74][luB;74][lui;74][lqB;74][1 Cg;74][lwi;74][lwB;73][dlg;72][lvi;72][luy;72][lrg;72][lxg;72][lqw;72][lwy;72][lCi;71][lvB;71][clg;71][lCB;71][lqx;71][lqv;71][flg;71][lqu;71][dli;70][lvy;70][lxy;70][wyg;70][ri;70][lxi;70][cl i;70][dlB;70][lwC;69][lxB;69][luC;69][elg;69][fly;69][fli;69][lrB;69][lpg;69][clB;69][lgt;69][lv C;69][luw;69][flB;69][qwg;69][ltg;69][cly;69][clq;69][vyg;69][flq;68][wyB;68][wyi;68][uyg;68][lny;68][dly;68][lry;68][qvg;68][lty;68][lgs;68][lgr;68][dlq;68][lnq;68][lxC;68][eli;68][nwy;68][1 yj;68][luv;68][lpq;68][ryg;68][log;67][ely;67][lmg;67][dlu;67][loq;67][lgj;67][qwB;67][elq;67][1 sg;67][lsy;67][lsw;67][nqw;67][lpy;67][qwi;67][lpi;67][luj;66][lmy;66][lnu;66][qvB;66][elB;66][clu;66][elu;66][lpB;66][qvi;66][lsu;66][lsC;66][lux;66][ltC;66][flw;66][flC;66][uyB;66][vyB;66][lwj;66][vyi;66][uyi;66][lru;66][lqy;66][lCy;66][lrC;66][qrg;66][clC;66][lsi;66][nvy;66][loy;66][xyg;66][nqv;66][nuy;66][lti;66][ltB;66][dlC;65][ryi;65][qug;65][lpC;65][lqC;65][lmi;65][lCj;65][swy;65][dlw;65][ryB;65][lnw;65][qsw;65][lou;65][lmq;65][uvy;65][pyg;65][ltw;65][lnC;65][ls B;65][flu;65][dlv;65][loi;65][dfl;65][lpu;65][loC;65][lyz;65][flv;65][dls;65][lwx;65][dyg;65][lv w;65][lmB;65][wCg;64][qri;64][ltu;64][qrB;64][uwg;64][nry;64][vCg;64][quB;64][nyg;64][lrw; 64][fix;64][elw;64][lqz;64][uwy;64][pqg;64][elC;64][uvg;64][lyk;64][loB;64][lyh;64][lmw;64][dlx;64][lak;64][axg;64][clv;64][lgi;64][lyA;64][luz;64][lrx;64][xyi;64][qui;64][lwz;64][dlj;64][l vx;64][ltx;64][lsv;64][lnv;64][uCg;64][lmu;64][dlt;64][lvj;64][nqu;64][nxy;64][ngr;64][lsx;64][c lx;63][lpw;63][lwk;63][elv;63][nwg;63][lxj;63][nwC;63][lgj;63][lqA;63][lmC;63][ltv;63][lgh;63][xyB;63][ngx;63][quv;63][luh;63][luk;63][low;63][dln;63][nug;63][lng;63][lwh;63][liB;63][cdl; 63][dlr;63][dyB;63][suy;63][dyi;63][fr;63][lwA;63][pyB;63][nuC;63][nvC;63][luA;63][svy;63][clw;63][clj;63][cls;63][qsv;63][uwB;63][pqB;63][lnx;63][nyi;63][uvB;63][ruy;63][pyi;63][pgi;63][quw;63][del;63][dqg;62][nuv;62][npy;62][nuw;62][clt;62][elx;62][ngg;62][vwy;62][rug;62][1 nr;62][dlo;62][lpx;62][cfl;62][cln;62][rCg;62][lgh;62][uCB;62][lCz;62][wyj;62][lgB;62][fln;62][elt;62][lmx;62][qxi;62][npq;62][vCB;62][uwi;62][fny;62][vCi;62][dny;62][lgz;62][wCB;62][qv w;62][wCi;62][uxy;62][cyg;62][lCh;62][ljB;62][lrj;62][lCk;62][nyB;62][lgk;62][efl;62][lrv;62][uCi;62][lrs;62][lgA;62][elr;62][ouy;62][nwi;62][lmv;62][fyg;62][qvx;62][swC;62][pug;62][lox;62][clr;62][sug;62][nui;62][swg;62][uvi;62][uvC;62][dlp;62][qxB;62][lni;62][fls;62][fwy;61][rsy; 61][vxy;61][myg;61][oug;61][dnq;61][lCA;61][rwy;61][flj;61][wxy;61][nuB;61][qsu;61][syg;61][ngi;61][qru;61][dlm;61][qrw;61][ogg;61][qrx;61][qwj;61][dwy;61][cqv;61][puy;61][lvz;61][cv y;61][rxy;61][pqu;61][noq;61][mug;61][lpv;61][duy;61][dvy;61][ruB;61][nwB;61][dsy;61][dvg; 61][cel;61][clp;61][cny;61][elj;61][uwC;61][dqB;61][lnB;61][lrt;61][flt;61][dug;61][dgi;61][els; 61][dlh;61][dlz;61][suC;61][cug;61][flp;61][nvg;61][lih;61][nrg;61][cuy;61][swi;61][nrC;61][lvh ;61][lvk;61][oyg;61][lij;61][dlk;61][nru;61][lov;60][pqv;60][ngs;60][oqu;60][clo;60][lns;60][clm ;60][flo;60][qwx;60][puB;60][pqw;60][nqB;60][mny;60][lvA;60][qsx;60][eln;60][qsg;60][lps;60][dwg;60][dlA;60][liA;60][qux;60][rwg;60][lmr;60][elp;60][cnq;60][fvy;60][cqg;60][dqv;60][qrs ;60][clz;60][svC;60][rCi;60][rCB;60][noy;60][qwy;60][lxz;60][cyi;60][fyB;60][dnu;60][fnq;60][syi;60][fqv;60][sxy;60][fyi;60][liz;60][nsy;60][rvy;59][oqi;59][pwy;59][mqv;59][mqw;59][cyB; 59][rui;59][lst;59][mwy;59][1 kB;59][myi;59][lzB;59][lik;59][lhB;59][lxh;59][lxk;59][psy;59][pC g;59][myB;59][nsw;59][dng;59][sui;59][oqB;59][qtw;59][nsu;59][suw;59][qvj;59][qvC;59][uxg; 59][uyj;59][dgw;59][nou;59][ouB;59][dxy;59][qsi;59][fqw;59][dqs;59][elo;59][twy;59][vyj;59][euy;59][nCg;59][pqs;59][swB;59][clh;59][pqx;59][pvy;59][clk;59][lnt;59][qwC;59][lnp;59][lmt; 59][suB;59][wCy;59][elm;59][qrv;59][fry;59][lxA;59][cgi;59][duB;59][cqu;59][nty;59][pui;59][1 or;59][eyg;59][dry;59][lpj;59][flm;59][clA;59][npC;59][npu;59][lpt;59][nri;59][vwg;59][xCg;59][lAB;59][wyz;59][muB;59][owy;59][cxy;59][lms;59][lrz;59][nvi;59][dnw;59][syB;59][pxy;59][dsg;59][muy;59][dqu;59][ruC;59][qvy;59][cwy;59][ltj;59][ogw;59][ewy;59][dnC;57][cqs;57][m vy;57][cqB;57][mxy;57][fnw;57][dnv;57][wyk;57][vCy;57][lmp;57][nux;57][lrh;57][lrk;57][ryj; 57][eqv;57][oyB;57][dui;57][oui;57][oyi;57][eug;57][wyh;57][nsC;57][nCy;57][drg;57][dvi;57][nxC;57][lot;57][qsB;57][csy;57][tvy;57][flz;57][dvB;57][sCg;57][dCg;57][fxy;57][cuB;57][oqx; 57][cui;57][nyj;57][fwg;57][cvg;57][qtv;57][oqv;57][cqw;57][cqr;57][qwz;57][uCy;57][elz;57][nCi;57][mqu;57][far;57][oxy;57][dpy;57][wxg;57][dfy;57][wyA;57][dsi;57][cnu;57][dni;57][pu C;57][cqx;57][dwB;57][dgr;57][dqx;57][dwi;57][cry;57][lsj;57][mqg;57][flh;57][lpr;57][noC;57][twg;57][mnq;57][rwB;57][nrB;57][vwC;57][fqg;57][lno;57][ngt;57][eny;57][suv;57][lrA;57][1 mn;57][ouC;57][flk;57][rsC;57][mui;57][equ;57][eqw;57][nvB;57][rwi;57][mwg;57][tyg;57][ev y;57][nxg;56][dvC;56][pCB;56][eyi;56][nqC;56][ngy;56][mrg;56][mry;56][qwk;56][nuj;56][uxB ;56][cuC;56][doy;56][pCi;56][dnr;56][vxC;56][fvg;56][lmo;56][fuy;56][qrC;56][dns;56][lnj;56][fsy;56][lmj;56][oCg;56][cnC;56][quC;56][fnv;56][dsC;56][elh;56][nrw;56][xyj;56][mnu;56][pw g;56][rxg;56][dpg;56][Cyg;56][sCi;56][ntw;56][dpq;56][elk;56][flA;56][mgr;56][duC;56][txy;56][owg;56][quj;56][uxC;56][mvg;56][pvg;56][qrj;56][rvg;56][ery;56][mqx;56][qtx;56][elA;56][cv C;56][fnC;56][rxC;56][cpq;56][fnu;56][mqB;56][fwi;56][nwj;56][qwA;56][nvw;56][dog;56][nsv ;56][rCy;56][cvi;56][enu;56][enq;56][xCi;56][sxC;56][rwC;56][fgi;56][vwi;56][uxi;56][fug;56][eqr;56][lop;56][cCg;56][ovy;56][wCj;56][vxg;56][dnB;56][dxg;56][fwB;56][eyB;56][vwB;56][c dy;56][qwh;56][mqi;56][fqB;56][dfn;56][muC;56][dsB;56][eui;56][euB;56][los;56][nCB;56][loj; 56][dsw;56][dsu;56][dCi;56][exy;56][dsv;56][ltz;56][pry;56][mnw;56][pqr;56][dfg;56][uyz;56][nqj;56][vCj;56][qry;56][dwC;56][qyg;56][ntv;56][ntu;56][dyj;56][dCB;56][qvz;56][uCj;56][nrx; 56][qxy;56][duv;56][quy;56][sCB;56][cng;56][vyz;56][lpz;56][psC;56][xCB;56][svg;56][mwi;56][frg;56][crg;56][mvC;56][fqs;56][rsg;56][rty;56][mwB;56][cpy;56][ory;56][egg;56][drB;56][d ri;56][fnr;56][cvB;56][ogs;56][qtg;56][tuy;56][doq;56][lpk;56][dnx;56][qvk;56][wxi;56][nxi;56][uyk;56][uwj;56][lph;56][wxC;56][twB;56][msy;56][vyh;56][pgj;56][lsk;56][rsu;56][nyz;56][qrt ;56][dmy;56][tug;56][fvB;56][cxg;56][qxC;56][fvC;56][lsh;56][mqs;56][cwg;56][lsz;56][dno;56][mwC;56][fng;56][tyB;56][csC;56][lth;56][ltk;56][uvw;56][vyk;56][xCy;56][nwx;56][mvB;56][uyh;56][pvC;56][ewg;56][mrB;56][mnC;56][qxj;56][npg;56][psu;56][pyj;56][fqx;56][fqu;56][dt y;56][nrs;56][fpy;56][ntC;56][cnv;56][psg;55][tyi;55][mnr;55][eqx;55][uyA;55][mnv;55][sCy;55][fpq;55][CyB;55][csu;55][csg;55][ltA;55][cuv;55][pwB;55][egi;55][pvB;55][qvA;55][ruw;55][q vh;55][qCg;55][euC;55][mvi;55][cni;55][epy;55][now;55][cfy;55][epq;55][mCg;55][owi;55][lsA ;55][cdg;55][esy;55][fuB;55][osy;55][pwi;55][rvC;55][mri;55][rvB;55][ogr;55][nrv;55][dpB;55][twi;55][dpi;55][vyA;55][twC;55][tvC;55][Cyi;55][pwC;55][qsC;55][owB;55][lpA;55][fvi;55][p xg;55][cCi;55][wgi;55][oCB;55][wxB;55][oCi;55][ryz;55][cdq;55][dfq;55][nyk;55][nyh;55][dnp ;55][dey;55][erg;55][dnt;55][pvi;55][pqC;55][ryk;55][nxB;55][fwC;55][qyB;55][nCj;55][cCB;55][pqt;55][egs;55][enw;55][rxi;55][rxB;55][doi;55][dqt;55][drC;55][rvi;55][muw;55][tvg;55][dfi ;55][cnx;55][puw;55][mpq;55][msw;55][qyi;55][eqB;55][qtB;55][dmg;55][xyz;55][duw;55][qtu; 55][fni;55][svw;55][mxg;55][osu;55][svi;55][cri;55][nvx;55][nuz;55][owC;55][euv;55][doB;55][dtg;55][foy;55][ryh;55][frB;55][dmq;55][lmz;55][dfB;55][cdn;55][rsi;55][wgj;55][uvj;55][nyA;55][dxi;55][wiB;55][dqy;54][ryA;54][tuB;54][lmk;54][lmh;54][mrC;54][ugj;54][qsy;54][rCj;54][l oz;54][arz;54][lnz;54][dru;54][cnB;54][vxB;54][vxi;54][quz;54][rsw;54][fCg;54][ati;54][coq;54 ][nwz;54][sty;54][rsB;54][dqj;54][muv;54][npi;54][fsw;54][nvj;54][ygi;54][fuC;54][ouw;54][dx B;54][fui;54][cwi;54][svB;54][ovC;54][prg;54][osC;54][nuk;54][opq;54][pxC;54][nrt;54][fri;54][ugi;54][csi;54][pty;54][dxC;54][crB;54][tuC;54][dnj;54]

The percent of host bacterial strains (as classified by MLST) that are infected by two phages of a phage combination are provided below. This trait is referred to as “at least 2 phage % coverage”. The phage combinations are ordered in descending performance grade.

[lwy;39][dly;38][luw;38][lry;38][lvw;37][quy;37][lqw;37][dlw;37][lqy;37][clw;37][lrw;37][lvy;37][dlv;37][lCy;36][qry;36][lnw;36][luy;36][dlr;36][lgr;36][dqy;36][qwy;36][lwC;36][dlq ;36][clv;36][duw;36][luC;36][lrv;36][lqu;36][luv;36][lpr;35][dlu;35][dln;35][dlp;35][lrC;35][wC y;35][lpv;35][lqC;35][clr;35][lqv;35][lnr;35][qxy;35][rwy;35][lnu;35][lru;35][clu;35][lpy;35][lw x;35][uCy;35][cdl;35][dlC;35][lnv;34][lpw;34][uwy;34][qvy;34][nvw;34][uvw;34][quw;34][lvC; 34][lpu;34][lxy;34][ngy;34][ruw;34][lmu;34][dgw;34][dwy;34][lny;34][cly;34][nrw;34][vCy;34][rCy;34][lsy;33][lmr;33][lnp;33][pwy;33][dvy;33][duy;33][lnC;33][dlx;33][clp;33][vwy;33][rwC ;33][lpq;33][lsw;32][dlm;32][xCy;32][cln;32][qsy;32][muw;32][puw;32][qrw;32][lvx;32][lux;32][dry;32][rvC;32][clx;32][ouw;32][uwC;32][vwC;32][dqv;32][dqu;32][lnx;32][low;32][duv;32][quC;32][qvw;32][wxy;32][lmv;32][ely;32][lmn;32][nuw;32][clq;32][lnq;32][dls;32][pwC;31][ln o;31][lmw;31][rvy;31][qwC;31][dlo;31][cuv;31][dru;31][elw;31][lps;31][qwx;31][dny;31][nwx; 31][elr;31][dgr;31][qrv;31][dpy;31][clC;31][nrv;31][lsu;31][nCy;31][lrs;31][pqw;31][lrx;31][lqx ;31][pry;31][sCy;31][ltw;31][cls;31][lmy;31][lmq;31][dnw;31][dwC;31][pqC;31][lpC;31][mwy; 31][wxC;30][rsw;30][fln;30][clo;30][cqw;30][dsw;30][clm;30][lns;30][dmy;30][cel;30][owC;30][dpq;30][lxC;30][dnv;30][drC;30][dnu;30][flw;30][cnu;30][flu;30][muy;30][cwy;30][loy;30][elu ;30][puy;30][qyg;30][lng;30][lpx;30][quv;30][lmC;30][qyB;30][elv;30][qyi;30][cuy;30][fr;30][q ru;30][del;30][ruy;30][lou;30][lnB;30][dvC;30][lsC;30][cqu;30][cnv;30][oqw;30][nuy;30][ruC;30][lms;30][owy;30][lgs;30][cdq;30][qvC;30][cry;30][fly;30][duC;30][nqC;30][muv;30][dxy;30][nqu;30][pqr;30][dnr;30][cdy;30][qrC;30][lov;30][vwB;28][dsu;28][puC;28][fuy;28][elq;28][cfl;28][dqx;28][lni;28][mry;28][uxy;28][lor;28][nvx;28][pqu;28][dfl;28][eln;28][ltu;28][elp;28][lsv;28][suw;28][dnq;28][vwg;28][lrt;28][qxC;28][lop;28][qux;28][vwi;28][dwg;28][doq;28][nru;28][f lp;28][dnx;28][svw;28][lnt;28][lmp;28][uvC;28][pvC;28][pvy;28][dnp;28][dwi;28][cdn;28][dwB ;28][qsC;28][cwg;28][dey;28][cpy;28][flv;28][uvy;28][nwy;28][nuC;28][nry;28][loq;28][cwi;28][cuC;28][uxC;28][rwg;28][lpt;28][flq;28][dug;28][rwB;28][dnC;28][lmx;28][mvy;28][now;28][c ny;28][dlt;28][cvC;28][pwg;28][dmq;28][Cyg;28][pwB;28][pqv;28][csu;28][vxy;28][uwg;28][ds y;28][CyB;28][Cyi;28][fwy;28][doy;28][ltv;28][loC;28][nrC;28][cqr;28][mqu;28][mwC;28][duB ;28][rwi;28][tuy;28][dxC;28][nuv;28][ewy;28][fwC;28][nux;28][dui;28][mnw;27][qtu;27][flm;27][lmo;27][uwi;27][elx;27][rxy;27][cvy;27][los;27][coq;27][npu;27][dfy;27][nwC;27][fqu;27][df q;27][mqw;27][uwB;27][euy;27][dqs;27][ogr;27][elm;27][els;27][pxy;27][pwi;27][drg;26][cxy;26][qCg;26][ngr;26][mnu;26][eqw;26][elC;26][nrx;26][cdg;26][nsy;26][twy;26][fqw;26][ery;26][mrC;26][nsw;26][nvy;26][nqw;26][drB;26][cpq;26][qsu;26][clt;26][muC;26][dns;26][dri;26][lty; 26][evy;26][lox;26][dty;26][lsx;26][oqu;26][dlB;26][lwB;26][nxy;26][nxC;26][cqv;26][equ;26][rsu;26][dsC;26][cqx;26][fpy;26][suy;26][pxC;26][cnq;26][fnu;26][ouC;26][wxB;26][ovy;25][cn x;25][rvg;25][swC;25][rvB;25][dli;25][enw;25][opq;25][rvi;25][prg;25][ovC;25][lnz;25][mgr;25][euv;25][cnC;25][psu;25][flC;25][dsv;25][fnw;25][lwi;25][epy;25][msw;25][npy;25][eny;25][in j;25][fuC;25][cfy;25][lwg;25][twC;25][msy;25][dlg;25][swy;25][vxC;25][wxg;25][uwj;25][nvC; 25][suC;25][crB;25][wxi;25][lrB;25][enu;25][fry;25][tuC;25][crg;25][rxC;25][mxy;25][qvx;25][ory;25][nsu;25][fvy;25][lwj;25][csy;25][elo;25][dpg;24][lmt;24][lrj;24][nqv;24][rui;24][pqx;24][dpB;24][dqt;24][fls;24][mnr;24][qrx;24][dno;24][lrg;24][dfn;24][euC;24][cuB;24][egr;24][dvB;24][dlj;24][dvg;24][dxB;24][mpq;24][flo;24][rug;24][nrs;24][mvC;24][mnv;24][oqv;24][lug;24][psC;24][suv;24][rsC;24][fnr;24][dCg;24][nsC;24][cui;24][dpi;24][qsw;24][pty;24][lpj;24][lpB;24][dvi;24][dCi;24][dCB;24][ouy;24][exy;24][csC;24][ruB;24][lri;24][rty;24][qtw;24][cri;24][qrs; 24][ngs;24][dxg;24][cug;24][fpq;24][luB;24][mny;24][npC;23][flx;23][lwA;23][fvC;23][rsy;23][enq;23][ltx;23][ngx;23][lwh;23][luj;23][lwz;23][dlz;23][lrA;23][cqs;23][lrh;23][lrk;23][npq;23][mnq;23][epq;23][dyi;23][cyB;23][fny;23][lpg;23][lrz;23][cyg;23][efl;23][mqv;23][dyg;23][lui;23][mnC;23][lwk;23][dyB;23][dxi;23][psy;23][dlh;23][lyj;23][dmg;23][dyj;23][fnv;23][pqs;23][d nB;23][fnC;23][mwg;23][dlk;23][eqv;23][lpk;23][lpA;23][lph;23][uyg;23][lyB;23][lpz;23][uxi;23][dqg;23][uxg;23][esy;23][dlA;23][ltC;23][sxy;23][clg;23][cvB;23][lpi;23][uxB;23][mqx;23][c yi;23][noy;23][cvg;23][fqr;23][lgt;23][oxy;22][vxB;22][cCg;22][clB;22][lyi;22][lvB;22][mwB;22][ntu;22][nsv;22][ewg;22][ntw;22][cli;22][lyg;22][rxB;22][cCB;22][uyi;22][luA;22][fsw;22][qu g;22][mqs;22][lot;22][uyB;22][tvy;22][fnq;22][lvj;22][uCg;22][eqx;22][qrt;22][dqB;22][nou;22][dgi;22][dng;22][dnt;22][lst;22][luk;22][rxi;22][rxg;22][cCi;22][lyz;22][lyk;22][uvg;22][lyh;22][lCB;22][pvg;22][wCg;22][fqv;22][wyg;22][pvB;22][uCi;22][svC;22][cqg;22][nwB;22][lyA;22][vxg;22][luh;22][lvg;22][luz;22][vxi;22][pvi;22][cvi;22][wyB;22][mwi;22][wCB;22][uCB;22][uv i;22][tvC;22][dni;22][wyi;22][pui;22][wCi;22][fqx;22][pug;22][lmj;21][lvA;21][cqB;21][puB;21][quB;21][uvB;21][nty;21][lmg;21][pqt;21][egs;21][cng;21][lCg;21][nug;21][nwg;21][dgj;21][fx y;21][lvi;21][nuB;21][lCi;21][lmB;21][qui;21][svy;21][npg;21][clj;21][sxC;21][noC;21][elt;21][noq;21][lvz;21][lvk;21][lvh;21][lxg;21][qtv;21][qsv;21][foy;21][uyA;21][cnB;21][nwi;21][lxB;21][txy;21][nrt;21][oqx;21][uyj;21][cgi;21][qsx;21][pxg;21][cxg;21][uvj;21][nrB;21][rCg;20][lqB ;20][nyB;20][lmz;20][erg;20][nui;20][lsg;20][lCj;20][lxi;20][lmk;20][clA;20][lmh;20][lmi;20][u yz;20][nvB;20][owB;20][clk;20][clh;20][qwg;20][uyh;20][npi;20][owg;20][rCB;20][clz;20][cni; 20][wCj;20][uyk;20][nrg;20][lag;20][lij;19][eug;19][xCB;19][wyj;19][awB;19][nat;19][eyg;19][qwi;19][nyg;19][ryB;19][lgi;19][ryg;19][ntC;19][lsB;19][rCi;19][uCj;19][eyB;19][owi;19][nCB ;19][nvg;19][osy;19][fui;19][egg;19][mCg;19][xCg;19][wyk;19][wyA;19][flt;19][lCk;19][ryi;19][fsy;19][nyi;19][wyh;19][euB;19][nCg;19][elB;19][lCz;19][fwg;19][lCA;19][eyi;19][lgh;19][nri; 19][wyz;19][quj;19][lCh;19][pCg;19][pCB;19][pyB;18][lgk;18][pCi;18][loB;18][pyg;18][nxg;18][lxA;18][lqz;18][mui;18][lqA;18][fwB;18][eqB;18][elg;18][quz;18][nvi;18][nCi;18][xCi;18][fu g;18][qtx;18][nxB;18][nwj;18][lsj;18][fuB;18][eui;18][lsi;18][lxj;18][ryh;18][mug;18][myg;18][osu;18][egi;18][ryj;18][lxz;18][nqB;18][ryz;18][lxh;18][lxk;18][muB;18][mqg;18][dnj;18][ryA;18][ntv;18][fwi;18][loj;18][dfg;18][log;18][qrg;18][eli;18][rCj;18][vyB;18][mrg;18][vyg;18][xyB; 18][pyi;18][ryk;18][ngg;18][vyi;17][myB;17][fCg;17][sty;17][swg;17][xyg;17][swB;17][rsB;17][dfB;17][loi;17][elj;17][osC;17][qrB;17][vCg;17][doB;17]I[vCB;17][mri;17][rsg;17][mvg;17][oq s;17][nxi;17][dog;17][mrB;17][fqs;17][pyj;17][svB;17][vCi;17][mvi;17][gB;17][myi;17][doi;17][qri;17][svg;17][mvB;17][lsA;17][dfi;17][vyj;17][qwh;17][lsz;17][nwz;17][syg;17][loz;17][mxg ;17][dsB;17][ngi;17][mqB;17][xyi;17][vyA;17][qwj;17][syB;17][dsg;17][vyk;16][frg;16][gqwz;16][lsk;16][qxB;16][rsi;16][vyz;16][qxg;16][csg;16][nuj;16][tug;16][qwk;16][mi;16][gqwA;16][v yh;16][lsh;16][tuB;16][svi;16][elA;16][ugi;16][nyj;16][fri;16][vCj;16][frB;16][swi;16][xyj;16][n vj;16][pqg;16][fqg;16][elh;15][qvg;15][ygi;15][lgi;15][qrj;15][pqB;15][csi;15][elz;15][fyg;15][el k;15][qxi;15][dsi;15][flB;15][twg;15][qrz;15][oyg;15][twi;15][flg;15][oyB;15][twB;15][wgi;15][suB;15][syi;15][psg;15][dtg;15][tyi;15][nyk;15][fyB;15][sCB;15][pgi;15][qsg;15][fng;15][nuk;15][oyi;15][fvg;15][fgB;15][nyz;15][qvB;15][nyh;15][sug;15][sCg;15][nuz;15][tyB;15][nCj;15][s ui;15][flj;15][tyg;15][nyA;15][oui;14][qsB;14][ngj;14][fvi;14][oug;14][qvi;14][xyz;14][fgi;14][f vB;14][fli;14][fyi;14][liB;14][tvg;14][qxj;14][wiB;14][oCg;14][oCB;14][ouB;14][ltB;14][pgj;14][ltg;13][qvj;13][qvA;13][qvh;13][lti;13][sCi;13][flA;13][fni;13][oCi;13][flz;12][qvk;12][qsi;12][qvz;12][qtg;12][flk;12][flh;12][qtB;12][ltj;12][qti;12][ogg;11][oqB;11][ltz;10][oi;10][ltk;10][lt A;10][lth;10][wgj;9][lgj;9][lij;9][ugj;8][ljB;8][lzB;6][lgz;6][lgk;6][lkB;6][lhB;6][lAB;6][lgh;5][l gA;5][liz;5][lik;5][lih;4][liA;4]

The percent of host bacterial strains (as classified by MLST) that are infected by three phages of a phage combination are provided below. This trait is referred to as “at least 3 phage % coverage”. The phage combinations are ordered in descending performance grade.

[ruw;20][duw;20][dru;20][dwC;19][puw;18][cwy;18][uvw;18][lpw;18][cdy;18][lrw;18][d lw;18][lpr;18][duC;17][clw;17][lru;17][duv;17][dqy;17][dwy;17][dlu;17][dlr;17][lrv;16][lvw;16][lqy;16][duy;16][cdl;16][drC;16][lmp;16][lnp;16][cuy;16][cqw;16][lmw;16][ruy;16][luw;16][cd q;16][dlp;16][lCy;15][dry;15][lwx;15][cuC;15][lpu;15][clr;15][dxC;15][quy;15][lrx;15][cry;15][dqu;15][clp;15][dlm;15][dgw;15][wCy;15][cuv;15][pwC;15][cpy;15][dnw;15][dln;15][lpx;15][ln w;15][dlC;15][uwC;15][cln;15][lpv;15][pry;15][uCy;15][uwy;15][rwC;15][lux;14][euC;14][clu; 14][uvy;14][elw;14][rwy;14][del;14][wxC;14][cvy;14][euv;14][dlx;14][lnr;14][cdn;14][dey;14][1 mn;14][lor;14][vwy;14][muw;14][qwy;14][pwy;14][clm;14][cly;14][lny;14][qwC;14][quC;14][e wy;14][ruC;14][dpy;14][cqr;14][lnu;14][rCy;14][qru;14][lpC;14][dlv;14][dvC;14][rvy;14][puy;14][clC;14][rxC;14][lop;14][luv;14][lmy;14][dly;14][enw;14][luy;14][cqu;14][vwC;14][lmr;14][mwC;14][lpy;14][dgr;14][pvy;13][elC;13][lnv;13][lqC;13][dmy;13][uxC;13][dxy;13][eln;13][ux y;13][dmq;13][qrC;13][lwC;13][qry;13][cxy;13][lvx;13][dnp;12][lmu;12][lmv;12][lwy;12][euy; 12][dnr;12][lnC;12][quw;12][lnx;12][lry;12][wxy;12][lnq;12][qrw;12][dnu;12][rxy;12][elp;12][d vy;12][dlq;12][eqw;12][puC;12][cvC;12][lmC;12][rsu;12][cpq;12][epy;12][low;12][dpq;12][vxC ;12][dnC;12][svw;12][ory;12][nuw;12][mqw;12][nrw;12][lmx;12][mwy;12][clv;12][muC;12][cel ;12][clx;12][ery;12][pqr;12][fuC;12][nru;12][cnC;12][lrC;12][muv;12][lov;12][pqw;12][elr;12][pxC;12][clq;12][suv;12][npu;12][mnw;12][qvy;12][uvC;12][npC;11][vxy;11][exy;11][nwx;11][c ny;11][xCy;11][nwC;11][nvw;11][elv;11][pvC;11][qux;11][elx;11][elm;11][nCy;11][cqv;11][px y;11][qrx;11][lns;11][muy;11][npy;11][qvw;11][pqu;11][rvC;11][lrs;11][vCy;11][evy;11][elu;11][mqu;11][mrC;11][luC;11][dqx;11][pqC;11][lmq;11][cnx;11][rsw;11][mpq;11][ngy;11][equ;11][lqu;11][flw;11][mry;11][flx;11][lsv;11][rsy;11][lpq;11][dnx;11][egr;11][qxC;11][nuv;11][fwC;11][ouy;11][flu;11][nrv;11][nwy;11][lxC;11][cfy;11][svy;11][cnu;11][lvy;11][mnr;11][mny;11][q uv;11][nux;11][mgr;11][nrx;11][mvC;11][cqx;11][dfl;10][qvC;10][mqv;10][enu;10][lno;10][ovy ;10][dfy;10][lou;10][cnq;10][lqw;10][loy;10][dqv;10][dsv;10][lgr;10][fqu;10][mnC;10][qwx;10][nqC;10][ely;10][ouw;10][qrv;10][qxy;10][lox;10][flp;10][suw;10][mnv;10][sx;10][fr;10][fuy;10][dny;10][eqv;10][dnv;10][epq;10][dsw;10][lmo;10][eqx;10][dlo;10][mvy;10][lps;10][lvC;10][nuy;10][fwy;10][dnq;10][dsu;10][psu;10][npq;10][fxy;10][cnv;10][lsu;10][flC;10][pqx;10][lxy;10][fry;10][lsw;10][suy;10][mnu;10][mxy;10][clo;10][pqv;10][osu;10][nqw;10][csy;10][doy;10][qvx;10][mnq;10][eny;10][dfq;9][dls;9][csu;9][cfl;9][fqr;9][elq;9][nry;9][qsy;9][mqx;9][osy;9][nv y;9][enq;9][flm;9][psy;9][owy;9][nvx;9][swy;9][los;9][elo;9][fvC;9][cls;9][lqx;9][nrC;9][fpy;9][nsv;9][dty;9][fqw;9][nuC;9][efl;9][lsy;9][loC;9][flv;9][dsy;9][rty;9][oxy;9][fly;8][lms;8][sCy;8][nqr;8][fvy;8][msw;8][nxC;8][fsw;8][nqu;8][csC;8][nxy;8][lqv;8][lrt;8][nrs;8][lty;8][fqx;8][tuy;8][nvC;8][dsC;8][sxy;8][rsC;8][esy;8][dlt;8][sty;8][fsy;8][qsv;8][pty;8][sxC;8][fpq;8][tuC;8][twy;8][fnw;8][now;8][twC;8][nou;8][swC;8][ngx;8][cqs;8][fqv;8][ltu;8][ltC;8][noy;8][owC;8][fln;8][e ls;8][qrs;8][dqt;8][ovC;8][svC;8][dno;8][tvy;8][lpt;8][nqv;8][dfn;7][ouC;7][qsw;7][flt;7][flq;7][n sw;7][ntw;7][lnt;7][lsC;7][suC;7][qsu;7][qrt;7][fls;7][loq;7][nsu;7][ogr;7][lgt;7][fnu;7][ntv;7][lqs ;7][ltw;7][clt;7][nrt;7][qsx;6][osC;6][qsC;6][lst;6][txy;6][dns;6][flo;6][msy;6][fqs;6][lty;6][ntu;6][egs;6][qtu;6][dnt;6][noC;6][psC;6][oqu;6][qtw;6][foy;6][oqv;6][ds;6][ltx;6][lmt;6][opq;6][fnr; 6][pqt;6][nsy;6][fnv;6][mqs;6][elt;6][coq;6][oqx;6][nty;6][ogs;6][fnC;6][tvC;6][doq;6][pqs;6][nt C;5][lot;5][atv;5][fny;5][noq;5][atx;5][oaw;5][nat;5][dCg;5][nsC;5][lyg;5][Cyg;5][cnB;5][cwg;5 ][lgB;5][lnB;5][fnq;5][dCB;5][ngs;5][qyg;5][lyB;5][qyB;5][CyB;5][nyB;4][qCg;4][nyg;4][ngg;4][wCB;4][nCB;4][lng;4][lmg;4][cdg;4][ygi;4][lmB;4][nxB;4][pyB;4][cCg;4][clB;4][nwg;4][lpB; 4][cCB;4][dyB;4][pyg;4][dyg;4][lpg;4][wCg;4][dwg;4][lqB;4][lqg;4][dwB;4][nCg;4][nwB;4][lC B;4][ewg;4][nqB;4][lCg;4][myB;4][myg;4][cng;4][lyi;4][lvg;4][lwg;4][qyi;4][lsB;4][lwB;4][dlg; 4][nxg;4][pqg;4][wiB;4][lmi;4][ryB;4][wxg;4][lrg;4][prg;4][lrB;4][mvg;4][pvg;4][vyg;4][wyB;4][ryg;4][mqg;4][wyg;4][mrg;4][lpi;4][cwi;4][pCB;4][clg;4][lvB;4][dlB;4][vyB;4][lxB;4][pvB;4][liB;4][npg;4][wgi;4][mug;4][mvB;4][mrB;4][dqB;4][dCi;4][dqg;4][Cyi;4][myi;4][dnB;4][cyB;4][sug;4][lgi;4][pyi;4][pqB;4][dng;4][cyg;4][cxg;4][mqB;4][pCg;4][mCg;4][wxB;4][rsB;3][nxi;3][pyj;3][mxg;3][crB;3][drB;3][qrj;3][rvg;3][vxB;3][rsg;3][fug;3][crg;3][mwB;3][svg;3][npi;3][dp B;3][wxi;3][pgj;3][cni;3][dmg;3][lnj;3][mvi;3][psg;3][pCi;3][vxg;3][pvi;3][uxB;3][dxg;3][eyB;3][rxB;3][mqi;3][csg;3][pwB;3][cCi;3][dxB;3][svB;3][pxg;3][rvB;3][mri;3][xCB;3][xCg;3][muB; 3][qrg;3][eyg;3][qrB;3][uyg;3][rCB;3][qvg;3][cqg;3][ruB;3][vCg;3][uvg;3][drg;3][pgi;3][oyg;3][nyi;3][cqB;3][pug;3][elg;3][dyi;3][lsg;3][rCg;3][lni;3][uxg;3][xyg;3][nwi;3][ljB;3][qvB;3][elB;3][ryi;3][ngi;3][log;3][vyi;3][syB;3][uyB;3][loB;3][puB;3][qwB;3][rwg;3][uvB;3][qwg;3][suB;3][wCi;3][nvB;3][mwg;3][dwi;3][lvi;3][luB;3][vCB;3][rxg;3][dpg;3][lCi;3][xyB;3][lgi;3][pwg;3][c ug;3][nvg;3][lxg;3][lug;3][rug;3][nrB;3][rwB;3][syg;3][nrg;3][lri;3][oyB;3][ryj;3][nCi;3][loi;3][1 xj;3][dfg;3][oug;3][ouB;3][lgj;3][qsg;3][sCB;3][qug;3][uyj;3][lrj;3][nug;3][vyj;3][dri;3][swB;3][cvB;3][ngj;3][uwB;3][xyi;3][rsi;3][quB;3][cri;3][frB;3][dgi;3][uvj;3][dug;3][dvg;3][dxi;3][uwg; 3][fvB;3][dfB;3][qwj;3][rCi;3][cyi;3][fyB;3][syi;3][clj;3][qri;3][uCg;3][ogg;3][eqB;3][qxg;3][svi ;3][erg;3][fuB;3][vwB;3][lmj;3][oCg;3][eug;3][oyi;3][fwB;3][fwg;3][nyj;3][lxi;3][ugi;3][swg;3][cuB;3][cvg;3][dvB;3][flB;3][cli;3][fvg;3][uyi;3][vxi;3][wyj;3][fCg;3][dni;3][lui;3][vCi;3][quj;3][lwi;3][dli;3][lsj;3][rwi;3][mui;3][oCB;3][uCB;3][flg;3][sCg;3][euB;3][dsB;3][qsB;3][qvj;3][eqg; 3][rui;3][lsi;3][rxi;3][rvi;3][frg;3][mwi;3][fyg;3][sui;3][oqB;3][qvi;3][xCi;3][nvi;3][qxB;3][dsg;3][lpj;3][wyi;3][nri;3][uxi;3][lyj;3][dpi;3][uvi;3][nuB;3][pui;3][duB;3][pwi;3][vwg;3][lgj;3][qxi;2][lCz;2][oCi;2][fvi;2][sCi;2][luz;2][luj;2][lwj;2][fui;2][qwi;2][eli;2][ltg;2][fri;2][csi;2][fli;2][dgj; 2][lnz;2][ugj;2][rCj;2][qrz;2][quz;2][nwz;2][nvj;2][dfi;2][qui;2][ltB;2][dtg;2][dlj;2][lwz;2][nuz;2][lvj;2][doB;2][xyz;2][wgj;2][lmz;2][lqz;2][nCj;2][lyz;2][qtB;2][lCj;2][lgz;2][ryz;2][dnj;2][uwi; 2][cgi;2][cvi;2][wyz;2][lrz;2][vwi;2][lzB;2][oqi;2][vyz;2][qtg;2][lpz;2][qsi;2][lxz;2][dyj;2][fyi;2][qvz;2][clz;2][fng;2][lij;2][swi;2][qxj;2][dog;2][fwi;2][dlz;2][nwj;2][xyj;2][dsi;2][qwz;2][owg;2][elz;2][twg;2][eyi;2][tyg;2][nuj;2][fqg;2][oui;2][dui;2][wCj;2][cui;2][fqB;2][loj;2][fiz;2][eui;2][d vi;2][tyB;2][uyz;2][twB;2][owB;2][nyz;2][nui;2][lsz;2][lvz;2][uCi;2][uwj;2][lwk;2][tug;2][lgk;2][lgA;2][clA;2][qwA;2][lxk;2][fgi;2][lik;2][ltj;2][tyi;2][lCk;2][tvg;2][qvk;2][lAB;2][flj;2][lwA;2][fik;2][elk;2][qti;2][nyk;2][wyA;2][twi;2][ryA;2][loz;2][qwk;2][lti;2][lrA;2][lmk;2][tuB;2][lsk;2][nuk;2][lrk;2][wyk;2][lkB;2][lA;2]1[doi;2][vyk;2][vCj;2][lyk;2][liz;2][nyA;2][uCj;2][eai;2][elj;2 ][ryk;2][lgk;2][owi;2][lvk;2][dlk;2][lyA;2][fni;2][lpA;2][lCA;2][luk;2][uyk;2][clk;2][dlA;2][ltz;2][liA;2][lpk;2][lxA;1][flA;1][vyA;1][qvA;1][lsA;1][uyA;1][ltA;1][ltk;1][luA;1][elA;1][lvA;1][lr h;1][lph;1][lhB;1][elh;1][nyh;1][lih;1][lyh;1][lxh;1][qvh;1][lgh;1][lvh;1][lwh;1][dlh;1][luh;1][uy h;1][lCh;1][qwh;1][clh;1][lmh;1][wyh;1][lth;1][flh;1][ryh;1][lgh;1][lsh;1][vyh;1]

4 Phage Combinations

4 phage combinations were analyzed in silico for their ability to lyse the 352 different strains of Klebsiella pneumoniae bacteria as classified by MLST, based on the ability of the single phage to lyse a bacteria of a particular capsule type as assessed by a liquid media assay.

The combinations that lyse the highest number of bacterial strains (as classified by MLST) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[luyg;85][lwyg;85][lqwg;84][lvyg;84][lqvg;84][lqug;84][lxyg;83][luyi;83][luyB;83][lqxg ;83][flyg;83][lqwi;83][lwyi;83][lqvi;83][lqui;82][lqwB;82][lvyi;82][lwyB;82][lqvB;82][lquB;82][lryg;82][lgrg;82][luCg;82][lwCg;82][ltyg;82][luwg;82][lvyB;82][lgtg;82][lvCg;82][luvg;82][lqx i;82][lpyg;81][clyg;81][flgg;81][flyi;81][lqxB;81][lxyi;81][lpqg;81][dlyg;81][clqg;81][lxyB;81][1 ogg;81][flyB;81][dlqg;81][lryi;80][flqi;80][loyg;80][lmyg;80][lrug;80][lgri;80][lxCg;80][lgtB;79][luwi;79][clyi;79][lvCi;79][lryB;79][luvB;79][luxg;79][dlug;79][luvi;79][lqyg;79][luwB;79][lw Ci;79][flwg;79][lpqi;79][lygi;79][flqB;79][loug;79][luCB;79][luCi;79][lgrB;79][clug;79][lgsg;79][clqi;79][elyg;79][lgti;79][lpqB;79][lpug;79][lgsi;79][lpyi;79][clyB;79][flCg;79][ltCg;79][ltyB; 79][lrCg;79][ltyi;79][dlqi;79][lCyg;79][elqg;79][lvCB;79][lsug;79][flvg;79][logi;79][lsyg;79][cl qB;79][dlyi;79][flug;79][elug;79][lwCB;79][lqgi;78][dlyB;78][lygj;78][lswg;78][lmyi;78][lwxg; 78][lvwg;78][dlqB;78][lpCg;78][loqB;78][lpyB;78][lnyg;78][uvyg;78][lyiB;78][lrui;78][lsyi;78][loCg;78][lwgi;78][lmyB;78][lruB;78][lygz;78][dlwg;78][flwi;78][elyi;78][lrwg;78][lxCi;78][loy i;78][dlvg;78][clCg;78][lgsB;78][lmqg;78][ltug;78][dlCg;78][elqi;78][lygB;78][lqiB;78][lqyi;78][ltwg;78][lugi;78][lmug;78][lqCg;78][nwyg;78][lygh;78][cluB;77][lmwg;77][lsCg;77][lsui;77][1 ygA;77][lygk;77][lswi;77][lxCB;77][clui;77][ltvg;77][luxi;77][lqyB;77][lqgj;77][louB;77][loyB; 77][lugj;77][loui;77][dlui;77][flxg;77][flCi;77][lrxg;77][luxB;77][dluB;77][flvi;77][flwB;77][dfl g;77][elui;77][luiB;77][lngg;77][lvxg;77][elwg;77][lpwg;77][lmqi;77][dlxg;77][uvyB;77][lCyi;77][luvy;77][lqgh;77][nqwg;77][elqB;77][lwiB;77][lrCi;77][lqgB;77][elyB;77][flCB;77][lpui;77][lwgj;77][lpuB;77][lqgz;77][lugh;77][uwyg;77][lugz;77][flrg;77][ltxg;77][lsyB;77][flui;77][fluB; 77][luwy;77][lsuB;77][flvB;77][eluB;77][clvg;77][lugB;77][lowg;77][lyjB;77][lnyi;77][lnug;77][quvg;76][lnwg;76][lnuy;76][lagk;76][laCi;76][lrCB;76][lwyj;76][lsCi;76][lswB;76][lwxi;76][dl tg;76][lsvg;76][swyg;76][nvyg;76][lvwi;76][lwgh;76][nwyi;76][lrvg;76][lgjB;76][lwgz;76][linqi; 76][elvg;76][elCg;76][uvyi;76][lujB;76][lyih;76][lugk;76][ltCB;76][ltCi;76][luyj;76][lwgB;76][l vgi;76][lqgA;76][lugA;76][lqwj;76][clxg;76][lquw;76][lmqB;76][clCi;76][lpCi;76][ruyg;76][lCy B;76][lgsw;76][lquv;76][ltui;76][loCi;76][qswg;76][dlCi;76][lqCB;76][lmwi;76][lmuB;76][lmui; 76][dfli;76][ngwi;76][lpxg;76][ltuB;76][lnyB;76][lpCB;76][luxy;76][nqvg;76][clCB;76][nuyg;76][flwy;76][flxi;76][clwg;76][lvwB;76][lmvg;76][lwgA;76][lyiz;76][lyiA;76][elwi;76][lwxB;76][lwgk;76][lyij;76][dlwi;76][lswy;76][dlsg;76][lCgi;76][lrwi;76][dlvi;76][lmCg;76][dlrg;76][lqij; 75][lquj;75][lqvx;75][lqvj;75][lvxi;75][lqih;75][lrxi;75][lgiz;75][lqiA;75][lrwB;75][flxy;75][swy i;75][lgtx;75][lqux;75][dlwB;75][cluy;75][eluy;75][flxB;75][qswi;75][quvB;75][uwyB;75][dflB; 75][dlCB;75][lsvi;75][vwyg;75][dlog;75][dlvB;75][ouyg;75][nwyB;75][lsuy;75][lwjB;75][flri;75][lnqB;75][lyik;75][lsxg;75][lpvg;75][luih;75][loCB;75][lmwB;75][lmxg;75][lykB;75][lyzB;75][elxg;75][loxg;75][lnqu;75][lnqv;75][lnqw;75][lyhB;75][lnwy;75][cflg;75][flqw;75][lnui;75][lviB ;75][lvgj;75][ltwB;75][ltwi;75][lgsx;75][clvi;75][lqxj;75][lCgj;75][lsCB;75][delg;75][lowi;75][el vi;75][lpwi;75][cdlg;75][clxi;75][lgrx;75][clvB;75][ltvB;75][lgsv;75][eltg;75][ruyB;75][dluy;75][ltxy;75][nqwB;75][lovg;75][flpg;75][lgtw;75][clqu;75][louy;75][elwB;75][nqvi;75][lqwx;75][lq vw;75][elqu;75][lyAB;75][clrg;75][dlpg;75][quwg;75][lngx;75][cltg;75][lnvy;75][lnvg;75][elrg; 75][puyg;75][lnwi;75][lnCg;75][luvC;75][lgsu;75][lxyj;75][lgkB;75][nvyi;75][lrxB;75][flqv;75][lqzB;75][dlxi;75][flvy;75][nuvy;75][lgik;75][lCiB;75][dlgi;75][dlsi;75][frB;75][qvwg;75][nryg; 75][flqx;75][lruy;75][lrtg;75][quvi;75][lnuB;75][lvxB;75][ltvi;75][suyg;75][luij;75][luiz;75][elCi ;75][luiA;75][lukB;75][luzB;75][uwyi;75][svyg;75][luhB;75][lvgh;75][lvgz;75][lvgB;75][lwxy;75][lwih;75][lvyj;75][ltwy;74][lgru;74][lrxy;74][clwi;74][ruyi;74][wxyg;74][lsxy;74][lsxi;74][lgtv ;74][ltxi;74][qrug;74][rxyg;74][lswC;74][lsvB;74][clxB;74][qsvg;74][ltxB;74][nxyg;74][lmrg;74][lrvi;74][lCgB;74][lCgh;74][lwiz;74][lwij;74][qvxg;74][elqv;74][lxgi;74][swyB;74][lCgz;74][ef lg;74][rwyg;74][lwiA;74][flsg;74][lsvy;74][loqu;74][clsg;74][lmvi;74][uvCg;74][lpwB;74][lmCi ;74][luik;74][lowB;74][lqAB;74][clqv;74][lrgi;74][luAB;74][uxyg;74][lvwy;74][lvxy;74][lrsg;74][lghB;74][flog;74][fltg;74][lnxy;74][fluy;74][dlxB;74][nuyi;74][flny;74][dlgj;74][luwC;74][qsw B;74][lvgA;74][dlqu;74][lvgk;74][vxyg;74][lwhB;74][lmxi;74][clxy;74][lwik;74][dwyg;74][qrw g;74][lnwB;74][lxgj;74][dltB;74][dlri;74][pqvg;74][loqx;74][dlsB;74][pvyg;74][dlti;74][ltvy;74][clgi;74][fvyg;74][fwyg;74][dlmg;74][ouyB;74][lwyz;74][lpxi;74][dvyg;74][lgrw;74][qsvi;74][l mqw;74][lwCj;74][lwzB;74][lwkB;74][clwB;74][lpuy;74][svyi;74][clvy;74][quwB;74][lvjB;74][dliB;74][qrxg;74][flqu;74][elvB;74][lmvB;74][lmwy;74][lqwC;74][luyz;74][cfli;74][luCj;74][nq rg;74][clqx;74][elwy;74][lCgA;74][clpg;74][lpqx;74][nqvB;74][lpqu;74][pqug;74][ltuy;74][elqw ;74][elxi;74][rvyg;74][nuyB;74][lnxg;74][flyj;74][lorg;74][lrvB;74][nvyB;74][lCjB;74][elCB;74][cvyg;74][lsuC;74][nuwy;74][lmCB;74][lawy;74][lawz;74][lCgk;74][luCy;74][lriB;73][latu;73][lsuw;73][elri;73][clsi;73][lvCj;73][efli;73][lpxB;73][fqvg;73][cqvg;73][lgtj;73][lrgj;73][lnqs;73][clri;73][dlqv;73][nquv;73][luyh;73][dloi;73][lngt;73][lpqw;73][lxiB;73][lwAB;73][elqx;73][dln g;73][lptg;73][dlxy;73][flsi;73][lnCi;73][clgj;73][deli;73][lmtg;73][cflB;73][lquy;73][ouyi;73][lu xC;73][lquC;73][lmuy;73][lsvC;73][luwj;73][lvih;73][vwyB;73][vwyi;73][lmxy;73][suyi;73][luy k;73][nqug;73][lqvC;73][elvy;73][nwCg;73][dlgB;73][qvwi;73][ngxg;73][lsxC;73][flpi;73][dlqw ;73][flnq;73][celg;73][dlwy;73][cuyg;73][clog;73][dlgh;73][puyB;73][dlqx;73][lwyh;73][lmqx;73][qruB;73][qvwB;73][lqwk;73][nuC;73][flmg;73][lnrg;73][nryi;73][lwyk;73][lrsi;73][lnvi;73][1 pxy;73][lmxB;73][loxi;73][clyj;73][clmg;73][oqug;73][loqw;73][cdli;73][elpg;73][dlrB;73][lpvi; 73][lsxB;73][cliB;73][lwCy;73][duyg;73][dlgz;73][rsyg;73][lrwy;73][flgi;73][quwi;73][npyg;72][lnvC;72][dqvg;72][flqj;72][lnyj;72][flng;72][flry;72][ngri;72][dlgA;72][suyB;72][lvCy;72][dlgk ;72][uvCB;72][dfly;72][qwxg;72][svyB;72][suvy;72][eluC;72][dlvy;72][dlqs;72][lqvz;72][fliB;72][elxB;72][lqwh;72][lqwA;72][lqxC;72][lnty;72][lnwC;72][qvxi;72][flqr;72][lvxC;72][clny;72][lnqr;72][nuvg;72][nswy;72][uwCg;72][pwyg;72][lwyA;72][ngsw;72][flsy;72][luyA;72][lxCj;72][flqs;72][qwyg;72][lviz;72][lxgh;72][floi;72][flpB;72][lnry;72][dljB;72][puyi;72][lvij;72][lxgz; 72][nruy;72][ltxC;72][lxgB;72][lviA;72][clng;72][ltyj;72][lqxy;72][elxy;72][elti;72][clqw;72][cd 1B;72][elqt;72][clqt;72][lquz;72][dluC;72][qsvB;72][lryj;72][lrgz;72][lrgh;72][lrgB;72][lCij;72][1 ouC;72][lotg;72][lmri;72][muyg;72][nvCg;72][lCih;72][flvC;72][lovi;72][lpvB;72][nuwg;72][nx yi;72][lruC;72][lpwy;72][clnq;72][dlpi;72][flwC;72][dloB;72][clqs;72][clwy;72][pquB;72][clgj; 72][elog;72][wygi;72][pqwg;72][lmpg;72][loxB;72][loxy;72][qsug;72][lowy;72][lqvy;72][qvyg; 72][qrvg;72][qrui;72][lgrj;72][clrB;72][lprg;72][fryg;72][mqvg;72][lqxz;72][lpsg;72][clti;72][lqs t;72][cltB;72][cluC;72][lnpq;72][flsB;72][fltB;72][lstg;72][flti;72][flty;72][lrgk;72][owyg;72][lrg A;72][lrsB;72][lnoq;72][lmrB;72][nuCg;72][qvCg;72][nwyj;72][flxC;72][clgz;72][lrti;72][lrtB;72][clgB;72][dlny;72][nryB;72][vxyi;72][dlmi;72][lrvy;72][lmqv;72][uvCi;72][lxjB;72][lCiz;72][qvxB;72][flgj;72][lCiA;72][dlyj;72][elty;72][flqt;72][celi;72][dlsy;72][clgh;72][pqvi;72][mwyg; 72][lrsy;72][npqg;72][lvhB;72][pqvB;72][wygj;72]

The percent of host bacterial strains (as classified by MLST) that are infected by two phages of a phage combination are provided below. This trait is referred to as “at least 2 phage % coverage”. The phage combinations are ordered in descending performance grade.

[lqwy;49][lqvy;49][lnwy;48][dlwy;48][lwCy;47][lnuw;47][lvwy;47][lrwy;46][dlvy;46][lu wy;46][lnqw;46][dlny;46][dlqw;46][lqvw;46][lqxy;46][lquy;46][lvCy;46][lquw;45][lnwC;45][lq vC;45][lqwC;45][lnry;45][luwC;45][dlqv;45][lruy;45][lvwC;45][dlsy;45][lgrw;45][clwy;45][luC y;44][lpuy;44][lnvy;44][lnuC;44][luvy;44][lpwy;44][lswy;44][lnqr;44][lnuv;44][lwxy;44][dlnq;44][lgru;44][lxCy;44][lrvy;44][lgrv;44][dluy;44][lqwx;44][lquC;44][lnuy;44][lquv;44][clqw;44][l asw;44][lpaw;44][luxy;43][dlas;43][lvxy;43][dlxy;43][clvy;43][lpqu;43][lnqu;43][luvC;43][lnv C;43][lnxy;43][dlqu;43][dfly;43][lnpy;43][lgsu;43][lowy;43][lnqv;43][lqux;43][lsuw;43][lsuy;43][lruC;43][lrsy;42][clqv;42][lmuy;42][dlqx;42][lqxC;42][lswC;42][clny;42][lgrs;42][ltwy;42][lsu C;42][cluy;42][loqw;42][lgsv;42][flwy;42][flry;42][dluC;42][lpuC;42][cluC;42][lsuv;42][luxC;42][elwy;42][lwxC;42][lsvy;42][lmwy;42][dluv;42][lmqu;42][loqv;42][dlty;42][lqvx;42][lnpq;41][clvC;41][lgtw;41][clqu;41][louy;41][dlsu;41][loqu;41][lmqv;41][fluy;41][clnq;41][lrxy;41][lwy B;41][lmvy;41][clsy;41][flvy;41][lwyi;41][fluC;41][clqs;41][flqw;41][clxy;41][dlwB;41][dflq;41][lngx;41][lwyg;41][elqw;41][flqv;40][luwi;40][dlyB;40][lvxC;40][lgrx;40][luwg;40][louC;40][d lwi;40][lsvC;40][nquw;40][lmqw;40][clqx;40][dlwg;40][dlyj;40][ltwC;40][ltuy;40][ltuC;40][luw B;40][dlgt;39][flwC;39][lnsy;39][lpqx;39][nuwy;39][lwyj;39][cfly;39][lpxy;39][lnxC;39][eluy;39][ltvy;39][dlng;39][flny;39][lgtu;39][elvy;39][flqr;39][lqwg;39][lqwB;39][dlyi;39][dlyg;39][flq u;39][dlrB;39][dlvB;39][lgtv;39][lvwB;39][lsxy;39][lnug;39][lwyA;39][lqwi;39][lnwg;39][flnq; 39][lwyk;39][lwyh;39][dlni;39][lryB;39][lgrt;39][lnrg;39][lwyz;39][rwB;39][lnwB;39][lmxy;38][nvwy;38][flvC;38][lnuB;38][lryj;38][elqu;38][lrxC;38][lrwi;38][dlvg;38][lvwi;38][lwCB;38][l mny;38][dlrg;38][dlvi;38][clwg;38][lryi;38][flpy;38][lrwg;38][lnui;38][dlri;38][lvwg;38][luwj;38][elxy;38][lnwi;38][lryg;38][loxy;38][lwCg;38][eluC;38][dlui;38][flsy;38][luCB;38][lnyB;38][cl wi;38][clwB;38][qwyg;38][lqwh;38][dluB;38][lnvB;38][luCi;38][lnvg;38][lCyB;38][cdlg;38][dl ug;38][luCg;38][eluv;38][lnyg;38][ltvC;38][lqwj;38][lvyB;38][lwCi;38][flpq;37][dlqg;37][loqx; 37][lnyi;37][cdlB;37][clvg;37][lnCg;37][lqwA;37][lnty;37][lvyg;37][lgsx;37][cdli;37][lnvi;37][1 nCB;37][lqwz;37][lnoq;37][luyi;37][luyB;37][luvB;37][lqwk;37][lgrB;37][lqug;37][lwCj;37][lq yB;37][luyg;37][lrCB;37][luvg;37][dlpB;37][lCyg;37][dlqB;37][dlqj;37][lvyi;37][lqyg;37][quwg ;37][elqv;37][lngg;37][lvyj;37][lCyi;37][clrg;37][lgrg;37][lqyi;37][clui;37][uwyi;37][clrB;37][lq rj;37][flxy;37][lqCg;37][lrCg;37][lnqB;37][dlpg;37][clvB;37][luvi;37][clvi;37][luyj;37][lrCi;37][dlpi;37][wCyg;37][lrvB;37][lnCi;37][clri;37][rwyg;37][lnqs;37][lvyk;37][dlCB;37][luvj;37][flqs; 37][uwyg;37][lgri;37][clty;37][rwyi;37][lquB;37][lvyh;37][luCj;37][lqCB;37][rwyB;37][lqui;37][quwi;37][lvyz;37][dlqi;37][lruB;36][lquj;36][luyz;36][lvCB;36][lswg;36][uwyB;36][dlCi;36][dl Cg;36][dlsg;36][lnqi;36][lpvB;36][luyk;36][lrgB;36][lrvg;36][elqx;36][efly;36][elvC;36][lwgB;36][nquv;36][luyh;36][dgwg;36][lrug;36][lqCi;36][lmqx;36][nruy;36][cluB;36][luyA;36][lqvB;36][lqvg;36][quwB;36][clug;36][nuwg;36][lvCg;36][uwCg;36][lpyB;36][lprg;36][clqt;36][clng;36][lqvi;36][lpwB;36][lugB;36][floy;36][lxyi;36][dwyi;36][lquz;36][lxyg;36][dlsB;36][clyg;36][dw yB;36][dwyg;36][luxg;36][lquk;36][lpvg;36][sxC;36][vCi;36][dlgB;36][lqvj;36][lrui;36][lsyg;36][ltxy;36][lxyB;36][lswB;36][nqug;36][lrvi;36][nuvC;35][lpyi;35][clyB;35][lpwi;35][lqvz;35][l pyg;35][clyi;35][lvCj;35][nswy;35][lwxg;35][lqvk;35][duyg;35][lpvi;35][qrwg;35][dnyg;35][lpu g;35][dlxB;35][luxi;35][luxB;35][lpwg;35][dlsi;35][lpuB;35][dlxg;35][dlxi;35][flqx;35][lsyB;35][lpyj;35][lwxi;35][nuvy;35][lwxB;35][lswi;35][lxyj;35][lsyi;35][lrsg;35][lsuB;35][lnxg;35][rgi; 35][pwyg;35][dlmB;35][lgtx;35][lwgi;35][clpg;35][elyB;35][vwyg;35][nuyB;35][lvxg;35][duyB; 35][qvyg;35][lsug;35][lpui;35][ngsw;35][vCyg;35][clni;35][nuyg;35][clqg;35][lsty;35][lngt;35][grwi;35][qrwB;35][lmuB;35][vwyi;35][lvxB;35][lugi;35][vwyB;35][flng;35][nqui;35][dlmg;35][qvwg;34][lmrg;34][lpsg;34][lmui;34][clsB;34][dryg;34][clCg;34][lpqg;34][dvyg;34][ltwi;34][elr B;34][lxyz;34][elyg;34][lnxi;34][dlgi;34][clpi;34][ltwB;34][dvyB;34][dvyi;34][lvxi;34][lpqB;34][lsui;34][lrsB;34][clsg;34][lmrB;34][lmug;34][nuyi;34][dlmi;34][lnyj;34][clCB;34][qvwB;34][it wg;34][clCi;34][clxg;34][dloB;34][clsi;34][lwiB;34][lriB;34][clqB;34][lpgj;34][elug;34][qvwi;34][elty;34][lpqi;34][eluB;34][lowB;34][lvgB;34][flxC;34][lsCg;34][elyi;34][dqvg;34][lygB;34][e lrg;34][lpgi;34][clyj;34][clqi;34][elwB;34][nryB;34][delB;34][nuCg;34][luiB;33][elui;33][qwxg; 33][lqxg;33][nuvg;33][flwg;33][lowi;33][lmri;33][lsCB;33][delg;33][dliB;33][lmwB;33][qrvg;33][ruCg;33][lmwg;33][lmvB;33][lpCB;33][nryg;33][lowg;33][dlog;33][uxyB;33][elwg;33][pqw g;33][lrxB;33][clxB;33][elri;33][lmvg;33][wxyg;33][flty;33][wxyi;33][flwB;33][lrxg;33][nouy;33][ltxC;33][clxi;33][lrsi;33][mwyg;33][dloi;33][uxyg;33][clgB;33][quxg;33][lgsg;33][cwyg;32][l mqg;32][lsCi;32][lmwi;32][deli;32][elwi;32][celg;32][lqxB;32][lpsi;32][nryi;32][quvg;32][lsvg; 32][lpCi;32][rvyg;32][nwyB;32][lrxi;32][dflg;32][elvB;32][flwi;32][lngj;32][elyj;32][fluB;32][lp Cg;32][dflB;32][rvyB;32][qrug;32][lgst;32][lqxi;32][uxyi;32][dfli;32][rvyi;32][lvgi;32][ngrg;32][lxCg;32][lmyB;32][lgsB;32][elqg;32][lmCg;32][nwyg;32][oqwg;32][flui;32][flug;32][elqB;32][lmyg;32][clgj;32][lmCB;32][lmqB;32][lxCB;32][lxCi;32][lygi;32][clgi;32][puyg;32][lmsg;32][el vg;32][lmvi;32][uvCg;32][lsvB;32][cuyg;32][clog;32][ruyg;32][qrui;31][lmqi;31][uvCB;31][flrg ;31][nwyi;31][elqt;31][cflB;31][muyg;31][puyi;31][lpxB;31][dxyg;31][clmi;31][wygB;31][loyB; 31][lqxh;31][louB;31][lqgB;31][qruB;31][pqug;31][ruyi;31][lmCi;31][cflg;31][lrtB;31][ltuB;31][lqxj;31][loug;31][nvyB;31][puyB;31][lCgB;31][nsvy;31][quvi;31][frB;31][dsyg;31][uvCi;31][l qsi;31][lpxg;31][celi;31][quvB;31][flqt;31][ruyB;31][nwCg;31][dltB;31][loqt;31][elvi;31][clmg; 31][ltug;31][lovB;31][dlti;31][cfli;31][owyg;31][lmyi;31][qvCg;31][nwCi;31][nqwg;31][flyg;31][qsug;31][loui;31][lqxz;31][lyiB;31][uvyg;31][lrtg;31][lrti;31][suvy;31][lxgB;31][elsg;31][lovg; 31][ngri;31][lqxA;31][elqi;31][lsvi;31][nqwB;31][lpxi;31][loyi;31][nvyg;31][fwyg;31][lviB;31][dltg;31][lqxk;31][vxyB;31][loyg;31][elpg;31][lgsj;31][lsxg;31][flyB;31][pquB;30][fwyB;30][lm xg;30][ixCj;30][flri;30][wygi;30][nvyi;30][vxyg;30][flyi;30][flvB;30][elpi;30][itui;30][isxB;30][suyB;30][fwyi;30][pqui;30][iwij;30][flyj;30][fqwg;30][uvyB;30][lovi;30][uvyi;30][flvg;30][swC g;30][rxyg;30][nqvB;30][mqwg;30][qgi;30][cliB;30][wgj;30][cltg;30][suyg;30][pvyg;30][Cgi; 30][flpB;30][nvCg;30][eiCB;30][lmxB;30][npyg;30][logg;30][flqg;30][lorg;30][elqj;30][flpg;30][clti;30][pvyB;30][mvyg;30][vxyi;30][lmpg;30][swyg;30][loCB;30][lxgi;30][swyB;30][nqvg;30][twyg;30][ngwi;30][loqB;30][nxyB;28][lori;28][pvyi;28][loqj;28][pxyg;28][rxyi;28][nwyj;28][cv yB;28][dliij;28][pvg;28][nxyg;28][itvB;28][lmxi;28][flvi;28][cltB;28][loCg;28][loCi;28][lsxi;28 ][elCg;28][cvyg;28][suyi;28][logi;28][flqB;28][lptg;28][pqvB;28][npqg;28][lCiB;28][lwjB;28][s wyi;28][nxyi;28][cqvg;28][elxg;28][ltvi;28][qswg;28][nqvi;28][ltvg;28][lrgj;28][wyiB;28][oqug; 28][dlgj;28][cvyi;28][elxB;28][flpi;28][flgj;28][flqi;28][elCi;28][lxiB;28][luij;28][qswB;28][qvx g;28][elxi;28][fnyg;28][qvxB;28][pqvi;28][lugj;28][cxyg;28][flCg;28][lqiB;28][loxg;28][loxB;28][qrsg;28][qtwg;28][flCB;28][lmog;28][qrxB;28][oqui;28][flsB;28][flCi;28][elgi;28][lrjB;28][it yB;28][ltyi;28][flsg;28][qrxg;28][ngxg;28][oquB;28][dljB;28][flmg;28][cqvi;28][loxi;28][lwzB; 27]

The percent of host bacterial strains (as classified by MLST) that are infected by three phages of a phage combination are provided below. This trait is referred to as “at least 3 phage % coverage”. The phage combinations are ordered in descending performance grade.

[dluv;28][dluy;28][lrwy;28][lquy;27][luwy;27][lquw;27][luCy;27][lrvy;27][lgrw;26][dlqu ;26][dlwy;26][dlqw;26][lpwy;26][dluC;26][lvwy;26][lwCy;26][dlvy;26][lgrv;25][lqwy;25][lwxy ;25][lquC;25][luwC;25][clqu;25][lruC;25][lvwC;25][cluy;25][lqvw;25][clwy;25][dlqv;25][lruy;24][lquv;24][dlxy;24][lgru;24][clqw;24][lvCy;24][lmwy;24][lqwC;24][luvy;24][lwxC;24][lrxy;24][luvC;24][lpqw;24][cluC;23][lmqw;23][lpxy;23][lnuy;23][elwy;23][lqwx;23][lpuC;23][clvy;23][clvC;23][lmuy;23][lpuy;23][dlny;23][lnuw;23][eluy;23][lmvy;23][dlsu;23][luxy;23][lxCy;23][1 owy;23][nuwy;23][lqvy;23][lpqu;23][eluv;23][lnqu;23][elvy;23][lsuw;23][clxy;23][nvwy;23][cl qv;22][lrxC;22][dlqx;22][nruy;22][lnsy;22][lnry;22][lvxy;22][lqxy;22][elqu;22][lmqu;22][lnuC;22][fluy;22][flqu;22][lmny;22][lnuv;22][lqvC;22][elqw;22][dlnq;22][luxC;22][clny;22][loqw;22][flwy;22][elqv;21][lngs;21][lnxC;21][lnvy;21][lgrx;21][lvxC;21][lnwy;21][lqux;21][nquw;21][flp y;21][nuvy;21][eluC;21][lmxy;21][lnqr;21][lqxC;21][dlsy;21][lswy;21][clqx;21][lnpy;21][lsuy;21][flqw;21][lnvC;21][lrsy;21][lnwC;21][lmqx;20][lpwg;20][clsy;20][lqvx;20][nuvC;20][flwC;20][elvC;20][lnqv;20][clnq;20][lnxy;20][lpwB;20][elxy;20][ltuy;19][lpwi;19][flpq;19][nquv;19][lp qx;19][flvy;19][dflq;19][cfly;19][lnpq;19][louC;19][cwyg;19][flry;19][lnqw;19][lswC;19][dfly;19][lmqv;19][lsuC;19][flqr;19][loqu;19][dlqs;19][louy;19][clwB;19][lgsu;19][lsxy;19][suvy;19][fl ny;19][lnoq;19][lrwg;19][lrwB;19][ltwy;19][dlug;18][dlwB;18][clwg;18][lrwi;18][lsuv;18][lvwg ;18][lngx;18][dluB;18][lgrs;18][nswy;18][dlwi;18][lsvy;18][lvwB;18][elqx;18][clwi;18][dlwg;18][lprg;18][lwxB;18][duyB;18][duyg;18][clty;18][lgsw;18][cuyg;18][dlrg;18][loxy;18][lsxC;18][1 vwi;18][lgtu;18][dlrB;18][dwyi;18][dwyg;18][efly;18][dwyB;18][lnwB;18][fluC;18][dlui;18][lw xg;18][lrvi;17][lwxi;17][lnwi;17][lnwg;17][lmwg;17][flnq;17][lmwB;17][clqs;17][dlpg;17][cdlB ;17][dlty;17][lrvg;17][lnty;17][dlpB;17][ltwC;17][clng;17][luwg;17][lrui;17][lruB;17][loqv;17][f lxC;17][lqyi;17][lrvB;17][lqyB;17][clni;17][flvC;17][lrug;17][dlri;17][flxy;17][lsvC;17][luwB;17][luwj;17][lqyg;17][dlni;17][dlxB;17][flqv;17][floy;17][dryg;17][luwi;17][cdli;17][clpg;17][nsv y;17][cdlg;17][clrB;17][flax;17][lnat;17][dlng;17][ltvy;17][dlpi;17][lmwi;17][uwyB;16][ltxy;16][dlCi;16][dgwg;16][vwyg;16][dCB;16][cvyg;16][dCg;16][dlmB;16][lrxB;16][lmpg;16][dlmg;16][lpxB;16][elty;16][lnuB;16][lCyg;16][vwyB;16][lgsx;16][lpug;16][nouy;16][lgrt;16][wCyg;16][ruyB;16][clug;16][uwyg;16][clmg;16][clpi;16][cluB;16][ruyg;16][lCyB;16][clrg;16][uwCg;16][lgtw;16][cvyB;16][lCyi;16][qwyg;16][dlxg;16][ruyi;16][lpui;16][lpuB;16][ltuC;16][clqt;16][cxy g;16][lori;16][elwB;16][lrxi;16][ngsw;16][vwyi;16][dlxi;16][pwyg;16][lnug;16][dlgt;16][lpxi;16][dxyg;16][clyB;16][dlvB;16][clyj;16][loqx;16][lnrg;16][lpxg;16][dlmi;16][lnxg;16][clri;16][lorg ;16][uwyi;16][cvyi;16][elwi;16][lrxg;16][dlvg;16][lgsv;16][elwg;16][nuwg;15][clmi;15][clyi;15][ruCg;15][lnxi;15][dlyg;15][lnyB;15][dlvi;15][rwyB;15][lnvB;15][ltvC;15][lpvB;15][luyB;15][d elg;15][dlyB;15][deli;15][c1CB;15][luyg;15][dvyg;15][ltxC;15][lnui;15][rwyg;15][clui;15][wxyg ;15][clyg;15][dvyB;15][lpvi;15][dlyi;15][flsy;15][delB;15][lpvg;15][elCi;15][quwB;15][lowB;15][quwg;15][elpg;15][dlqg;15][lsty;15][clvB;15][luyi;15][e1CB;15][puyg;15][lnvg;15][lowg;15][d lqi;15][rvyB;15][uvyg;15][clCg;15][clxg;15][cCi;15][qrug;15][dlqB;15][pCg;15][lpCB;15][elC g;15][lqCi;15][lqCB;15][clxB;15][puyi;15][lqCg;15][luxB;15][rvyg;15][lnyg;15][qrui;15][puyB; 15][wxyi;15][lnyi;15][uvyB;15][rwyi;15][qruB;15][lnCB;15][luyj;15][lnyj;15][elt;15][uvyi;15][dvyi;15][rvyi;15][lpCi;14][lowi;14][qrwg;14][luvj;14][lwCg;14][lnCg;14][clvg;14][lmrg;14][lnv i;14][luxi;14][qvwB;14][lxCB;14][luyA;14][qrwB;14][lnCi;14][lmyB;14][clxi;14][lwCi;14][luvi ;14][quwi;14][luxg;14][luvB;14][mqwg;14][lmyg;14][lmri;14][clqB;14][lpyj;14][lwCB;14][lvxB ;14][lpyB;14][qvyg;14][cqvg;14][elxB;14][lmug;14][luvg;14][qvwg;14][lmrB;14][lpyg;14][dlyj; 14][lgtv;14][pqwg;14][elpi;14][flty;14][qvwi;14][celg;14][lwyg;14][luyz;14][lmCB;14][lmCg;14][pvyg;14][elxg;14][lmui;14][clqi;14][qwxg;14][lryB;14][dqvg;14][lpyi;14][nwyi;14][lwyj;14][lr CB;14][mwyg;14][lrCg;14][lmyi;14][nwyg;14][cqvi;14][mxB;14][loqt;14][celi;14][elrB;14][qr wi;14][luCB;14][lmuB;14][lxCg;14][lwyB;14][lqug;14][lwyi;14][elrg;14][lryg;14][luyh;14][lqu B;14][luyk;14][clgj;14][nwyB;14][lvxg;14][vCyg;14][lvxi;14][clvi;14][clqg;14][pvyi;14][luCg;14][pvyB;14][lqui;13][luCi;13][lmxi;13][nwCi;13][elxi;13][lxCi;13][elvB;13][lwCj;13][uvCi;13][dnyg;13][qvCg;13][lmvB;13][lryi;13][uxyi;13][lngj;13][uxyg;13][lmvg;13][uvCB;13][pquB;13][lmxg;13][rxyg;13][lnqB;13][lgst;13][dloB;13][lryj;13][lgtx;13][muyg;13][dlog;13][elri;13][nwC g;13][lmvi;13][uvCg;13][lmCi;13][pqug;13][pqui;13][rxyi;13][flqs;13][uxyB;13][dlgj;13][lrCi;13][elvg;13][npyg;12][lovi;12][lwyz;12][dloi;12][lrsB;12][lwyh;12][qrxi;12][qrxB;12][lovB;12][1 wyA;12][lxCj;12][lwyk;12][clog;12][pxyg;12][qrxg;12][inqi;12][lmqg;12][lmqB;12][lquj;12][lo vg;12][eluB;12][elvi;12][luCj;12][lngg;12][elug;12][loyB;12][quxg;12][lmqi;12][vxyg;12][elui;12][lrsi;12][ouyi;12][nwyj;12][elyi;12][flpi;12][lsvB;12][vxyi;12][elyB;12][nqwg;12][lrsg;12][nu Cg;12][quvi;12][lvyj;12][ouyg;12][quvB;12][flxB;12][qrvg;12][loxB;12][ngwi;12][nuyg;12][flp g;12][nqwB;12][quvg;12][lswB;12][flpB;12][nuvg;12][owyg;12][flwB;12][loyg;12][lqwi;12][ely g;12][larg;12][lvyB;12][lawB;12][lxyB;12][vxyB;12][larB;12][flug;12][ouyB;12][lpB;12[[lpg ;12][elyj;12][flqt;12][nuyB;12][lvyg;12][nxyB;12][lqwg;12][flwg;12][lqxB;11][lsvg;11][svyB;11][elqi;11][lvCg;11][flxi;11][lvyi;11][mqvg;11][flri;11][lsvi;11][lquz;11][lxyg;11][dflB;11][loxg ;11][loug;11][lgri;11][lqwj;11][rsyi;11][flwi;11][pqxg;11][flCg;11][loyi;11][flxg;11][elqg;11][df lg;11][lvCB;11][loui;11][louB;11][mqvi;11][lpgj;11][lquk;11][flrg;11][flui;11][fluB;11][mqvB;11][lmog;11][elqB;11][flCB;11][lswg;11][lpqi;11][firB;11][dlsB;11][svyg;11][rsyg;11][flmg;11][svyi;11][nxyg;11][loxi;11][nuyi;11][loCg;11][clsg;11][ltyB;11][qvxi;11][flCi;11][elqj;11][elog;11][nvyB;11][lpsi;11][swyB;11][lvyz;11][loCB;11][mvyg;11][lvyk;11][lgrj;11][fwyg;11][lswi;11][fryg;11][lvyh;11][lpsg;11][lvCi;11][cflg;11][ltyg;11][lsyB;11][nvCg;11][fwyB;11][dlsg;11][lqx g;11][cflB;11][lxyj;11][nryB;11][qvxB;11][lxyi;11][pqvg;11][dsyg;11][qvxg;11][nryg;11][nxyi; 11][pqvi;11][pqvB;11][lvCj;11][nvyg;11][dfli;11][swyg;11][clsB;10][lqxi;10][flyB;10][lqwz;10][loCi;10][fqwg;10][lqwh;10][lqwA;10][suyg;10][ltui;10][npqg;10][lsuB;10][nryi;10][ltyi;10][fw yi;10][nvyi;10][lsug;10][lqwk;10][dlsi;10][flyg;10][ngxg;10][lsyg;10][lsxi;10][tvyg;10][swyi;10][lsxg;10][lsxB;10][psyg;10][flng;10][flvi;10][ltyj;10][dltg;10][suyB;10][ltCg;10][lxyz;10][ltug;10][lsui;10][lmsg;10][ngxi;10][ltCB;10][lgsB;10][flqB;10][lrti;10][lrtB;10][lsyi;10][flvB;10][flvg; 10][flqg;10][nqug;10][cfli;10][lrtg;10][lqxj;10][ltuB;10][lsCB;10][clsi;10][lqvB;10][lptg;10][lqv g;10][flyj;10][flyi;10][dltB;10][suyi;10][eflB;9][fvyg;9][lgtB;9][eflg;9][qswB;9][twyg;9][fqvi;9][cltB;9][fqvB;9][lgtg;9][dlti;9][nqui;9][fvyi;9][lsCg;9][ltvi;9][loqB;9][swCg;9][fvyB;9][lqvi;9][l qsg;9][lqvj;9][fqvg;9][lstg;9][ltxB;9][elsg;9][ngrg;9][ltvg;9][ltvB;9][logg;9][flqi;9][lqxA;9][ltCi; 9][qswg;9][lmtg;9][ltxi;9][ltwg;9][ltxg;9][qrsg;9][ltwB;9][nqvg;9][cltg;9][ngri;9][oqwg;9][flti;9][logi;9][fltB;9][lgsj;9][qsvB;9][qsvi;9][lsCi;9][fltg;9][qswi;9][efli;9][lqxz;9][lotg;9][lgti;9][nqvB ;9][lgtj;9][lqxh;9][qsvg;9][lgsi;9][lqxk;9][flgj;9][oquB;8][oqui;8][oqug;8][lqvk;8][floi;8][flsB;8][floB;8]

The percent of host bacterial strains (as profiled by MLST) that are infected by four phages of a phage combination are provided below. This trait is referred to as “at least 4 phage % coverage”. The phage combinations are ordered in descending performance grade.

[clwy;12][dlwy;11][lruy;11][lnpy;11][dluC;11][dluy;11][lnuw;11][cluy;11][lpuy;11][lpw y;11][clny;11][dluv;11][lnwy;10][lmny;10][lnwC;10][cluC;10][clqw;10][lrxC;10][lwCy;10][lm wy;10][lnuv;10][lruC;10][eluC;10][lwxC;10][dlqw;10][lpuC;10][luwy;10][dlqu;10][lrwy;10][lu wC;10][dlny;10][lquy;10][clnq;10][lnpq;10][lnry;9][lvwC;9][luvy;9][lmuy;9][clvy;9][lgru;9][lux C;9][eluv;9][lpqw;9][elvC;9][lvwy;9][suvy;9][luxy;9][dlnq;9][luCy;9][lqwy;9][elwy;9][lsuv;9][l nqw;9][lnuy;9][louy;9][fluC;9][dlxy;9][lpqu;9][lpxy;9][clqu;9][clxy;9][lrvy;9][lrxy;9][lnvy;9][lq wC;9][lmqw;9][lmxy;8][lgrw;8][clvC;8][lgrx;8][lxCy;8][lmqu;8][flxC;8][dlqx;8][lnxy;8][nruy;8][lqux;8][lqvy;8][eluy;8][dlvy;8][flwC;8][lvxC;8][lpqx;8][lwxy;8][elqw;8][lnqr;8][nuwy;8][lquC ;8][lmvy;8][lnuC;8][lvCy;8][lnvC;8][lnxC;8][nvwy;8][nuvy;8][lsvy;8][lxC;8][cfly;8][dfly;8][l mqv;8][lqxy;8][lngx;8][lnqu;8][lrsy;8][lvxy;8][elxy;8][luvC;8][clqx;8][lsuw;8][dlsu;8][lquw;8][f lxy;7][fluy;7][lqwx;7][lsuy;7][loxy;7][lnqv;7][lowy;7][elqu;7][lqvw;7][clqv;7][lmqx;7][nquw;7][elvy;7][dlty;7][clsy;7][elqx;7][flry;7][lqvC;6][flvC;6][clty;6][flwy;6][lswy;6][dflq;6][dlqv;6][dlq t;6][lgrv;6][lquv;6][lqvx;6][dlsy;6][flpy;6][nouy;6][nuvC;6][lnty;6][ltuC;6][louC;6][lgsv;6][clqt; 6][lgrs;6][lsxC;6][nquv;6][nsvy;6][clqs;6][ltwC;6][efly;6][flqu;6][lswC;6][flqr;6][flqx;6][lsvC;6][ltuy;6][lnsy;6][lsxy;6][elqv;6][flpq;5][lgrt;5][lsuC;5][lgsu;5][lsty;5][ltwy;5][loqu;5][flvy;5][flty; 5][ltvy;5][loqx;5][ltxC;5][flqw;5][lnoq;5][lgsw;5][dlqs;5][flny;5][lgsx;5][loqv;5][flsy;5][flqt;5][l txy;5][lgtu;5][flqv;5][flnq;5][nswy;5][lngt;5][lngs;5][lgtw;5][flqs;5][floy;5][lgtx;5][lygB;5][elty; 5][ltvC;5][elqt;4][lCyB;4][lnqB;4][lpyg;4][lqyB;4][loqw;4][lngg;4][lCyg;4][lmyB;4][lCgB;4][lm pg;4][lgst;4][lqyg;4][lqgB;4][lgtv;4][ngsw;4][lnyB;4][lmyg;4][lnyg;4][lpyB;4][pvyg;4][pvyB;4][npyg;4][lmvB;4][clwB;4][lwyg;4][lprg;4][wygB;4][lvyg;4][lpvB;4][lpqg;4][lmCg;4][lmvg;4][cl CB;4][lmCB;4][lpCB;4][lpvg;4][lvyB;4][lwCg;4][dlyg;4][dwyg;4][lrgB;4][lwyB;4][clyg;4][lryg; 4][lqCB;4][dlwB;4][cdlB;4][clwg;4][cwyg;4][dlCB;4][clgB;4][lygi;4][lwgB;4][clyB;4][lnCB;4][lpqB;4][lyiB;4][cdlg;4][dlgB;4][lnCg;4][wCyg;4][lmyi;4][dlyB;4][lmqg;4][nwCg;4][dlCg;4][lqC g;4][lsxB;4][npqg;4][lmrB;4][dlwg;4][clCg;4][lpyi;4][lpCg;4][lmrg;4][lpgi;4][mvyg;4][dwyB;4][lmqB;4][lvgB;4][lryB;4][lwCB;4][lrxB;3][nqvB;3][lmvi;3][nvyB;3][lpvi;3][lnwB;3][rsyg;3][clr g;3][rvyg;3][lmCi;3][lpwB;3][lrvB;3][lmxB;3][clmg;3][lrsg;3][pqvg;3][vxyg;3][lrgi;3][lCiB;3][d lrB;3][uxyg;3][dlng;3][dlpg;3][svyg;3][elwB;3][e1CB;3][clxB;3][celg;3][muyg;3][lmri;3][ruyB;3][lpxB;3][lriB;3][suyg;3][delg;3][lovg;3][puyB;3][rxyg;3][clpg;3][lsvB;3][nryg;3][dlmg;3][nqrg; 3][nwyB;3][puyg;3][lnvg;3][rwyg;3][lnrg;3][nqwB;3][lvxB;3][luyg;3][nqwg;3][pxyg;3][lnyi;3][1 ugB;3][dnyg;3][lpuB;3][lpwg;3][lmqi;3][pvyi;3][lpqi;3][uvyB;3][lCyi;3][lqyi;3][elyB;3][lgrB;3][nwyg;3][lsug;3][loyg;3][lryi;3][lmug;3][uxyB;3][lvgi;3][loqt;3][clsB;3][ruyg;3][lrug;3][delB;3][elyg;3][lpCi;3][elwg;3][lrwg;3][clqB;3][lsyg;3][luxB;3][luvB;3][lrxg;3][luxg;3][lovB;3][lruB;3][clrB;3][lvxg;3][loyB;3][dlpB;3][lmwg;3][lvCB;3][lrsB;3][pqvB;3][mwyg;3][nryB;3][dgwg;3][1 mog;3][dlmB;3][lsyB;3][lsuB;3][lnvB;3][lqiB;3][lqwB;3][lxyg;3][lqvB;3][lmuB;3][svyB;3][lpxg ;3][nqvg;3][lpug;3][lmwB;3][lmxg;3][clng;3][lqgi;3][lmsg;3][dlqB;3][elCg;3][uvyg;3][qvyg;3][l uyB;3][lrCg;3][lviB;3][lsxg;3][lqvg;3][lrwB;3][lpsg;3][mqvg;3][lqwg;3][lvyi;3][vxyB;3][lgrg;3][mqvB;3][lnqi;3][dlqg;3][vCyg;3][lnwg;3][lxyB;3][rwyB;3][lrCB;3][dryg;3][clqg;3][lsvg;3][nvy g;3][pwyg;3][lrvg;3][psyg;3][lCgi;3][qwyg;3][rvyB;3][luvg;3][lvCg;3][lxgB;3][dlrg;3][suyB;3][mqvi;3][cuyg;3][lpyj;3][mqwg;3][duyB;3][lgrj;3][qruB;3][qrxB;3][cxyg;3][nxyB;3][dlri;3][lmxi; 3][cvyB;3][nwCi;3][qrwg;3][lrjB;3][fvyg;3][clgi;3][oqvg;3][lpsi;3][ouyB;3][svyi;3][rvyi;3][clmi ;3][dwyi;3][cljB;3][ruCg;3][dvyg;3][fwyg;3][lpxi;3][dsyg;3][qvxB;3][ngxg;3][nryi;3][rxyi;3][dx yg;3][quxg;3][rwyi;3][nqug;3][suyi;3][vwyB;3][nuvg;3][uwCg;3][cflB;3][lnCi;3][flpB;3][puyi;3][nari;3][lnyj;3][lovi;3][uvCB;3][nvCg;3][elxB;3][pquB;3][lryj;3][asvB;3][wygi;3][pawg;3][lox B;3][qsug;3][dlpi;3][qrvg;3][fryg;3][qrwB;3][elrB;3][clri;3][fwyB;3][qrsg;3][dvyB;3][lrsi;3][cli B;3][duyg;3][pqvi;3][pqxg;3][lnvi;3][elpg;3][rsyi;3][uxyi;3][cdli;3][fvyB;3][lngj;3][clpi;3][eflB; 3][elsg;3][qvCg;3][flsB;3][wyiB;3][flmg;3][vxyi;3][dlmi;3][lxjB;3][swCg;3][lxiB;3][lrgj;3][lpgj; 3][dlsB;3][qrxg;3][lsvi;3][elqg;3][flvg;3][lrxi;3][lsCB;3][cflg;3][loxg;3][elxg;3][dlyi;3][flug;3][1 oCB;3][lmui;3][lvxi;3][lmwi;3][dlCi;3][flCg;3][nuyg;3][lrwi;3][dlsg;3][dlwi;3][lvwB;3][lwxB;3][elug;3][flxB;3][flpg;3][dlgi;3][loug;3][luCB;3][clug;3][lgsg;3][flwg;3][lnuB;3][nvyi;3][lwCi;3][luwB;3][flrB;3][clyi;3][luvi;3][ouyg;3][dlug;3][dlvB;3][vwyg;3][dflB;3][uwyB;3][quvB;3][lvCi; 3][loqB;3][lvwg;3][lwxg;3][flwB;3][lpui;3][flxg;3][lyjB;3][eluB;3][lnug;3][clvg;3][flvB;3][uwy g;3][fluB;3][flrg;3][lswg;3][lqgj;3][luxi;3][lxCB;3][cluB;3][dluB;3][lsui;3][dflg;3][lsCg;3][louB; 3][dlxg;3][loyi;3][elqB;3][lwiB;3][lygj;3][lgjB;3][swyg;3][quvg;3][lugi;3][lswB;3][lgsB;3][lqCi; 3][dlvg;3][elvg;3][lsyi;3][clCi;3][clxg;3][uvyi;3][loCg;3][lwgi;3][lrui;3][flCB;3][lrCi;3][nqvi;3][luiB;3][qrug;3][lqug;3][nxyg;3][luwg;3][clwi;3][ruyi;3][wxyg;3][lsxi;3][flsg;3][clsg;3][dlxB;3][l qxB;3][lxyi;3][uvCg;3][logg;3][elrg;3][flyB;3][qsvg;3][lorg;3][lxCg;3][luCg;3][nuyB;3][lqxg;3][luyi;3][lnxg;3][flyg;3][elvB;3][pqug;3][eflg;3][swyB;3][lxgi;3][qvxg;3][lwyi;3][lqvi;3][lquB;3][l rvi;3][lpwi;3][cvyg;3][clvB;3][lgri;3][dliB;3][lgtB;2][elCi;2][luCi;2][lygz;2][ngxi;2][lswi;2][dlqj ;2][qvwB;2][lCzB;2][elpi;2][lqwj;2][lwyj;2][pqui;2][wxyi;2][lwyz;2][qtwg;2][wygj;2][luyj;2][lpt g;2][lCgz;2][ltCB;2][oquB;2][lsCi;2][lvyz;2][floB;2][lvyj;2][dltB;2][lwgz;2][dltg;2][elui;2][clog; 2][nwyi;2][ltwg;2][qvxi;2][dlui;2][lvwi;2][luzB;2][lqui;2][lwxi;2][vwyi;2][qrxi;2][flvi;2][loqj;2][lnxi;2][flwi;2][fvyi;2][ltyg;2][quwB;2][cfli;2][flCi;2][luyz;2][lxgj;2][flqB;2][fwyi;2][lqwz;2][flui ;2][celi;2][cvyi;2][lori;2][nwyj;2][uvCi;2][lxyj;2][efli;2][loui;2][lCjB;2][lvzB;2][clui;2][lvjB;2][l uvj;2][twyg;2][lowg;2][elyi;2][lujB;2][lwgj;2][fnyg;2][fqvB;2][lxyz;2][qrwi;2][nqui;2][cqvg;2][l xCi;2][lwCj;2][lwzB;2][lqgz;2][clgz;2][lqwi;2][elxi;2][uwyi;2][lugz;2][fqvg;2][elvi;2][lyij;2][dlx i;2][swyi;2][lwjB;2][luwi;2][lqij;2][flyi;2][qrui;2][flsi;2][lugj;2][lrtg;2][cltB;2][nuCg;2][elog;2][f lri;2][qswB;2][lvgj;2][logi;2][ltwB;2][flog;2][clvi;2][lqxj;2][lCgj;2][flqg;2][lqvj;2][dloB;2][lquj; 2][nuwg;2][dlsi;2][lnwi;2][nxyi;2][qvwg;2][fqwg;2][quvi;2][cltg;2][lmtg;2][lrgz;2][quwg;2][dlo g;2][lqzB;2][lquz;2][flgz;2][clyj;2][lowB;2][oqug;2][clni;2][lnui;2][clgj;2][lvgz;2][dlgj;2][flng;2][loCi;2][lrtB;2][lgsj;2][dlvi;2][dlyj;2][flxi;2][lgtg;2][owyg;2][ouyi;2][clsi;2][dvyi;2][ltCg;2]

5 Phage Combinations

5 phage combinations were analyzed in silico for their ability to lyse the 352 different strains of Klebsiella pneumoniae bacteria as classified by MLST, based on the ability of the single phage to lyse a bacteria of a particular capsule type as assessed by a liquid media assay.

The combinations that lyse the highest number of bacterial strains (as classified by MLST) are provided herein below. This trait is referred to as “at least 1 phage % coverage”. The number following each combination refers to the Percent trait performance—in this case the percent of bacteria strains as profiled by their capsule type that are targeted by the phage combination. The combinations are listed in descending performance grade.

[luvyg;90][luwyg;89][lquvg;89][lquwg;88][luvyi;88][lruyg;88][luvyB;88][louyg;88][flwy g;88][luxyg;88][flvyg;88][lqvxg;88][lquvi;88][flxyg;88][fluyg;88][cluyg;88][lwxyg;88][flqvg;88][lquvB;88][lrxyg;87][lgtxg;87][lgrug;87][lqvwg;87][loqug;87][luvCg;87][lqwxg;87][lvwyg;87][lquxg;87][eluyg;87][flqwg;87][luygi;87][ltvyg;87][ltxyg;87][lgrxg;87][lsuyg;87][lpuyg;87][lwyg i;87][luwyi;87][lgtvg;87][luwyB;87][lvxyg;87][dluyg;87][lmuyg;86][flqug;86][lswyg;86][lgtwg; 86][luyiB;86][flqxg;86][clqug;86][lqwgi;86][lpqug;86][lgswg;86][lquwi;86][flqvi;86][lqvgi;86][l uygj;86][flwyi;86][lvygi;86][flvyi;86][lquwB;86][ltuyg;86][ltwyg;86][elqug;86][luxyB;86][lwyg j;86][lqvxi;86][lqviB;86][loqxg;86][lpxyg;86][lruyi;86][luwCg;86][clqvg;86][flxyi;86][lquyg;86][elwyg;86][flqvB;86][lgsug;86][elqvg;86][lqwiB;86][flqwi;86][luxyi;86][fivyB;86][dlqug;86][lr vyg;86][lwyiB;86][lgswi;86][lruyB;86][louyi;86][lpqxg;86][lgrwg;86][lqvyg;86][luygz;86][lnuy g;86][luCyg;86][lgsvg;86][luygB;86][lrwyg;86][clvyg;86][flryg;86][lsvyg;86][luygh;86][louyB; 86][lmwyg;86][flwyB;86][lqwyg;86][clxyg;86][lwCyg;85][lgrvg;85][dlqvg;85][cluyB;85][flqxi; 85][flqwB;85][lqvgj;85][lvyiB;85][lgtug;85][lpwyg;85][fluyB;85][elqwg;85][lgruB;85][lvCyg;85][luyjB;85][lgsxg;85][dlwyg;85][lquxi;85][lwxyi;85][lgrui;85][loqui;85][lsuyi;85][luygA;85][lu ygk;85][eluyi;85][loquB;85][lqwgj;85][lquxB;85][cluyi;85][lqxgi;85][dlvyg;85][lmxyg;85][fluyi ;85][lowyg;85][lqvwi;85][clqxg;85][lqugi;85][lwygB;85][lwygz;85][lqwCg;85][lqvxB;85][elvyg ;85][lmqwg;85][lqwxi;85][lmvyg;85][lswyi;85][flxyB;85][lgrxi;85][lxygi;85][loxyg;85][lvygj;85][loqwg;85][dlxyg;85][lnwyg;85][lqvCg;85][lpqwg;85][lgsvi;85][flqrg;85][lquiB;85][lwygh;85][lqxiB;85][lnvyg;85][lmqug;85][dlqxg;85][luxCg;85][dluyB;85][dluyi;85][lpvyg;85][elqvi;85][lru Cg;85][louCg;85][lgsxi;85][lgtxi;85][lquCg;85][lgtvi;85][elqxg;85][lrxyi;85][dlqwg;85][elxyg;85][lqwgh;85][clwyg;85][lqwgz;85][lmqxg;85][fltyg;85][loqvg;85][lgsui;85][clqvi;85][logtg;85][clqui;85][clquB;85][lqwgB;85][luvCB;85][lgtxB;85][elqui;85][luvCi;85][lsxyg;85][lmqvg;85][fl vCg;85][lpuyB;85][lpqui;85][lqxyg;85][lnqvg;85][lqvgz;85][lnqwg;85][lqvgh;85][flqxB;85][lpq uB;85][lwygA;85][lvxCg;85][lvwyi;85][flygi;85][lqvwB;85][dflyg;85][lwxyB;85][lwygk;85][lo vyg;85][lvxyi;85][lsuyB;85][lgrxB;85][lpqvg;85][flqui;85][lqugj;85][eluyB;85][lgtvB;85][lgswB ;85][floyg;85][lqwxB;85][lpuyi;85][lvygB;85][lvygz;85][lvygh;85][lsvyi;85][lqvgB;85][flquB;85][flpyg;85][lqwgk;84][luyih;84][lqvjB;84][lvxyB;84][lquyB;84][lnuyi;84][lquyi;84][lqvgk;84][f lqtg;84][lmwyi;84][lqugh;84][lqvgA;84][cflyg;84][ltuyB;84][lsuCg;84][dluCg;84][lotyg;84][lsu wg;84][lqxCg;84][lngxg;84][flryi;84][lqwjB;84][lpuCg;84][lrxCg;84][lqvyi;84][clqwg;84][clqxi; 84][lqxgj;84][clqvB;84][lqwyi;84][dlqui;84][dlquB;84][flyiB;84][lrygi;84][flsyg;84][lqwgA;84][lxygj;84][lrxyB;84][lxyiB;84][lxCyg;84][lwyjB;84][lgrwi;84][ltvCg;84][flqri;84][loqxi;84][lgtwi ;84][ltvyB;84][cluCg;84][lmuyi;84][ltxyB;84][elwyi;84][nuvyg;84][llmuyB;84][lrtyg;84][ltxCg;8 4][lgtwB;84][flwCg;84][lnxyg;84][lvygA;84][lwxCg;84][lvygk;84][flogg;84][lnqug;84][lqrtg;84][elqwi;84][ltxyi;84][lpqtg;84][flpqg;84][ltuyi;84][lswCg;84][lvwCg;84][lswyB;84][suvyg;84][lv wyB;84][lgsvB;84][lmqwi;84][lqujB;84][clvyi;84][lqugB;84][lgsuB;84][clxyi;84][lqugz;84][ltvy i;84][lpqxi;84][elquB;84][clqtg;84][luCyB;84][clvyB;84][lpqwi;84][luCyi;84][lqvCi;84][lgsxB;84][lpqxB;84][elvyi;84][eluCg;84][lgstg;84][dluvg;84][lqugk;84][flxCg;84][lmqvi;84][lruwg;84][clxyB;84][dlqtg;84][lnuyB;84][lgrgi;84][cltyg;84][lpxyi;84][lwyih;84][clqxB;84][luyzB;84][luv wg;84][loqxB;84][loqwi;84][lqxgz;84][lqxgB;84][lqrvi;84][flygj;84][lqxgh;84][fluvg;84][luyiA; 84][lmqxi;84][luyiz;84][luyij;84][firyB;84][lqwCi;84][lmwyB;84][lrsyg;84][dltyg;84][lnqvi;84][luykB;84][dlqvi;84][flnyg;84][eltyg;84][lvyjB;84][luwCB;84][ltwyi;84][ltwyB;84][lxygB;84][lq vyB;84][lptyg;84][lrvyi;84][lxygh;84][lmvyi;84][lqugA;84][lngwi;84][lsvCg;84][flqsg;84][lxygz ;84][ltwCg;84][lmqwB;84][lwCgi;84][lnwyi;84][lsuvg;84][flqrB;84][lrwyi;84][luwCi;84][lmxyi; 84][lsxyi;84][luyhB;84][lsvyB;84][elqvB;84][elqtg;84][fluCg;84][dflgg;84][lvCgi;84][lgtui;84][l oryg;84][lgtuB;84][lgrwB;84][eluvg;84][lqwyB;84][elqxi;84][ltygi;83][lmryg;83][clryg;83][loxC g;83][lrygj;83][flygB;83][luwgi;83][lgtyg;83][flvCi;83][eflyg;83][lqvij;83][lpwyi;83][lngxi;83][l oqvi;83][lgtgi;83][xygA;83][elwyB;83][lmqxB;83][lswCi;83][lqwCB;83][dflyi;83][lpqwB;83][d lqxi;83][elryg;83][lruxg;83][lruvg;83][loxyi;83][flpqi;83][lgrsg;83][lpxyB;83][nuwyg;83][lqwiA ;83][lquCB;83][lvCyi;83][flqyg;83][dlqvB;83][lmuCg;83][ltuvg;83][cluvg;83][lwyij;83][flygz;83][lmqvB;83][flmyg;83][lwCyi;83][qxjB;83][floyi;83][ryiB;83][flsyi;83][lvyih;83][lowyi;83][c lqwi;83][lpxCg;83][eluwg;83][lgrvB;83][loqwB;83][lqxgk;83][logrg;83][lqvih;83][lqviz;83][lqx yi;83][flogi;83][lstyg;83][dlwyi;83]

The percent of host bacterial strains (as classified by MLST) that are infected by two phages of a phage combination are provided below. This trait is referred to as “at least 2 phage % coverage”. The phage combinations are ordered in descending performance grade.

[lnwyB;51][lqwyB;51][lwygB;51][luwgB;51][lnwyg;51][lqwyg;51][lngwg;50][lnwyi;50][lqwyi;50][lnuwg;50][lnryg;50][lgryg;50][luwgi;50][dlryg;50][lrygB;50][dlnyg;50][dlygB;50][lq wgB;50][lnqwB;50][lnwCg;49][lqvyB;49][luvgB;49][lnuCg;49][lnqrg;49][dlwyB;49][lngwi;49][lqvyg;49][lwygi;49][lvwyB;49][lgryi;49][luwyB;49][lqugB;49][luygB;49][luwyg;49][lnuyg;49][lqrgB;49][dlwyg;49][dlqyg;49][lnvyB;49][lnqug;49][lnvyg;49][lvygB;49][lqvyi;49][lnuvg;49][lv wyi;49][lrCyg;49][dlwyi;49][lrwyB;49][lvwyg;49][lpqyg;49][luwyi;49][lwCyB;49][lqvwB;48][1 rwyg;48][dlsyg;48][luwiB;48][lwCyg;48][lnuyB;48][lrygi;48][dlpyg;48][lwyiB;48][lnuyi;48][lu CgB;48][lrwyi;48][dlvyB;48][dlygi;48][lqvwg;48][lnvyi;48][lqwgi;48][luvgi;48][lgswg;48][lwC yi;48][lswyg;48][lquwg;48][lqvgB;48][luwCB;48][dlqwg;48][dlvyi;48][luwCi;48][dlqwB;48][lq vwi;48][luwCg;48][lnquB;48][luygi;48][lnqui;48][lswyB;48][lquwB;48][lvCyB;48][lquwi;48][l wCgB;48][dlvyg;48][larCg;48][dlarg;48][lqugi;48][lnavg;48][laxyg;48][luCgi;47][dlpag;47][lqv Cg;47][lgswB;47][lryiB;47][lnvCg;47][dlqsg;47][lvCyg;47][dlyiB;47][lqwCB;47][lqwCg;47][lp Cyg;47][lswyi;47][lvCyi;47][lgrwg;47][lgrgi;47][lqxyi;47][lnpyg;47][dlsyi;47][lqxyB;47][lrugi;47][dlqwi;47][lquyg;47][lgrwB;47][lvwCB;47][lqygi;47][lqvCB;47][lnqvB;47][lvygi;47][luviB;46][lqwCi;46][clqyg;46][dlqvg;46][lgswi;46][lqvCi;46][lquyi;46][lgrwi;46][lvwCg;46][lquyB;46][lpygB;46][lqwiB;46][lpwyB;46][dlCyg;46][dlqvB;46][lnqvi;46][lvwCi;46][lnxyg;46][lswCg;46][clwyg;46][lpqvg;46][luvwB;46][lsuwB;46][clnyg;46][lquCg;46][lnxyB;46][luvwi;46][lwCgi;46][lpvyB;46][lruyB;46][lruyg;46][clryg;46][lpwyi;46][lpqwB;46][dlwCg;46][dlqgi;46][lqvgi;46][lsuwg;46][dluwg;46][luyiB;46][luCyB;46][luCyg;46][luCyi;46][luvwg;46][lvCgB;46][lvyiB;46][lpqvB;46][lnpqg;46][lpwyg;46][lpqwg;46][dlqvi;46][lsuwi;46][lruyi;46][lsuCg;45][lsuCB;45][clwyi;45][lgrsg;45][lpqvi;45][lpvyg;45][dluyi;45][lquCB;45][lrwCg;45][dluyB;45][luCiB;45][in xyi;45][cdlyg;45][lpvyi;45][lsuCi;45][luvCi;45][luvCB;45][lgriB;45][clqwg;45][dloyg;45][lswC B;45][lxygB;45][lpqCg;45][clwyB;45][lpqwi;45][lquCi;45][luvyg;45][lgsug;45][lgrug;45][clryi; 45][iquiB;45][cingg;45][luxyi;45][clygB;45][clvyg;45][luvCg;45][lpryg;45][lquxg;45][dluyg;45][lpuyg;45][luxyg;45][luvyB;45][luvyi;45][lpqgB;45][lquvg;45][lpuyB;45][dlqsi;45][lpuyi;45][lq uvB;45][lsuyg;45][lgrvg;45][lgruB;45][clvyB;45][lquxi;45][lruCg;45][lwyij;45][lgrui;45][lsuyi;45][lpygi;45][lrsyg;45][clvyi;45][lgsuB;45][lwxyi;45][lvxyB;45][lwxyB;45][lxCyg;45][dlqug;45][lwxyg;45][lruCB;45][ixCyB;45][clqrg;45][lruCi;45][lquvi;45][lowyB;45][luxyB;45][clqvg;45][it wyB;45][ltwyi;45][iwCiB;45][iswCi;45][lgsvg;45][lvxyi;45][logyg;45][dlvCg;45][xCyi;45][lqs ui;45][ciCyg;45][lqwxg;45][dlsug;45][lpqug;45][lvxyg;45][lrvyB;45][lpqrg;45][grvB;45][suyB; 45][flqyg;44][iqviB;44][cluwg;44][dlqui;44][dlquB;44][dlxyi;44][dloqg;44][dlxyg;44][loqwg;44][clqwB;44][dflyB;44][clygi;44][luwxg;44][lqwxi;44][lxygi;44][ivCgi;44][lwygj;44][lrvyi;44][lo qwB;44][dlqiB;44][lowyg;44][lrsyB;44][lpqsg;44][flnyg;44][dlqCg;44][ingxg;44][lrvyg;44][lsvy g;44][clsyg;44][dflyg;44][clqsg;44][lowyi;44][lgrvi;44][lpqui;44][iquxB;44][pvCg;44][grsB;44][ltwyg;44][clqwi;44][ipquB;44][iqwxB;44][isuvB;44][dlxyB;44][lsuvg;44][loqCg;44][flngg;44][cluyi;44][cdlqg;44][clsyi;44][lgrsi;44][lruwB;44][luvxg;44][lgtwg;44][lpqgi;44][clqCg;44][lou wg;44][lmwyg;44][imwyB;44][flwyB;44][clqrB;44][lpuwg;44][lrsyi;44][lrvCg;44][lruwi;44][loq wi;44][lpsyg;44][clwCg;44][luxCi;44][dluvg;44][isvyB;44][loCyg;44][dluvB;44][clpqg;44][puC i;44][lruwg;44][finyB;44][clpyg;44][clqri;44][iqxCg;44][ipwCg;44][cluCi;44][figyB;44][pyiB;44][elryg;44][igtwB;44][cluyB;44][dluvi;44][ipuCB;44][luwgj;44][dflyi;44][flwyg;44][gsvB;44][dlmyg;44][lgtwi;44][lmryg;44][ingxB;44][cluyg;44][lsuvi;44][luxCg;44][ipuCg;44][dlqxg;44][cl qvi;44][clqvB;44][isvCg;44][fluwg;44][lqvxg;44][dlrug;43][cluCg;43][delyg;43][cluCB;43][clqu g;43][flqwg;43][iqxCi;43][lsvyi;43][lmuyi;43][lpuvg;43][imuyB;43][nuwyg;43][flgyi;43][clqui; 43][clqsi;43][clvCg;43][ingxi;43][lrsug;43][clqgi;43][flwyi;43][lmryi;43][qxCB;43][elryi;43][el wyB;43][cluvg;43][lavxB;43][flnyi;43][lawij;43][ltuwg;43][lavxi;43][lmuwg;43][svCB;43][vC iB;43][lgsvi;43][luxCB;43][dltyg;43][flCyg;43][lwyjB;43][dluxg;43][dluCB;43][dlqxi;43][dluCg ;43][lmwyi;43][dlygj;43][dlqxB;43][lqwgj;43][dluCi;43][dloug;43][nuwyi;43][lrxyg;43][fluCi;43][lmquB;43][lwxCi;43][dflqg;43][lmqvg;43][lwxCg;43][lwxCB;43][elwyg;43][elygi;43][loqvB ;43][lmqwg;43][lsvCi;43][ltwCg;43][flvwg;43][lowCg;43][lmqug;43][lpsyi;43][lrxyB;43][lxyiB; 43][flngi;43][clpqi;43][lrxyi;43][flqwB;43][flryg;43][clvCi;43][ltuCg;43][lmqui;43][fluCB;43][1 muyg;43][clquB;43][lqxgB;43][clrug;43][lmgrg;43][loqvg;43][loqug;43][firyB;43][lpqsi;43][lsx yg;43][lsuxg;42][dlxCg;42][flygB;42][luwjB;42][lntyg;42][flwCB;42][eluwg;42][elpyg;42][dflq B;42]

The percent of host bacterial strains (as classified by MLST) that are infected by three phages of a phage combination are provided below. This trait is referred to as “at least 3 phage % coverage”. The phage combinations are ordered in descending performance grade.

[lruvg;31][cluwg;31][dluwg;31][lruwB;30][lruwg;30][luvwg;30][dluvB;30][luvwB;30][dl uvg;30][lruwi;30][dluvi;28][luvwi;28][luwxg;28][dlwyB;28][dluyi;28][dlrug;28][dluyB;28][cluv g;28][dlwyg;28][dlwyi;28][dluyg;28][lrwCg;28][dluCg;28][dlqwB;28][lrwyi;28][dlqwi;28][dlqw g;28][lrwyg;28][lpuwg;28][dluCB;28][dluCi;28][lrwyB;28][lquwi;28][lquyB;28][dluxg;28][clwy B;28][luCyg;28][lwCyB;28][lwCyg;28][dlCyg;28][lquwB;28][lquyi;28][clrug;28][luwyg;28][dlq ug;28][lwxyB;28][dlqui;28][lquwg;28][dlquB;28][lwCyi;28][luwyB;28][lrvyg;28][luCyB;28][lrv yB;28][luwyi;28][luCyi;28][lquyg;28][dlqCg;27][lrvyi;27][lgrwi;27][luwCg;27][lgrwg;27][clqw B;27][luwCB;27][lwxyi;27][lrvCg;27][lwxyg;27][clwyg;27][clwCg;27][lmuwg;27][luwCi;27][lq rwB;27][clwyi;27][dlwCg;26][lruxg;26][dlvyi;26][clqwi;26][lpuvg;26][cdlyg;26][dlqyg;26][elu wi;26][clqwg;26][cluxg;26][eluwg;26][dlvyB;26][dlvyg;26][dlxyB;26][lqwCg;26][lpwyg;26][lq uCg;26][lwxCB;26][elrug;26][lruCg;26][cluyB;26][lgrvg;26][lpwyB;26][lvwyB;26][clquB;26]l[1 vwCg;26][lruCB;26][lvwCi;26][dlxyg;26][cdlqg;26][lvwyi;26][lqwCi;26][lquCB;26][lvwyg;26][lqwyg;26][flrwg;26][lpwyi;26][lvwCB;26][lgrvB;26][dlxyi;26][clqug;26][lqwyB;26][lqwyi;26][clCyg;26][lqwCB;26][lquCi;26][cluyg;26][dlqvi;25][lqwxB;25][lruyg;25][luvyB;25][lqrvi;25][cl uCg;25][lpuxg;25][dlqvg;25][lquvB;25][clqui;25][elwyi;25][clqyg;25][dlvCg;25][lruCi;25][elwy B;25][lgrug;25][lwxCg;25][lgruB;25][luvyg;25][lquvg;25][cluCB;25][lmuvg;25][dflwg;25][lwx Ci;25][lrxyB;25][cluyi;25][elwyg;25][lruyB;25][louwg;25][dlqvB;25][luvxg;25][clsug;25][lqwxi ;25][lqvwi;25][lqvwB;25][luvyi;25][lruyi;25][lrxyg;25][lgryi;25][lgryg;25][luvCB;25][lpwCg;25][lvCyi;25][lquvi;25][lnuwg;25][luxyB;25][lqwxg;25][luvCg;25][lpryg;25][clryi;25][lqvwg;25][dlqrg;25][dlryg;25][lvCyB;25][delyg;25][clryg;25][lgrui;25][lmwCg;25][lmwCi;25][lvCyg;25][c luCi;25][eluyB;24][lpxyB;24][dlmyg;24][dlqxB;24][lrxyi;24][nuwyg;24][lmwyi;24][luxCB;24][clvCg;24][luvCi;24][clxyB;24][fluwg;24][lsuwg;24][lrCyg;24][clxyi;24][lmwyB;24][lxCyB;24][dlmag;24][flvwg;24][dlnyg;24][delag;24][flrug;24][clxyg;24][lmwyg;24][lmuyg;24][lrxCB;24][clqrB;24][clvCi;24][dloug;24][nuwyi;24][dlsug;24][luxyg;24][eluyg;24][lsuwB;24][clqCg;24][el uyi;24][ltuwg;24][lxCyg;24][lorug;24][lpuyg;24][eluvB;24][nvwyg;24][lpqrg;24][clmyg;24][lnu yB;24][lvxyB;24][clxCg;24][lpqwg;24][luxyi;24][dlxCg;24][clqri;24][elqwi;24][elqwg;24][lmuy B;24][flqug;24][fluyg;24][lrxCg;24][elqwB;24][dlqxi;24][lpuyB;24][lpqwB;24][clvyB;24][celyg ;24][lpqwi;24][luxCg;24][dlqxg;24][clqrg;24][eluvg;24][lpuCB;24][lnquB;24][dflvg;24][lpxyg;24][lpuCg;24][lxCyi;24][lmuCg;23][clvyg;23][lqvyg;23][lpuCi;23][lpqug;23][elqug;23][lnqug;23][lnqui;23][lqvyi;23][lqvyB;23][lmryg;23][lnuyi;23][lnuyg;23][lmvyg;23][lmqwg;23][elvyg;23][lmqwi;23][lpuyi;23][lmqwB;23][lpquB;23][eluvi;23][lqxyB;23][lvxyg;23][lsuwi;23][nruyg;23][1 quxB;23][elvyi;23][lmvyB;23][clpyg;23][lmuyi;23][elqui;23][lpxyi;23][lmqug;23][lpvyg;23][lrx Ci;23][flquB;23][dlpyg;23][lpvyB;23][luxCi;23][clvyi;23][lpqyg;23][lvxCB;23][fluyB;23][elqu B;23][elvyB;23][elryi;23][lpCyg;23][lpxCg;23][lqxyg;23][lvxyi;23][flwyB;23][lvxCg;23][flqui; 23][lgrxB;23][eluCB;23][lmvyi;23][eluxg;23][flwyg;23][lquxg;23][lnuvg;23][elryg;23][fluvg;23][lqxyi;23][clqvB;23][clnyg;23][lnwyB;23][lpvyi;23][lowyg;23][lowCg;23][elxyB;23][lmquB;23][lowyB;23][flwyi;23][lmryi;23][clmqg;23][lgrCg;23][fluyi;23][eluCg;23][eluCi;23][lpqui;23][is uyB;23][lowyi;23][lsuyg;23][elxyi;22][celqg;22][lqvCB;22][lmqui;22][lqvCi;22][lgrxg;22][clqx B;22][clqxg;22][lqxCB;22][lnwyg;22][lpvCg;22][lrsug;22][lnuCg;22][clqvi;22][nuvyB;22][elxy g;22][lvxCi;22][lquxi;22][nuvyg;22][dlpqg;22][lrsyB;22][lmgrg;22][lnryg;22][elxCg;22][clqxi;22][lqvCg;22][clqvg;22][lnwyi;22][fluvB;22][flwxg;22][elwCg;22][fluvi;22][lnwCg;22][loqwB;22][dlsyg;22][flpyB;22][lopyg;22][lovyB;22][louvg;22][elgri;22][loqwi;22][lnvyB;22][lmpyg;22][clngg;22][lswCB;22][lmxyB;22][elvCi;22][flgyi;22][flgyg;22][elvCg;22][nuvyi;22][lmvCg;22][figyB;22][dlsyi;22][elqrg;22][lqxCi;22][lpqCg;22][lnqwB;22][lmgri;22][lmuxg;22][loqwg;22][is wyB;22][elqvi;22][flpyg;22][loryg;22][flqwg;22][lsuyi;22][flqwB;22][elqvB;22][lgrxi;22][lqvxB ;22][lovyg;22][lqxCg;22][flqwi;22][lrsyg;22][elqvg;22][lswCg;21][cflyg;21][lngwi;21][flpyi;21][lnxyg;21][lnqvB;21][dflyB;21][lpqvB;21][lnvCg;21][dflgg;21][lnvyg;21][lnvyi;21][clpqg;21][fl wCg;21][dflqi;21][lgsuB;21][flCyg;21][lsuCg;21][flwCi;21][logrg;21][lnxyi;21][lrsyi;21][clsyg; 21][elqxB;21][lovyi;21][dflyi;21][flrCg;21][lswCi;21][lmqxB;21][lmoyg;21][lqvxg;21][lsuCB;21][logyg;21][cloyg;21][clpqi;21][nquvg;21][lnxyB;21][lswyi;21][cflyB;21][lmxCg;21][flmqg;21][suwyg;21][dflyg;21][dflqB;21][loryi;21][flpCg;21][lgsug;21][lnqwg;21][lpqvg;21][flwCB;21][lswyg;21][lmxyg;21][lmpqg;21][lnqrg;21][dflCg;21][ltuyB;21][louCB;21][lpsyg;21][rsuyg;21][l qvxi;21][flryg;21][logri;21]

The percent of host bacterial strains (as classified by MLST) that are infected by four phages of a phage combination are provided below. This trait is referred to as “at least 4 phage % coverage”. The phage combinations are ordered in descending performance grade.

[dlrug;15][lruwi;15][lruwg;15][lruwB;15][dluwg;15][lpuwg;15][dlwCg;14][cluwg;14][clr ug;14][clpyg;13][lnuwg;13][lpwCg;13][dlqyg;13][dlpyg;13][elwCg;13][luwxg;12][dlCyg;12][cl uyB;12][lnpyg;12][dluCg;12][lpwyB;12][lpwyg;12][dluCB;12][dlxCg;12][clqyg;12][clnyg;12][l uvwB;12][clwyB;12][dlryg;12][clwCg;12][cluyg;12][luvwg;12][clwyg;12][dluyg;12][dluyB;12][lruxg;12][clwyi;12][luvwi;12][cdlyg;12][dluxg;12][clmyg;12][lpryg;12][lquyB;12][lruyB;12][dl qug;12][dlquB;12][lruyi;12][dlpqg;12][lnwyi;12][lquyg;12][lmuwg;12][cluyi;12][dluCi;12][dlm yg;12][clpqg;12][clryg;12][dluyi;12][lnwyg;12][lpuyB;12][lpwyi;12][lpuyi;12][lnwCg;12][lnwy B;12][clCyg;12][dlqCg;12][lruyg;12][lpCyg;12][lpuyg;12][luCyB;11][dlwyB;11][clryi;11][cdlqg ;11][elpyg;11][lpqwg;11][lpuvg;11][lpuCB;11][dluvi;11][lrCyg;11][dluvg;11][lmwyB;11][lnuyB ;11][dlqrg;11][clmqg;11][dlwyi;11][cluCg;11][dlnyg;11][lmpyg;11][lpuxg;11][lvwyg;11][dluvB; 11][dlwyg;11][dlqui;11][lpuCg;11][lpqwB;11][cluCB;11][lnuyg;11][luCyg;11][clpqi;11][eluwg; 11][lpxCg;11][clvyB;11][clvyg;11][luwyB;11][lruvg;11][lmwyg;11][lpuCi;11][luwyg;11][lwxC B;11][dlmqg;11][lwxCg;11][lvwyB;11][lmwCg;11][lquyi;11][clqCg;11][lvwCB;11][clquB;11][1 ruCi;11][lmwyi;11][lgryg;11][lrxCi;11][dlvCg;11][loryi;11][clqwi;11][lquCB;11][clqrg;11][lquC g;11][lruCg;11][clqrB;11][clqwB;11][lgrCg;11][lmwCi;11][lrwCg;11][dloyg;11][lvwyi;11][lnry g;11][lrwyB;11][clxCg;11][luwyi;11][lpqwi;11][lmqwg;11][lqwyi;11][luwCg;11][lpqyg;11][clq wg;11][lrxCg;11][lnuCg;11][luwCB;11][lruCB;11][eluwi;11][clqug;11][lqwyB;11][lorug;11][clu xg;11][loryg;11][lpqCg;11][elxCg;11][lwxCi;11][luCyi;11][clngg;11][lnuyi;11][lrxCB;11][clxy B;11][lnxyB;11][lrwyg;11][lqwyg;11][lmqwB;11][lvwCg;11][lnuvg;11][cluCi;11][elpqg;11][clv yi;11][lmqwi;10][lxCyg;10][lqxCg;10][elvCg;10][dlqxg;10][lnvyg;10][lpvyg;10][lngwi;10][luw Ci;10][lmuyi;10][dlqwg;10][lquCi;10][lrwyi;10][dlqwi;10][clqui;10][lgryi;10][lmuyB;10][eluCg; 10][clqxB;10][lnxyg;10][lpqui;10][lmuxg;10][lwCyg;10][celyg;10][lpqrg;10][dlqxB;10][lpvyB;10][lvwCi;10][lgrui;10][dlvyB;10][clqri;10][lmpqg;10][lgruB;10][lxCyB;10][lwCyB;10][lnqwB;10][dlqwB;10][dlvyg;10][lgrug;10][dflwg;10][lpvCg;10][fluxg;10][dlxyg;10][lnvyB;10][elrug;10][lnpqg;10][eluxg;10][dlxyB;10][lopyg;10][lmuyg;10][flwxg;10][nuwyg;10][lwCyi;10][cloyg;10][clxyg;10][lqxCB;10][lmuCg;10][dflCg;10][lpqug;10][nvwyg;10][delyg;10][eluCB;10][lpvyi;10][lpquB;10][luvxg;10][lmxCg;10][lnqwg;10][eluvB;10][lxCyi;10][firug;10][eluCi;10][eluvi;10][lr vyi;10][lqvyB;10][lgrwB;10][lngxB;10][lrsug;10][nuwyi;10][clqvB;10][eluvg;10][lmryg;10][lnv Cg;10][elvCi;10][lqwCB;10][dlqxi;10][elryg;10][celqg;10][cluvg;10][lpxyB;10][dlxyi;10][lowy B;10][fluwg;10][delqg;10][lmquB;10][lnvyi;10][loCyg;10][lqxCi;10][rsuyg;10][dlvyi;10][lmqui; 10][lnxyi;10][clvCg;10][lrvyB;10][luxCi;10][luxCB;10][lpxyi;10][lqxyB;10][lqwCi;10][elwyB;10][flrwg;10][lpqxB;10][lmryi;10][luvyg;10][lowyg;10][louyi;10][lmqug;10][lqvwg;10][lquwB;10][clxyi;10][lqwCg;10][clqxg;10][Irvyg;10][lpqxg;10][lwxyB;10][lgrwg;10][lqvyg;10][elwyg;10][lclavg;10][louyB;10][lpxyg;10][elwyi;10][luxCg;10][lpaxi;10][lavwB;10][louyg;10][lquwg;10][lwxyg;10][luvyi;10][luvyB;10][lqxyg;10][flwCB;9][flqyg;9][lqvwi;9][luxyi;9][lnqrg;9][logyg;9][dlqvB;9][lgrxB;9][suvyi;9][eluyB;9][lowyi;9][lmxyg;9][flqCg;9][nruyg;9][lgtyg;9][lmxyi;9][lu xyB;9][suvyB;9][lqwxB;9][flwCg;9][elqwB;9][louvg;9][lqxyi;9][lvCyg;9][elqrg;9][lmuvg;9][lou wg;9][lmgrg;9][lmxyB;9][lrxyi;9][lrvCg;9][lwxyi;9][lvCyB;9][lmvCg;9][lgrxi;9][flCyg;9][lsuvi; 9][elqwg;9][ltCyg;9][dlqvg;9][lqvyi;9][lvxCg;9][clqvi;9][lvxCi;9][lnquB;9][lvxCB;9][lgrwi;9][s uvyg;9][lsuvg;9][eluyg;9][lrxyg;9][lgrxg;9][lqwxg;9][lsuvB;9][lngxg;9][lmgri;9][lquwi;9][figyB; 9][elryi;9][lnqug;9][clvCi;9][clqxi;9][dloug;9][luxyg;9][lrxyB;9][elqwi;9][flwCi;9][lquxi;9][luv Cg;9][nuvyi;9][elquB;9][dltCg;9][lqvCB;9][elqxB;9][luvCi;9][lmvyB;9][dflyB;9][elvyB;9][clsy g;9][luvCB;9][lquxg;9][lquxB;9][loxCg;9][fluCi;9][lmqxB;9][cflyg;9][lmqvB;9][lmqvg;9][lvCyi ;9][lpqvi;9][elqxg;9][lnqui;9][lowCg;9][flpCg;9][fluCB;9][dflyg;9][flvwg;9][eluyi;9][lngxi;9][dl ogg;9][lmvyi;9][lmvyg;9][lqwxi;9][elvyg;9][lmqxg;9][elgri;9][loqCg;9][lmqvi;9][nuvyB;9][suw yg;9][dlqvi;9][dltyg;9][flgyi;9][nuvyg;9][fluCg;9][elxyB;9][cflyB;9][elqug;9][elxyg;9][lpqvg;9][lqvCg;9][lpqvB;9][louxg;9][lmqxi;9][loxyg;8][clogg;8][lvxyi;8][clsyi;8][dlsug;8][flpyB;8][lgrvg ;8][fluvi;8][flxCB;8][ltuyg;8][flryg;8][lsvyg;8][dflqB;8][flmyg;8][lsuwB;8][flpyg;8][dlsyg;8][lsv yi;8][lgrvB;8][loxyi;8][eloyg;8][lnqvB;8][flnyB;8][ilnqvg;8][lpsyg;8][flxCi;8][lrtyB;8][cltyB;8][1 moyg;8][cflyi;8][lovyB;8][lrsyB;8][lovyi;8][clqsg;8][lrtyi;8][flrCg;8][fluvB;8][ltvyg;8][lntyg;8][flmyi;8][cflqB;8].

### Example 5

A 4-Phage Cocktail Selected to Target KPC-2 Strain

The in vivo efficacy evaluation in the peritonitis infection model was conducted with immunocompetent mice and measured the survival of a lethal infection. The phage cocktail was tested as a monotherapy and also in combination with tigecycline. The efficacy study included tigecycline tested alone as a quality control reference agent, and was administered once at 1 h post infection. The phage cocktail was administered twice (BID) per day with 12 h intervals (ql2h) starting at 1 h post-infection for a total of five days. The administration dosing routes included subcutaneous (SC), intraperitoneal (IP) and intravenous (IV). Biosamples, including serum, liver, kidneys and spleen tissues, were collected on Day 8 post-infection from all phage cocktail treated animals that survived the infection and three control animals that survived the infection.

The carbapenem resistant K. pneumoniae strain, BAA-1705 (also referred to herein as KPC-2 strain), was obtained from the American Type Culture Collection (ATCC). Susceptibility of the strain is summarized in FIG. 5 and was determined following the recommended assay conditions of the Clinical Laboratory Standards Institute (CLSI) M07-A10 microdilution procedure and the CLSI M100 interpretive criteria. The species identity was confirmed by 16S analysis. The antimicrobial resistance genes (AMR) of the K. pneumoniae strain BAA-1705) were identified from whole genome sequence analysis by CosmosID.

The following cocktail was generated to target the Klebsiella pneumonia strain BAA-1705: CT-227-KP4M1-20 (SEQ ID NO: 11), CT-227-KP4M1-11 (SEQ ID NO: 9), KP10-89 (SEQ ID NO: 24) and KPL6a-30 (SEQ ID NO: 30) and tested in the KPC-2 mouse peritonitis infection model.

Results

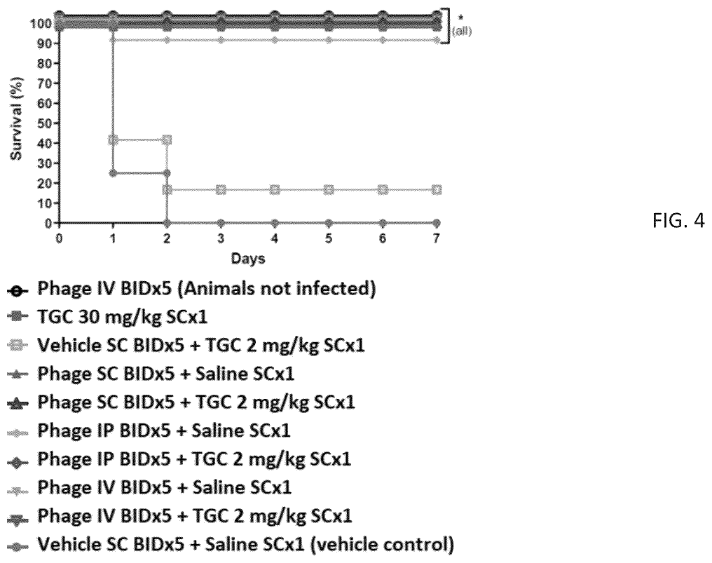

Animals treated with the cocktail showed 100% survival, as illustrated in FIG. 4.

### Example 6

Essential Genes for the Phage Lytic Cycle

Materials and Methods: Gene Analysis

According to certain embodiments, the phages' genomes are reduced to create synthetic phages with smaller genomes without significantly hampering their essential functionality (e.g., the ability to infect and lyse a host bacteria). According to certain embodiments, such a reduced genome can then more readily accommodate a heterogenous molecule of DNA that otherwise, if added to the original full genomic DNA, may be challenging due to the limited DNA encapsulation capacity of a phage (see for example Pires, D. P., Monteiro, R., Mil-Homens, D. et al. Designing P. aeruginosa synthetic phages with reduced genomes. Sci Rep 11, 2164 (2021). www(dot)doi(dot)org/10.1038/s41598-021-81580-2).

Additionally or alternatively, the genetic sequences of the selected phages can be modified or optimized, e.g. for expression in a suitable producer cell line, provided the essential genes are relatively conserved.

According to certain embodiments, the following exemplary method for finding the likely essential genes is used. A gene X was defined as essential if it was recognized and assigned a function by PATRIC (www(dot)docs(dot)patricbrc(dot)org/). In addition, if the gene's function is unknown by PATRIC (e.g. “hypothetical protein” or “phage protein”) the following test is performed: given a phage genome, for a gene X, count the number of homologs (global amino acid similarity of 30% or more using blastp) in all publicly available phage genomes infecting the same species (num.homologs(gene X)). Subsequently, the mean and standard deviation of number of homologs for each gene in the genomes that were found to contain gene X were computed, and the z-score for gene X was calculated as follows:

$$z ( gene X ) = \frac{\begin{matrix}[ num . homologs ( gene X ) ] - \\ [ mean num . homologs ( \begin{matrix}each gene in each genome \\ containing gene X\end{matrix} ) ]\end{matrix}}{\begin{matrix}standard deviation num . homologs \\ ( each gene in each genome containing gene X )\end{matrix}}$$

Genes with z-scores over −1 were defined essential. All other genes were defined non-essential.

Below, is a list of essential genes for each phage. Each gene is represented by square brackets, containing the following data fields separated by semi-columns: first, the gene's start coordinate, end coordinate, and strand to relate to with relation to the phage genome sequence as presented in the sequence listing (+ is the strand given in the sequence listing). Second, the gene's function. (“HP” denotes a hypothetical protein and “PP” denotes an unclassified phage protein).

Essential genes of phage CT-227-KP4M1-11: [66:561:−;Endolysin][589:898:−;HP][905:1352:−;HP][1348:1585:−;HP][1584:1911:−;HP][1907:2204:−;HP][2200:2563:−;HP][2559:3150:−;HP][3189:3783:−;HP][3812:4040:−;HP][4026:4290:−;HP][4308:4599:−;HP][5524:5974:−;HP][5973:6531:−;HP][6709:6934:−;HP][6988:7222:−;HP][7205:7574:−;HP][7570:7723:−;HP][9625:9976:−;HP][9992:10178:−;HP][10177:10357:−;HP][10343:10523:−;HP][10598:11054:−;HP][11053:11290:−;HP][11286:11961:−;Deoxynucleotide monophosphate kinase][12028:12631:−;Tail tube terminator protein][12630:13458:−;HP][13457:13907:−;HP][13956:14595:+;Baseplate wedge protein gp53][14591:16322:+;Baseplate central spike complex protein gp5][16324:16828:+;HP][16828:17122:+;Baseplate puncturing device gp5.4][17121:19089:+;Baseplate wedge protein gp6][19085:22184:+;Baseplate wedge protein gp7][22176:23202:+;Baseplate wedge protein gp8][23269:24181:+;Baseplate protein gp9][24180:26004:+;Baseplate wedge protein gp10][26005:26677:+;Baseplate wedge protein gp11][26676:28023:+;HP][28031:29798:+;HP][29854:30796:+;Neck protein gp13][30799:31576:+;Neck protein gp14][31617:32442:+;Tail completion protein gp15][32438:32948:+;Terminase %2C small subunit][32925:34764:+;Terminase %2C large subunit][34797:36771:+;Tail sheath protein][37584:38076:+;Tail tube protein gp19][38180:39749:+;Portal protein][39748:39973:+;HP][39972:40398:+;Prohead assembly protein gp68][40397:41048:+;Prohead core protein protease][41080:41893:+;Capsid assembly scaffolding protein][41913:43473:+;Major capsid protein][43554:44835:+;Capsid vertex protein][44864:45413:−;HP][45412:46420:−;RNA ligase 2][46430:46688:−;HP][46801:47128:−;HP][47127:47355:−;HP][47433:48558:−;Highly immunogenic outer capsid protein][48567:49362:−;HP][49939:50923:+;ATP-dependent DNA helicase uvsW][50919:51162:+;ATP-dependent DNA helicase uvsW][51221:51389:−;HP][51419:51836:−;Recombination protein uvsY][51877:52288:−;Baseplate wedge protein gp25][52284:52914:−;HP][52965:53724:+;HP][53720:54863:+;Baseplate central spike complex protein gp27][54834:55362:+;Baseplate hub assembly protein gp28][55358:57092:+;HP][57101:58154:+;Baseplate tail-tube junction protein gp48][58153:59080:+;Baseplate tail-tube junction protein gp54][59101:59380:−;HP][59443:61570:−;HP][61621:61801:−;HP][61797:63270:−;HP][63269:63476:−;HP][63517:64360:−;HP][64543:64744:−;HP][64740:64911:−;HP][64921:65200:−;HP][65212:65698:−;HP][65738:66098:−;HP][66191:66704:−;HP][66743:67112:−;HP][67233:67410:−;HP][67777:68026:−;Lysis inhibition accessory protein][68141:68465:−;Capsid assembly protein Gp31][68522:68831:−;HP][68827:69028:−;HP][69027:69597:−;Deoxycytidylate deaminase][69597:69942:−;HP][69941:70178:−;HP][70167:70452:−;HP][70463:71357:−;Polynucleotide kinase][71356:71569:−;HP][71579:71867:−;HP][71863:72217:−;HP][72204:72714:−;HP][72776:73928:−;T4 RNA ligase 1][73921:74338:−;Endonuclease II][74365:75508:−;HP][75564:77811:−;Ribonucleoside-diphosphate reductase large subunit][77801:78107:−;HP][78103:78310:−;HP][78306:79182:−;Thymidylate synthase][79174:79492:−;HP][79475:80075:−;Dihydrofolate reductase][80082:80247:−;HP][80329:80650:−;HP][80642:80732:−;HP][80728:81007:−;HP][81064:81343:−;HP][81433:81655:−;HP][81696:81954:−;HP][82080:82998:−;Single-stranded DNA-binding protein][83066:83729:−;Protein Gp59][83725:84025:−;RNA polymerase-associated protein Gp33][84005:84278:−;Double-stranded DNA-binding protein][84288:85215:−;Ribonuclease H][85290:89136:+;Long-tail fiber proximal subunit][89135:90272:+;Long-tail fiber protein gp35][90329:91022:+;Long-tail fiber protein gp36][91031:95660:+;HP][95696:96224:+;HP][96265:96922:+;Holin][96922:97195:−;10 kDa anti-sigma factor][97263:97530:−;HP][97526:97853:−;HP][97849:98128:−;HP][98217:98520:−;HP][98519:98882:−;HP][98869:99049:−;HP][99045:99387:−;HP][99386:100031:−;Middle transcription regulatory protein motA][100155:100419:−;HP][100646:102035:−;HP][102040:102181:−;HP][102177:102285:−;HP][102350:102806:−;Nucleoid disruption protein][102864:102954:−;HP][102950:103142:−;HP][103152:103269:−;HP][103329:103569:−;HP][103633:104212:−;Endonuclease IV][104153:104450:−;HP][104492:105404:−;HP][105404:107573:−;Protein rIIA][107583:107793:−;HP][108074:108227:−;HP][108267:110106:−;HP][110136:110505:−;HP][110583:110832:−;HP][110828:110918:−;HP][110917:111094:−;HP][111080:111416:−;HP][111409:111775:−;HP][111774:111990:−;HP][112054:112462:−;HP][112525:112693:−;HP][112692:113382:−;Exodeoxyribonuclease][113374:113629:−;HP][113635:114967:−;ATP-dependent DNA helicase dda][114963:115278:−;HP][115277:116033:−;HP][116112:116778:−;HP][116850:117024:−;HP][117525:117717:−;HP][117795:118023:−;Small outer capsid protein][118053:118191:−;HP][118190:118712:−;HP][118778:118970:+;HP][118988:120011:−;DNA primase][120049:120214:−;HP][120229:120439:−;HP][120435:120729:−;Protein spackle][120739:121018:−;HP][121083:121317:−;HP][121326:122700:−;ATP-dependent helicase 41][122775:123126:−;HP][123115:124291:−;Recombination and repair protein][124373:124595:−;HP][124621:125458:−;HP][125454:126189:−;Deoxycytidylate 5-hydroxymethyltransferase][126197:126404:−;Immunity protein][126414:126858:−;HP][126913:129622:−;DNA-directed DNA polymerase][129714:130077:−;Translation repressor protein][130079:130649:−;Sliding-clamp-loader gp44 subunit][130645:131608:−;Sliding-clamp-loader gp44 subunit][131656:132358:−;DNA polymerase clamp][132393:132771:−;15 kDa RNA polymerase-binding protein][132781:132976:−;HP][132972:134613:−;Exonuclease subunit 2][134651:135674:−;Exonuclease subunit 1][135744:136941:−;DNA alpha-glucosyltransferase][137010:137268:−;HP][137341:137539:−;HP][137516:137819:−;HP][137820:138051:−;HP][138034:138583:−;RNA polymerase sigma factor][138649:138895:−;HP][138891:139170:−;HP][139157:139382:−;HP][139374:139659:−;HP][139651:139909:−;HP][139905:140238:−;HP][140302:140545:−;HP][140541:140730:−;HP][140915:141218:−;HP][141214:141556:−;HP][141552:141765:−;HP][141773:141872:−;HP][141882:142353:−;HP][142324:142558:−;HP][142529:144368:−;Anaerobic ribonucleoside-triphosphate reductase][144364:144838:−;Recombination endonuclease VII][144874:145183:−;HP][145175:145628:−;HP][145611:145770:−;HP][145748:146138:−;HP][146139:146310:−;HP][146328:146517:−;HP][146509:146779:−;Glutaredoxin][146741:147032:−;HP][147021:147195:−;HP][147187:147700:−;HP][147696:148029:−;HP][148025:148340:−;HP][148336:148573:−;HP][148593:149568:−;HP][149638:150427:−;HP][150484:150751:−;HP][150747:150912:−;HP][151274:151472:−;HP][151474:151795:−;HP][151791:152127:−;HP][152123:152549:−;HP][152545:152692:−;HP][152734:153712:−;HP][153829:154069:−;HP][154258:155269:−;HP][155265:155688:−;HP][155880:156462:−;HP][156458:156950:−;HP][156952:157174:−;HP][157163:157355:−;HP][157357:157624:−;HP][157721:158204:−;HP][158308:158695:−;HP][158681:158969:−;Antiholin][158980:159193:−;HP][159239:159614:−;HP][160073:160673:−;HP][160672:160840:−;HP][160864:161332:−;HP][161340:161733:−;HP][161729:162050:−;HP][162049:162595:−;HP][162602:163061:−;Endoribonuclease RegB][163124:163400:−;HP][163399:163762:−;HP][163770:164262:−;HP][164243:164555:−;HP][164551:165085:−;HP][165164:165470:−;HP][165660:166233:−;Internal protein III][166314:166689:−;HP]

Essential genes of phage 395-D2-3-4: [2551:4477:−;Baseplate wedge protein gp6][4508:6575:−;HP][6576:8346:−;Baseplate central spike complex protein gp5][8342:8702:−;Baseplate wedge protein gp53][9055:9415:+;HP][9430:10285:+;HP][10321:11110:−;HP][11479:12010:+;Tail tube terminator protein][12009:12705:+;Deoxynucleotide monophosphate kinase][13031:13655:+;HP][13662:14202:+;HP][14266:14407:+;HP][14496:14637:+;HP][14647:14983:+;HP][14979:15273:+;HP][15265:15517:+;HP][15759:16167:+;HP][16177:16561:+;H P][16562:16820:+;HP][16848:17172:+;HP][17171:17699:+;HP][18686:18887:+;HP][20042:20300:+;HP][20383:20662:+;HP][20733:20985:+;HP][20981:21128:+;HP][21390:21645:+;HP][22378:23533:+;HP][23815:24181:+;HP][24161:24509:+;HP][24505:24937:+;HP][24938:25202:+; HP][25274:25538:+;HP][25574:25895:+;HP][25950:26349:+;HP][26416:26812:+;L-alanyl-D-glutamate peptidase][26843:27302:+;HP][27301:27469:+;HP][27527:28874:+;HP][28911:29109:+;HP][29121:29676:+;HP][29740:30382:+;HP][30449:30845:+;HP][30855:31002:+;HP][30998:31589:+; HP][31598:31922:+;HP][32370:32844:+;HP][32846:33134:+;HP][33126:33735:+;HP][33751:34144:+;HP][34250:34532:+;HP][34503:34926:+;HP][34997:35912:+;HP][36088:37147:+;HP][37215:37485:+;HP][38089:38317:+;HP][38313:38691:+;HP][39508:40516:+;HP][40512:40758: +;HP][40757:41015:+;HP][41011:41923:+;HP][41981:42260:+;HP][42680:42968:+;HP][43193: 43709:+;HP][43708:44029:+;HP][44036:46253:+;HP][46324:46942:+;HP][47093:47564:+;HP][47579:47864:+;HP][48221:48446:+;HP][48453:48909:+;HP][48943:49336:−;Baseplate wedge protein gp25][49336:49891:−;HP][49887:50628:−;HP][50656:51520:−;Baseplate tail-tube junction protein gp54][51529:52618:−;Baseplate tail-tube junction protein gp48][52627:54415:−;HP][54411:54987:−;HP][55056:56166:+;HP][56162:56540:−;HP][56848:57139:−;HP][57876:59406:−;HP][59386:59917:−;HP][59997:60219:−;HP][60220:60484:−;HP][60492:61011:−;HP][61007:61448:−;HP][62148:62442:−;HP][62567:62837:−;HP][62836:63199:−;HP][63209:63776:−;HP][63817:64237:−;HP][64246:64528:−;HP][64842:65094:−;HP][65104:65446:−;Capsid assembly protein Gp31][65461:66019:−;Deoxycytidylate deaminase][66234:66486:−;HP][66482:66770:−;HP][66822:67707:−;Polynucleotide kinase][67703:67877:−;HP][68172:68484:−;HP][68492:69650:−;T4 RNA ligase 1][69630:69999:−;Endonuclease II][70562:71738:−;Ribonucleoside-diphosphate reductase subunit beta][71774:74027:−;Ribonucleoside-diphosphate reductase large subunit][74066:74942:−;Thymidylate synthase][74943:75522:−;HP][75518:75776:−;HP][75772:76003:−;HP][75999:76290:−;HP][76349:78059:−;HP][78132:78324:−;HP][78323:80021:−;Exonuclease subunit 2][80278:81325:−;Exonuclease subunit 1][81680:81896:−;HP][81892:82420:−;RNA polymerase sigma factor][82724:83660:+;Ribonuclease H][83669:83933:+;Double-stranded DNA-binding protein][83936:84182:+;RNA polymerase-associated protein Gp33][84259:84838:+;Protein Gp59][84853:85825:+;HP][85880:86150:+;HP][86176:86842:+;DNA polymerase clamp][86900:87899:+;Sliding-clamp-loader gp44 subunit][87901:88465:+;Sliding-clamp-loader gp44 subunit][88468:88831:+;Translation repressor protein][88899:89187:+;HP][89559:92259:+;DNA-directed DNA polymerase][92333:93491:+;Recombination and repair protein][93528:93849:+;HP][93858:95295:+;ATP-dependent helicase 41][95693:95948:+;HP][96040:96277:+;HP][96646:97378:+;HP][97383:97872:+;HP][97868:98087:+;HP][98142:98403:+;HP][98406:99432:+;DNA primase][99439:99976:+;HP][100157:100448:+;HP][100444:101773:+;ATP-dependent DNA helicase dda][101797:102106:+;HP][102106:102391:+;HP][102387:103059:+;Exodeoxyribonuclease][103055:103337:+;HP][103553:103778:+;HP][103774:104077:+;HP][104786:105116:+;HP][105155:105752:+;HP][105748:106027:+;HP][106013:106598:+;HP][106620:107061:+;HP][107060: 107225:+;HP][107292:109191:+;HP][109190:110477:+;HP][110548:111103:+;HP][111106:111343:+;HP][111353:113615:+;HP][113614:114514:+;HP][114559:114934:+;HP][114908:115346:+;HP][115382:115631:+;HP][115675:115810:+;HP][115784:116117:+;HP][116113:116341:+; HP][116421:117219:+;HP][117218:117509:+;HP][117775:118423:−;Holin][118945:122659:−;HP][122667:123336:−;Long-tail fiber protein gp36][123385:124510:−;Long-tail fiber protein gp35][124509:128283:−;Long-tail fiber proximal subunit][128362:128662:−;HP][128658:128940:−;Glutaredoxin][128899:129568:−;HP][129610:130570:−;HP][130639:131131:−;HP][131795:131987:−;HP][132319:132526:−;HP][132525:133020:−;HP][133327:135451:−;Anaerobic ribonucleoside-triphosphate reductase][135770:136253:−;Recombination endonuclease VII][136285:136555:−;Glutaredoxin][136563:137415:−;DNA adenine methylase][137700:139134:−;HP][139161:139611:−;HP][139620:139881:−;HP][140470:141058:−;HP][141047:141566:−;HP][141569:142589:−;RNA ligase 2][142592:143117:−;HP][143116:143473:−;HP][143474:143801:−;HP][143797:143974:−;HP][143970:144288:−;HP][144320:145196:−;HP][145310:146150:+;HP][146199:146664:+;HP][146696:147215:−;HP][147223:147505:−;HP][147515:148049:−;Highly immunogenic outer capsid protein][148077:148647:−;HP][148686:148998:−;HP][149090:149714:−;HP][149943:151284:+;ATP-dependent DNA helicase uvsW][151292:151526:+;ATP-dependent DNA helicase uvsW][151827:152004:−;HP][152003:152423:−;Recombination protein uvsY][152727:154017:−;Capsid vertex protein][154049:154688:−;HP][154779:156354:−;Major capsid protein][156373:157162:−;Capsid assembly scaffolding protein][157194:157842:−;Prohead core protein protease][157844:158249:−;Prohead assembly protein gp68][158499:160074:−;Portal protein][160129:160615:−;Tail tube protein gp19][160663:162655:−;Tail sheath protein][162674:164504:−;Terminase %2C large subunit][164472:165012:−;Terminase %2C small subunit][165011:165788:−;Tail completion protein gp15][166526:167273:−;Neck protein gp14][167282:168209:−;Neck protein gp13][168247:169999:−;Fibritin][170012:171398:−;Short tail fiber protein gp12][171407:172076:−;Baseplate wedge protein gpl1][172075:173893:−;Baseplate wedge protein gp10][173889:174753:−;Baseplate protein gp9][174762:175755:−;Baseplate wedge protein gp8][175755:176415:−;Baseplate wedge protein gp7]

Essential genes of phage CT-120-2-11: [224:749:+;HP][758:2360:+;HP][2405:3716:+;HP][3705:4473:+;HP][4472:5603:+;HP][5654:6164:+;HP][6277:7237:+;HP][7328:7616:+;HP][7669:8086:+;HP][8091:8469:+;HP][8461:8899:+; HP][8888:9323:+;HP][9309:9996:+;HP][10048:10702:+;HP][10778:11105:+;HP][11455:14422: +;HP][14424:14769:+;HP][14839:15592:+;Tail tip protein L][15593:16331:+;HP][16305:16908:+;HP][16995:20442:+;HP][24262:24727:−;HP][24763:25420:−;HP][25479:26526:−;HP][27021:27981:−;HP][28056:28458:−;HP][28549:30586:+;HP][30582:30990:+;HP][31167:31896:+;HP][31899:32136:+;HP][32200:32437:+;HP][32433:32685:+;HP][32780:33911:+;HP][33949:34438:+;HP][34434:35016:+;HP][35142:35358:+;HP][35359:35842:+;Lysozyme][35838:36264:+;HP][36333:36795:−;HP][36798:38367:−;HP][38492:38930:−;HP][38930:39047:−;HP][39102:39312:−;HP][39384:40077:−;HP][40073:40301:−;HP][40373:40751:−;HP][40750:40990:−;HP][40986:41181:−;HP][41253:41535:−;HP][42199:42733:+;HP][42735:43041:+;HP][43113:43266:+;HP][44099:44312:+;HP][44324:44582:+;HP][44578:44776:+;HP][44849:45221:+;HP][45230:45533:+;HP][45532:45778:+;HP][45774:45960:+;HP][46040:46259:+;HP][46258:46747:+;HP][46822:47143:+;HP][47208:47400: +;HP][47511:47724:+;HP][47720:48296:+;HP][48365:48602:+;HP][48589:48823:+;HP]

Essential genes of phage KP10-85: [373:931:+;Deoxycytidylate deaminase][946:1288:+;Capsid assembly protein Gp31][1298:1550:+;HP][1865:2147:+;HP][2156:2579:+;HP][2853:3186:+;HP][3196:3559:+;HP][3558:3828:+;HP][3954:4248:+;HP][4877:5318:+;HP][5314:5578:+;HP][5579:5801:+;HP][5881:6412:+;HP][6392:7922:+;HP][8657:8930:+;HP][9343:9643:+;HP][9629:10007:+;HP][10003: 11113:−;HP][11182:11758:+;HP][11754:13536:+;HP][13546:14620:+;Baseplate tail-tube junction protein gp48][14629:15493:+;Baseplate tail-tube junction protein gp54][15521:16265:+;HP][16261:16816:+;HP][16816:17209:+;Baseplate wedge protein gp25][17243:17699:−;HP][17706:17931:−;HP][18288:18573:−;HP][18588:19059:−;HP][19210:19828:−;HP][19899:22116:−;HP][22123:22444:−;HP][22443:22959:−;HP][23184:23472:−;HP][23678:23957:−;HP][24015:24927:−;HP][24923:25181:−;HP][25180:25426:−;HP][25422:26430:−;HP][26429:26807:−;HP][26803:27031:−;HP][27635:27908:−;HP][28273:29335:−;HP][29511:30426:−;HP][30497:30920:−;HP][30891:31173:−;HP][31279:31672:−;HP][31688:32297:−;HP][32479:32695:−;HP][32687:32975:−;HP][32977:33451:−;HP][33757:34081:−;HP][34090:34681:−;HP][34834:35230:−;HP][35297:35939:−;HP][36003:36558:−;HP][36569:36767:−;HP][36803:38150:−;HP][38159:38684:−;HP][38683:39142:−;HP][39173:39569:−;L-alanyl-D-glutamate peptidase][39636:40038:−;HP][40092:40416:−;HP][40455:40719:−;HP][40788:41049:−;HP][41050:41482:−;HP][41662:42010:−;HP][41990:42356:−;HP][42638:43784:−;HP][43794:44001:−;HP][43997:44261:−;HP][44832:45084:−;HP][45155:45434:−;HP][45913:46171:−;HP][47326:47527:−;HP][48514:49042:−;HP][49041:49365:−;HP][49393:49651:−;HP][49652:50036:−;HP][50046:50454:−;HP][50696:50948:−;HP][50940:51234:−;HP][51230:51566:−;HP][51576:51717:−;HP][51806:51947:−;HP][52012:52552:−;HP][52559:53183:−;HP][53509:54205:−;Deoxynucleotide monophosphate kinase][54204:54735:−;Tail tube terminator protein][55104:55893:+;HP][55929:56784:−;HP][56799:57159:−;HP][57512:57872:+;Baseplate wedge protein gp53][57868:59638:+;Baseplate central spike complex protein gp5][59639:61706:+;HP][61737:63663:+;Baseplate wedge protein gp6][63743:66830:+;Baseplate wedge protein gp7][66830:67823:+;Baseplate wedge protein gp8][67832:68696:+;Baseplate protein gp9][68692:70510:+;Baseplate wedge protein gp10][70509:71178:+;Baseplate wedge protein gp11][71187:72573:+;Short tail fiber protein gp12][72583:74335:+;Fibritin][74373:75300:+;Neck protein gp13][75309:76056:+;Neck protein gp14][76135:76960:+;Tail completion protein gp15][76959:77499:+;Terminase %2C small subunit][77467:79297:+;Terminase %2C large subunit][79316:81308:+;Tail sheath protein][81356:81842:+;Tail tube protein gp19][81897:83472:+;Portal protein][83723:84128:+;Prohead assembly protein gp68][84130:84778:+;Prohead core protein protease][84810:85599:+;Capsid assembly scaffolding protein][85618:87187:+;Major capsid protein][87272:87908:+;HP][87940:89230:+;Capsid vertex protein][89534:89954:+;Recombination protein uvsY][89953:90130:+;HP][90734:90968:−;ATP-dependent DNA helicase uvsW][90976:92317:−;ATP-dependent DNA helicase uvsW][92546:93125:+;HP][93217:93529:+;HP][93568:94138:+;HP][94166:94700:+;Highly immunogenic outer capsid protein]194710:94986:+;Highly immunogenic outer capsid protein][94999:95518:+;HP][95550:96015:−;HP][96064:96904:−;HP][97018:97894:+;HP][97926:98244:+;HP][98240:98417:+;HP][98413:98740:+;HP][98741:99098:+;HP][99097:99622:+;HP][99625:100645:+;RNA ligase 2][100634:101216:+;HP][101805:102066:+;HP][102075:102525:+;HP][102552:103986:+;HP][104271:105123:+;DNA adenine methylase][105131:105407:+;Glutaredoxin][105438:105921:+;Recombination endonuclease VII][106240:108364:+;Anaerobic ribonucleoside-triphosphate reductase][108670:109168:+;HP][109167:109374:+;HP][109706:109901:+;HP][109897:110425:+;HP][110421:110913:+;HP][110982:111942:+;HP][111984:112653:+;HP][112612:112894:+; Glutaredoxin][112890:113190:+;HP][113269:117043:+;Long-tail fiber proximal subunit][117042:118167:+;Long-tail fiber protein gp35][118216:118885:+;Long-tail fiber protein gp36][118893:122622:+;HP][123303:123951:+;Holin][124217:124508:−;HP][124507:125305:−;HP][125385:125613:−;HP][125609:125939:−;HP][125913:126051:−;HP][126322:126571:−;HP][126607:127045:−;HP][127019:127394:−;HP][127439:128339:−;HP][128338:130600:−;HP][130610:130847:−;HP][130850:131405:−;HP][131476:132763:−;HP][132762:134661:−;HP][134728:134893:−;HP][134892:135333:−;HP][135355:135940:−;HP][135926:136205:−;HP][136201:136798:−;HP][136837:137167:−;HP][137875:138178:−;HP][138615:138897:−;HP][138893:139565:−;Exodeoxyribonuclease][139561:139846:−;HP][139846:140155:−;HP][140179:141508:−;ATP-dependent DNA helicase dda][141504:141795:−;HP][141976:142513:−;HP][142520:143546:−;DNA primase][143549:143810:−;HP][143865:144084:−;HP][144080:144569:−;HP][144574:145306:−;HP][145675:145912:−;HP][146003:146258:−;HP][146650:148087:−;ATP-dependent helicase 41][148096:148417:−;HP][148452:149610:−;Recombination and repair protein][149684:152384:−;DNA-directed DNA polymerase][152756:153044:−;HP][153112:153475:−;Translation repressor protein][153478:154042:−;Sliding-clamp-loader gp44 subunit][154044:155043:−;Sliding-clamp-loader gp44 subunit][155101:155767:−;DNA polymerase clamp][155793:156063:−;HP][156118:157090:−;HP][157105:157684:−;ProteinGp59][157761:158007:−;RNApolymerase-associated protein Gp33][158010:158274:−;Double-stranded DNA-binding protein][158283:159219:−;Ribonuclease H][159523:160051:+;RNA polymerase sigma factor][160047:160263:+;HP][160618:161665:+;Exonuclease subunit 1][161922:163614:+;Exonuclease subunit 2][164376:164571:+;HP][164644:166354:+;HP][166413:166704:+;HP][166700:166931:+;HP][166927:167185:+;HP][167181:167760:+;HP][167761:168637:+;Thymidylate synthase]1168676:170929:+;Ribonucleoside-diphosphate reductase large subunit][170965:172141:+;Ribonucleoside-diphosphate reductase subunit beta][172436:172802:+;Endonuclease II]1172782:173940:+;T4 RNA ligase 1][173948:174260:+;HP][174256:174574:+;HP][174558:174732:+;HP][174728:175613:+;Polyn ucleotide kinase][175665:175953:+;HP]

Essential genes of phage KP10-87: [236:860:−;HP][1186:1882:−;Deoxynucleotide monophosphate kinase][1881:2412:−;Tail tube terminator protein][2781:3570:+;HP][3606:4461:−;HP][4476:4944:−;HP][5189:5549:+;Baseplate wedge protein gp53][5545:7315:+;Baseplate central spike complex protein gp5][7319:9386:+;HP][9417:11343:+;Baseplate wedge protein gp6][11423:14510:+;Baseplate wedge protein gp7][14510:15503:+;Baseplate wedge protein gp8][15512:16376:+;Baseplate protein gp9][16372:18190:+;Baseplate wedge protein gp10][18189:18858:+;Baseplate wedge protein gp11][18867:20253:+;Short tail fiber protein gp12][20266:22018:+;Fibritin][22056:22983:+;Neck protein gp13][22992:23739:+;Neck protein gp14][23819:24644:+;Tail completion protein gp15][24643:25183:+;Terminase %2C small subunit][25151:26981:+;Terminase %2C large subunit][27000:28992:+;Tail sheath protein][29040:29526:+;Tail tube protein gp19][29581:31156:+;Portal protein][31406:31811:+;Prohead assembly protein gp68][31813:32461:+;Prohead core protein protease][32493:33282:+;Capsid assembly scaffolding protein][33301:34870:+;Major capsid protein][34955:35591:+;HP][35623:36913:+;Capsid vertex protein][37217:37637:+;Recombination protein uvsY][37636:37813:+;HP][38142:38376:−;ATP-dependent DNA helicase uvsW][38384:39725:−;ATP-dependent DNA helicase uvsW][39954:40578:+;HP][40670:40982:+;HP][41021:41591:+;HP][42691:42967:+;Highly immunogenic outer capsid protein][42980:43499:+;HP][43531:43996:−;HP][44045:44885:−;HP][44999:45875:+;HP][45907:46225:+;HP][46221:46398:+;HP][46394:46721:+;HP][46722:47079:+;HP][47078:47603:+;HP][47606:48626:+;RNA ligase 2][48615:49197:+;HP][49786:50047:+;HP][50056:50506:+;HP][50533:51967:+;HP][52252:53104:+;DNA adenine methylase][53112:53388:+;Glutaredoxin][53419:53902:+;Recombination endonuclease VII][53898:56022:+;Anaerobic ribonucleoside-triphosphate reductase][56328:56826:+;HP][56825:57032:+;HP][57364:57559:+;HP][57555:58083:+;HP][58079:58571:+;HP][58640:59600:+;HP][59642:60311:+;HP][60270:60552:+;Glutaredoxin][60548 :60848:+;HP][60927:64701:+;Long-tail fiber proximal subunit][64700:65825:+;Long-tail fiber protein gp35][65874:66543:+;Long-tail fiber protein gp36][66551:70517:+;HP][71199:71847:+;Holin][72113:72404:−;HP][72403:73201:−;HP][73281:73509:−;HP][73505:73838:−;HP][73812:73947:−;HP][73991:74240:−;HP][74276:74714:−;HP][74688:75063:−;HP][75108:76008:−;HP][76007:78269:−;HP][78279:78516:−;HP][78519:79074:−;HP][79145:80432:−;HP][80431:82330:−;HP][82397:82562:−;HP][82561:83002:−;HP][83024:83609:−;HP][83595:83874:−;HP][83870:84467:−;HP][84506:84836:−;HP][85545:85848:−;HP][85844:86069:−;HP][86285:86567:−;HP][86563:87235:−;Exodeoxyribonuclease][87231:87516:−;HP][87516:87825:−;HP][87849:89178:−;ATP-dependent DNA helicase dda][89174:89465:−;HP][90518:91055:−;HP][91062:92088:−;DNA primase][92091:92352:−;HP][92407:92626:−;HP][92622:93111:−;HP][93116:93848:−;HP][94211:94448:−;HP][94539:94794:−;HP][95384:96821:−;ATP-dependent helicase 41][96830:97151:−;HP][97187:98345:−;Recombination and repair protein][98419:101119:−;DNA-directed DNA polymerase][101491:101779:−;HP][101847:102210:−;Translation repressor protein][102213:102777:−;Sliding-clamp-loader gp44 subunit][102779:103778:−;Sliding-clamp-loader gp44 subunit][103836:104502:−;DNA polymerase clamp][104528:104798:−;HP][104853:105825:−;HP][105840:106419:−;Protein Gp59][106496:106742:−;RNA polymerase-associated protein Gp33][106745:107009:−;Double-stranded DNA-binding protein][107018:107954:−;Ribonuclease H][108258:108786:+;RNA polymerase sigma factor][108782:108998:+;HP][109353:110400:+;Exonuclease subunit 1][110657:112355:+;Exonuclease subunit 2][112354:112546:+;HP][112619:114329:+;HP][114388:114679:+;HP][114675:114906:+;HP][114902:115160:+;HP][115156:115735:+;HP][115736:116612:+;Thymidylate synthase][116651:118904:+;Ribonucleoside-diphosphate reductase large subunit][118940:120116:+;Ribonucleoside-diphosphate reductase subunit beta][120411:120777:+;Endonuclease H1][120757:121915:+;T4 RNA ligase 1][121923:122235:+;HP][122530:122704:+;HP][122700:123585:+;Polynucleotide kinase][123637:123925:+;HP][123921:124173:+;HP][124388:124946:+;Deoxycytidylate deaminase][124961:125303:+;Capsid assembly protein Gp31][125313:125565:+;HP][125879:126161:+;HP][126170:126593:+;HP][126634:127201:+; HP][127211:127574:+;HP][127573:127843:+;HP][127967:128261:+;HP][128890:129331:+;HP][129327:129591:+;HP][129592:129814:+;HP][129894:130425:+;HP][130405:131935:+;HP][133293:133584:+;HP][133892:134270:+;HP][134266:135376:−;HP][135445:136021:+;HP][136017:137799:+;HP][137809:138883:+;Baseplate tail-tube junction protein gp48][138892:139756:+;Baseplate tail-tube junction protein gp54][139784:140528:+;HP][140524:141079:+;HP][141079:141472:+;Baseplate wedge protein gp25][141506:141962:−;HP][141969:142194:−;HP][142551:142836:−;HP][142851:143322:−;HP][143473:144091:−;HP][144162:146379:−;HP][146386:146707:−;HP][146706:147222:−;HP][147447:147735:−;HP][147941:148220:−;HP][148278:148812:−;HP][148793:149717:−;HP][149713:149971:−;HP][149970:150216:−;HP][150212:151220:−;HP][151219:151597:−;HP][151593:151821:−;HP][152425:152695:−;HP][152763:153822:−;HP][153998:154913:−;HP][154984:155407:−;HP][155378:155660:−;HP][155766:156159:−;HP][156175:156784:−;HP][156966:157254:−;HP][157256:157730:−;HP][158178:158502:−;HP][158511:159102:−;HP][159098:159245:−;HP][159312:159954:−;HP][160018:160573:−;HP][160585:160783:−;HP][160820:162167:−;HP][162176:162701:−;HP][162700:163159:−;HP][163190:163586:−;L-alanyl-D-glutamate peptidase][163654:164056:−;HP][164110:164434:−;HP][164473:164737:−;HP][164806:165067:−;HP][165068:165500:−;HP][165947:166295:−;HP][166275:166620:−;HP][166616:167762:−;HP][167772:167979:−;HP][167975:168239:−;HP][168810:169062:−;HP][169133:169412:−;HP][169495:169753:−;HP][170908:171109:−;HP][172096:172624:−;HP][172623:172947:−;HP][172975:173233:−;HP][173234:173618:−;HP][173628:174036:−;HP][174278:174530:−;HP][174522:174816:−;HP][174812:175148:−;HP][175158:175299:−;HP][175594:175951:−;HP]

Essential genes of phage KP10-94: [1027:1675:+;Holin][1941:2232:−;HP][2231:3029:−;HP][3109:3337:−;HP][3333:3666:−;HP][3640:3778:−;HP][3822:4071:−;HP][4107:4545:−;HP][4519:4894:−;HP][4939:5839:−;HP][5838:8100:−;HP][8110:8347:−;HP][8350:8905:−;HP][8976:10263:−;HP][10262:12161:−;HP][12228:12393:−;HP][12392:12833:−;HP][12855:13449:−;HP][13435:13714:−;HP][13710:14307:−;HP][14346:14676:−;HP][15385:15688:−;HP][15684:15909:−;HP][16125:16407:−;HP][16403:17075:−;Exodeoxyribonuclease][17071:17356:−;HP][17356:17665:−;HP][17689:19018:−;ATP-dependent DNA helicase dda][19014:19305:−;HP][20358:20895:−;HP][20902:21928:−;DNA primase][21931:22192:−;HP][22247:22466:−;HP][22462:22951:−;HP][22956:23688:−;HP][24051:24288:−;HP][24381:24636:−;HP][25028:26465:−;ATP-dependent helicase 41][26474:26795:−;HP][26832:27990:−;Recombination and repair protein][28064:30764:−;DNA-directed DNA polymerase][31136:31424:−;HP][31492:31855:−;Translation repressor protein][31858:32422:−;Sliding-clamp-loader gp44 subunit][32424:33423:−;Sliding-clamp-loader gp44 subunit][33481:34147:−;DNA polymerase clamp][34173:34443:−;HP][34498:35470:−;HP][35485:36064:−;Protein Gp59][36141:36387:−;RNA polymerase-associated protein Gp33][36390:36654:−;Double-stranded DNA-binding protein][36663:37599:−;Ribonuclease H][37903:38431:+;RNA polymerase sigma factor][38427:38643:+;HP][38998:40045:+;Exonuclease subunit 1][40302:42000:+;Exonuclease subunit 2][41999:42191:+;HP][42264:43974:+;HP][44033:44324:+;HP][44320:44551:+;HP][44547:448 05:+;HP][44801:45380:+;HP][45381:46257:+;Thymidylate synthase][46296:48549:+;Ribonucleoside-diphosphate reductase large subunit][48585:49761:+;Ribonucleoside-diphosphate reductase subunit beta][50056:50422:+;Endonuclease II][50402:51560:+;T4 RNA ligase 1][51568:51880:+;HP][52175:52349:+;HP][52345:53230:+;Polynucleotide kinase][53283:53571:+;HP][53567:53819:+;HP][54034:54592:+;Deoxycytidylate deaminase][54607:54949:+;Capsid assembly protein Gp31][54959:55211:+;HP][55525:55807:+;HP][55816:56239:+;HP][56280:56847:+;HP][56857 :57220:+;HP][57219:57489:+;HP][57614:57908:+;HP][58537:58978:+;HP][58974:59238:+;HP][59239:59461:+;HP][59541:60072:+;HP][60052:61582:+;HP][62940:63231:+;HP][63539:63917:+;HP][63913:65023:−;HP][65092:65668:+;HP][65664:67446:+;HP][67456:68530:+;Baseplate tail-tube junction protein gp48][68539:69403:+;Baseplate tail-tube junction protein gp54][69431:70175:+;HP][70171:70726:+;HP][70726:71119:+;Baseplate wedge protein gp25][71153:71609:−;HP][71616:71841:−;HP][72198:72483:−;HP][72498:72969:−;HP][73120:73738:−;HP][73807:76024:−;HP][76031:76352:−;HP][76351:76867:−;HP][77092:77380:−;HP][77586:77865:−;HP][77923:78835:−;HP][78831:79089:−;HP][79088:79334:−;HP][79330:80338:−;HP][80337:80715:−;HP][80711:80939:−;HP][81543:81813:−;HP][81881:82940:−;HP][83116:84031:−;HP][84102:84525:−;HP][84496:84778:−;HP][84884:85277:−;HP][85293:85902:−;HP][86084:86372:−;HP][86374:86848:−;HP][87145:87469:−;HP][87478:88069:−;HP][88065:88212:−;HP][88222:88618:−;HP][88685:89327:−;HP][89391:89946:−;HP][89958:90156:−;HP][90191:91538:−;HP][91547:92072:−;HP][92071:92530:−;HP][92561:92957:−;L-alanyl-D-glutamate peptidase][93024:93426:−;HP][93480:93804:−;HP][93843:94107:−;HP][94176:94437:−;HP][94438:94870:−;HP][95050:95398:−;HP][95378:95723:−;HP][95719:96874:−;HP][97607:97862:−;HP][97861:98008:−;HP][98004:98256:−;HP][98327:98606:−;HP][98689:98947:−;HP][100102:100303:−;HP][101290:101818:−;HP][101817:102141:−;HP][102169:102427:−;HP][102428:102812:−;HP][102822:103230:−;HP][103472:103724:−;HP][103716:104010:−;HP][104006:104342:−;HP][104352:104493:−;HP][104788:105328:−;HP][105335:105959:−;HP][106285:106981:−;Deoxynucleotide monophosphate kinase][106980:107511:−;Tail tube terminator protein][107880:108669:+;HP][108705:109560:−;HP][109575:109935:−;HP][110288:110648:+;Baseplate wedge protein gp53][110644:112414:+;Baseplate central spike complex protein gp5][112415:114482:+;HP][114513:116439:+;Baseplate wedge protein gp6][116519:119606:+;Baseplate wedge protein gp7][119606:120599:+;Baseplate wedge protein gp8][120608:121472:+;Baseplate protein gp9][121468:123286:+;Baseplate wedge protein gp10][123285:123954:+;Baseplate wedge protein gp11][123963:125349:+;Short tail fiber protein gp12][125359:127111:+;Fibritin][127149:128076:+;Neck protein gp13][128085:128832:+;Neck protein gp14][129570:130347:+;Tail completion protein gp15][130346:130886:+;Terminase %2C small subunit][130854:132684:+;Terminase %2C large subunit][132703:134695:+;Tail sheath protein][134743:135229:+;Tail tube protein gp19][135284:136859:+;Portal protein][137110:137515:+;Prohead assembly protein gp68][137517:138165:+;Prohead core protein protease][138197:138986:+;Capsid assembly scaffolding protein][139005:140574:+;Major capsid protein][140659:141295:+;HP][141327:142617:+;Capsid vertex protein][142918:143338:+;Recombination protein uvsY][143337:143514:+;HP][143948:144179:−;ATP-dependent DNA helicase uvsW][144187:145528:−;ATP-dependent DNA helicase uvsW][145757:146336:+;HP][146428:146740:+;HP][146779:147349:+;HP][148449:148725:+; Highly immunogenic outer capsid protein][148738:149257:+;HP][149288:149753:−;HP][149802:150642:−;HP][150756:151632:+;HP][151664:151982:+;HP][151978:152155:+;HP][152151:152478:+;HP][152479:152836:+;HP][152835:153360:+;HP][153363:154383:+;RNA ligase 2][154386:154905:+;HP][154894:155482:+;HP][156071:156332:+;HP][156341:156791:+;HP][156818:158252:+;HP][158537:159389:+;DNA adenine methylase][159397:159673:+;Glutaredoxin][159704:160187:+;Recombination endonuclease VII][160183:162307:+;Anaerobic ribonucleoside-triphosphate reductase][162614:163109:+;HP][163108:163315:+;HP][164145:164340:+;HP][164336:164864:+;HP][164860:165352:+;HP][165421:166381:+;HP][166423:167092:+;HP][167051:167333:+; Glutaredoxin][167329:167635:+;HP][167707:171481:+;Long-tail fiber proximal subunit][171480:172605:+;Long-tail fiber protein gp35][172654:173323:+;Long-tail fiber protein gp36]

Essential genes of phage KP10-80: [231:432:+;HP][431:1562:+;HP][1613:2123:+;HP][2236:3205:+;HP][3681:3969:+;HP][4022:4439:+;HP][4444:4822:+;HP][4814:5252:+;HP][5241:5673:+;HP][5659:6346:+;HP][6398:7052:+; HP][7128:7455:+;HP][7805:10775:+;HP][10777:11122:+;HP][11191:11944:+;Tail tip protein L][11945:12683:+;HP][12657:13260:+;HP][13347:16773:+;HP][20590:21055:−;HP][21091:21748:−;HP][21807:22854:−;HP][23349:24309:−;HP][24384:24786:−;HP][24877:26914:+;HP][26910:27318:+;HP][27495:28224:+;HP][28227:28464:+;HP][28528:2 8765:+;HP][28761:29013:+;HP][29108:30239:+;HP][30277:30766:+;HP][30762:31344:+;HP][31470:31686:+;HP][31687:32170:+;Lysozyme][32166:32592:+;HP][32661:33123:−;HP][33126:34695:−;HP][34820:35258:−;HP][35258:35375:−;HP][35430:35640:−;HP][35712:36405:−;HP][36401:36629:−;HP][36701:37079:−;HP][37078:37318:−;HP][37314:37509:−;HP][37581:37863:−;HP][38527:39061:+;HP][39063:39369:+;HP][39441:39594:+;HP][40427:40640:+;HP][40652:40910:+;HP][40906:41104:+;HP][41177:41549:+;HP][41558:41861:+;HP][41860:42106:+;HP][42102:42288:+;HP][42368:42587:+;HP][42586:43075:+;HP][43150:43471:+;HP][43536:43728: +;HP][43839:44052:+;HP][44048:44624:+;HP][44693:44930:+;HP][44917:45151:+;HP][45467: 45731:+;HP][45810:46335:+;HP][46344:47946:+;HP][47991:49302:+;HP]

Essential genes of phage KP10-4: [68:713:+;HP][762:1227:+;HP][1259:1778:−;HP][1791:2067:−;Highly immunogenic outer capsid protein][2077:2611:−;Highly immunogenic outer capsid protein][2639:3209:−;HP][3248:3560:−;HP][3651:4230:−;HP][4459:5800:+;ATP-dependent DNA helicase uvsW][5808:6042:+;ATP-dependent DNA helicase uvsW][6369:6546:−;HP][6545:6965:−;Recombination protein uvsY][7269:8559:−;Capsid vertex protein][8646:10215:−;Major capsid protein][10234:11023:−;Capsid assembly scaffolding protein][11055:11703:−;Prohead core protein protease][11705:12110:−;Prohead assembly protein gp68][12361:13936:−;Portal protein][13991:14477:−;Tail tube protein gp19][14525:16517:−;Tail sheath protein][16536:18366:−;Terminase %2C large subunit][18334:18877:−;Terminase %2C small subunit][18873:19698:−;Tail completion protein gp15][19778:20525:−;Neck protein gp14][20534:21461:−;Neck protein gp13][21499:23251:−;Fibritin][23261:24647:−;Short tail fiber protein gp12][24656:25325:−;Baseplate wedge protein gp11][25324:27142:−;Baseplate wedge protein gp10][27138:28002:−;Baseplate protein gp9][28011:29004:−;Baseplate wedge protein gp8][29004:32091:−;Baseplate wedge protein gp7][32171:34097:−;Baseplate wedge protein gp6][34128:36195:−;HP][36196:37966:−;Baseplate central spike complex protein gp5][37962:38322:−;Baseplate wedge protein gp53][38675:39035:+;HP][39050:39905:+;HP][39941:40730:−;HP][41099:41630:+;Tail tube terminator protein][41629:42325:+;Deoxynucleotide monophosphate kinase][42652:43276:+;HP][43283:43823:+;HP][43888:44029:+;HP][44118:44259:+;HP][44269:44605:+;HP][44601:44895:+;HP][44887:45139:+;HP][45381:45789:+;HP][45799:46183:+;H P][46184:46442:+;HP][46470:46803:+;HP][46792:47320:+;HP][48307:48508:+;HP][49663:49924:+;HP][50404:50683:+;HP][50754:51006:+;HP][51758:52016:+;HP][52012:52219:+;HP][52229:53375:+;HP][53371:53719:+;HP][53696:54050:+;HP][54499:54931:+;HP][54932:55193:+; HP][55262:55526:+;HP][55565:55889:+;HP][55943:56345:+;HP][56412:56808:+;L-alanyl-D-glutamate peptidase][56839:57298:+;HP][57297:57822:+;HP][57831:59178:+;HP][59214:59412:+;HP][59423:59978:+;HP][60041:60683:+;HP][60750:61146:+;HP][61299:61890:+;HP][61899:62223:+; HP][62520:62994:+;HP][62996:63284:+;HP][63276:63492:+;HP][63674:64283:+;HP][64299:64692:+;HP][64798:65080:+;HP][65051:65474:+;HP][65545:66460:+;HP][66636:67695:+;HP][67762:68032:+;HP][68636:68864:+;HP][68860:69238:+;HP][69237:70245:+;HP][70241:70487: +;HP][70486:70750:+;HP][70746:71658:+;HP][71716:71995:+;HP][72201:72489:+;HP][72714: 73230:+;HP][73229:73550:+;HP][73557:75774:+;HP][75845:76463:+;HP][76614:77085:+;HP][77100:77385:+;HP][77742:77967:+;HP][77974:78430:+;HP][78464:78857:−;Baseplate wedge protein gp25][78857:79412:−;HP][79408:80152:−;HP][80180:81044:−;Baseplate tail-tube junction protein gp54][81053:82127:−;Baseplate tail-tube junction protein gp48][82137:83931:−;HP][83927:84503:−;HP][84572:85682:+;HP][85678:86056:−;HP][86364:86655:−;HP][88013:89543:−;HP][90624:90846:−;HP][90847:91111:−;HP][91107:91548:−;HP][92248:92542:−;HP][92668:92938:−;HP][92937:93300:−;HP][93310:93877:−;HP][93918:94341:−;HP][94350:94632:−;HP][94945:95197:−;HP][95207:95549:−;Capsid assembly protein Gp31][95564:96119:−;Deoxycytidylate deaminase][96337:96589:−;HP][96585:96873:−;HP][96925:97810:−;Polynucleotide kinase][97806:97980:−;HP][98275:98587:−;HP][98595:99753:−;T4 RNA ligase 1][99733:100099:−;Endonuclease II][100394:101570:−;Ribonucleoside-diphosphate reductase subunit beta][101606:103859:−;Ribonucleoside-diphosphate reductase large subunit][103898:104774:−;Thymidylate synthase][104775:105354:−;HP][105350:105608:−;HP][105604:105835:−;HP][105831:106122:−;HP][106181:107891:−;HP][107964:108156:−;HP][108155:109853:−;Exonuclease subunit 2][110110:111157:−;Exonuclease subunit 1][111512:111728:−;HP][111724:112252:−;RNA polymerase sigma factor][112556:113492:+;Ribonuclease H][113501:113765:+;Double-stranded DNA-binding protein][113768:114014:+;RNA polymerase-associated protein Gp33][114091:114670:+;Protein Gp59][114685:115657:+;HP][115712:115982:+;HP][116008:116674:+;DNA polymerase clamp][116732:117731:+;Sliding-clamp-loader gp44 subunit][117733:118297:+;Sliding-clamp-loader gp44 subunit][118300:118663:+;Translation repressor protein][118729:119017:+;HP][119389:122089:+;DNA-directed DNA polymerase][122163:123321:+;Recombination and repair protein][123358:123679:+;HP][123688:125125:+;ATP-dependent helicase 41][125517:125772:+;HP][125863:126100:+;HP][126469:127201:+;HP][127206:127695:+;HP][127691:127910:+;HP][127965:128226:+;HP][128229:129255:+;DNA primase][129262:129799:+;HP][129980:130271:+;HP][130267:131596:+;ATP-dependent DNA helicase dda][131620:131929:+;HP][131929:132214:+;HP][132210:132882:+;Exodeoxyribonuclease][132878:133160:+;HP][133597:133900:+;HP][134609:134939:+;HP][134978:135575:+;HP][135571:135850:+;HP][135836:136421:+;HP][136443:136884:+;HP][136883:137048:+;HP][137115: 139014:+;HP][139013:140300:+;HP][140371:140926:+;HP][140929:141166:+;HP][141176:143438:+;HP][143437:144337:+;HP][144382:144757:+;HP][144731:145169:+;HP][145205:145454:+;HP][145498:145729:+;HP][145725:145863:+;HP][145837:146167:+;HP][146163:146391:+; HP][146471:147269:+;HP][147268:147559:+;HP][147825:148473:−;Holin][149157:153174:−;HP][153182:153851:−;Long-tail fiber protein gp36][153900:155025:−;Long-tail fiber protein gp35][155024:158798:−;Long-tail fiber proximal subunit][158877:159177:−;HP][159173:159455:−;Glutaredoxin][159414:160083:−;HP][160125:161085:−;HP][161154:161646:−;HP][161642:162170:−;HP][162166:162361:−;HP][163191:163398:−;HP][163397:163895:−;HP][164201:166325:−;Anaerobic ribonucleoside-triphosphate reductase][166321:166804:−;Recombination endonuclease VII][166835:167111:−;Glutaredoxin][167119:167971:−;DNA adenine methylase][168256:169690:−;HP][169717:170167:−;HP][170176:170437:−;HP][171026:171608:−;HP][171597:172617:−;RNA ligase 2][172620:173145:−;HP][173144:173501:−;HP][173502:173829:−;HP][173825:174002:−;HP][173998:174316:−;HP][174348:175224:−;HP]

Essential genes of phage CT-227-KP4M1-7: [1474:2113:−;Baseplate wedge protein gp53][2220:2916:+;HP][2999:3356:+;HP][3355:4183:+;HP][4182:4785:+;Tail tube terminator protein][4852:5527:+;Deoxynucleotide monophosphate kinase][5523:5760:+;HP][5759:6215:+;HP][6290:6470:+;HP][6456:6636:+;HP][6635:6821:+;H P][6837:7188:+;HP][9563:9716:+;HP][9712:10081:+;HP][10064:10298:+;HP][10352:10577:+; HP][10755:11313:+;HP][11312:11762:+;HP][13575:13866:+;HP][13884:14148:+;HP][14134:14362:+;HP][14391:14985:+;HP][15024:15615:+;HP][15611:15974:+;HP][15970:16267:+;HP][16263:16590:+;HP][16589:16826:+;HP][16822:17269:+;HP][17276:17585:+;HP][17614:18109: +;Endolysin][18109:18589:+;HP][18670:19243:+;Internal protein III][19438:19741:+;HP][19927:20353:+;HP][20349:20661:+;HP][20642:20987:+;HP][20995:21358:+;HP][21357:21633:+;HP][21696:22155:+;Endoribonuclease RegB][22162:22708:+;HP][22707:23028:+;HP][23024:23417:+;HP][23425:23893:+;HP][23916 :24084:+;HP][24083:24683:+;HP][24663:24858:+;HP][25142:25526:+;HP][25572:25785:+;HP][25796:26084:+;Antiholin][26070:26457:+;HP][26561:27050:+;HP][27141:27408:+;HP][27410 :27602:+;HP][27591:27813:+;HP][27815:28316:+;HP][28508:28931:+;HP][28927:29938:+;HP][30488:30704:+;HP][30781:31759:+;HP][31801:31948:+;HP][31944:32370:+;HP][32366:32702:+;HP][32698:33019:+;HP][33392:33557:+;HP][33553:33820:+;HP][33877:34666:+;HP][34736:35711:+;HP][35731:35968:+;HP][35964:36279:+;HP][36275:36608:+;HP][36604:37117:+;H P][37109:37283:+;HP][37272:37563:+;HP][37525:37795:+;Glutaredoxin][37787:37976:+;HP][37994:38165:+;HP][38166:38556:+;HP][38676:39129:+;HP][39121:39430:+;HP][39466:39940: +;Recombination endonuclease VII][39936:41775:+;Anaerobic ribonucleoside-triphosphate reductase][41746:41986:+;HP][41951:42584:+;HP][42573:43044:+;HP][43054:43153:+;HP][43161:43374:+;HP][43370:43712:+;HP][43708:44011:+;HP][44196:44385:+;HP][44381:44624:+; HP][44688:45021:+;HP][45017:45275:+;HP][45267:45378:+;HP][45543:45768:+;HP][45755:46034:+;HP][46030:46276:+;HP][46342:46891:+;RNA polymerase sigma factor][46874:47105:+;HP][47106:47409:+;HP][47386:47584:+;HP][47657:47915:+;HP][47984 :49181:+;DNA alpha-glucosyltransferase][49251:50274:+;Exonuclease subunit 1][50312:51953:+;Exonuclease subunit 2][51949:52144:+;HP][52154:52532:+;15 kDa RNA polymerase-binding protein][52567:53269:+;DNA polymerase clamp][53317:54280:+;Sliding-clamp-loader gp44 subunit][54276:54846:+;Sliding-clamp-loader gp44 subunit][54848:55211:+;Translation repressor protein][56019:58728:+;DNA-directed DNA polymerase][58797:59241:+;HP][59251:59458:+;Immunity protein][59466:60201:+;Deoxycytidylate 5-hydroxymethyltransferase][60197:61034:+;HP][61060:61282:+;HP][61364:62540:+;Recombination and repair protein][62529:62880:+;HP][62955:64329:+;ATP-dependent helicase 41][64338:64572:+;HP][64637:64916:+;HP][64926:65220:+;Protein spackle][65216:65426:+;HP][65441:65606:+;HP][65644:66667:+;DNA primase][66685:66877:−;HP][66943:67465:+;HP][67464:67602:+;HP][67632:67860:+;Small outer capsid protein][67938:68130:+;HP][68631:68805:+;HP][68875:69577:+;HP][69657:70413:+;HP][70412:70727:+;HP][70723:72055:+;ATP-dependent DNA helicase dda][72061:72316:+;HP][72308:72998:+;Exodeoxyribonuclease][72997:73165:+;HP][73228:73669:+;HP][73733:73949:+;HP][73948:74314:+;HP][74307:74643:+;HP][74629:74806:+;HP][74805:74895:+;HP][74891:75140:+;HP][75218:75584:+;HP][75614:77453:+;HP][77500:77653:+; HP][77792:77999:+;HP][78009:80178:+;Protein rIIA][80178:81090:+;HP][81132:81429:+;HP][81370:81949:+;Endonuclease IV][82578:83370:+;DNA adenine methylase][83427:83544:+;HP][83554:83746:+;HP][83742:83832:+;HP][83890:84346:+;Nucleoid disruption protein][84411:84519:+;HP][84515:84656:+;HP][84661:86050:+;HP][86290:86935:+;Middle transcription regulatory protein motA][86934:87276:+;HP][87272:87452:+;HP][87439:87802:+;HP][87801:88104:+;HP][88468 :88795:+;HP][88791:89058:+;HP][89125:89398:+;10 kDa anti-sigma factor][89398:90055:−;Holin][90096:90624:−;HP][90661:95098:−;HP][95107:95770:−;Long-tail fiber protein gp36][95827:96964:−;Long-tail fiber protein gp35][96963:100809:−;Long-tail fiber proximal subunit][100884:101811:+;Ribonuclease H][101821:102094:+;Double-stranded DNA-binding protein][102074:102374:+;RNA polymerase-associated protein Gp33][102370:103033:+;Protein Gp59][103100:104018:+;Single-stranded DNA-binding protein][104144:104402:+;HP][104443:104665:+;HP][104755:105034:+;HP][105090:105369:+; HP][105365:105455:+;HP][105447:105768:+;HP][105862:106462:+;Dihydrofolate reductase][106445:106763:+;HP][106755:107631:+;Thymidylate synthase][107627:107834:+;HP][107830:108136:+;HP][108126:110373:+;Ribonucleoside-diphosphate reductase large subunit][110429:111572:+;HP][111599:112016:+;Endonuclease II][112012:112627:+;HP][112620:113772:+;T4 RNA ligase 1][113834:114344:+;HP][114331:114685:+;HP][114681:114969:+;HP][114979:115192:+;HP][115191:116085:+;Polynucleotide kinase][116096:116381:+;HP][116370:116607:+;HP][116606:116951:+;HP][116951:117521:+; Deoxycytidylate deaminase][117520:117721:+;HP][117717:118026:+;HP][118083:118407:+;Capsid assembly protein Gp31][118522:118771:+;Lysis inhibition accessory protein][118966:119110:−;HP][119138:119315:+;HP][119436:119805:+;HP][119845:120328:+;HP][120340:120619:+;HP][120629:120800:+;HP][120796:120997:+;HP][121180:122023:+;HP][122064:122271:+;HP][122270:123743:+;HP][123739:123919:+;HP][123970:126097:+;HP][126160:126439:+;HP][126460:127387:−;Baseplate tail-tube junction protein gp54][127386:128439:−;Baseplate tail-tube junction protein gp48][128448:130182:−;HP][130178:130706:−;Baseplate hub assembly protein gp28][130677:131820:−;Baseplate central spike complex protein gp27][131816:132575:−;HP][132626:133256:+;HP][133252:133663:+;Baseplate wedge protein gp25][133704:134121:+;Recombination protein uvsY][134151:134319:+;HP][134378:134621:−;ATP-dependent DNA helicase uvsW][134617:135601:−;ATP-dependent DNA helicase uvsW][136178:136973:+;HP][136982:137825:+;Highly immunogenic outer capsid protein][137903:138131:+;HP][138127:138457:+;HP][138562:138820:+;HP][138830:139838:+; RNA ligase 2][139837:140386:+;HP][140416:141697:−;Capsid vertex protein][141778:143338:−;Major capsid protein][143358:144171:−;Capsid assembly scaffolding protein][144203:144854:−;Prohead core protein protease]1144853:145279:−;Prohead assembly protein gp68][145278:145503:−;HP][145502:147071:−;Portal protein][147175:147667:−;Tail tube protein gp19][148480:150454:−;Tail sheath protein][150487:152326:−;Terminase %2C large subunit][152303:152813:−;Terminase %2C small subunit][152809:153634:−;Tail completion protein gp15][153675:154452:−;Neck protein gp14][154455:155397:−;Neck protein gp13][155453:157220:−;HP][157228:158575:−;HP][158574:159246:−;Baseplate wedge protein gp11][159247:161071:−;Baseplate wedge protein gp10][161070:161982:−;Baseplate protein gp9][162048:163074:−;Baseplate wedge protein gp8][163066:166165:−;Baseplate wedge protein gp7][166161:168129:−;Baseplate wedge protein gp6][168128:168422:−;Baseplate puncturing device gp5.4][168422:168926:−;HP][168928:169258:−;Baseplate central spike complex protein gp5]

Essential genes of phage 395-D2-3-3: [319:1063:+;HP][1059:1614:+;HP][1614:2007:+;Baseplate wedge protein gp25][2041:2497:−;HP][2504:2729:−;HP][3086:3371:−;HP][3386:3857:−;HP][4008:4626:−;HP][4695:6912:−;HP][6919:7240:−;HP][7239:7755:−;HP][7980:8268:−;HP][8474:8753:−;HP][8811:9345:−;HP][9326:10250:−;HP][10246:10504:−;HP][10503:10749:−;HP][10745:11753:−;HP][11752:12130:−;HP][12126:12354:−;HP][12958:13228:−;HP][13296:14355:−;HP][14531:15446:−;HP][15517:15940:−;HP][15911:16193:−;HP][16299:16692:−;HP][16708:17317:−;HP][17499:17787:−;HP][17789:18263:−;HP][18711:19035:−;HP][19044:19635:−;HP][19631:19778:−;HP][19845:20487:−;HP][20551:21106:−;HP][21118:21316:−;HP][21353:22700:−;HP][22709:23234:−;HP][23233:23692:−;HP][23723:24119:−;L-alanyl-D-glutamate peptidase][24187:24589:−;HP][24643:24967:−;HP][25006:25270:−;HP][25339:25600:−;HP][25601:26033:−;HP][26480:26828:−;HP][26808:27153:−;HP][27149:28295:−;HP][28305:28512:−;HP][28508:28772:−;HP][29343:29595:−;HP][29666:29945:−;HP][30028:30286:−;HP][31441:31642:−;HP][32629:33157:−;HP][33156:33480:−;HP][33508:33766:−;HP][33767:34151:−;HP][34161:34569:−;HP][34811:35063:−;HP][35055:35349:−;HP][35345:35681:−;HP][35691:35832:−;HP][36127:36667:−;HP][36674:37298:−;HP][37624:38320:−;Deoxynucleotide monophosphate kinase][38319:38850:−;Tail tube terminator protein][39219:40008:+;HP][40044:40899:−;HP][40914:41382:−;HP][41627:41987:+;Baseplate wedge protein gp53][41983:43753:+;Baseplate central spike complex protein gp5][43757:45824:+;HP][45855:47781:+;Baseplate wedge protein gp6][47861:50948:+;Baseplate wedge protein gp7][50948:51941:+;Baseplate wedge protein gp8][51950:52814:+;Baseplate protein gp9][52810:54628:+;Baseplate wedge protein gp10][54627:55296:+;Baseplate wedge protein gp11][55305:56691:+;Short tail fiber protein gp12][56704:58456:+;Fibritin][58494:59421:+;Neck protein gp13][59430:60177:+;Neck protein gp14][60257:61082:+;Tail completion protein gp15][61081:61621:+;Terminase %2C small subunit][61589:63419:+;Terminase %2C large subunit][63438:65430:+;Tail sheath protein][65478:65964:+;Tail tube protein gp19][66019:67594:+;Portal protein][67844:68249:+;Prohead assembly protein gp68][68251:68899:+;Prohead core protein protease][68931:69720:+;Capsid assembly scaffolding protein][69739:71308:+;Major capsid protein][71399:72038:+;HP][72070:73360:+;Capsid vertex protein][73664:74084:+;Recombination protein uvsY][74083:74260:+;HP][74694:74928:−;ATP-dependent DNA helicase uvsW][74936:76277:−;ATP-dependent DNA helicase uvsW][76506:77085:+;HP][77177:77489:+;HP][77528:78098:+;HP][79198:79474:+;Highly immunogenic outer capsid protein][79487:80006:+;HP][80038:80503:−;HP][80552:81392:−;HP][81506:82382:+;HP][82414:82732:+;HP][82728:82905:+;HP][82901:83228:+;HP][83229:83586:+;HP][83585:84110:+;HP][84113:85133:+;RNA ligase 2][85122:85704:+;HP][86293:86554:+;HP][86563:87013:+;HP][87040:88474:+;HP][88759:89611:+;DNA adenine methylase][89619:89895:+;Glutaredoxin][89926:90409:+;Recombination endonuclease VII][90405:92529:+;Anaerobic ribonucleoside-triphosphate reductase][92835:93333:+;HP][93332:93539:+;HP][93871:94066:+;HP][94062:94590:+;HP][94586:95078:+;HP][95147:96107:+;HP][96149:96818:+;HP][96777:97059:+;Glutaredoxin][97055 :97355:+;HP][97434:101208:+;Long-tail fiber proximal subunit][101207:102332:+;Long-tail fiber protein gp35][102381:103050:+;Long-tail fiber protein gp36][103058:107024:+;HP][107706:108354:+;Holin][108620:108911:−;HP][108910:109708:−;HP][109788:110016:−;HP][110012:110345:−;HP][110319:110454:−;HP][110498:110747:−;HP][110783:111221:−;HP][111195:111570:−;HP][111615:112515:−;HP][112514:114776:−;HP][114786:115023:−;HP][115026:115581:−;HP][115652:116939:−;HP][116938:118837:−;HP][118904:119069:−;HP][119068:119509:−;HP][119531:120116:−;HP][120102:120381:−;HP][120377:120974:−;HP][121013:121343:−;HP][122052:122355:−;HP][122351:122576:−;HP][122792:123074:−;HP][123070:123742:−;Exodeoxyribonuclease][123738:124023:−;HP][124023:124332:−;HP][124356:125685:−;ATP-dependent DNA helicase dda][125681:125972:−;HP][127025:127562:−;HP][127569:128595:−;DNA primase][128598:128859:−;HP][128914:129133:−;HP][129129:129618:−;HP][129623:130355:−;HP][130718:130955:−;HP][131046:131301:−;HP][131891:133328:−;ATP-dependent helicase 41][133337:133658:−;HP][133694:134852:−;Recombination and repair protein][134926:137626:−;DNA-directed DNA polymerase][137998:138286:−;HP][138354:138717:−;Translation repressor protein][138720:139284:−;Sliding-clamp-loader gp44 subunit][139286:140285:−;Sliding-clamp-loader gp44 subunit][140343:141009:−;DNA polymerase clamp][141035:141305:−;HP][141360:142332:−;HP][142347:142926:−;Protein Gp59][143003:143249:−;RNA polymerase-associated protein Gp33][143252:143516:−;Double-stranded DNA-binding protein][143525:144461:−;Ribonuclease H][144765:145293:+;RNA polymerase sigma factor][145289:145505:+;HP][145860:146907:+;Exonuclease subunit 1][147164:148856:+;Exonuclease subunit 2][149618:149813:+;HP][149886:151596:+;HP][151655:151946:+;HP][151942:152173:+;HP][152169:152427:+;HP][152423:153002:+;HP][153003:153879:+;Thymidylate synthase][153918:156171:+;Ribonucleoside-diphosphate reductase large subunit][156207:157383:+;Ribonucleoside-diphosphate reductase subunit beta][157678:158044:+;Endonuclease H]1158024:159182:+;T4 RNA ligase 1][159190:159502:+;HP][159797:159971:+;HP][159967:160852:+;Polynucleotide kinase][160904:161192:+;HP][161188:161440:+;HP][161655:162213:+;Deoxycytidylate deaminase][162228:162570:+;Capsid assembly protein Gp31][162580:162832:+;HP][163147:163429:+;HP][163438:163858:+;HP][163899:164466:+; HP][164476:164839:+;HP][164838:165108:+;HP][165232:165526:+;HP][166155:166596:+;HP][166592:166856:+;HP][166857:167079:+;HP][167159:167690:+;HP][167670:169200:+;HP][170558:170849:+;HP][171157:171535:+;HP][171531:172641:−;HP][172710:173286:+;HP][173282:175064:+;HP][175074:176148:+;Baseplate tail-tube junction protein gp48]

Essential genes of phage KP10-61: [531:1101:−;HP][1140:1452:−;HP][1544:2123:−;HP][2352:3693:+;ATP-dependent DNA helicase uvsW][3701:3935:+;ATP-dependent DNA helicase uvsW][4262:4439:−;HP][4438:4858:−;Recombination protein uvsY][5162:6452:−;Capsid vertex protein][6539:8108:−;Major capsid protein][8127:8916:−;Capsid assembly scaffolding protein][8948:9596:−;Prohead core protein protease][9598:10003:−;Prohead assembly protein gp68][10254:11829:−;Portal protein][11884:12370:−;Tail tube protein gp19][12418:14410:−;Tail sheath protein][14429:16259:−;Terminase %2C large subunit][16227:16767:−;Terminase %2C small subunit][16766:17591:−;Tail completion protein gp15][17671:18418:−;Neck protein gp14][18427:19354:−;Neck protein gp13][19392:21144:−;Fibritin][21154:22540:−;Short tail fiber protein gp12][22549:23218:−;Baseplate wedge protein gp11][23217:25035:−;Baseplate wedge protein gp10][25031:25895:−;Baseplate protein gp9][25904:26897:−;Baseplate wedge protein gp8][26897:29984:−;Baseplate wedge protein gp7][30064:31990:−;Baseplate wedge protein gp6][32021:34088:−;HP][34092:35862:−;Baseplate central spike complex protein gp5][35858:36218:−;Baseplate wedge protein gp53][36571:36931:+;HP][36946:37801:+;HP][37837:38626:−;HP][38995:39526:+;Tail tube terminator protein][39525:40221:+;Deoxynucleotide monophosphate kinase][40547:41171:+;HP][41178:41718:+;HP][41783:41924:+;HP][42013:42154:+;HP][42164:42500:+;HP][42496:42790:+;HP][42782:43034:+;HP][43276:43684:+;HP][43694:44078:+;H P][44079:44337:+;HP][44365:44689:+;HP][44688:45216:+;HP][46203:46404:+;HP][48297:48576:+;HP][48647:48899:+;HP][49470:49734:+;HP][49730:49937:+;HP][49947:51093:+;HP][51375:51741:+;HP][51721:52069:+;HP][52249:52681:+;HP][52682:52943:+;HP][53012:53276:+; HP][53315:53639:+;HP][53693:54095:+;HP][54162:54558:+;L-alanyl-D-glutamate peptidase][54589:55048:+;HP][55047:55428:+;HP][55582:56929:+;HP][56966:57164:+;HP][57175:57730:+;HP][57794:58436:+;HP][58503:58899:+;HP][59052:59643:+;HP][59652:59976:+; HP][60282:60756:+;HP][60758:61046:+;HP][61038:61254:+;HP][61436:62045:+;HP][62061:62454:+;HP][62560:62842:+;HP][62813:63236:+;HP][63307:64222:+;HP][64398:65460:+;HP][65824:66097:+;HP][66701:66929:+;HP][66925:67303:+;HP][67302:68310:+;HP][68306:68552: +;HP][68551:68809:+;HP][68805:69732:+;HP][69775:70054:+;HP][70260:70545:+;HP][70572: 71088:+;HP][71087:71408:+;HP][71415:73632:+;HP][74075:74321:+;HP][74472:74943:+;HP][74958:75243:+;HP][75600:75825:+;HP][75832:76288:+;HP][76322:76715:−;Baseplate wedge protein gp25][76715:77270:−;HP][77266:78010:−;HP][78038:78902:−;Baseplate tail-tube junction protein gp54][78911:79985:−;Baseplate tail-tube junction protein gp48][79995:81777:−;HP][81773:82349:−;HP][82418:83528:+;HP][83524:83902:−;HP][83888:84188:−;HP][84613:84886:−;HP][85621:87151:−;HP][87131:87662:−;HP][87742:87964:−;HP][87965:88229:−;HP][88225:88666:−;HP][89295:89589:−;HP][89715:89985:−;HP][89984:90347:−;HP][90357:90924:−;HP][90965:91388:−;HP][91397:91679:−;HP][91994:92246:−;HP][92256:92598:−;Capsid assembly protein Gp31][92613:93171:−;Deoxycytidylate deaminase][93386:93638:−;HP][93634:93922:−;HP][93975:94860:−;Polynucleotide kinase][94856:95030:−;HP][95014:95332:−;HP][95328:95832:−;HP][95821:96139:−;HP][96147:97305:−;T4 RNA ligase 1][97285:97651:−;Endonuclease II][97946:99122:−;Ribonucleoside-diphosphate reductase subunit beta][99158:101411:−;Ribonucleoside-diphosphate reductase large subunit][101450:102326:−;Thymidylate synthase][102327:102906:−;HP][102902:103160:−;HP][103156:103387:−;HP][103383:103674:−;HP][103733:105443:−;HP][105516:105711:−;HP][106473:108165:−;Exonuclease subunit 2][108422:109469:−;Exonuclease subunit 1][109824:110040:−;HP][110036:110564:−;RNA polymerase sigma factor][110868:111804:+;Ribonuclease H][111813:112077:+;Double-stranded DNA-binding protein][112080:112326:+;RNA polymerase-associated protein Gp33]1112403:112982:+;Protein Gp59][112997:113969:+;HP][114024:114294:+;HP][114320:114986:+;DNA polymerase clamp][115044:116043:+;Sliding-clamp-loader gp44 subunit][116045:116609:+;Sliding-clamp-loader gp44 subunit][116612:116975:+;Translation repressor protein][117043:117331:+;HP][117703:120403:+;DNA-directed DNA polymerase][120477:121635:+;Recombination and repair protein][121670:121991:+;HP][122000:123437:+;ATP-dependent helicase 41][123829:124084:+;HP][124178:124415:+;HP][124784:125516:+;HP][125521:126010:+;HP][126006:126225:+;HP][126280:126541:+;HP][126544:127570:+;DNA primase][127577:128114:+;HP][128295:128586:+;HP][128582:129911:+;ATP-dependent DNA helicase dda][129935:130244:+;HP][130244:130529:+;HP][130525:131197:+;Exodeoxyribonuclease][131193:131475:+;HP][131912:132215:+;HP][132924:133254:+;HP][133293:133890:+;HP][133886:134165:+;HP][134151:134736:+;HP][134758:135199:+;HP][135198:135363:+;HP][135430: 137329:+;HP][137328:138615:+;HP][138686:139241:+;HP][139244:139481:+;HP][139491:141753:+;HP][141752:142652:+;HP][142697:143072:+;HP][143046:143484:+;HP][143520:143769:+;HP][144040:144178:+;HP][144152:144482:+;HP][144478:144706:+;HP][144786:145584:+; HP][145583:145874:+;HP][146140:146788:−;Holin][147471:151299:−;HP][151307:151976:−;Long-tail fiber protein gp36][152025:153150:−;Long-tail fiber protein gp35][153149:156923:−;Long-tail fiber proximal subunit][157002:157302:−;HP][157298:157580:−;Glutaredoxin][157539:158208:−;HP][158250:159210:−;HP][159279:159771:−;HP][159767:160295:−;HP][160291:160486:−;HP][160818:161025:−;HP][161024:161522:−;HP][161828:163952:−;Anaerobic ribonucleoside-triphosphate reductase][164271:164754:−;Recombination endonuclease VII][164785:165061:−;Glutaredoxin][165069:165921:−;DNA adenine methylase][166206:167640:−;HP][167667:168117:−;HP][168126:168387:−;HP][168976:169558:−;HP][169547:170567:−;RNA ligase 2][170570:171095:−;HP][171094:171451:−;HP][171452:171779:−;HP][171775:171952:−;HP][171948:172266:−;HP][172298:173174:−;HP][173288:174128:+;HP][174177:174642:+;HP][174674:175193:−;HP][175206:175482:−;Highly immunogenic outer capsid protein]

Essential genes of phage KP10-3: [1787:3854:−;HP][3858:5628:−;Baseplate central spike complex protein gp5][5624:5984:−;Baseplate wedge protein gp53][6229:6697:+;HP][6712:7567:+;HP][7603:8392:−;HP][8761:9292:+;Tail tube terminator protein][9291:9987:+;Deoxynucleotide monophosphate kinase][10313:10937:+;HP][10944:11484:+;HP][11549:11690:+;HP][11779:11920:+;HP][11930:12266:+;HP][12262:12556:+;HP][12548:12800:+;HP][13042:13450:+;HP][13460:13844:+;H P][13845:14103:+;HP][14131:14455:+;HP][14454:14982:+;HP][15969:16170:+;HP][17325:17583:+;HP][17666:17945:+;HP][18016:18268:+;HP][18264:18411:+;HP][18410:18665:+;HP][19398:20553:+;HP][20549:20894:+;HP][20874:21222:+;HP][21669:22101:+;HP][22102:22363:+; HP][22432:22696:+;HP][22735:23059:+;HP][23113:23515:+;HP][23583:23979:+;L-alanyl-D-glutamate peptidase][24010:24469:+;HP][24468:24993:+;HP][25002:26349:+;HP][26386:26584:+;HP][26596:27151:+;HP][27215:27857:+;HP][27924:28071:+;HP][28067:28658:+;HP][28667:28991:+; HP][29439:29913:+;HP][29915:30203:+;HP][30385:30994:+;HP][31010:31403:+;HP][31509:31791:+;HP][31762:32185:+;HP][32256:33171:+;HP][33347:34409:+;HP][34582:34777:+;HP][34773:35046:+;HP][35650:35878:+;HP][35874:36252:+;HP][36251:37259:+;HP][37255:37501: +;HP][37500:37758:+;HP][37754:38666:+;HP][38724:39003:+;HP][39423:39711:+;HP][39936: 40452:+;HP][40451:40772:+;HP][40779:42996:+;HP][43065:43683:+;HP][43834:44305:+;HP][44320:44605:+;HP][44962:45187:+;HP][45194:45650:+;HP][45684:46077:−;Baseplate wedge protein gp25][46077:46632:−;HP][46628:47372:−;HP][47400:48264:−;Baseplate tail-tube junction protein gp54][48273:49347:−;Baseplate tail-tube junction protein gp48][49357:51139:−;HP][51135:51711:−;HP][51780:52890:+;HP][52886:53264:−;HP][53572:53863:−;HP][55221:56751:−;HP][56731:57262:−;HP][57342:57564:−;HP][57565:57829:−;HP][57825:58266:−;HP][58895:59189:−;HP][59313:59583:−;HP][59582:59945:−;HP][59955:60522:−;HP][60563:60983:−;HP][60992:61274:−;HP][61589:61841:−;HP][61851:62193:−;Capsid assembly protein Gp31][62208:62766:−;Deoxycytidylate deaminase][62981:63233:−;HP][63229:63517:−;HP][63569:64454:−;Polynucleotide kinase][64450:64624:−;HP][64919:65231:−;HP][65239:66397:−;T4 RNA ligase 1][66377:66743:−;Endonuclease H1][67038:68214:−;Ribonucleoside-diphosphate reductase subunit beta][68250:70503:−;Ribonucleoside-diphosphate reductase large subunit][70542:71418:−;Thymidylate synthase][71419:71998:−;HP][71994:72252:−;HP][72248:72479:−;HP][72475:72766:−;HP][72825:74535:−;HP][74608:74803:−;HP][75565:77257:−;Exonuclease subunit 2][77514:78561:−;Exonuclease subunit 1][78916:79132:−;HP][79128:79656:−;RNA polymerase sigma factor][79960:80896:+;Ribonuclease H][80905:81169:+;Double-stranded DNA-binding protein][81172:81418:+;RNA polymerase-associated protein Gp33][81495:82074:+;Protein Gp59][82089:83061:+;HP][83116:83386:+;HP][83412:84078:+;DNA polymerase clamp][84136:85135:+;Sliding-clamp-loader gp44 subunit][85137:85701:+;Sliding-clamp-loader gp44 subunit][85704:86067:+;Translation repressor protein][86135:86423:+;HP][86795:89495:+;DNA-directed DNA polymerase][89569:90727:+;Recombination and repair protein][90764:91085:+;HP][91094:92531:+;ATP-dependent helicase 41][92923:93178:+;HP][93269:93506:+;HP][93872:94604:+;HP][94609:95098:+;HP][95094:95313:+;HP][95368:95629:+;HP][95632:96658:+;DNA primase][96665:97202:+;HP][97383:97674:+;HP][97670:98999:+;ATP-dependent DNA helicase dda][99023:99332:+;HP][99332:99617:+;HP][99613:100285:+;Exodeoxyribonuclease][100281: 100563:+;HP][100779:101004:+;HP][101000:101303:+;HP][102012:102342:+;HP][102381:102978:+;HP][102974:103253:+;HP][103239:103824:+;HP][103846:104287:+;HP][104286:104451:+;HP][104518:106417:+;HP][106416:107703:+;HP][107774:108329:+;HP][108332:108569:+; HP][108579:110841:+;HP][110840:111740:+;HP][111785:112160:+;HP][112134:112572:+;HP][112608:112857:+;HP][112901:113039:+;HP][113013:113346:+;HP][113342:113570:+;HP][113650:114448:+;HP][114447:114738:+;HP][115004:115652:−;Holin][116332:120154:−;HP][120162:120831:−;Long-tail fiber protein gp36][120880:122005:−;Long-tail fiber protein gp35][122004:125778:−;Long-tail fiber proximal subunit][125857:126157:−;HP][126153:126435:−;Glutaredoxin][126394:127063:−;HP][127105:128065:−;HP][128134:128626:−;HP][128622:129150:−;HP][129146:129341:−;HP][129673:129880:−;HP][129879:130377:−;HP][130682:132812:−;Anaerobic ribonucleoside-triphosphate reductase][133131:133614:−;Recombination endonuclease VHJ][133645:133921:−;Glutaredoxin][133929:134781:−;DNA adenine methylase][135066:136500:−;HP][136527:136977:−;HP][136986:137247:−;HP][137824:138406:−;HP][138395:139415:−;RNA ligase 2][139418:139943:−;HP][139942:140299:−;HP][140300:140627:−;HP][140623:140800:−;HP][140796:141114:−;HP][141146:142022:−;HP][142136:142976:+;HP][143025:143490:+;HP][143522:144041:−;HP][144054:144330:−;Highly immunogenic outer capsid protein][144340:144874:−;Highly immunogenic outer capsid protein][144902:145472:−;HP][145511:145823:−;HP][145915:146494:−;HP][146723:148064:+;ATP-dependent DNA helicase uvsW][148072:148306:+;ATP-dependent DNA helicase uvsW][148909:149086:−;HP][149085:149505:−;Recombination protein uvsY][149809:151099:−;Capsid vertex protein][151131:151767:−;HP][151852:153421:−;Major capsid protein][153440:154229:−;Capsid assembly scaffolding protein][154261:154909:−;Prohead core protein protease][154911:155316:−;Prohead assembly protein gp68][155566:157141:−;Portal protein][157196:157682:−;Tail tube protein gp19][157730:159722:−;Tail sheath protein][159741:161571:−;Terminase %2C large subunit][161539:162079:−;Terminase %2C small subunit][162078:162903:−;Tail completion protein gp15][162983:163730:−;Neck protein gp14][163739:164666:−;Neck protein gp13][164704:166456:−;Fibritin][166469:167855:−;Short tail fiber protein gp12][167864:168533:−;Baseplate wedge protein gp11][168532:170350:−;Baseplate wedge protein gp10][170346:171210:−;Baseplate protein gp9][171219:172212:−;Baseplate wedge protein gp8][172212:175299:−;Baseplate wedge protein gp7]

Essential genes of phage 395-D2-3-1: [121:400:−;HP][396:993:−;HP][1032:1362:−;HP][2071:2374:−;HP][2811:3093:−;HP][3089:3761:−;Exodeoxyribonuclease][3757:4042:−;HP][4042:4351:−;HP][4375:5704:−;ATP-dependent DNA helicase dda][5700:5991:−;HP][7044:7581:−;HP][7588:8614:−;DNA primase][8617:8878:−;HP][8933:9152:−;HP][9148:9637:−;HP][9642:10374:−;HP][10740:10977:−;HP][11068:11323:−;HP][11715:13152:−;ATP-dependent helicase 41][13161:13482:−;HP][13519:14677:−;Recombination and repair protein][14751:17451:−;DNA-directed DNA polymerase][17823:18111:−;HP][18179:18542:−;Translation repressor protein][18545:19109:−;Sliding-clamp-loader gp44 subunit][19111:20110:−;Sliding-clamp-loader gp44 subunit][20168:20834:−;DNA polymerase clamp][20860:21130:−;HP][21185:22157:−;HP][22172:22751:−;Protein Gp59][22828:23074:−;RNA polymerase-associated protein Gp33][23077:23341:−;Double-stranded DNA-binding protein][23350:24286:−;Ribonuclease H][24590:25118:+;RNA polymerase sigma factor][25114:25330:+;HP][25685:26732:+;Exonuclease subunit 1][26989:28687:+;Exonuclease subunit 2][28686:28878:+;HP][28951:30661:+;HP][30720:31011:+;HP][31007:31238:+;HP][31234:31492:+;HP][31488:32067:+;HP][32068:32944:+;Thymidylate synthase][32983:35236:+;Ribonucleoside-diphosphate reductase large subunit][35272:36448:+;Ribonucleoside-diphosphate reductase subunit beta][36743:37109:+;Endonuclease H1][37089:38247:+;T4 RNA ligase 1][38453:38780:+;HP][38748:39084:+;HP][39080:39398:+;HP][39546:40431:+;Polynucleotide kinase][40483:40771:+;HP][40767:41019:+;HP][41234:41792:+;Deoxycytidylate deaminase][41807:42149:+;Capsid assembly protein Gp31][42159:42411:+;HP][42726:43008:+;HP][43017:43440:+;HP][43481:44048:+;HP][44058 :44421:+;HP][44420:44690:+;HP][44815:45109:+;HP][45809:46250:+;HP][46246:46765:+;HP][46773:47037:+;HP][47038:47260:+;HP][47340:47871:+;HP][47851:49381:+;HP][50739:51030:+;HP][51338:51716:+;HP][51712:52822:−;HP][52891:53467:+;HP][53463:55251:+;HP][55261:56344:+;Baseplate tail-tube junction protein gp48][56353:57217:+;Baseplate tail-tube junction protein gp54][57245:57986:+;HP][57982:58537:+;HP][58537:58930:+;Baseplate wedge protein gp25][58964:59420:−;HP][59427:59652:−;HP][60008:60293:−;HP][60308:60779:−;HP][60930:61548:−;HP][61617:63834:−;HP][63841:64162:−;HP][64161:64677:−;HP][64902:65190:−;HP][65610:65889:−;HP][65947:66859:−;HP][66855:67113:−;HP][67112:67358:−;HP][67354:68362:−;HP][68361:68739:−;HP][68735:68963:−;HP][69567:69840:−;HP][69836:70031:−;HP][70205:71267:−;HP][71443:72358:−;HP][72429:72852:−;HP][72823:73105:−;HP][73211:73604:−;HP][73620:74229:−;HP][74221:74509:−;HP][74511:74985:−;HP][75433:75757:−;HP][75766:76357:−;HP][76353:76500:−;HP][76567:77209:−;HP][77273:77828:−;HP][77840:78038:−;HP][78075:79422:−;HP][79480:79648:−;HP][79647:80106:−;HP][80137:80533:−;L-alanyl-D-glutamate peptidase][80601:81003:−;HP][81057:81381:−;HP][81420:81684:−;HP][81753:82014:−;HP][82015:82447:−;HP][82627:82825:−;HP][82945:83311:−;HP][83593:84748:−;HP][85481:85736:−;HP][85998:86145:−;HP][86141:86393:−;HP][86464:86743:−;HP][86826:87084:−;HP][88239:88440:−;HP][88824:89358:−;HP][89350:89686:−;HP][89693:89954:−;HP][89950:90331:−;HP][90327:90741:−;HP][91250:91547:−;HP][91806:92145:−;HP][92155:92296:−;HP][92385:92526:−;HP][92592:93132:−;HP][93153:93780:−;HP][94106:94802:−;Deoxynucleotide monophosphate kinase][94801:95332:−;Tail tube terminator protein][95701:96490:+;HP][96526:97381:−;HP][97396:97756:−;HP][98109:98469:+;Baseplate wedge protein gp53][98465:100235:+;Baseplate central spike complex protein gp5][100239:102306:+;HP][102337:104263:+;Baseplate wedge protein gp6][104343:107430:+;Baseplate wedge protein gp7][107430:108423:+;Baseplate wedge protein gp8][108432:109296:+;Baseplate protein gp9][109292:111110:+;Baseplate wedge protein gp10][111109:111778:+;Baseplate wedge protein gp11][111787:113173:+;Short tail fiber protein gp12][113183:114935:+;Fibritin][114973:115900:+;Neck protein gp13][116516:117263:+;Neck protein gp14][117342:118167:+;Tail completion protein gp15][118166:118706:+;Terminase %2C small subunit][118674:120504:+;Terminase %2C large subunit][120523:122515:+;Tail sheath protein][122563:123049:+;Tail tube protein gp19][123104:124679:+;Portal protein][124929:125334:+;Prohead assembly protein gp68][125336:125984:+;Prohead core protein protease][126016:126805:+;Capsid assembly scaffolding protein][126824:128393:+;Major capsid protein][128484:129123:+;HP][129155:130445:+;Capsid vertex protein][130741:131161:+;Recombination protein uvsY][131160:131337:+;HP][131796:132030:−;ATP-dependent DNA helicase uvsW][132038:133379:−;ATP-dependent DNA helicase uvsW][133608:134232:+;HP][134324:134636:+;HP][134675:135245:+;HP][135273:135807:+; Highly immunogenic outer capsid protein][135817:136093:+;Highly immunogenic outer capsid protein][136106:136625:+;HP][136656:137121:−;HP][137170:138010:−;HP][138124:139000:+;HP][139032:139350:+;HP][139346:139523:+;HP][139519:139846:+;HP][139847:140204:+;HP][140203:140728:+;HP][140731:141751:+;RNA ligase 2][141754:142273:+;HP][142262:142850:+;HP][143438:143699:+;HP][143708:144158:+;HP][144185:145619:+;HP][145904:146756:+;DNA adenine methylase][146762:147032:+;Glutaredoxin][147064:147547:+;Recombination endonuclease VII][147866:149990:+;Anaerobic ribonucleoside-triphosphate reductase][150295:150793:+;HP][150792:150999:+;HP][151331:151526:+;HP][151522:152050:+;HP][152046:152538:+;HP][152607:153567:+;HP][153609:154278:+;HP][154237:154519:+; Glutaredoxin][154515:154815:+;HP][154894:158668:+;Long-tail fiber proximal subunit][158667:159792:+;Long-tail fiber protein gp35][159841:160510:+;Long-tail fiber protein gp36][160518:164223:+;HP][164744:165392:+;Holin][165658:165949:−;HP][165948:166746:−;HP][166826:167054:−;HP][167050:167383:−;HP][167357:167492:−;HP][167536:167785:−;HP][167821:168259:−;HP][168233:168608:−;HP][168653:169553:−;HP][169552:171814:−;HP][171824:172061:−;HP][172064:172619:−;HP][172690:173977:−;HP][173976:175875:−;HP][175942:176107:−;HP][176106:176547:−;HP]

Essential genes of phage CT-227-KP4M1-8: [45:318:+;HP][1129:2137:+;HP][2133:2379:+;HP][2378:2636:+;HP][2632:3544:+;HP][3602:3881:+;HP][4301:4589:+;HP][4814:5330:+;HP][5329:5650:+;HP][5657:7874:+;HP][7943:8561:+; HP][8712:9183:+;HP][9198:9483:+;HP][9840:10065:+;HP][10072:10528:+;HP][10562:10955:−;Baseplate wedge protein gp25][10955:11510:−;HP][11506:12250:−;HP][12278:13142:−;Baseplate tail-tube junction protein gp54][13151:14225:−;Baseplate tail-tube junction protein gp48][14235:16017:−;HP][16013:16589:−;HP][16658:17768:+;HP][17764:18142:−;HP][18450:18741:−;HP][20097:21627:−;HP][22708:22930:−;HP][22931:23195:−;HP][23203:23722:−;HP][23718:24159:−;HP][24801:25095:−;HP][25220:25490:−;HP][25489:25852:−;HP][25862:26429:−;HP][26470:26893:−;HP][26902:27184:−;HP][27499:27751:−;HP][27761:28103:−;Capsid assembly protein Gp31][28118:28676:−;Deoxycytidylate deaminase][28891:29143:−;HP][29139:29427:−;HP][29479:30364:−;Polynucleotide kinase][30360:30534:−;HP][30518:30836:−;HP][30832:31168:−;HP][31136:31463:−;HP][31669:32827:−;T4 RNA ligase 1][32807:33176:−;Endonuclease II][33739:34915:−;HP][34953:37200:−;Ribonucleoside-diphosphate reductase large subunit][37240:38116:−;Thymidylate synthase][38117:38696:−;HP][38692:38950:−;HP][38946:39177:−;HP][39173:39464:−;HP][39523:41233:−;HP][41306:41504:−;HP][42044:43742:−;Exonuclease subunit 2][43999:45046:−;Exonuclease subunit 1][45401:45617:−;HP][45613:46141:−;RNA polymerase sigma factor][46445:47381:+;Ribonuclease H][47390:47654:+;Double-stranded DNA-binding protein][47657:47903:+;RNA polymerase-associated protein Gp33][47980:48559:+;Protein Gp59][48574:49546:+;HP][49601:49871:+;HP][49897:50563:+;DNA polymerase clamp][50621:51620:+;Sliding-clamp-loader gp44 subunit][51622:52186:+;Sliding-clamp-loader gp44 subunit][52189:52552:+;Translation repressor protein][52620:52908:+;HP][53280:55980:+;DNA-directed DNA polymerase][56054:57212:+;Recombination and repair protein][57249:57570:+;HP][57579:59016:+;ATP-dependent helicase 41][59408:59663:+;HP][59754:59991:+;HP][60357:61089:+;HP][61094:61583:+;HP][61579:61798:+;HP][61853:62114:+;HP][62117:63143:+;DNA primase][63150:63687:+;HP][63868:64159:+;HP][64155:65484:+;ATP-dependent DNA helicase dda][65508:65817:+;HP][65817:66102:+;HP][66098:66770:+;Exodeoxyribonuclease][66766:67048:+;HP][67485:67788:+;HP][68496:68826:+;HP][68865:69462:+;HP][69458:69737:+;HP][69723:70308:+;HP][70330:70771:+;HP][70770:70935:+;HP][71002:72901:+;HP][72900:74187:+; HP][74258:74813:+;HP][74816:75053:+;HP][75063:77325:+;HP][77324:78224:+;HP][78269:78644:+;HP][78618:79056:+;HP][79092:79341:+;HP][79385:79523:+;HP][79497:79830:+;HP][79826:80054:+;HP][80134:80932:+;HP][80931:81222:+;HP][81488:82136:−;Holin][82819:86842:−;HP][86850:87519:−;Long-tail fiber protein gp36][87568:88693:−;Long-tail fiber protein gp35][88692:92466:−;Long-tail fiber proximal subunit][92545:92845:−;HP][92841:93123:−;Glutaredoxin][93082:93751:−;HP][93793:94753:−;HP][94822:95314:−;HP][95350:95554:−;HP][95886:96093:−;HP][96092:96587:−;HP][96894:99018:−;Anaerobic ribonucleoside-triphosphate reductase][100123:100396:−;Glutaredoxin][100404:101256:−;DNA adenine methylase][101541:102975:−;HP][103002:103452:−;HP][103461:103722:−;HP][104311:104899:−;HP][104888:105407:−;HP][105410:106430:−;RNA ligase 2][106433:106958:−;HP][106957:107314:−;HP][107315:107642:−;HP][107638:107815:−;HP][107811:108129:−;HP][108161:109037:−;HP][109151:109991:+;HP][110040:110505:+;HP][110537:111056:−;HP][111069:111345:−;Highly immunogenic outer capsid protein][111355:111889:−;Highly immunogenic outer capsid protein][111917:112487:−;HP][112526:112838:−;HP][112929:113508:−;HP][113737:115078:+;ATP-dependent DNA helicase uvsW][115086:115320:+;ATP-dependent DNA helicase uvsW][115780:115957:−;HP][115956:116376:−;Recombination protein uvsY][116680:117970:−;Capsid vertex protein][118002:118638:−;HP][118723:120292:−;Major capsid protein][120311:121100:−;Capsid assembly scaffolding protein][121132:121780:−;Prohead core protein protease][121782:122187:−;Prohead assembly protein gp68][122438:124013:−;Portal protein][124068:124554:−;Tail tube protein gp19][124602:126594:−;Tail sheath protein][126613:128443:−;Terminase %2C large subunit][128411:128951:−;Terminase %2C small subunit][128950:129775:−;Tail completion protein gp15][129854:130601:−;Neck protein gp14][131217:132144:−;Neck protein gp13][132182:133934:−;Fibritin][133944:135330:−;Short tail fiber protein gp12][135339:136008:−;Baseplate wedge protein gp11][136007:137825:−;Baseplate wedge protein gp10][137821:138685:−;Baseplate protein gp9][138694:139687:−;Baseplate wedge protein gp8][139687:142774:−;Baseplate wedge protein gp7][142854:144780:−;Baseplate wedge protein gp6][144811:146878:−;HP][146882:148652:−;Baseplate central spike complex protein gp5][148648:149008:−;Baseplate wedge protein gp53][149253:149721:+;HP][149736:150591:+;HP][150627:151416:−;HP][151785:152316:+;Tail tube terminator protein][152315:153011:+;Deoxynucleotide monophosphate kinase][153337:153961:+;HP][153968:154508:+;HP][154573:154693:+;HP][154793:154934:+; HP][154944:155280:+;HP][155276:155570:+;HP][155562:155814:+;HP][156056:156464:+;HP][156474:156858:+;HP][156859:157117:+;HP][157145:157469:+;HP][157468:157996:+;HP][159055:159436:+;HP][159410:159764:+;HP][159944:160376:+;HP][160377:160638:+;HP][160707:160971:+;HP][161010:161334:+;HP][161388:161790:+;HP][161857:162253:+;L-alanyl-D-glutamate peptidase][162284:162743:+;HP][162742:163267:+;HP][163276:164623:+;HP][164659:164857:+;HP][164868:165423:+;HP][165487:166129:+;HP][166196:166343:+;HP][166339:166930:+; HP][166939:167263:+;HP][167569:168043:+;HP][168045:168333:+;HP][168325:168541:+;HP][168723:169332:+;HP][169348:169741:+;HP][169847:170129:+;HP][170100:170523:+;HP][170594:171509:+;HP][171685:172747:+;HP]

Essential genes of phage KP10-95: [203:566:−;HP][553:733:−;HP][729:1071:−;HP][1070:1715:−;Middle transcription regulatory protein motA][1839:2103:−;HP][2330:3719:−;HP][3724:3865:−;HP][3861:3969:−;HP][4034:4490:−;Nucleoid disruption protein][4548:4638:−;HP][4634:4826:−;HP][4836:4953:−;HP][5013:5253:−;HP][5317:5896:−;Endonuclease IV][5837:6134:−;HP][6176:7088:−;HP][7088:9257:−;Protein rIIA][9267:9477:−;HP][9759:9912:−;HP][9952:11791:−;HP][11821:12190:−;HP][12268:12517:−;HP][12513:12603:−;HP][12602:12779:−;HP][12765:13101:−;HP][13094:13460:−;HP][13459:13675:−;HP][13739:14180:−;HP][14243:14411:−;HP][14410:15100:−;Exodeoxyribonuclease][15092:15347:−;HP][15353:16685:−;ATP-dependent DNA helicase dda][16681:16996:−;HP][16995:17751:−;HP][17831:18497:−;HP][18569:18743:−;HP][19244:19436:−;HP][19514:19742:−;Small outer capsid protein][19772:19910:−;HP][19909:20431:−;HP][20497:20689:+;HP][20707:21730:−;DNA primase][21768:21933:−;HP][21948:22158:−;HP][22154:22448:−;Protein spackle][22458:22737:−;HP][22802:23036:−;HP][23045:24419:−;ATP-dependent helicase 41][24494:24845:−;HP][24834:26010:−;Recombination and repair protein][26092:26314:−;HP][26340:27177:−;HP][27173:27908:−;Deoxycytidylate 5-hydroxymethyltransferase][27916:28123:−;Immunity protein][28133:28577:−;HP][28632:31341:−;DNA-directed DNA polymerase][31433:31796:−;Translation repressor protein][31798:32368:−;Sliding-clamp-loader gp44 subunit][32364:33327:−;Sliding-clamp-loader gp44 subunit][33375:34077:−;DNA polymerase clamp][34112:34490:−;15 kDa RNA polymerase-binding protein][34500:34695:−;HP][34691:36332:−;Exonuclease subunit 2][36370:37393:−;Exonuclease subunit 1][37463:38660:−;DNA alpha-glucosyltransferase][38729:38987:−;HP][39060:39258:−;HP][39235:39538:−;HP][39539:39770:−;HP][39753:40302:−;RNA polymerase sigma factor][40368:40614:−;HP][40610:40889:−;HP][40876:41101:−;HP][41093:41378:−;HP][41370:41628:−;HP][41624:41957:−;HP][42021:42264:−;HP][42260:42449:−;HP][42634:42937:−;HP][42933:43275:−;HP][43271:43484:−;HP][43492:43591:−;HP][43601:44072:−;HP][44043:44277:−;HP][44248:46087:−;Anaerobic ribonucleoside-triphosphate reductase][46083:46557:−;Recombination endonuclease VII][46593:46902:−;HP][46894:47347:−;HP][47330:47489:−;HP][47467:47857:−;HP][47858:48029:−;HP][48047:48236:−;HP][48228:48498:−;Glutaredoxin][48460:48751:−;HP][48740:48914:−;HP][48906:49419:−;HP][49415:49748:−;HP][49744:50059:−;HP][50055:50292:−;HP][50312:51287:−;HP][51357:52146:−;HP][52203:52470:−;HP][52466:52631:−;HP][52993:53191:−;HP][53193:53514:−;HP][53510:53846:−;HP][53842:54268:−;HP][54264:54411:−;HP][54453:55431:−;HP][55548:55788:−;HP][55977:56988:−;HP][56984:57407:−;HP][57599:58181:−;HP][58177:58669:−;HP][58671:58893:−;HP][58882:59074:−;HP][59076:59343:−;HP][59440:59923:−;HP][60027:60414:−;HP][60400:60688:−;Antiholin][60699:60912:−;HP][60958:61333:−;HP][61617:61812:−;HP][61792:62392:−;HP][62391:62559:−;HP][62583:63051:−;HP][63059:63452:−;HP][63448:63769:−;HP][63768:64314:−;HP][64321:64780:−;Endoribonuclease RegB][64843:65119:−;HP][65118:65481:−;HP][65489:65981:−;HP][65962:66274:−;HP][66270:66804:−;HP][66883:67189:−;HP][67379:67952:−;Internal protein III][68033:68513:−;HP][68513:69008:−;Endolysin][69036:69345:−;HP][69352:69799:−;HP][69795:70032:−;HP][70031:70358:−;HP][70354:70651:−;HP][70647:71010:−;HP][71006:71597:−;HP][71636:72230:−;HP][72259:72487:−;HP][72473:72737:−;HP][72755:73046:−;HP][73971:74421:−;HP][74420:74978:−;HP][75156:75381:−;HP][75435:75669:−;HP][75652:76021:−;HP][76017:76170:−;HP][78072:78423:−;HP][78439:78625:−;HP][78624:78804:−;HP][78790:78970:−;HP][79045:79501:−;HP][79500:79737:−;HP][79733:80408:−;Deoxynucleotide monophosphate kinase][80475:81078:−;Tail tube terminator protein][81077:81905:−;HP][81904:82354:−;HP][82403:83042:+;Baseplate wedge protein gp53][83038:84769:+;Baseplate central spike complex protein gp5][84771:85275:+;HP][85275:85569:+;Baseplate puncturing device gp5.4][85568:87536:+;Baseplate wedge protein gp6][87532:90631:+;Baseplate wedge protein gp7][90623:91649:+;Baseplate wedge protein gp8][91716:92628:+;Baseplate protein gp9][92627:94451:+;Baseplate wedge protein gp10][94452:95124:+;Baseplate wedge protein gp11][95123:96470:+;HP][96478:98245:+;HP][98301:99243:+;Neck protein gp13][99246:100023:+;Neck protein gp14][100064:100889:+;Tail completion protein gp15][100885:101395:+;Terminase %2C small subunit][101372:103211:+;Terminase %2C large subunit][103244:105218:+;Tail sheath protein][106031:106523:+;Tail tube protein gp19][106627:108196:+;Portal protein][108195:108420:+;HP][108419:108845:+;Prohead assembly protein gp68][108844:109495:+;Prohead core protein protease][109527:110340:+;Capsid assembly scaffolding protein][110360:111920:+;Major capsidprotein][112001:113282:+;Capsidvertexprotein][113311:113860:−;HP][113859:114867:−;RNA ligase 2][114877:115135:−;HP][115248:115575:−;HP][115574:115802:−;HP][115880:117005:−;Highly immunogenic outer capsid protein][117014:117809:−;HP][118386:119370:+;ATP-dependent DNA helicase uvsW][119366:119609:+;ATP-dependent DNA helicase uvsW][119668:119836:−;HP][119866:120283:−;Recombination protein uvsY][120324:120735:−;Baseplate wedge protein gp25][120731:121361:−;HP][121412:122171:+;HP][122167:123310:+;Baseplate central spike complex protein gp27][123281:123809:+;Baseplate hub assembly protein gp28][123805:125539:+;HP][125548:126601:+;Baseplate tail-tube junction protein gp48][126600:127527:+;Baseplate tail-tube junction protein gp54][127548:127827:−;HP][127890:130017:−;HP][130068:130248:−;HP][130244:131717:−;HP][131716:131923:−;HP][131964:132807:−;HP][132990:133191:−;HP][133187:133358:−;HP][133368:133647:−;HP][133659:134145:−;HP][134185:134545:−;HP][134638:135151:−;HP][135190:135559:−;HP][135680:135857:−;HP][136224:136473:−;Lysis inhibition accessory protein][136588:136912:−;Capsid assembly protein Gp31][136969:137278:−;HP][137274:137475:−;HP][137474:138044:−;Deoxycytidylate deaminase][138044:138389:−;HP][138388:138625:−;HP][138614:138899:−;HP][138910:139804:−;Polynucleotide kinase][139803:140016:−;HP][140026:140314:−;HP][140310:140664:−;HP][140651:141161:−;HP][141223:142375:−;T4 RNA ligase 1][142368:142785:−;Endonuclease 11][142812:143955:−;HP][144011:146258:−;Ribonucleoside-diphosphate reductase large subunit][146248:146554:−;HP][146550:146757:−;HP][146753:147629:−;Thymidylate synthase][147621:147939:−;HP][147922:148522:−;Dihydrofolate reductase][148529:148694:−;HP][148776:149097:−;HP][149089:149179:−;HP][149175:149454:−;HP][149511:149877:−;HP][149880:150102:−;HP][150143:150401:−;HP][150527:151445:−;Single-stranded DNA-binding protein][151513:152176:−;Protein Gp59][152172:152472:−;RNA polymerase-associated protein Gp33][152452:152725:−;Double-stranded DNA-binding protein][152735:153662:−;Ribonuclease H][153737:157583:+;Long-tail fiber proximal subunit][157582:158719:+;Long-tail fiber protein gp35][158776:159469:+;Long-tail fiber protein gp36][159478:164107:+;HP][164143:164671:+;HP][164712:165369:+;Holin][165369:165642:−; 10 kDa anti-sigma factor][165710:165977:−;HP][165973:166300:−;HP][166296:166575:−;HP]

Essential genes of phage PKP-33: [36:180:+;HP][1798:2665:−;HP][2720:3041:−;HP][3053:3563:−;HP][3575:3977:−;HP][4430:4619:−;HP][4670:4958:−;HP][4969:5359:−;HP][5318:5786:−;HP][5769:5997:−;HP][5989:6331:−;HP][6327:6576:−;HP][6572:6854:−;HP][6853:7159:−;HP][7241:7670:−;HP][7733:7853:−;HP][7855:8224:−;HP][8232:8466:−;HP][8476:9307:−;Serine/threonine-protein phosphatase][9306:9669:−;HP][9668:10073:−;HP][10174:10459:−;HP][10451:10856:−;HP][11403:11817:−;L-alanyl-D-glutamate peptidase][11813:12476:−;Holin][12633:13245:−;HP][13254:14016:−;Deoxynucleoside-5′-monophosphate kinase][14012:14132:−;HP][14254:14716:−;HP][14681:15395:−;HP][15503:15896:−;HP][16015:16450:−;HP][16442:16715:−;HP][16707:17031:−;HP][17077:17515:−;HP][17516:17726:−;HP][17722:18028:−;HP][18017:18284:−;HP][18252:18621:−;HP][18607:18835:−;HP][18821:19364:−;HP][19363:19636:−;HP][19639:19837:−;HP][19949:20120:−;HP][20281:20662:−;HP][20652:21009:−;HP][21106:21298:−;HP][21416:21518:−;HP][21855:22365:−;HP][22596:22935:−;HP][22937:23132:−;HP][24659:24773:−;HP][24844:25060:−;HP][25059:25437:−;HP][25457:25598:−;HP][26612:26792:−;HP][26778:26970:−;HP][28680:29175:−;HP][29185:29395:−;HP][29394:29592:−;HP][29721:30015:−;HP][30243:30408:−;HP][31723:32185:−;HP][32249:33254:−;HP][33253:33664:−;HP][33793:34078:−;HP][34067:34229:−;HP][34228:34711:−;HP][34710:34986:−;HP][35322:36003:−;Thymidylate synthase][36173:36737:−;Dihydrofolate reductase][36736:37897:−;HP][37993:40318:−;Ribonucleoside-diphosphate reductase large subunit][40446:40656:−;HP][40670:40784:−;HP][40785:41535:−;HP][41844:43674:+;Anaerobic ribonucleoside-triphosphate reductase][43734:43947:+;HP][43946:44780:+;HP][44763:44928:+;HP][44914:45316:+;HP][45312:45708:+;HP][46306:49096:+;Putative replication origin binding protein][49082:49367:+;HP][49399:50095:+;HP][50381:50786:+;HP][50798:51095:+;HP][51145:51454:+;HP][51537:51699:+;HP][51655:51931:+;HP][51930:52893:+;DNA ligase][53092:53842:+;HP][53844:54603:+;Putative transcription factor D5][54687:56166:+;HP][56162:57047:+;HP][57114:59685:+;DNA polymerase][60171:61530:+;putative helicase D10][61732:62086:+;HP][62088:62862:+;putative ssDNA-binding protein][62901:63894:+;putative exonuclease subunit 1][63877:65713:+;putative exonuclease subunit 2][65712:66195:+;Protein D14][66191:67070:+;T5 flap endonuclease][67069:67513:+;Deoxyuridine 5′-triphosphate nucleotidohydrolase][67554:69597:−;HP][69596:70016:−;L-shaped tail fiber protein p132][70019:78383:−;HP][78410:81257:−;putative baseplate hub protein][81253:81868:−;Distal tail protein][81982:86440:−;putative tape measure protein][86524:86851:−;HP][86924:87347:−;HP][87350:88238:−;Minor tail protein][88247:89381:−;Tail tube protein][89404:89890:−;Tail tube terminator protein][89889:90600:−;Tail completion protein][90650:91163:−;HP][91221:92604:−;Major capsid protein][92617:93214:−;Prohead protease][93412:94132:−;HP][94128:95340:−;Portal protein][95339:95780:−;Nicking endonuclease][96013:97330:−;Terminase %2C large subunit][97329:97803:−;putative terminase %2C small subunit][97813:99790:−;Receptor-binding protein][99903:100128:+;HP][100727:101444:−;5′-deoxynucleotidase][101516:101969:−;HP][102032:103715:−;Protein A1][103798:104026:−;HP][104022:104292:−;HP][104359:104764:−;Protein A2][105062:105323:−;HP][105490:105679:−;HP][105694:105862:−;HP][106702:106933:−;HP][106932:107280:−;HP][107276:107804:−;HP][107858:108353:−;HP][108418:109384:−;HP][109456:109795:−;HP][109940:110054:+;HP][110053:110224:+;HP]

Essential genes of phage 395-D2-3-2: [257:905:+;HP][947:1616:+;HP][1575:1857:+;Glutaredoxin][1853:2153:+;HP][2232:6006:+;Lo ng-tail fiber proximal subunit][6005:7130:+;Long-tail fiber protein gp35][7179:7848:+;Long-tail fiber protein gp36][7856:11585:+;HP][12266:12914:+;Holin][13180:13471:−;HP][13470:14268:−;HP][14348:14576:−;HP][14572:14902:−;HP][14876:15014:−;HP][15010:15241:−;HP][15285:15534:−;HP][15570:16008:−;HP][15982:16357:−;HP][16402:17302:−;HP][17301:19563:−;HP][19573:19810:−;HP][19813:20368:−;HP][20439:21726:−;HP][21725:23624:−;HP][23691:23856:−;HP][23855:24296:−;HP][24318:24903:−;HP][24889:25168:−;HP][25164:25761:−;HP][25800:26130:−;HP][26839:27142:−;HP][27579:27861:−;HP][27857:28529:−;Exodeoxyribonuclease][28525:28810:−;HP][28810:29119:−;HP][29143:30472:−;ATP-dependent DNA helicase dda][30468:30759:−;HP][30940:31477:−;HP][31484:32510:−;DNA primase][32513:32774:−;HP][32829:33048:−;HP][33044:33533:−;HP][33538:34270:−;HP][34639:34876:−;HP][34968:35223:−;HP][35615:37052:−;ATP-dependent helicase 41][37061:37382:−;HP][37420:38578:−;Recombination and repair protein][38652:41352:−;DNA-directed DNA polymerase][41724:42012:−;HP][42080:42443:−;Translation repressor protein][42446:43010:−;Sliding-clamp-loader gp44 subunit][43012:44011:−;Sliding-clamp-loader gp44 subunit][44069:44735:−;DNA polymerase clamp][44761:45031:−;HP][45086:46058:−;HP][46073:46652:−;Protein Gp59][46729:46975:−;RNA polymerase-associated protein Gp33][46978:47242:−;Double-stranded DNA-binding protein][47251:48187:−;Ribonuclease H][48491:49019:+;RNA polymerase sigma factor][49015:49231:+;HP][49586:50633:+;Exonuclease subunit 1][50890:52582:+;Exonuclease subunit 2][53344:53539:+;HP][53612:55322:+;HP][55381:55672:+;HP][55668:55899:+;HP][55895:56153:+;HP][56149:56728:+;HP][56729:57605:+;Thymidylate synthase][57644:59897:+;Ribonucleoside-diphosphate reductase large subunit][59933:61109:+;Ribonucleoside-diphosphate reductase subunit beta][61404:61770:+;Endonuclease II][61750:62908:+;T4 RNA ligase 1][63114:63441:+;HP][63409:63745:+;HP][63741:64059:+;HP][64043:64211:+;HP][64207:65092:+;Polynucleotide kinase][65144:65432:+;HP][65428:65680:+;HP][65895:66453:+;Deoxycytidylate deaminase][66468:66810:+;Capsid assembly protein Gp31][66820:67072:+;HP][67387:67669:+;HP][67678:68101:+;HP][68142:68709:+;HP][68719 :69082:+;HP][69081:69351:+;HP][69476:69770:+;HP][70399:70840:+;HP][70836:71100:+;HP][71101:71323:+;HP][71403:71934:+;HP][71914:73444:+;HP][74179:74452:+;HP][74865:75165:+;HP][75151:75529:+;HP][75525:76635:−;HP][76704:77280:+;HP][77276:79058:+;HP][79068:80142:+;Baseplate tail-tube junction protein gp48][80151:81015:+;Baseplate tail-tube junction protein gp54][81043:81787:+;HP][81783:82338:+;HP][82338:82731:+;Baseplate wedge protein gp25][82765:83221:−;HP][83228:83453:−;HP][83810:84095:−;HP][84110:84581:−;HP][84732:85350:−;HP][85421:87638:−;HP][87645:87966:−;HP][87965:88481:−;HP][88706:88994:−;HP][89200:89479:−;HP][89537:90449:−;HP][90445:90703:−;HP][90702:90948:−;HP][90944:91952:−;HP][91951:92329:−;HP][92325:92553:−;HP][93157:93430:−;HP][93794:94856:−;HP][95032:95947:−;HP][96018:96441:−;HP][96412:96694:−;HP][96800:97193:−;HP][97209:97818:−;HP][98000:98216:−;HP][98208:98496:−;HP][98498:98972:−;HP][99278:99602:−;HP][99611:100202:−;HP][100355:100751:−;HP][100818:101460:−;HP][101524:102079:−;HP][102090:102288:−;HP][102324:103671:−;HP][103680:104205:−;HP][104204:104663:−;HP][104694:105090:−;L-alanyl-D-glutamate peptidase][105157:105559:−;HP][105613:105937:−;HP][105976:106240:−;HP][106309:106570:−;HP][106571:107003:−;HP][107183:107531:−;HP][107511:107877:−;HP][108159:109305:−;HP][109315:109522:−;HP][109518:109782:−;HP][110353:110605:−;HP][110676:110955:−;HP][111435:111693:−;HP][112848:113049:−;HP][114036:114564:−;HP][114563:114887:−;HP][114915:115173:−;HP][115174:115558:−;HP][115568:115976:−;HP][116218:116470:−;HP][116462:116756:−;HP][116752:117088:−;HP][117098:117239:−;HP][117328:117469:−;HP][117534:118074:−;HP][118081:118705:−;HP][119031:119727:−;Deoxynucleotide monophosphate kinase][119726:120257:−;Tail tube terminator protein][120626:121415:+;HP][121451:122306:−;HP][122321:122681:−;HP][123034:123394:+;Baseplate wedge protein gp53][123390:125160:+;Baseplate central spike complex protein gp5][125164:127231:+;HP][127262:129188:+;Baseplate wedge protein gp6][129268:132355:+;Baseplate wedge protein gp7][132355:133348:+;Baseplate wedge protein gp8][133357:134221:+;Baseplate protein gp9][134217:136035:+;Baseplate wedge protein gp10][136034:136703:+;Baseplate wedge protein gp11][136712:138098:+;Short tail fiber protein gp12][138108:139860:+;Fibritin][139898:140825:+;Neck protein gp13][140834:141581:+;Neck protein gp14][141660:142485:+;Tail completion protein gp15][142484:143024:+;Terminase %2C small subunit][142992:144822:+;Terminase %2C large subunit][144841:146833:+;Tail sheath protein][146881:147367:+;Tail tube protein gp19][147422:148997:+;Portal protein][149248:149653:+;Prohead assembly protein gp68][149655:150303:+;Prohead core protein protease][150335:151124:+;Capsid assembly scaffolding protein][151143:152712:+;Major capsid protein][152797:153433:+;HP][153465:154755:+;Capsid vertex protein][155059:155479:+;Recombination protein uvsY][155478:155655:+;HP][156259:156493:−;ATP-dependent DNA helicase uvsW][156501:157842:−;ATP-dependent DNA helicase uvsW][158071:158650:+;HP][158742:159054:+;HP][159093:159663:+;HP][159691:160225:+; Highly immunogenic outer capsid protein][160235:160511:+;Highly immunogenic outer capsid protein][160524:161043:+;HP][161075:161540:−;HP][161589:162429:−;HP][162543:163419:+;HP][163451:163769:+;HP][163765:163942:+;HP][163938:164265:+;HP][164266:164623:+;HP][164622:165147:+;HP][165150:166170:+;RNA ligase 2][166159:166741:+;HP][167330:167591:+;HP][167600:168050:+;HP][168077:169511:+;HP][169796:170648:+;DNA adenine methylase][170656:170932:+;Glutaredoxin][170963:171446:+;Recombination endonuclease VII][171765:173889:+;Anaerobic ribonucleoside-triphosphate reductase][174195:174693:+;HP][174692:174899:+;HP][175231:175426:+;HP][175422:175950:+;HP][175946:176438:+;HP]

Essential genes of phage 395-D2-3-5: [1217:1670:−;Spanin %2C inner membrane subunit][1759:2026:−;Terminase %2C small subunit gp18][2029:2233:−;HP][4479:8442:−;Peptidoglycan transglycosylase gp16][8460:10704:−;Internal virion protein gp15][10706:11300:−;Internal virion protein gp14][11302:11692:−;HP][11678:12104:−;HP][12176:14582:−;Tail tubular protein gp12][14597:15188:−;Tail tubular protein gp11][15572:16616:−;Minor capsid protein][16772:17705:−;Capsid assembly scaffolding protein][17806:19414:−;Portal protein][19424:19745:−;HP][19771:20023:−;HP][20027:20273:−;HP][20366:20480:−;HP][20458:21373:−;Exonuclease][21548:21758:−;Fusion protein 5.5/5.7][21754:22060:−;Fusion protein 5.5/5.7][22078:24193:−;DNA-directed DNA polymerase][24557:24770:−;HP][24866:26381:−;DNA primase/helicase][26745:27201:−;Endolysin][27193:27655:−;Endonuclease I][27654:28353:−;Single-stranded DNA-binding protein gp2.5][29241:29499:−;HP][29667:30684:−;DNA ligase][30778:31054:−;Inhibitor of dGTPase][31879:34534:−;T7 RNA polymerase][34604:35714:−;Protein kinase 0.7][35885:36083:−;HP][36257:36458:−;HP][36522:36981:−;HP][38539:39172:−;Terminase %2C large subunit gp19]

Essential genes of phage KP10-93: [123:1185:−;HP][1361:2276:−;HP][2347:2770:−;HP][2741:3023:−;HP][3129:3522:−;HP][3538:4147:−;HP][4329:4545:−;HP][4537:4825:−;HP][4827:5301:−;HP][5607:5931:−;HP][5940:6531:−;HP][6527:6674:−;HP][6741:7383:−;HP][7447:8002:−;HP][8013:8211:−;HP][8247:9594:−;HP][9603:10128:−;HP][10127:10586:−;HP][10617:11013:−;L-alanyl-D-glutamate peptidase][11080:11482:−;HP][11536:11860:−;HP][11899:12163:−;HP][12232:12493:−;HP][12494:12926:−;HP][13106:13460:−;HP][13434:13815:−;HP][14874:15402:−;HP][15401:15725:−;HP][15753:16011:−;HP][16012:16396:−;HP][16406:16814:−;HP][17056:17308:−;HP][17300:17594:−;HP][17590:17926:−;HP][17936:18077:−;HP][18177:18297:−;HP][18362:18902:−;HP][18909:19533:−;HP][19859:20555:−;Deoxynucleotide monophosphate kinase][20554:21085:−;Tail tube terminator protein][21454:22243:+;HP][22279:23134:−;HP][23149:23509:−;HP][23862:24222:+;Baseplate wedge protein gp53][24218:25988:+;Baseplate central spike complex protein gp5][25992:28059:+;HP][28090:30016:+;Baseplate wedge protein gp6][30096:33183:+;Baseplate wedge protein gp7][33183:34176:+;Baseplate wedge protein gp8][34185:35049:+;Baseplate protein gp9][35045:36863:+;Baseplate wedge protein gp10][36862:37531:+;Baseplate wedge protein gp11][37540:38926:+;Short tail fiber protein gp12][38936:40688:+;Fibritin][40726:41653:+;Neck protein gp13][41662:42409:+;Neck protein gp14][42489:43314:+;Tail completion protein gp15][43313:43853:+;Terminase %2C small subunit][43821:45651:+;Terminase %2C large subunit][45670:47662:+;Tail sheath protein][47710:48196:+;Tail tube protein gp19][48251:49826:+;Portal protein][50077:50482:+;Prohead assembly protein gp68][50484:51132:+;Prohead core protein protease][51164:51953:+;Capsid assembly scaffolding protein][51972:53541:+;Major capsid protein][53626:54262:+;HP][54294:55584:+;Capsid vertex protein][55888:56308:+;Recombination protein uvsY][56307:56484:+;HP][56944:57178:−;ATP-dependent DNA helicase uvsW][57186:58527:−;ATP-dependent DNA helicase uvsW][58756:59335:+;HP][59426:59738:+;HP][59777:60347:+;HP][60375:60909:+;Highly immunogenic outer capsid protein][60919:61195:+;Highly immunogenic outer capsid protein][61208:61727:+;HP][61759:62224:−;HP][62273:63113:−;HP][63227:64103:+;HP][64135:64453:+;HP][64449:64626:+;HP][64622:64949:+;HP][64950:65307:+;HP][65306:65831:+;HP][65834:66854:+;RNA ligase 2][66857:67376:+;HP][67365:67953:+;HP][68542:68803:+;HP][68812:69262:+;HP][69289:70723:+;HP][71008:71860:+;DNA adenine methylase][71868:72141:+;Glutaredoxin][73246:75370:+;Anaerobic ribonucleoside-triphosphate reductase][75678:76173:+;HP][76172:76379:+;HP][76711:76915:+;HP][76951:77443:+;HP][77512:78472:+;HP][78514:79183:+;HP][79142:79424:+;Glutaredoxin][79420:79720:+;HP][79799 :83573:+;Long-tail fiber proximal subunit][83572:84697:+;Long-tail fiber protein gp35][84746:85415:+;Long-tail fiber protein gp36][85423:89254:+;HP][89934:90582:+;Holin][90848:91139:−;HP][91138:91936:−;HP][92016:92244:−;HP][92240:92573:−;HP][92547:92685:−;HP][92729:92978:−;HP][93014:93452:−;HP][93426:93801:−;HP][93846:94746:−;HP][94745:97007:−;HP][97017:97254:−;HP][97257:97812:−;HP][97883:99170:−;HP][99169:101068:−;HP][101135:101300:−;HP][101299:101740:−;HP][101762:102347:−;HP][102333:102612:−;HP][102608:103205:−;HP][103244:103574:−;HP][104283:104586:−;HP][105023:105305:−;HP][105301:105973:−;Exodeoxyribonuclease][105969:106254:−;HP][106254:106563:−;HP][106587:107916:−;ATP-dependent DNA helicase dda][107912:108203:−;HP][108384:108921:−;HP][108928:109954:−;DNA primase][109957:110218:−;HP][110273:110492:−;HP][110488:110977:−;HP][110982:111714:−;HP][112080:112317:−;HP][112408:112663:−;HP][113055:114492:−;ATP-dependent helicase 41][114501:114822:−;HP][114859:116017:−;Recombination and repair protein][116091:118791:−;DNA-directed DNA polymerase][119163:119451:−;HP][119519:119882:−;Translation repressor protein][119885:120449:−;Sliding-clamp-loader gp44 subunit][120451:121450:−;Sliding-clamp-loader gp44 subunit][121508:122174:−;DNA polymerase clamp][122200:122470:−;HP][122525:123497:−;HP][123512:124091:−;ProteinGp59][124168:124414:−;RNApolymerase-associated protein Gp33][124417:124681:−;Double-stranded DNA-binding protein][124690:125626:−;Ribonuclease H][125930:126458:+;RNA polymerase sigma factor][126454:126670:+;HP][127025:128072:+;Exonuclease subunit 1][128329:130027:+;Exonuclease subunit 2][130567:130765:+;HP][130838:132548:+;HP][132607:132898:+;HP][132894:133125:+;HP][133121:133379:+;HP][133375:133954:+;HP][133955:134831:+;Thymidylate synthase][134871:137118:+;Ribonucleoside-diphosphate reductase large subunit][137156:138332:+;HP][138895:139264:+;Endonuclease II][139244:140402:+;T4 RNA ligase 1][140608:140935:+;HP][140903:141239:+;HP][141235:141553:+;HP][141537:141711:+;HP][141707:142592:+;Polynucleotide kinase][142644:142932:+;HP][142928:143180:+;HP][143395:143953:+;Deoxycytidylate deaminase][143968:144310:+;Capsid assembly protein Gp31][144320:144572:+;HP][144887:145169:+;HP][145178:145598:+;HP][145780:146206:+; HP][146216:146579:+;HP][146578:146848:+;HP][146973:147267:+;HP][147909:148350:+;HP][148346:148865:+;HP][148873:149137:+;HP][149138:149360:+;HP][150441:151971:+;HP][153327:153618:+;HP][153926:154304:+;HP][154300:155410:−;HP][155479:156055:+;HP][156051:157833:+;HP][157843:158917:+;Baseplate tail-tube junction protein gp48][158926:159790:+;Baseplate tail-tube junction protein gp54][159818:160562:+;HP][160558:161113:+;HP][161113:161506:+;Baseplate wedge protein gp25][161540:161996:−;HP][162003:162228:−;HP][162585:162870:−;HP][162885:163356:−;HP][163507:164125:−;HP][164194:166411:−;HP][166418:166739:−;HP][166738:167254:−;HP][167479:167767:−;HP][168187:168466:−;HP][168524:169436:−;HP][169432:169690:−;HP][169689:169935:−;HP][169931:170939:−;HP][171750:172023:−;HP]

Essential genes of phage KP10-14: [1820:2108:−;HP][2176:2539:−;Translation repressor protein][2542:3106:−;Sliding-clamp-loader gp44 subunit][3108:4107:−;Sliding-clamp-loader gp44 subunit][4165:4831:−;DNA polymerase clamp][4857:5127:−;HP][5182:6154:−;HP][6169:6748:−;Protein Gp59][6825:7071:−;RNA polymerase-associated protein Gp33][7074:7338:−;Double-stranded DNA-binding protein][7347:8283:−;Ribonuclease H][8587:9115:+;RNA polymerase sigma factor][9111:9327:+;HP][9682:10729:+;Exonuclease subunit 1][10986:12678:+;Exonuclease subunit 2][13440:13635:+;HP][13708:15418:+;HP][15477:15768:+;HP][15764:15995:+;HP][15991:16249:+;HP][16245:16824:+;HP][16825:17701:+;Thymidylate synthase][17740:19993:+;Ribonucleoside-diphosphate reductase large subunit][20029:21205:+;Ribonucleoside-diphosphate reductase subunit beta][21500:21866:+;Endonuclease II][21846:23004:+;T4 RNA ligase 1][23012:23324:+;HP][23619:23793:+;HP][23789:24674:+;Polynucleotide kinase][24726:25014:+;HP][25010:25262:+;HP][25477:26035:+;Deoxycytidylate deaminase][26050:26392:+;Capsid assembly protein Gp31][26402:26654:+;HP][26969:27251:+;HP][27260:27680:+;HP][27721:28288:+;HP][28298 :28661:+;HP][28660:28930:+;HP][29055:29349:+;HP][29990:30431:+;HP][30427:30946:+;HP][30954:31218:+;HP][31219:31441:+;HP][31521:32052:+;HP][32032:33562:+;HP][34920:35211:+;HP][35519:35897:+;HP][35893:37003:−;HP][37072:37648:+;HP][37644:39432:+;HP][39442:40525:+;Baseplate tail-tube junction protein gp48][40534:41398:+;Baseplate tail-tube junction protein gp54][41426:42167:+;HP][42163:42718:+;HP][42718:43111:+;Baseplate wedge protein gp25][43145:43601:−;HP][43608:43833:−;HP][44190:44475:−;HP][44490:44961:−;HP][45112:45730:−;HP][45801:48018:−;HP][48025:48346:−;HP][48345:48861:−;HP][49086:49374:−;HP][49794:50073:−;HP][50131:51043:−;HP][51039:51297:−;HP][51296:51542:−;HP][51538:52546:−;HP][52545:52923:−;HP][52919:53147:−;HP][53751:54024:−;HP][54389:55451:−;HP][55627:56542:−;HP][56613:57036:−;HP][57007:57289:−;HP][57395:57788:−;HP][57804:58413:−;HP][58595:58883:−;HP][58885:59359:−;HP][59807:60131:−;HP][60140:60731:−;HP][60727:60874:−;HP][60941:61583:−;HP][61647:62202:−;HP][62214:62412:−;HP][62449:63796:−;HP][63805:64330:−;HP][64329:64788:−;HP][64819:65215:−;L-alanyl-D-glutamate peptidase][65282:65684:−;HP][65738:66062:−;HP][66101:66365:−;HP][66434:66695:−;HP][66696:67128:−;HP][67308:67662:−;HP][67636:68017:−;HP][68309:69479:−;HP][69489:69696:−;HP][69692:69953:−;HP][70215:70362:−;HP][70358:70610:−;HP][70681:70960:−;HP][72456:72657:−;HP][73644:74172:−;HP][74171:74495:−;HP][74523:74781:−;HP][74782:75166:−;HP][75176:75584:−;HP][75826:76078:−;HP][76070:76364:−;HP][76360:76696:−;HP][76706:76847:−;HP][76936:77077:−;HP][77142:77682:−;HP][77689:78313:−;HP][78639:79335:−;Deoxynucleotide monophosphate kinase][79334:79865:−;Tail tube terminator protein][80234:81023:+;HP][81059:81914:−;HP][81929:82397:−;HP][82642:83002:+;Baseplate wedge protein gp53][82998:84768:+;Baseplate central spike complex protein gp5][84769:86836:+;HP][86867:88793:+;Baseplate wedge protein gp6][88873:91960:+;Baseplate wedge protein gp7][91960:92953:+;Baseplate wedge protein gp8][92962:93826:+;Baseplate protein gp9][93822:95640:+;Baseplate wedge protein gp10][95639:96308:+;Baseplate wedge protein gp11][96317:97703:+;Short tail fiber protein gp12][97716:99468:+;Fibritin][99506:100433:+;Neck protein gp13][100442:101189:+;Neck protein gp14][101269:102094:+;Tail completion protein gp15][102093:102633:+;Terminase %2C small subunit][102601:104431:+;Terminase %2C large subunit][104450:106442:+;Tail sheath protein][106490:106976:+;Tail tube protein gp19][107031:108606:+;Portal protein][108856:109261:+;Prohead assembly protein gp68][109263:109911:+;Prohead core protein protease][109943:110732:+;Capsid assembly scaffolding protein][110751:112320:+;Major capsid protein][112405:113041:+;HP][113073:114363:+;Capsid vertex protein][114667:115087:+;Recombination protein uvsY][115086:115263:+;HP][115592:115826:−;ATP-dependent DNA helicase uvsW][115834:117175:−;ATP-dependent DNA helicase uvsW][117404:118028:+;HP][118120:118432:+;HP][118471:119041:+;HP][120141:120417:+; Highly immunogenic outer capsid protein][120430:120949:+;HP][120980:121445:−;HP][121494:122334:−;HP][122448:123324:+;HP][123356:123674:+;HP][123670:123847:+;HP][123843:124170:+;HP][124171:124528:+;HP][124527:125052:+;HP][125055:125271:+;RNA ligase 2][125300:126074:+;RNA ligase 2][126077:126596:+;HP][126585:127173:+;HP][127768:128029:+;HP][128038:128488:+;HP][128515:129949:+;HP][130234:131086:+;DNA adenine methylase][131094:131370:+;Glutaredoxin][131401:131884:+;Recombination endonuclease VII][131880:134004:+;Anaerobic ribonucleoside-triphosphate reductase][134311:134806:+;HP][134805:135012:+;HP][135344:135539:+;HP][135535:136063:+;HP][136059:136551:+;HP][136620:137580:+;HP][137622:138291:+;HP][138250:138532:+; Glutaredoxin][138528:138828:+;HP][138907:142681:+;Long-tail fiber proximal subunit][142680:143805:+;Long-tail fiber protein gp35][143854:144523:+;Long-tail fiber protein gp36][149181:149829:+;Holin][150095:150386:−;HP][150385:151183:−;HP][151263:151491:−;HP][151487:151820:−;HP][151794:151932:−;HP][151976:152225:−;HP][152261:152699:−;HP][152673:153048:−;HP][153093:153993:−;HP][153992:156254:−;HP][156264:156501:−;HP][156504:157059:−;HP][157130:158417:−;HP][158416:160315:−;HP][160382:160547:−;HP][160546:160987:−;HP][161009:161594:−;HP][161580:161859:−;HP][161855:162452:−;HP][162491:162821:−;HP][163530:163833:−;HP][163829:164054:−;HP][164270:164552:−;HP][164548:165220:−;Exodeoxyribonuclease][165216:165501:−;HP][165501:165810:−;HP][165834:167163:−;ATP-dependent DNA helicase dda][167159:167450:−;HP][167631:168168:−;HP][168175:169201:−;DNA primase][169204:169465:−;HP][169520:169739:−;HP][169735:170224:−;HP][170229:170961:−;HP][171327:171564:−;HP][171655:171910:−;HP][172302:173739:−;ATP-dependent helicase 41][173748:174069:−;HP][174106:175264:−;Recombination and repair protein][175338:176682:−;DNA-directed DNA polymerase]

Essential genes of phage KP10-91: [854:1196:+;HP][1372:1540:+;HP][1555:1744:+;HP][1912:2173:+;HP][2512:2917:+;Protein A2][2983:3253:+;HP][3249:3477:+;HP][3560:5243:+;Protein A1][5306:5759:+;HP][5832:6549:+;5′-deoxynucleotidase][7151:7376:−;HP][7489:9466:+;Receptor-binding protein][9476:9950:+;putative terminase %2C small subunit][9949:11266:+;Terminase %2C large subunit][11499:11940:+;Nicking endonuclease][11939:13151:+;Portal protein][13147:13618:+;HP][13814:14411:+;Prohead protease][14424:15807:+;Major capsid protein][15865:16378:+;HP][16428:17139:+;Tail completion protein][17138:17624:+;Tail tube terminator protein][17647:18775:+;Tail tube protein][18784:19672:+;Minor tail protein][19675:20098:+;HP][20171:20498:+;HP][20582:25631:+;HP][25745:26360:+;Distal tail protein][26356:29203:+;putative baseplate hub protein][29230:39601:+;HP][39604:40024:+;L-shaped tail fiber protein p132][40023:42063:+;HP][42108:42552:−;Deoxyuridine 5′-triphosphate nucleotidohydrolase][42551:43430:−;T5 flap endonuclease][43426:43909:−;Protein D14][43908:45744:−;putative exonuclease subunit 2][45727:46720:−;putative exonuclease subunit 1][46759:47533:−;putative ssDNA-binding protein][47535:47889:−;HP][48091:49450:−;putative helicase D10][49936:52507:−;DNA polymerase][52574:53459:−;HP][53455:54934:−;HP][55018:55777:−;Putative transcription factor D5][55779:56529:−;HP][56728:57691:−;DNA ligase][57690:57966:−;HP][57922:58084:−;HP][58139:58664:−;HP][58623:58926:−;HP][58976:59273:−;HP][59285:59690:−;HP][59768:60284:−;HP][60276:60972:−;HP][61004:61307:−;HP][61275:64065:−;Putative replication origin binding protein][64663:65059:−;HP][65055:65457:−;HP][65443:65608:−;HP][65594:66425:−;HP][66424:66637:−;HP][66697:68527:−;Anaerobic ribonucleoside-triphosphate reductase][68836:69586:+;HP][69587:69701:+;HP][69715:69925:+;HP][70053:72378:+;Ribonu cleoside-diphosphate reductase large subunit][72474:73761:+;HP][73760:74324:+;HP][74494:75175:+;Thymidylate synthase][75181:75415:+;HP][75512:75788:+;HP][75787:76270:+;HP][76269:76431:+;HP][76420:76705:+;HP][76834:77245:+;HP][77244:78249:+;HP][78313:78775:+;HP][80130:80457:+; HP][80459:80636:+;HP][80864:81158:+;HP][81291:81489:+;HP][81488:81698:+;HP][81708:82203:+;HP][84038:84230:+;HP][84237:84396:+;HP][85267:85549:+;HP][85569:85947:+;HP][85946:86162:+;HP][86233:86347:+;HP][87778:87973:+;HP][87975:88314:+;HP][88313:88463: +;HP][88687:89197:+;HP][89534:89636:+;HP][89754:89946:+;HP][90043:90400:+;HP][90390: 90771:+;HP][91061:91265:+;HP][91261:91525:+;HP][91524:92067:+;HP][92053:92281:+;HP][92273:92636:+;HP][92604:92871:+;HP][92860:93166:+;HP][93162:93372:+;HP][93373:93811: +;HP][93857:94181:+;HP][94173:94446:+;HP][94438:94873:+;HP][94992:95385:+;HP][95493: 96192:+;HP][96157:96619:+;HP][96741:96861:+;HP][96857:97619:+;Deoxynucleoside-5′-monophosphate kinase][97628:98240:+;HP][98397:99060:+;Holin][99056:99470:+;L-alanyl-D-glutamate peptidase][100019:100424:+;HP][100416:100701:+;HP][100802:101204:+;HP][101204:101567:+;HP][101566:102397:+;Serine/threonine-protein phosphatase][102407:102641:+;HP][102649:103018:+;HP][103020:103140:+;HP][103203:103632:+;HP][103713:104019:+;HP][104018:104300:+;HP][104296:104545:+;HP][104541:104883: +;HP][104875:105103:+;HP][105086:105554:+;HP][105513:105903:+;HP][105914:106202:+;H P][106895:107297:+;HP][107309:107819:+;HP][107831:108152:+;HP][108209:109076:+;HP][110791:110935:−;HP][110931:111111:−;HP][111107:111200:−;HP][111199:111313:−;HP][111508:112474:+;HP][112539:113034:+;HP][113143:113491:+;HP]

Essential genes of phage KLP6a-30: [23:173:+;HP][320:2849:+;HP][3002:7841:+;HP][7891:8140:+;HP][8123:8462:+;HP][8448:9201:+;HP][9229:9442:−;HP][9503:10145:+;Neck protein gp14][10147:10846:+;HP][10848:11535:+;HP][11515:13726:+;HP][13778:15674:+;Tail sheath protein][15699:16488:−;HP][16555:17011:+;HP][17045:17579:+;Tail tube protein][17646:19329:+;Portal protein][19374:19539:+;HP][19548:19857:+;HP][19867:20533:+;HP][20578:21439:+;HP][2153 3:22856:+;Major capsid protein][22939:23587:+;HP][23647:23893:+;HP][23889:24225:+;HP][24571:24778:+;HP][24758:24989:+;HP][25004:25442:+;HP][25441:25882:+;HP][25883:26120:+;HP][26223:26646:+;H P][26712:26994:+;HP][26990:27434:+;HP][27457:27625:+;HP][27659:28388:+;HP][28388:29003:−;HP][29077:29578:+;HP][29615:30077:+;HP][30066:30792:+;HP][30788:31535:+;HP][31562:33080:+;ATP-dependent DNA helicase uvsW][33064:33448:−;HP][33771:34440:+;DNA polymerase processivity component][34525:35515:+;Sliding-clamp-loader gp44 subunit][35517:35940:+;HP][35968:36433:+;HP][36449:37316:+;HP][37387:38608:+;HP][38604:38829:+;HP][41018:41390:+;HP][41454:41829:+;HP][41837:42710:+;HP][42860:43037:+; HP][43029:45228:+;HP][45272:45596:+;HP][45689:45935:+;HP][46224:46593:+;HP][46612:46792:+;HP][46904:47354:+;HP][47313:48189:+;HP][48277:48964:+;HP][48960:49308:+;HP][49357:50107:+;HP][50155:50848:+;HP][50844:51120:+;HP][51100:51427:+;HP][51423:51738: +;HP][51855:52134:+;Transcription factor 1][52380:53982:+;HP][53978:54746:−;HP][55084:55327:+;HP][55330:56101:+;HP][56087:57206:+;HP][57208:59536:+;HP][59538:59694:+;HP][59690:60011:+;HP][60000:60282:+;HP][60379:60997:+;HP][60996:61635:+;HP][61743:62019:+;HP][62095:64864:+;HP][64917:65484:+;HP][65493:65733:+;HP][65787:66141: +;HP][66196:66805:+;HP][66801:67473:+;HP][67472:67664:+;HP][67660:68524:+;DNA primase][68523:68736:+;HP][68916:69405:+;HP][69461:69659:+;HP][69651:69825:+;HP][69890:70685:+;HP][70791:71634:+;HP][71720:74003:+;Ribonucleoside-diphosphate reductase large subunit][74033:74768:+;HP][74831:75125:+;HP][75126:76233:+;HP][76242:76467:+;HP][76561:77014:+;HP][77020:77389:+;HP][77371:77752:−;Baseplate wedge protein gp25][77815:79426:−;HP][79930:80734:−;HP][80783:81314:+;HP][81285:81774:+;HP][81812:82424:+;HP][82416:82713:+;HP][82699:82945:+;HP][82937:83180:+;HP][83189:83429:+;HP][83525:84572:+;HP][84615:85023:+;HP][85045:85963:−;HP][86046:86754:+;HP][86820:87555:+;HP][87579:87891:+;HP][88028:88922:+;HP][88996:89659:+;HP][89658:90702:+;HP][90698:91265:+;HP][91261:91813:+;HP][91812:92331:+;HP][92315:93401:+;Recombination and repair protein][93378:93708:+;HP][93714:95142:+;ATP-dependent helicase 41][95204:95540:+;HP][95553:95868:+;HP][95986:97180:+;HP][97176:97317:+;HP][97316:97529:+;HP][97988:99410:+;HP][99411:99669:+;HP][99634:99895:+;HP][99884:100547:+;HP][100549:102535:−;HP][102545:103934:−;HP][103930:104488:−;HP][104502:105471:−;HP][105524:106265:+;HP][106264:106882:+;HP][107089:107497:−;HP][107507:108014:−;HP][108006:108351:−;HP][108413:108809:−;HP][108805:109378:−;HP][109367:109826:−;HP][109827:110499:−;HP][110501:110714:−;HP][110777:111074:−;HP][111073:111703:−;HP][111695:112301:−;HP][112297:112525:−;HP][112524:112767:−;HP][112810:113215:−;HP][113218:113533:−;HP][113532:113823:−;HP][113865:115197:−;HP][115198:117109:−;HP][117157:117784:−;HP][117780:118266:−;HP][118309:118501:−;HP][118547:119051:−;HP][119139:119361:−;HP][119362:120172:−;HP][120150:120462:−;HP][120574:120985:−;HP][120962:121259:−;HP][121311:122898:−;HP][122931:125688:−;HP][125790:126129:−;HP][126139:126307:−;HP][126303:126522:−;HP][126521:127175:−;HP][127171:127558:−;HP][127623:128046:−;HP][128050:128614:−;HP][128631:129723:−;HP][129719:130109:−;HP][130189:131407:−;HP][131410:131791:−;HP][131787:131991:−;HP][131993:132878:−;HP][132877:133126:−;HP][133187:133979:−;HP][133975:134326:−;HP][134393:137390:−;DNA-directed DNA polymerase][137468:138041:+;HP][138387:138915:+;HP][138996:139452:−;HP][139520:140900:−;HP][140968:141601:−;HP][142987:143188:−;HP][143601:143880:−;HP][146191:146761:−;HP][147115:148897:+;Baseplate wedge protein gp6][148880:149732:+;HP][149736:151062:+;HP][151113:154122:+;HP][154154:156686:+;HP]

Essential genes of phage CT-227-KP4M1-19: [3987:7434:−;HP][7521:8124:−;HP][8098:8836:−;HP][8837:9590:−;Tail tip protein L][9659:10004:−;HP][10006:13102:−;HP][13452:13779:−;HP][13855:14509:−;HP][14564:15260:−;HP][15246:15678:−;HP][15667:16105:−;HP][16097:16475:−;HP][16480:16897:−;HP][16950:17256:−;HP][17347:18307:−;HP][18417:18936:−;HP][18987:20118:−;HP][20117:20885:−;HP][20874:22185:−;HP][22230:23832:−;HP][23841:24366:−;HP][24445:24709:−;HP][24702:24822:−;HP][25026:25260:−;HP][25247:25484:−;HP][25553:26129:−;HP][26125:26338:−;HP][26449:26641:−;HP][26706:27030:−;HP][27105:27594:−;HP][27593:27812:−;HP][27891:28077:−;HP][28073:28319:−;HP][28318:28621:−;HP][28630:29002:−;HP][29075:29273:−;HP][29269:29527:−;HP][29613:29934:−;HP][29944:30157:−;HP][30225:30450:−;HP][31095:31248:−;HP][31400:31706:−;HP][32906:33188:+;HP][33260:33455:+;HP][33451:33691:+;HP][33690:34068:+;HP][34140:34368:+;HP][34364:35057:+;HP][35129:35333:+;HP][35329:35512:+;HP][35512:35950:+;HP][36075:37644:+;HP][37647:38109:+;HP][38178:38604:−;HP][38600:39083:−;Lysozyme][39084:39300:−;HP][39426:40008:−;HP][40004:40493:−;HP][40530:41661:−;HP][41756:42008:−;HP][42004:42241:−;HP][42305:42542:−;HP][42545:43274:−;HP][43451:43859:−;HP][43858:45892:−;HP][45983:46385:+;HP][46460:47420:+;HP][47915:48962:+;HP][49021:49678:+;HP]

Essential genes of phage KP10-48: [658:886:−;HP][882:1152:−;HP][1219:1624:−;Protein A2][1963:2224:−;HP][2390:2579:−;HP][2594:2762:−;HP][3615:3963:−;HP][3959:4484:−;HP][4538:5033:−;HP][5098:6064:−;HP][6136:6475:−;HP][6619:6733:+;HP][6732:6903:+;HP][6899:7043:+;HP][8774:9626:−;HP][9701:10016:−;HP][10028:10538:−;HP][10550:10952:−;HP][11645:11933:−;HP][11944:12334:−;HP][12293:12761:−;HP][12744:12972:−;HP][12964:13306:−;HP][13302:13551:−;HP][13547:13829:−;HP][13828:14134:−;HP][14215:14644:−;HP][14707:14827:−;HP][14829:15198:−;HP][15206:15440:−;HP][15450:16281:−;Serine/threonine-protein phosphatase][16280:16643:−;HP][16642:17047:−;HP][17148:17433:−;HP][17425:17830:−;HP][18379:18793:−;L-alanyl-D-glutamate peptidase][18789:19452:−;Holin][19609:20221:−;HP][20230:20992:−;Deoxynucleoside-5′-monophosphate kinase][20988:21108:−;HP][21230:21692:−;HP][21657:22371:−;HP][22479:22872:−;HP][22991:23426:−;HP][23418:23718:−;HP][23683:24007:−;HP][24053:24491:−;HP][24492:24702:−;HP][24698:25004:−;HP][24993:25260:−;HP][25228:25591:−;HP][25583:25811:−;HP][25797:26340:−;HP][26339:26612:−;HP][26615:26813:−;HP][26925:27096:−;HP][27257:27638:−;HP][27628:27985:−;HP][28082:28274:−;HP][28991:29141:−;HP][29140:29479:−;HP][29481:29676:−;HP][31285:31399:−;HP][31470:31686:−;HP][31685:32063:−;HP][32083:32365:−;HP][33084:33243:−;HP][33250:33442:−;HP][35410:35905:−;HP][35915:36125:−;HP][36124:36322:−;HP][36451:36760:−;HP][36988:37165:−;HP][37167:37494:−;HP][38756:39218:−;HP][39282:40287:−;HP][40286:40697:−;HP][40826:41111:−;HP][41100:41262:−;HP][41261:41744:−;HP][41743:42019:−;HP][42116:42296:−;HP][42302:42983:−;Thymidylate synthase][43153:43717:−;Dihydrofolate reductase][43716:44874:−;HP][44970:47295:−;Ribonucleoside-diphosphate reductase large subunit][47423:47633:−;HP][47647:47761:−;HP][47762:48512:−;HP][48821:50651:+;Anaerobic ribonucleoside-triphosphate reductase][50711:50921:+;HP][50922:51756:+;HP][51739:51904:+;HP][51890:52292:+;HP][52288:52684:+;HP][53282:56072:+;Putative replication origin binding protein][56058:56343:+;HP][56375:57071:+;HP][57356:57761:+;HP][57773:58070:+;HP][58120:58429:+;HP][58512:58650:+;HP][58630:58906:+;HP][58905:59868:+;DNA ligase][60067:60817:+;HP][60819:61578:+;Putative transcription factor D5][61662:63141:+;HP][63137:64022:+;HP][64089:66660:+;DNA polymerase][67146:68505:+;putative helicase D10][68707:69061:+;HP][69063:69837:+;putative ssDNA-binding protein][69876:70869:+;putative exonuclease subunit 1][70852:72688:+;putative exonuclease subunit 2][72687:73170:+;Protein D14][73166:74045:+;T5 flap endonuclease][74044:74488:+;Deoxyuridine 5′-triphosphatenucleotidohydrolase][74532:76572:−;HP][76571:76991:−;L-shaped tail fiber protein p132][76994:87374:−;HP][87401:90248:−;putative baseplate hub protein][90244:90859:−;Distal tail protein][90973:96052:−;HP][96136:96463:−;HP][96536:96959:−;HP][96962:97850:−;Minor tail protein][97859:98987:−;Tail tube protein][99010:99496:−;Tail tube terminator protein][99495:100206:−;Tail completion protein][100256:100769:−;HP][100827:102210:−;Major capsid protein][102223:102820:−;Prohead protease][103019:103739:−;HP][103735:104947:−;Portal protein][104946:105387:−;Nicking endonuclease][105620:106937:−;Terminase %2C large subunit][106936:107410:−;putative terminase %2C small subunit][107420:109397:−;Receptor-binding protein][109510:109735:+;HP][110338:111055:−;5′-deoxynucleotidase][111128:111581:−;HP][111649:112804:−;Protein A1]

Essential genes of phage KP10-2: [25:1132:+;HP][1195:1474:+;HP][1495:2422:−;Baseplate tail-tube junction protein gp54][2421:3474:−;Baseplate tail-tube junction protein gp48][3483:5217:−;HP][5213:5741:−;Baseplate hub assembly protein gp28][5712:6855:−;Baseplate central spike complex protein gp27][6851:7610:−;HP][7661:8291:+;HP][8287:8698:+;Baseplate wedge protein gp25][8739:9156:+;Recombination protein uvsY][9186:9354:+;HP][9413:9656:−;ATP-dependent DNA helicase uvsW][9652:10636:−;ATP-dependent DNA helicase uvsW][11213:12008:+;HP][12017:13424:+;HP][13502:13730:+;HP][13729:14056:+;HP][14169 :14427:+;HP][14437:15445:+;RNA ligase 2][15444:15993:+;HP][16839:18120:−;Capsid vertex protein][18201:19761:−;Major capsid protein][19781:20594:−;Capsid assembly scaffolding protein][20626:21277:−;Prohead core protein protease][21276:21702:−;Prohead assembly protein gp68][21701:21926:−;HP][21925:23494:−;Portal protein][23598:24090:−;Tail tube protein gp19][24218:26192:−;Tail sheath protein][26225:28064:−;Terminase %2C large subunit][28041:28551:−;Terminase %2C small subunit][28547:29372:−;Tail completion protein gp15][29413:30190:−;Neck protein gp14][30193:31135:−;Neck protein gp13][31191:32958:−;HP][32966:34313:−;HP][34312:34984:−;Baseplate wedge protein gp11][34985:36809:−;Baseplate wedge protein gp10][36808:37720:−;Baseplate protein gp9][37787:38813:−;Baseplate wedge protein gp8][38805:41904:−;Baseplate wedge protein gp7][41900:43859:−;Baseplate wedge protein gp6][43858:44152:−;Baseplate puncturing device gp5.4][44152:44656:−;HP][44658:46389:−;Baseplate central spike complex protein gp5][46385:47024:−;Baseplate wedge protein gp53][47073:47523:+;HP][47522:48350:+;HP][48349:48952:+;Tail tube terminator protein][49019:49694:+;Deoxynucleotide monophosphate kinase][49690:49927:+;HP][49926:50382:+;HP][50457:50637:+;HP][50623:50803:+;HP][50802:50988:+;HP][51004:51355:+;HP][53257:53410:+;HP][53406:53775:+;HP][53758:53992:+;H P][54046:54271:+;HP][54622:55180:+;HP][55179:55629:+;HP][56554:56845:+;HP][56863:57127:+;HP][57113:57341:+;HP][57370:57964:+;HP][58003:58594:+;HP][58590:58953:+;HP][58949:59246:+;HP][59242:59569:+;HP][59568:59805:+;HP][59801:60248:+;HP][60255:60564:+; HP][60593:61088:+;Endolysin][61088:61568:+;HP][61649:62222:+;Internal protein III][62415:62721:+;HP][62801:63335:+;HP][63331:63643:+;HP][63624:63969:+;HP][63977:64340:+;HP][64339:64615:+;HP][64678:65137:+;Endoribonuclease RegB][65144:65690:+;HP][65689:66010:+;HP][66006:66399:+;HP][66407:66875:+;HP][66899 :67067:+;HP][67066:67666:+;HP][67646:67841:+;HP][68125:68500:+;HP][68546:68759:+;HP][68770:69058:+;Antiholin][69044:69431:+;HP][69535:70024:+;HP][70115:70382:+;HP][70384 :70576:+;HP][70565:70787:+;HP][70789:71281:+;HP][71277:71859:+;HP][72051:72474:+;HP][72470:73481:+;HP][74028:74244:+;HP][74321:75299:+;HP][75341:75488:+;HP][75484:75910:+;HP][75906:76242:+;HP][76238:76559:+;HP][76561:76759:+;HP][77122:77287:+;HP][77283:77550:+;HP][77607:78396:+;HP][78466:79441:+;HP][79461:79698:+;HP][79694:80009:+;H P][80005:80338:+;HP][80334:80844:+;HP][80840:81008:+;HP][80997:81288:+;HP][81250:81520:+;Glutaredoxin][81512:81701:+;HP][81990:82161:+;HP][82162:82552:+;HP][82530:82689: +;HP][82672:83125:+;HP][83117:83426:+;HP][83462:83936:+;Recombination endonuclease VII][83932:85771:+;Anaerobic ribonucleoside-triphosphate reductase][85742:85982:+;HP][85947:86580:+;HP][86569:87040:+;HP][87157:87370:+;HP][87366:87708:+;HP][87704:88007:+;HP][88193:88382:+;HP][88378:88621:+;HP][88685:89018:+; HP][89014:89272:+;HP][89264:89549:+;HP][89541:89766:+;HP][89753:90032:+;HP][90028:90274:+;HP][90340:90889:+;RNA polymerase sigma factor][90872:91103:+;HP][91104:91407:+;HP][91384:91582:+;HP][91655:91913:+;HP][91982 :93179:+;DNA alpha-glucosyltransferase][93248:94271:+;Exonuclease subunit 1][94309:95950:+;Exonuclease subunit 2][95946:96141:+;HP][96151:96529:+;15 kDa RNA polymerase-binding protein][96565:97267:+;DNA polymerase clamp][97315:98278:+;Sliding-clamp-loader gp44 subunit][98274:98844:+;Sliding-clamp-loader gp44 subunit][98846:99209:+;Translation repressor protein][99301:102010:+;DNA-directed DNA polymerase][102079:102523:+;HP][102533:102740:+;Immunity protein][102748:103483:+;Deoxycytidylate 5-hydroxymethyltransferase][103479:104316:+;HP][104342:104564:+;HP][104646:105822:+;Rec ombination and repair protein][105811:106162:+;HP][106237:107611:+;ATP-dependent helicase 41][107620:107854:+;HP][107919:108198:+;HP][108208:108502:+;Protein spackle][108498:108708:+;HP][108723:108888:+;HP][108926:109949:+;DNA primase][109967:110159:−;HP][110225:110747:+;HP][110746:110884:+;HP][110914:111142:+;Small outer capsid protein][111220:111412:+;HP][111913:112087:+;HP][112160:112826:+;HP][112906:113662:+; HP][113661:113976:+;HP][113972:115304:+;ATP-dependent DNA helicase dda][115310:115565:+;HP][115557:116247:+;Exodeoxyribonuclease][116246:116414:+;HP][116477:116885:+;HP][116949:117165:+;HP][117164:117530:+;HP][117523:117859:+;HP][117845:118022:+;HP][118021:118111:+;HP][118107:118356:+;HP][118434:118803:+;HP][118833: 120672:+;HP][120719:120872:+;HP][121011:121218:+;HP][121228:123397:+;Protein rIIA][123397:124309:+;HP][124351:124648:+;HP][124589:125168:+;Endonuclease IV][125232:125472:+;HP][125532:125649:+;HP][125659:125851:+;HP][125847:125937:+;HP][125995:126451:+;Nucleoid disruption protein][126516:126624:+;HP][126620:126761:+;HP][126766:128155:+;HP][128382:128646:+; HP][128770:129415:+;Middle transcription regulatory protein motA][129414:129756:+;HP][129752:129932:+;HP][129919:130282:+;HP][130281:130584:+; HP][130767:130893:+;HP][130876:131155:+;HP][131151:131478:+;HP][131474:131741:+;HP][131808:132081:+;10 kDa anti-sigma factor][132081:132738:−;Holin][132766:133306:−;HP][133343:137990:−;HP][137999:138692:−;Long-tail fiber protein gp36][138749:139886:−;Long-tail fiber protein gp35][139885:143731:−;Long-tail fiber proximal subunit][143807:144734:+;Ribonuclease H][144744:145017:+;Double-stranded DNA-binding protein][144997:145297:+;RNA polymerase-associated protein Gp33][145293:145956:+;Protein Gp59][146023:146941:+;Single-stranded DNA-binding protein][147067:147325:+;HP][147366:147588:+;HP][147591:147957:+;HP][148013:148292:+; HP][148288:148378:+;HP][148370:148697:+;HP][148775:148940:+;HP][148947:149547:+;Dih ydrofolate reductase][149530:149848:+;HP][149840:150716:+;Thymidylate synthase][150712:150919:+;HP][150915:151221:+;HP][151211:153458:+;Ribonucleoside-diphosphate reductase large subunit][153514:154657:+;HP][154684:155101:+;Endonuclease II][155094:156246:+;T4 RNA ligase 1][156308:156818:+;HP][156805:157159:+;HP][157155:157443:+;HP][157453:157666:+;HP][157665:158559:+;Polynucleotide kinase][158570:158855:+;HP][158844:159081:+;HP][159080:159425:+;HP][159425:159995:+; Deoxycytidylate deaminase][159994:160195:+;HP][160191:160500:+;HP][160557:160881:+;Capsid assembly protein Gp31][160995:161244:+;Lysis inhibition accessory protein][161610:161787:+;HP][161908:162277:+;HP][162317:162830:+;HP][162923:163283:+; HP][163322:163808:+;HP][163820:164099:+;HP][164109:164280:+;HP][164276:164477:+;HP][164660:165503:+;HP][165544:165751:+;HP][165750:167223:+;HP][167219:167399:+;HP]

Essential genes of phage KP10-84: [164:443:−;HP][439:1036:−;HP][1075:1405:−;HP][2114:2417:−;HP][2854:3136:−;HP][3132:3804:−;Exodeoxyribonuclease][3800:4085:−;HP][4085:4394:−;HP][4418:5747:−;ATP-dependent DNA helicase dda][5743:6034:−;HP][7087:7624:−;HP][7631:8657:−;DNA primase][8660:8921:−;HP][8976:9195:−;HP][9191:9680:−;HP][9685:10417:−;HP][10783:11020:−;HP][11111:11366:−;HP][11758:13195:−;ATP-dependent helicase 41][13204:13525:−;HP][13562:14720:−;Recombination and repair protein][14794:17494:−;DNA-directed DNA polymerase][17866:18154:−;HP][18222:18585:−;Translation repressor protein][18588:19152:−;Sliding-clamp-loader gp44 subunit][19154:20153:−;Sliding-clamp-loader gp44 subunit][20211:20877:−;DNA polymerase clamp][20903:21173:−;HP][21228:22200:−;HP][22215:22794:−;Protein Gp59][22871:23117:−;RNA polymerase-associated protein Gp33][23120:23384:−;Double-stranded DNA-binding protein][23393:24329:−;Ribonuclease H][24633:25161:+;RNA polymerase sigma factor][25157:25373:+;HP][25728:26775:+;Exonuclease subunit 1][27032:28730:+;Exonuclease subunit 2][28729:28921:+;HP][28994:30704:+;HP][30763:31054:+;HP][31050:31281:+;HP][31277:31535:+;HP][31531:32110:+;HP][32111:32987:+;Thymidylate synthase][33026:35279:+;Ribonucleoside-diphosphate reductase large subunit][35315:36491:+;Ribonucleoside-diphosphate reductase subunit beta][36786:37152:+;Endonuclease H1][37132:38290:+;T4 RNA ligase 1][38496:38823:+;HP][38791:39127:+;HP][39123:39441:+;HP][39589:40474:+;Polynucleotide kinase][40526:40814:+;HP][40810:41062:+;HP][41277:41835:+;Deoxycytidylate deaminase][41850:42192:+;Capsid assembly protein Gp31][42202:42454:+;HP][42769:43051:+;HP][43060:43483:+;HP][43524:44091:+;HP][44101 :44464:+;HP][44463:44733:+;HP][44858:45152:+;HP][45852:46293:+;HP][46289:46808:+;HP][46816:47080:+;HP][47081:47303:+;HP][47383:47914:+;HP][47894:49424:+;HP][50159:50432:+;HP][50857:51157:+;HP][51143:51521:+;HP][51517:52627:−;HP][52696:53272:+;HP][53268:55050:+;HP][55060:56134:+;Baseplate tail-tube junction protein gp48][56143:57007:+;Baseplate tail-tube junction protein gp54][57035:57779:+;HP][57775:58330:+;HP][58330:58723:+;Baseplate wedge protein gp25][58757:59213:−;HP][59220:59445:−;HP][59802:60087:−;HP][60102:60573:−;HP][60724:61342:−;HP][61413:63630:−;HP][63637:63958:−;HP][63957:64473:−;HP][64698:64986:−;HP][65192:65471:−;HP][65529:66441:−;HP][66437:66695:−;HP][66694:66940:−;HP][66936:67944:−;HP][67943:68321:−;HP][68317:68545:−;HP][69149:69422:−;HP][69786:70848:−;HP][71024:71939:−;HP][72009:72432:−;HP][72403:72685:−;HP][72791:73184:−;HP][73200:73809:−;HP][73991:74279:−;HP][74281:74755:−;HP][75061:75385:−;HP][75394:75985:−;HP][75981:76128:−;HP][76138:76534:−;HP][76601:77243:−;HP][77307:77862:−;HP][77873:78071:−;HP][78108:79455:−;HP][79464:79800:−;HP][79989:80448:−;HP][80479:80875:−;L-alanyl-D-glutamate peptidase][80942:81344:−;HP][81760:82024:−;HP][82093:82354:−;HP][82355:82787:−;HP][82967:83315:−;HP][83295:83661:−;HP][83943:85098:−;HP][85831:86086:−;HP][86348:86495:−;HP][86491:86743:−;HP][86814:87093:−;HP][87176:87434:−;HP][88589:88790:−;HP][89174:89708:−;HP][89700:90036:−;HP][90043:90304:−;HP][90300:90681:−;HP][90677:91091:−;HP][91600:91897:−;HP][92156:92495:−;HP][92505:92646:−;HP][92735:92876:−;HP][92942:93482:−;HP][93503:94130:−;HP][94456:95152:−;Deoxynucleotide monophosphate kinase][95151:95682:−;Tail tube terminator protein][96051:96840:+;HP][96876:97731:−;HP][97746:98106:−;HP][98459:98819:+;Baseplate wedge protein gp53][98815:100585:+;Baseplate central spike complex protein gp5][100589:102656:+;HP][102687:104613:+;Baseplate wedge protein gp6][104693:107780:+;Baseplate wedge protein gp7][107780:108773:+;Baseplate wedge protein gp8][108782:109646:+;Baseplate protein gp9][109642:111460:+;Baseplate wedge protein gp10][111459:112128:+;Baseplate wedge protein gp11][112137:113523:+;Short tail fiber protein gp12][113533:115285:+;Fibritin][115323:116250:+;Neck protein gp13][116866:117613:+;Neck protein gp14][117692:118517:+;Tail completion protein gp15][118516:119056:+;Terminase %2C small subunit][119024:120854:+;Terminase %2C large subunit][120873:122865:+;Tail sheath protein][122913:123399:+;Tail tube protein gp19][123454:125029:+;Portal protein][125279:125684:+;Prohead assembly protein gp68][125686:126334:+;Prohead core protein protease][126366:127155:+;Capsid assembly scaffolding protein][127174:128743:+;Major capsid protein][128834:129473:+;HP][129505:130795:+;Capsid vertex protein][131091:131511:+;Recombination protein uvsY][131510:131687:+;HP][132146:132380:−;ATP-dependent DNA helicase uvsW][132388:133729:−;ATP-dependent DNA helicase uvsW][133958:134537:+;HP][134629:134941:+;HP][134980:135550:+;HP][135578:136112:+; Highly immunogenic outer capsid protein][136122:136398:+;Highly immunogenic outer capsid protein][136411:136930:+;HP][136962:137427:−;HP][137476:138316:−;HP][138430:139306:+;HP][139338:139656:+;HP][139652:139829:+;HP][139825:140152:+;HP][140153:140510:+;HP][140509:141034:+;HP][141037:142057:+;RNA ligase 2][142046:142628:+;HP][143205:143466:+;HP][143475:143925:+;HP][143952:145386:+;HP][145671:146523:+;DNA adenine methylase][146531:146807:+;Glutaredoxin][146838:147321:+;Recombination endonuclease VII][147640:149764:+;Anaerobic ribonucleoside-triphosphate reductase][150070:150568:+;HP][150567:150774:+;HP][151106:151301:+;HP][151297:151825:+;HP][151821:152313:+;HP][152382:153342:+;HP][153384:154053:+;HP][154012:154294:+; Glutaredoxin][154290:154590:+;HP][154669:158443:+;Long-tail fiber proximal subunit][158442:159567:+;Long-tail fiber protein gp35][159616:160285:+;Long-tail fiber protein gp36][160293:163998:+;HP][164519:165167:+;Holin][165433:165724:−;HP][165723:166521:−;HP][166601:166829:−;HP][166825:167158:−;HP][167132:167267:−;HP][167311:167560:−;HP][167596:168034:−;HP][168008:168383:−;HP][168428:169328:−;HP][169327:171589:−;HP][171599:171836:−;HP][171839:172394:−;HP][172465:173752:−;HP][173751:175650:−;HP][175717:175882:−;HP][175881:176322:−;HP]

Essential genes of phage KP10-64: [1795:2662:−;HP][2717:3038:−;HP][3050:3560:−;HP][3572:3974:−;HP][4667:4955:−;HP][4965:5355:−;HP][5314:5782:−;HP][5765:5993:−;HP][5985:6327:−;HP][6323:6572:−;HP][6568:6850:−;HP][6849:7155:−;HP][7236:7665:−;HP][7728:7848:−;HP][7850:8219:−;HP][8227:8461:−;HP][8471:9302:−;Serine/threonine-protein phosphatase][9301:9619:−;HP][9654:10056:−;HP][10157:10442:−;HP][10434:10839:−;HP][11388:11802:−;L-alanyl-D-glutamate peptidase][11798:12461:−;Holin][12618:13230:−;HP][13239:14001:−;Deoxynucleoside-5′-monophosphate kinase][13997:14117:−;HP][14239:14701:−;HP][14666:15365:−;HP][15473:15866:−;HP][15985:16420:−;HP][16412:16685:−;HP][16677:17001:−;HP][17047:17485:−;HP][17486:17696:−;HP][17692:17998:−;HP][17987:18254:−;HP][18222:18585:−;HP][18577:18805:−;HP][18791:19334:−;HP][19333:19606:−;HP][19609:19807:−;HP][19919:20090:−;HP][20251:20632:−;HP][20622:20979:−;HP][21076:21268:−;HP][21386:21488:−;HP][21826:22336:−;HP][22567:22906:−;HP][22908:23103:−;HP][24630:24744:−;HP][24815:25031:−;HP][25030:25408:−;HP][25428:25701:−;HP][26177:26357:−;HP][26343:26535:−;HP][28245:28740:−;HP][28750:28960:−;HP][28959:29157:−;HP][29286:29580:−;HP][29808:29973:−;HP][31288:31750:−;HP][31814:32819:−;HP][32818:33229:−;HP][33358:33643:−;HP][33632:33794:−;HP][33793:34276:−;HP][34275:34551:−;HP][34648:34882:−;HP][34888:35569:−;Thymidylate synthase][35739:36303:−;HP][36302:37460:−;HP][37443:37962:−;HP][38031:40356:−;Ribonucleoside-diphosphate reductase large subunit][40438:40927:−;HP][40996:41206:−;HP][41220:41334:−;HP][41335:42085:−;HP][42393:44223:+;Anaerobic ribonucleoside-triphosphate reductase][44283:44496:+;HP][44495:45329:+;HP][45312:45477:+;HP][45463:45865:+;HP][45861:46257:+;HP][46855:49645:+;Putative replication origin binding protein][49631:49916:+;HP][49948:50644:+;HP][50929:51334:+;HP][51346:51643:+;HP][51693:52002:+;HP][52085:52223:+;HP][52203:52479:+;HP][52478:53441:+;DNA ligase][53640:54390:+;HP][54392:55151:+;Putative transcription factor D5][55235:56714:+;HP][56710:57595:+;HP][57662:60233:+;DNA polymerase][60719:62078:+;putative helicase D10][62280:62634:+;HP][62636:63410:+;putative ssDNA-binding protein][63449:64442:+;putative exonuclease subunit 1][64425:66261:+;putative exonuclease subunit 2][66260:66743:+;Protein D14][66739:67618:+;T5 flap endonuclease][67617:68061:+;Deoxyuridine 5′-triphosphate nucleotidohydrolase][68092:68281:+;HP][68505:72498:−;L-shaped tail fiber protein pb1][72497:72920:−;L-shaped tail fiber protein p132][72924:74955:−;putative central straight fiber][74982:77829:−;putative baseplate hub protein][77825:78440:−;Distal tail protein][78554:83012:−;putative tape measure protein][83096:83423:−;HP][83496:83919:−;HP][83923:84811:−;Minor tail protein][84820:85948:−;Tail tube protein][85971:86457:−;Tail tube terminator protein][86456:87167:−;Tail completion protein][87217:87730:−;HP][87788:89171:−;Major capsid protein][89184:89781:−;Prohead protease][89977:90694:−;HP][90690:91902:−;Portal protein][91901:92342:−;Nicking endonuclease][92575:93892:−;Terminase %2C large subunit][93891:94365:−;putative terminase %2C small subunit][94375:96355:−;Receptor-binding protein][96468:96693:+;HP][97307:98024:−;5′-deoxynucleotidase][98096:98549:−;HP][98613:100296:−;Protein A1][100378:100606:−;HP][100602:100872:−;HP][100938:101343:−;Protein A2][101682:101943:−;HP][102109:102298:−;HP][102313:102481:−;HP][102657:102999:−;HP][103650:103890:−;HP][103889:104237:−;HP][104233:104761:−;HP][104815:105310:−;HP][105375:106341:−;HP][106413:106752:−;HP][106896:107010:+;HP][107009:107183:+;HP]

Essential genes of phage CT-227-KP4M1-20: [61:301:+;HP][361:478:+;HP][488:680:+;HP][676:766:+;HP][824:1280:+;Nucleoid disruption protein][1345:1453:+;HP][1449:1590:+;HP][1595:2984:+;HP][3211:3475:+;HP][3599:4244:+; Middle transcription regulatory protein motA][4243:4585:+;HP][4581:4761:+;HP][4748:5111:+;HP][5110:5413:+;HP][5596:5722:+;H P][5705:5984:+;HP][5980:6307:+;HP][6303:6570:+;HP][6637:6910:+;10 kDa anti-sigma factor][6910:7567:−;Holin][7608:8136:−;HP][8173:12826:−;HP][12835:13528:−;Long-tail fiber protein gp36][13585:14722:−;Long-tail fiber protein gp35][14721:18567:−;Long-tail fiber proximal subunit][18642:19569:+;Ribonuclease H][19579:19852:+;Double-stranded DNA-binding protein][19832:20132:+;RNA polymerase-associated protein Gp33][20128:20791:+;Protein Gp59][20858:21776:+;Single-stranded DNA-binding protein][21902:22160:+;HP][22201:22423:+;HP][22426:22792:+;HP][22848:23127:+;HP][23123:23213:+;HP][23205:23526:+;HP][23608:23773:+;HP][23780:24380:+;Dihydrofolate reductase][24363:24681:+;HP][24673:25549:+;Thymidylate synthase][25545:25752:+;HP][25748:26054:+;HP][26044:28291:+;Ribonucleoside-diphosphate reductase large subunit][28347:29490:+;HP][29517:29934:+;Endonuclease II][29927:31079:+;T4 RNA ligase 1][31141:31651:+;HP][31638:31992:+;HP][31988:32276:+;HP][32286:32499:+;HP][32498:33392:+;Polynucleotide kinase][33403:33688:+;HP][33677:33914:+;HP][33913:34258:+;HP][34258:34828:+;Deoxycyti dylate deaminase][34827:35028:+;HP][35024:35333:+;HP][35390:35714:+;Capsid assembly protein Gp31][35828:36077:+;Lysis inhibition accessory protein][36443:36620:+;HP][36741:37110:+;HP][37150:37663:+;HP][38224:38584:+;HP][38623:39106:+;HP][39118:39397:+;HP][39407:39578:+;HP][39574:39775:+;HP][39958:40801:+;H P][40842:41049:+;HP][41048:42521:+;HP][42517:42697:+;HP][42748:44875:+;HP][44938:45217:+;HP][45238:46165:−;Baseplate tail-tube junction protein gp54][46164:47217:−;Baseplate tail-tube junction protein gp48][47226:48960:−;HP][48956:49484:−;Baseplate hub assembly protein gp28][49455:50598:−;Baseplate central spike complex protein gp27][50594:51353:−;HP][51404:52034:+;HP][52030:52441:+;Baseplate wedge protein gp25][52482:52899:+;Recombination protein uvsY][52929:53097:+;HP][53156:53399:−;ATP-dependent DNA helicase uvsW][53395:54928:−;ATP-dependent DNA helicase uvsW][54955:55750:+;HP][55759:57166:+;HP][57244:57472:+;HP][57468:57798:+;HP][57911 :58169:+;HP][58179:59187:+;RNA ligase 2][59186:59735:+;HP][59764:61045:−;Capsid vertex protein][61126:62686:−;Major capsid protein][62706:63519:−;Capsid assembly scaffolding protein][63551:64202:−;Prohead core protein protease][64201:64627:−;Prohead assembly protein gp68][64626:64848:−;HP][64847:66416:−;Portal protein][66520:67012:−;Tail tube protein gp19][67139:69113:−;Tail sheath protein][69146:70985:−;Terminase %2C large subunit][70962:71472:−;Terminase %2C small subunit][71468:72293:−;Tail completion protein gp15][72334:72955:−;Neck protein gp14][73114:74056:−;Neck protein gp13][74112:75879:−;HP][75887:77234:−;HP][77233:77905:−;Baseplate wedge protein gp11][77906:79730:−;Baseplate wedge protein gp10][79729:80641:−;Baseplate protein gp9][80708:81734:−;Baseplate wedge protein gp8][81726:84825:−;Baseplate wedge protein gp7][84821:86780:−;Baseplate wedge protein gp6][86779:87073:−;Baseplate puncturing device gp5.4][87073:87577:−;HP][87579:89310:−;Baseplate central spike complex protein gp5][89306:89945:−;Baseplate wedge protein gp53][89994:90444:+;HP][90443:91271:+;HP][91270:91873:+;Tail tube terminator protein][91940:92615:+;Deoxynucleotide monophosphate kinase][92611:92848:+;HP][92847:93303:+;HP][93673:93853:+;HP][93839:94019:+;HP][94018:94204:+;HP][94220:94571:+;HP][96472:96625:+;HP][96621:96990:+;HP][96973:97207:+;H P][97261:97486:+;HP][97664:98222:+;HP][98221:98671:+;HP][100058:100349:+;HP][100367: 100631:+;HP][100617:100845:+;HP][100874:101468:+;HP][101507:102098:+;HP][102094:102457:+;HP][102453:102750:+;HP][102746:103073:+;HP][103072:103309:+;HP][103305:103752:+;HP][103759:104068:+;HP][104096:104591:+;Endolysin][104591:105071:+;HP][105244:105778:+;HP][105774:106086:+;HP][106067:106559:+;HP][106567:106930:+;HP][106929:107211:+;HP][107274:107733:+;Endoribonuclease RegB][107740:108286:+;HP][108285:108606:+;HP][108602:108995:+;HP][109003:109471:+; HP][109495:109663:+;HP][109662:110262:+;HP][110721:111105:+;HP][111151:111364:+;HP][111375:111663:+;Antiholin][111649:112036:+;HP][112140:112629:+;HP][112720:112987:+; HP][112989:113181:+;HP][113170:113392:+;HP][113394:113886:+;HP][113882:114464:+;HP][114656:115079:+;HP][115075:116086:+;HP][116631:116847:+;HP][116924:117902:+;HP][117944:118091:+;HP][118087:118513:+;HP][118509:118845:+;HP][118841:119162:+;HP][119164:119362:+;HP][119724:119889:+;HP][119885:120152:+;HP][120209:120998:+;HP][121068: 122043:+;HP][122063:122300:+;HP][122296:122611:+;HP][122607:122940:+;HP][122936:123449:+;HP][123441:123615:+;HP][123604:123895:+;HP][123857:124127:+;Glutaredoxin][124119:124308:+;HP][124597:124768:+;HP][124769:125159:+;HP][125137:125296:+;HP][125279: 125732:+;HP][125724:126033:+;HP][126069:126543:+;Recombination endonuclease VII][126539:128378:+;Anaerobic ribonucleoside-triphosphate reductase][128349:128583:+;HP][128554:129025:+;HP][129035:129134:+;HP][129142:129355:+;HP][129351:129693:+;HP][129689:129992:+;HP][130177:130366:+;HP][130362:130605:+; HP][130669:131002:+;HP][130998:131256:+;HP][131248:131533:+;HP][131525:131750:+;HP][131737:132016:+;HP][132012:132258:+;HP][132324:132873:+;RNA polymerase sigma factor][132856:133087:+;HP][133088:133391:+;HP][133368:133566:+;HP][133639:133897:+; HP][133966:135163:+;DNA alpha-glucosyltransferase]1135233:136256:+;Exonuclease subunit 1][136294:137935:+;Exonuclease subunit 2][137931:138126:+;HP][138136:138514:+;15 kDa RNA polymerase-binding protein][138549:139251:+;DNA polymerase clamp][139299:140262:+;Sliding-clamp-loader gp44 subunit][140258:140828:+;Sliding-clamp-loader gp44 subunit][140830:141193:+;Translation repressor protein][141285:143994:+;DNA-directed DNA polymerase][144063:144507:+;HP][144517:144724:+;Immunity protein][144732:145467:+;Deoxycytidylate 5-hydroxymethyltransferase][145463:146300:+;HP][146326:146548:+;HP][146630:147806:+;Rec ombination and repair protein][147795:148146:+;HP][148221:149595:+;ATP-dependent helicase 41][149604:149838:+;HP][149903:150197:+;Protein spackle][150193:150403:+;HP][150418:150583:+;HP][150621:151644:+;DNA primase][151662:151854:−;HP][151920:152442:+;HP][152441:152579:+;HP][152609:152837:+;Small outer capsid protein][152914:153106:+;HP][153607:153781:+;HP][153851:154541:+;HP][154621:155377:+; HP][155376:155691:+;HP][155687:157019:+;ATP-dependent DNA helicase dda][157025:157280:+;HP][157272:157962:+;Exodeoxyribonuclease][157961:158129:+;HP][158192:158600:+;HP][158664:158880:+;HP][158879:159245:+;HP][159238:159574:+;HP][159560:159737:+;HP][159736:159826:+;HP][159822:160071:+;HP][160149:160518:+;HP][160548: 162387:+;HP][162434:162587:+;HP][162727:162934:+;HP][162944:165113:+;Protein rIIA][165113:166025:+;HP][166067:166364:+;HP][166305:166884:+;Endonuclease IV]

Essential genes of phage KP10-11: [122:413:+;HP][460:1054:+;HP][1093:1684:+;HP][1680:2043:+;HP][2039:2336:+;HP][2332:2659:+;HP][2658:2895:+;HP][2891:3338:+;HP][3345:3654:+;HP][3682:4177:+;Endolysin][4177: 4657:+;HP][4738:5311:+;Internal protein III][5504:5810:+;HP][5888:6422:+;HP][6418:6730:+;HP][7066:7429:+;HP][7428:7704:+;HP][7767:8226:+;Endoribonuclease RegB][8233:8779:+;HP][8778:9099:+;HP][9095:9488:+;HP][9496:9964:+;HP][9988:10156:+;H P][10155:10755:+;HP][10735:10930:+;HP][11214:11589:+;HP][11635:11848:+;HP][11859:12147:+;Antiholin][12133:12520:+;HP][12624:13113:+;HP][13204:13471:+;HP][13473:13665:+;H P][13654:13876:+;HP][13878:14379:+;HP][14571:14994:+;HP][14990:16001:+;HP][16548:16764:+;HP][16841:17819:+;HP][17861:18008:+;HP][18004:18430:+;HP][18426:18762:+;HP][18758:19079:+;HP][19452:19617:+;HP][19613:19880:+;HP][19937:20726:+;HP][20796:21771:+; HP][21791:22028:+;HP][22024:22339:+;HP][22335:22668:+;HP][22664:23174:+;HP][23328:23619:+;HP][23581:23851:+;Glutaredoxin][23843:24032:+;HP][24050:24221:+;HP][24222:24612:+;HP][24732:25185:+;HP][25177:25486:+;HP][25523:25997:+;Recombination endonuclease VII][25993:27832:+;Anaerobic ribonucleoside-triphosphate reductase][27803:28037:+;HP][28008:28479:+;HP][28596:28809:+;HP][28805:29108:+;HP][29293:29482:+;HP][29478:29721:+;HP][29785:30118:+;HP][30114:30372:+;HP][30364:30649:+; HP][30641:30866:+;HP][30853:31132:+;HP][31128:31374:+;HP][31440:31989:+;RNA polymerase sigma factor][31972:32212:+;HP][32213:32516:+;HP][32493:32691:+;HP][32764:33022:+;HP][33091 :34288:+;DNA alpha-glucosyltransferase][34358:35381:+;Exonuclease subunit 1][35419:37060:+;Exonuclease subunit 2][37056:37251:+;HP][37261:37639:+;15 kDa RNA polymerase-binding protein][37674:38376:+;DNA polymerase clamp][38424:39387:+;Sliding-clamp-loader gp44 subunit][39383:39953:+;Sliding-clamp-loader gp44 subunit][39955:40318:+;Translation repressor protein][40410:43119:+;DNA-directed DNA polymerase][43188:43632:+;HP][43642:43849:+;Immunity protein][43857:44592:+;Deoxycytidylate 5-hydroxymethyltransferase][44588:45425:+;HP][45451:45673:+;HP][45755:46931:+;Recombination and repair protein][46920:47271:+;HP][47346:48720:+;ATP-dependent helicase 41][48729:48963:+;HP][49028:49307:+;HP][49317:49611:+;Protein spackle][49607:49817:+;HP][49832:49997:+;HP][50035:51058:+;DNA primase][51076:51268:−;HP][54105:54627:+;HP][54626:54764:+;HP][54794:55031:+;Small outer capsid protein][55108:55300:+;HP][55762:55951:+;HP][56022:56688:+;HP][56765:57524:+;HP][57523:57838:+;HP][57834:59166:+;ATP-dependent DNA helicase dda][59172:59418:+;HP][59418:60105:+;Exodeoxyribonuclease][60104:60272:+;HP][60335:60743:+;HP][60807:61023:+;HP][61022:61388:+;HP][61381:61717:+;HP][61964:62213:+;HP][62291:62660:+;HP][62690:64529:+;HP][64576:64729:+;HP][64869:65076:+;HP][65086:67255:+; Protein rIIA][67255:68167:+;HP][68209:68506:+;HP][68447:69026:+;Endonuclease IV][69090:69330:+;HP][69390:69507:+;HP][69517:69709:+;HP][69705:69795:+;HP][69853:70309:+;Nucleoid disruption protein][70478:70619:+;HP][70624:72013:+;HP][72240:72504:+;HP][72627:73272:+;Middle transcription regulatory protein motA][73271:73613:+;HP][74034:74337:+;HP][74520:74646:+;HP][74748:74907:+;HP][74903 :75230:+;HP][75226:75493:+;HP][75560:75833:+;10 kDa anti-sigma factor][75833:76490:−;Holin][76518:77058:−;HP][77095:81700:−;HP][81709:82360:−;Long-tail fiber protein gp36][82417:83554:−;Long-tail fiber protein gp35][83553:87399:−;Long-tail fiber proximal subunit][87474:88401:+;Ribonuclease H][88411:88684:+;Double-stranded DNA-binding protein][88664:88964:+;RNA polymerase-associated protein Gp33][88960:89623:+;Protein Gp59][89690:90608:+;Single-stranded DNA-binding protein][90734:90992:+;HP][91033:91255:+;HP][91345:91624:+;HP][91680:91959:+;HP][91955:92045:+;HP][92037:92358:+;HP][92440:92605:+;HP][92612:93212:+;Dihydrofolate reductase][93195:93513:+;HP][93505:94381:+;Thymidylate synthase][94377:94584:+;HP][94580:94886:+;HP][94876:97123:+;Ribonucleoside-diphosphate reductase large subunit][97179:98322:+;HP][98349:98766:+;Endonuclease II][98762:99377:+;HP][99370:100522:+;T4 RNA ligase 1][100584:101094:+;HP][101081:101435:+;HP][101431:101719:+;HP][101729:101942:+;HP][101941:102835:+;Polynucleotide kinase][102846:103131:+;HP][103120:103357:+;HP][103356:103701:+;HP][103701:104271:+; Deoxycytidylate deaminase][104270:104471:+;HP][104467:104776:+;HP][104833:105157:+;Capsid assembly protein Gp31][105271:105520:+;Lysis inhibition accessory protein][105889:106066:+;HP][106188:106548:+;HP][106587:107076:+;HP][107088:107367:+; HP][107377:107548:+;HP][107544:107745:+;HP][107928:108771:+;HP][108812:109019:+;HP][109018:110491:+;HP][110487:110667:+;HP][110718:112845:+;HP][112908:113187:+;HP][113208:114135:−;Baseplate tail-tube junction protein gp54][114134:115184:−;Baseplate tail-tube junction protein gp48][115193:116927:−;HP][116923:117451:−;Baseplate hub assembly protein gp28][117422:118565:−;Baseplate central spike complex protein gp27][118561:119320:−;HP][119371:120001:+;HP][119997:120408:+;Baseplate wedge protein gp25][120449:120866:+;Recombination protein uvsY][120896:121064:+;HP][121123:121366:−;ATP-dependent DNA helicase uvsW][121362:122895:−;ATP-dependent DNA helicase uvsW][122922:123717:+;HP][123726:125133:+;HP][125212:125440:+;HP][125436:125763:+; HP][125873:126131:+;HP][126141:127149:+;RNA ligase 2][127148:127697:+;HP][127726:129007:−;Capsid vertex protein][129088:130648:−;Major capsidprotein][130668:131481:−;Capsid assembly scaffolding protein][131513:132164:−;Prohead core protein protease][132163:132589:−;Prohead assembly protein gp68][132588:132804:−;HP][132803:134372:−;Portal protein][134476:134968:−;Tail tube protein gp19][135095:137069:−;Tail sheath protein][137102:138941:−;Terminase %2C large subunit][138918:139428:−;Terminase %2C small subunit][139424:140249:−;Tail completion protein gp15][140290:141067:−;Neck protein gp14][141070:142012:−;Neck protein gp13][142069:143836:−;HP][143844:145191:−;HP][145190:145862:−;Baseplate wedge protein gp11][145863:147687:−;Baseplate wedge protein gp10][147686:148598:−;Baseplate protein gp9][148665:149691:−;Baseplate wedge protein gp8][149683:152782:−;Baseplate wedge protein gp7][152778:154737:−;Baseplate wedge protein gp6][154736:155030:−;Baseplate puncturing device gp5.4][155030:155534:−;HP][155536:157267:−;Baseplate central spike complex protein gp5][157263:157902:−;Baseplate wedge protein gp53][157951:158401:+;HP][158400:159228:+;HP][159227:159830:+;Tail tube terminator protein][159897:160572:+;Deoxynucleotide monophosphate kinase][160568:160805:+;HP][160804:161260:+;HP][161335:161515:+;HP][161501:161681:+; HP][161881:162232:+;HP][164136:164289:+;HP][164285:164654:+;HP][164637:164871:+;HP][164925:165150:+;HP][165328:165886:+;HP][165885:166335:+;HP]

Essential genes of phage KP10-89: [36:681:+;HP][730:1195:+;HP][1227:1746:−;HP][1759:2035:−;Highly immunogenic outer capsid protein][2045:2579:−;Highly immunogenic outer capsid protein][2607:3177:−;HP][3216:3528:−;HP][3620:4199:−;HP][4428:5769:+;ATP-dependent DNA helicase uvsW][5777:6011:+;ATP-dependent DNA helicase uvsW][6615:6792:−;HP][6791:7211:−;Recombination protein uvsY][7515:8805:−;Capsid vertex protein][8892:10461:−;Major capsid protein][10480:11269:−;Capsid assembly scaffolding protein][11301:11949:−;Prohead core protein protease][11951:12356:−;Prohead assembly protein gp68][12607:14182:−;Portal protein][14237:14723:−;Tail tube protein gp19][14771:16763:−;Tail sheath protein][16782:18612:−;Terminase %2C large subunit][18580:19120:−;Terminase %2C small subunit][19119:19944:−;Tail completion protein gp15][20023:20770:−;Neck protein gp14][21387:22314:−;Neck protein gp13][22352:24104:−;Fibritin][24114:25500:−;Short tail fiber protein gp12][25509:26178:−;Baseplate wedge protein gp11][26177:27995:−;Baseplate wedge protein gp10][27991:28855:−;Baseplate protein gp9][28864:29857:−;Baseplate wedge protein gp8][29857:32944:−;Baseplate wedge protein gp7][33024:34950:−;Baseplate wedge protein gp6][34981:37048:−;HP][37052:38822:−;Baseplate central spike complex protein gp5][38818:39178:−;Baseplate wedge protein gp53][39423:39891:+;HP][39906:40761:+;HP][40797:41586:−;HP][41955:42486:+;Tail tube terminator protein][42485:43181:+;Deoxynucleotide monophosphate kinase][43507:44131:+;HP][44138:44678:+;HP][44743:44884:+;HP][44973:45114:+;HP][45124:45460:+;HP][45456:45750:+;HP][45742:45994:+;HP][46236:46644:+;HP][46654:47038:+;H P][47039:47297:+;HP][47325:47649:+;HP][47648:48176:+;HP][49163:49364:+;HP][50519:50777:+;HP][51257:51536:+;HP][51607:51859:+;HP][52425:52683:+;HP][52679:52886:+;HP][52896:54042:+;HP][54038:54386:+;HP][54366:54714:+;HP][55163:55595:+;HP][55596:55857:+; HP][55926:56190:+;HP][56229:56553:+;HP][56607:57009:+;HP][57076:57472:+;L-alanyl-D-glutamate peptidase][57503:57962:+;HP][57961:58486:+;HP][58495:59842:+;HP][59878:60076:+;HP][60087:60642:+;HP][60706:61348:+;HP][61415:61811:+;HP][61821:61968:+;HP][61964:62555:+; HP][62564:62888:+;HP][63194:63668:+;HP][63670:63958:+;HP][64140:64749:+;HP][64765:65158:+;HP][65264:65546:+;HP][65517:65940:+;HP][66011:66926:+;HP][67102:68164:+;HP][68338:68533:+;HP][68529:68802:+;HP][69406:69634:+;HP][69630:70008:+;HP][70007:71015: +;HP][71011:71257:+;HP][71256:71514:+;HP][71510:72434:+;HP][72480:72759:+;HP][72965: 73253:+;HP][73478:73994:+;HP][73993:74314:+;HP][74321:76538:+;HP][76609:77227:+;HP][77378:77849:+;HP][77864:78149:+;HP][78506:78731:+;HP][78738:79194:+;HP][79228:79621: −;Baseplate wedge protein gp25][79621:80176:−;HP][80172:80916:−;HP][80944:81808:−;Baseplate tail-tube junction protein gp54][81817:82891:−;Baseplate tail-tube junction protein gp48][82901:84695:−;HP][84691:85267:−;HP][85336:86446:+;HP][86442:86820:−;HP][87128:87419:−;HP][88775:90305:−;HP][90285:90816:−;HP][90896:91118:−;HP][91119:91383:−;HP][91379:91820:−;HP][92520:92814:−;HP][92939:93209:−;HP][93208:93571:−;HP][93581:94148:−;HP][94189:94612:−;HP][94621:94903:−;HP][95217:95469:−;HP][95479:95821:−;Capsid assembly protein Gp31][95836:96394:−;Deoxycytidylate deaminase][96609:96861:−;HP][96857:97145:−;HP][97198:98083:−;Polynucleotide kinase][98079:98253:−;HP][98548:98860:−;HP][98868:100026:−;T4 RNA ligase 1][100006:100372:−;Endonuclease II][100667:101843:−;Ribonucleoside-diphosphate reductase subunit beta][101879:104132:−;Ribonucleoside-diphosphate reductase large subunit][104171:105047:−;Thymidylate synthase][105048:105627:−;HP][105623:105881:−;HP][105877:106108:−;HP][106104:106395:−;HP][106454:108164:−;HP][108237:108429:−;HP][108428:110126:−;Exonuclease subunit 2][110383:111430:−;Exonuclease subunit 1][111785:112001:−;HP][111997:112525:−;RNA polymerase sigma factor][112829:113765:+;Ribonuclease H][113774:114038:+;Double-stranded DNA-binding protein][114041:114287:+;RNA polymerase-associated protein Gp33][114364:114943:+;Protein Gp59][114958:115930:+;HP][115985:116255:+;HP][116281:116947:+;DNA polymerase clamp][117005:118004:+;Sliding-clamp-loader gp44 subunit][118006:118570:+;Sliding-clamp-loader gp44 subunit][118573:118936:+;Translation repressor protein][119004:119292:+;HP][119664:122364:+;DNA-directed DNA polymerase][122438:123596:+;Recombination and repair protein][123634:123955:+;HP][123964:125401:+;ATP-dependent helicase 41][125793:126048:+;HP][126140:126377:+;HP][126746:127478:+;HP][127483:127972:+;HP][127968:128187:+;HP][128242:128503:+;HP][128506:129532:+;DNA primase][129539:130076:+;HP][130257:130548:+;HP][130544:131873:+;ATP-dependent DNA helicase dda][131897:132206:+;HP][132206:132491:+;HP][132487:133159:+;Exodeoxyribonuclease][133155:133437:+;HP][133874:134177:+;HP][134886:135216:+;HP][135255:135852:+;HP][135848:136127:+;HP][136113:136698:+;HP][136720:137161:+;HP][137160:137325:+;HP][137392: 139291:+;HP][139290:140577:+;HP][140648:141203:+;HP][141206:141443:+;HP][141453:143715:+;HP][143714:144614:+;HP][144659:145034:+;HP][145008:145446:+;HP][145482:145731:+;HP][145775:145910:+;HP][145884:146217:+;HP][146213:146441:+;HP][146521:147319:+; HP][147318:147609:+;HP][147875:148523:−;Holin][149044:152755:−;HP][152763:153432:−;Long-tail fiber protein gp36][153481:154606:−;Long-tail fiber protein gp35][154605:158379:−;Long-tail fiber proximal subunit][158458:158758:−;HP][158754:159036:−;Glutaredoxin][158995:159664:−;HP][159706:160666:−;HP][160735:161227:−;HP][161248:161443:−;HP][161775:161982:−;HP][161981:162479:−;HP][162785:164909:−;Anaerobic ribonucleoside-triphosphate reductase][165228:165711:−;Recombination endonuclease VII][165742:166018:−;Glutaredoxin][166026:166878:−;DNA adenine methylase][167163:168597:−;HP][168624:169074:−;HP][169083:169344:−;HP][169933:170515:−;HP][170504:171524:−;RNA ligase 2][171527:172052:−;HP][172051:172408:−;HP][172409:172736:−;HP][172732:172909:−;HP][172905:173223:−;HP][173255:174131:−;HP].

### Example 7

Profiling the Synergistic Redundancy Effect

Materials and Methods:

20 phage combinations of 3 member each and 20 phage combinations of 4 members each were compiled. Those 40 combinations were used each in 79 TTM experiments against 79 single hosts (resulting in 3160 TTM measurements).

A replicator for frozen samples (VP408) was used to inoculate each of the 79 KP strains into a 96-well plate prefilled with 200 μL BHIS. Following an overnight incubation (15-16 h) at 37° C., without agitation, a 2 μL replicator (VP407) was used to dilute the ON starters 1:100 in a 96-well plate prefilled with 200 μL BHIS+1 mM ions. 10 μL of each tested cocktail at a final concentration of 1E+08 PFU/mL were added to each well. One plate served as NPC for which no phages were added. Plates were incubated for 16 hours in a plate reader (Freedom, EVO 75, Tecan) at 37° C. with agitation, and OD600 was measured every 30 min. The periodic OD readings were used to determine the TTM for each phage cocktail: target host couple.

Results

The 40 combinations were selected from a set of 10 phages. Those 10 phages ability/disability to infect each of the 79 used KP strains was already known. Therefore the level of redundancy of each combination with respect to each target bacteria was known and used to categorize the 3160 TTM experiments to redundancy categories 0 to 4 presented in FIG. 5. The level of redundancy of a combination with respect to a target host is the number of phages in the combination that are known to infect the target host individually. FIG. 5 presents evidence for the synergistic effect of phage redundancy on the time to mutant period of the target bacteria. The average TTM is significantly higher between combination populations with different redundancy level. The 3160 measured TTM results were first categorized according to the number of phages of the combination which were shown in a previous experiment to infect the particular host bacteria. Thus, 1522 of the experiments involved a 3 or 4 phage combination where none of the members previously showed ability to infect the tested host. 708 of the experiments involved a 3 or 4 phage combination where only one phage of the combination previously showed ability to infect the tested host. 504 of the experiments involved 3 or 4 phage combinations where two phages of the combination previously showed ability to infect the tested host etc. For each category, the Y-axis indicates the TTM in hours when growth of resistant host was observed. The boxes indicate 25, 50 and 75 percentiles. A Mann-Whitney U test (MWW) was used to compare the significance of difference by each to neighboring categories. The Figure demonstrates a synergistic effect resulting from combinations comprising more than one phage capable of infecting the same target host bacteria. This synergy between combination phage members is biologically explained by the difference in the infection mechanism of the cocktail members.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

1. A composition comprising at least two different strains of isolated bacteriophages, each capable of infecting a bacteria of the species Klebsiella pneumoniae, wherein at least one of said at least two different strains of isolated bacteriophages has a genomic nucleic acid sequence at least 90% identical to one of the nucleic acid sequence as set forth in SEQ ID NOs: 1-30 and/or comprises the essential genes as set forth in Example 6.

2. The composition of claim 1, wherein a first of said at least two different strains of isolated bacteriophages has a genomic nucleic acid sequence at least 90% identical to the nucleic acid sequence as set forth in SEQ ID NO: 1 and/or comprises the essential genes of phage KP10-11 as set forth in Example 6, and a second of said at least two different strains of isolated bacteriophages has a genomic nucleic acid sequence at least 90% identical to the nucleic acid sequence as set forth in SEQ ID NO: 2 and/or comprises the essential genes of phage KP10-48 as set forth in Example 6.

3. The composition of claim 2, comprising at least three different strains of isolated bacteriophages, wherein a third of said at least three different strains of isolated bacteriophages has a genomic nucleic acid sequence at least 90% identical to the nucleic acid sequence as set forth in SEQ ID NO: 3 and/or comprises the essential genes of phage 395D2-3-2 as set forth in Example 6.

4. The composition of claim 1, comprising:
(i) a bacteriophage having a genomic nucleic acid sequence at least 90% identical to SEQ ID NO: 1 and/or comprising the essential genes of phage KP10-11 as set forth in Example 6;
(ii) a bacteriophage having a genomic nucleic acid sequence at least 90% identical to SEQ ID NO: 2 and/or comprising the essential genes of phage KP10-48 as set forth in Example 6;
(iii) a bacteriophage having a genomic nucleic acid sequence at least 90% identical to SEQ ID NO: 3 and/or comprising the essential genes of phage 395D2-3-2 as set forth in Example 6;
(iv) a bacteriophage having a genomic nucleic acid sequence at least 90% identical to SEQ ID NO: 4 and/or comprising the essential genes of phage 395D2-3-1 as set forth in Example 6;
(v) a bacteriophage having a genomic nucleic acid sequence at least 90% identical to SEQ ID NO: 22 and/or comprising the essential genes of phage KP10-85 as set forth in Example 6; and
(vi) a bacteriophage having a genomic nucleic acid sequence at least 90% identical to SEQ ID NO: 25 and/or comprising the essential genes of phage KP10-91 as set forth in Example 6.

5. The composition of claim 1, wherein said at least two different strains of isolated bacteriophages in combination target at least 100 different strains of Klebsiella pneumoniae.

6. The composition of claim 1, wherein at least 100 different strains of Klebsiella pneumonia are targeted by each of said at least two different strains.

7-8. (canceled)

9. The composition of claim 1, wherein said at least one bacteriophage is genetically modified such that the genome thereof comprises a heterologous sequence.

10. The composition of claim 9, wherein said heterologous sequence encodes a therapeutic agent or a diagnostic agent.

11. The composition of claim 1, comprising no more than 10 different bacteriophage strains.

12. A recombinant bacteriophage capable of infecting bacteria of the species Klebsiella pneumoniae, wherein said bacteriophage (i) has a genomic nucleic acid sequence at least 90% identical to one of the nucleic acid sequences as set forth in SEQ ID NOs: 1-30 and/or (ii) comprises the essential genes of a bacteriophage as set forth in Example 6, and wherein said bacteriophage is genetically modified such that the genome thereof comprises a heterologous sequence.

13. The recombinant bacteriophage of claim 12, wherein said heterologous sequence encodes a therapeutic agent or a diagnostic agent.

14-16. (canceled)

17. The composition of claim 1, being formulated for oral delivery or rectal delivery.

18-19. (canceled)

20. A method of treating a disease associated with a Klebsiella pneumoniae infection in a subject in need thereof, comprising administering to the subject a therapeutically effective amount of a composition comprising at least one isolated bacteriophage strain capable of infecting bacteria of the species Klebsiella pneumonia causing the infection, wherein said at least one bacteriophage strain (i) has a genomic nucleic acid sequence at least 95% identical to one of the nucleic acid sequences set forth in SEQ ID NOs: 1-30 and/or (ii) comprises the essential genes of a bacteriophage as set forth in Example 6, thereby treating the disease associated with a Klebsiella pneumoniae infection.

21. A method of treating a disease associated with a Klebsiella pneumoniae infection in a subject in need thereof, comprising administering to the subject a therapeutically effective amount of the composition of claim 1, thereby treating the disease associated with a Klebsiella pneumoniae infection.

22. The method of claim 20, wherein the disease is an inflammatory bowel disease (IBD).

23. The method of claim 22, wherein said inflammatory bowel disease is ulcerative colitis (UC) or Crohn's disease.

24. (canceled)

25. The method of claim 20, wherein said administering comprises orally administering or rectally administering.

26. The method of claim 20, wherein said composition comprises no more than 10 different bacteriophage strains.

27. The method of claim 20, further comprising identifying the strain of Klebsiella pneumoniae colonizing the subject prior to the administering.

28. The method of claim 20, wherein said at least one bacteriophage strain is genetically modified such that the genome thereof comprises a heterologous sequence which encodes a therapeutic or diagnostic agent.

29-30. (canceled)
